(19) United States
(12) Patent Application Publication
(10) Pub. No.: US 2023/0406841 A1
(43) Pub. Date: Dec. 21, 2023

(54) NOVEL COMPOUND AND ORGANIC LIGHT EMITTING DEVICE COMPRISING THE SAME

(71) Applicant: LG CHEM, LTD., Seoul (KR)

(72) Inventors: Min Woo JUNG, Daejeon (KR); Dong Hoon LEE, Daejeon (KR); Sang Duk SUH, Daejeon (KR); Jungha LEE, Daejeon (KR); Su Jin HAN, Daejeon (KR); Seulchan PARK, Daejeon (KR); Sunghyun HWANG, Daejeon (KR)

(21) Appl. No.: 18/038,274

(22) PCT Filed: Feb. 22, 2022
(86) PCT No.: PCT/KR2022/002578

§ 371 Date: May 23, 2023

(30) Foreign Application Priority Data
Feb. 22, 2021 (KR) 10-2021-0023634
Feb. 21, 2022 (KR) 10-2022-0022275

(51) Int. Cl. C07D 403/10; C07D 403/14; C07D 407/14; H10K 50/11; C07D 409/14; H10K 85/60

(52) U.S. Cl. CPC C07D 403/10; C07D 403/14; C07D 407/14; H10K 50/11; C07D 409/14; H10K 85/6572; C07B 2200/05

(57) ABSTRACT

Provided is a novel compound Chemical Formula 1:

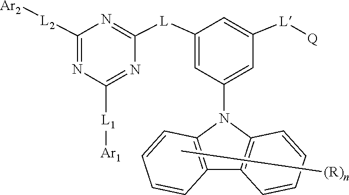

wherein: L is a single bond, a substituted or unsubstituted C₆₋₆₀ arylene, or a substituted or unsubstituted C₂₋₆₀ heteroarylene containing one or more of N, O and S; L₁ and L₂ are each independently a single bond or a substituted or unsubstituted C₆₋₆₀ arylene, Ar₁ and Ar₂ are each independently a substituted or unsubstituted C₆₋₆₀ aryl or C₂₋₆₀ heteroaryl containing one or more of N, O and S, L′ is a single bond or a substituted or unsubstituted C₆₋₆₀ arylene, Q is a substituted or unsubstituted C₆₋₆₀ aryl, R is deuterium or deuterium-substituted phenyl, and n is 1 to 8, and an organic light emitting device including the same. Organic light emitting devices that include the compound of Chemical Formula 1 as a host material for the light emitting layer exhibit remarkably improved lifetime characteristics without reducing luminous efficiency.

### CROSS-REFERENCE TO RELATED APPLICATION(S)

This application is a National Stage Application of International Application No. PCT/KR2022/002578 filed on Feb. 22, 2022, which claims the benefit of Korean Patent Application No. 10-2021-0023634 filed on Feb. 22, 2021 and Korean Patent Application No. 10-2022-0022275 filed on Feb. 21, 2022 in the Korean Intellectual Property Office, the entire contents of which are incorporated herein by reference.

### TECHNICAL FIELD

The present disclosure relates to a novel compound and an organic light emitting device comprising the same.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer can be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuous need to develop a new material for the organic material used in the organic light emitting device as described above.

### PRIOR ART LITERATURE

### Patent Literature

(Patent Literature 0001) Korean Unexamined Patent Publication No. 10-2000-0051826

### BRIEF DESCRIPTION

### Technical Problem

It is an object of the present disclosure to provide a novel compound and an organic light emitting device comprising the same.

### Technical Solution

According to an aspect of the present disclosure, provided is a compound of the following Chemical Formula 1:

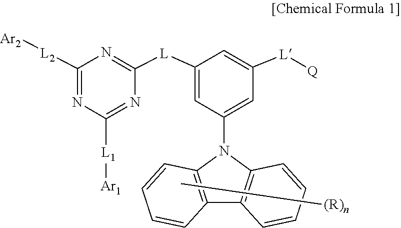

wherein, in Chemical Formula 1:
L is a single bond, a substituted or unsubstituted C₆₋₆₀ arylene, or a substituted or unsubstituted C₂₋₆₀ heteroarylene containing one or more heteroatoms of N, O and S;
L₁ and L₂ are each independently a single bond or a substituted or unsubstituted C₆₋₆₀ arylene;
Ar₁ and Ar₂ are each independently a substituted or unsubstituted C₆₋₆₀ aryl or a substituted or unsubstituted C₂₋₆₀ heteroaryl containing one or more heteroatoms of N, O and S;
L′ is a single bond or a substituted or unsubstituted C₆₋₆₀ arylene;
Q is a substituted or unsubstituted C₆₋₆₀ aryl;
R is deuterium or phenyl substituted with deuterium; and
n is an integer of 1 to 8.

According to another aspect of the present disclosure, provided is an organic light emitting device comprising: a first electrode; a second electrode that is opposite to the first electrode; and one or more organic material layers that are between the first electrode and the second electrode, wherein one or more layers of the organic material layers comprises the compound of Chemical Formula 1.

### Advantageous Effects

The above-mentioned compound of Chemical Formula 1 can be used as a material of an organic material layer in an organic light emitting device, and can improve the efficiency, achieve low driving voltage and/or improve lifetime characteristics in the organic light emitting device.

### BRIEF DESCRIPTION OF THE DRAWINGS

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, an electron blocking layer 7, a light emitting layer 3, an electron transport layer 8, an electron injection layer 9, and a cathode 4.

### DETAILED DESCRIPTION

Hereinafter, embodiments of the present disclosure will be described in more detail to facilitate understanding of the invention.

### Definition of Terms

As used herein, the notation [symbol] and [symbol] mean a bond linked to another substituent group.

As used herein, the term “substituted or unsubstituted” means being unsubstituted or substituted with one or more substituents selected from the group consisting of deuterium, a halogen group, a nitro group, a hydroxy group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphine group, and a heteroaryl containing at least one of N, O and S atoms, or being unsubstituted or substituted with a substituent to which two or more substituents of the above-exemplified substituents are linked. For example, “a substituent in which two or more substituents are linked” can be a biphenyl group. Namely, a biphenylyl group can be an aryl group, or it can also be interpreted as a substituent in which two phenyl groups are linked. In one example, the term “substituted or unsubstituted” can be understood to mean “being unsubstituted or substituted with one or more, for example 1 to 5 substituents selected from the group consisting of deuterium, halogen, nitrile, a C₁₋₁₀ alkyl, a C₁₋₁₀ alkoxy and a C₆₋₂₀ aryl”. Further, the term “substituted with one or more substituents” can be understood to mean, for example, “being substituted with 1 to 5 substituents, or “being substituted with 1 to 2 substituents”.

In the present disclosure, the carbon number of a carbonyl group is not particularly limited, but is preferably 1 to 40. Specifically, it can be a substituent group having the following structure, but is not limited thereto:

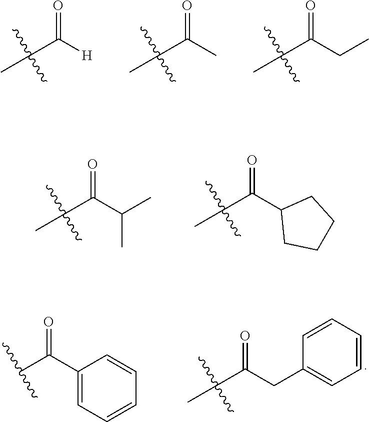

In the present disclosure, an ester group can have a structure in which oxygen of the ester group can be substituted by a straight-chain, branched-chain, or cyclic alkyl group having 1 to 25 carbon atoms, or an aryl group having 6 to 25 carbon atoms. Specifically, it can be a substituent group having the following structure, but is not limited thereto:

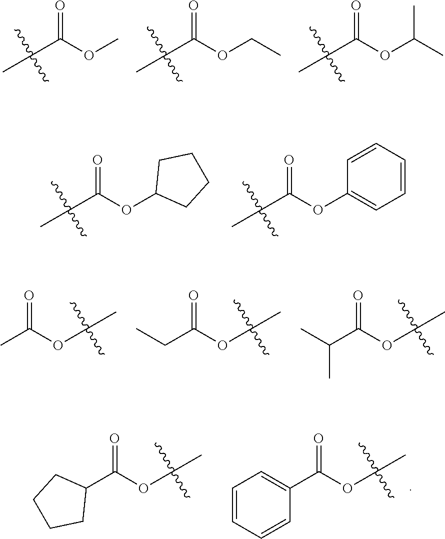

In the present disclosure, the carbon number of an imide group is not particularly limited, but is preferably 1 to 25. Specifically, it can be a substituent group having the following structure, but is not limited thereto:

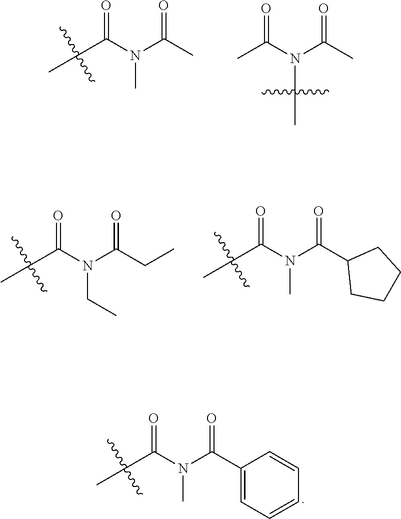

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but is not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluorine, chlorine, bromine, or iodine.

In the present disclosure, the alkyl group can be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, specific examples of the alkyl group include methyl, ethyl, propyl, n-propyl, isopropyl, butyl, n-butyl, isobutyl, tert-butyl, sec-butyl, 1-methyl-butyl, 1-ethylbutyl, pentyl, n-pentyl, isopentyl, neopentyl, tert-pentyl, 1-ethyl-propyl, 1,1-dimethylpropyl, hexyl, n-hexyl, 1-methylpentyl, 2-methylpentyl, 4-methyl-2-pentyl, 3,3-dimethylbutyl, 2-ethylbutyl, heptyl, n-heptyl, isohexyl, 1-methylhexyl, 2-methylhexyl, 3-methylhexyl, 4-methylhexyl, 5-methylhexyl, cyclopentylmethyl, cyclohexylmethyl, octyl, n-octyl, tert-octyl, 1-methylheptyl, 2-ethylhexyl, 2,4,4-trimethyl-1-pentyl, 2,4,4-trimethyl-2-pentyl, 2-propylpentyl, n-nonyl, 2,2-dimethylheptyl, and the like, but are not limited thereto.

In the present disclosure, the alkenyl group can be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to still another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it can be a monocyclic aryl group or a polycyclic aryl group having aromaticity. According to one embodiment, the carbon number of the aryl group is 6 to 30. According to one embodiment, the carbon number of the aryl group is 6 to 20. The aryl group can be a phenyl group, a biphenyl group, a terphenyl group or the like as the monocyclic aryl group, but is not limited thereto. The polycyclic aryl group includes a naphthyl group, an anthracenyl group, a phenanthrenyl group, a triphenylenyl, pyrenyl group, a perylenyl group, a chrysenyl group, and the like, but is not limited thereto.

In the present disclosure, a heteroaryl is a heteroaryl containing one or more heteroatoms of O, N, Si and S as a heteroatom, and the carbon number thereof is not particularly limited, but is preferably 2 to 60. Examples of the heteroaryl group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group, the arylamine group and the arylsilyl group is the same as the aforementioned examples of the aryl group. In the present disclosure, the alkyl group in the aralkyl group, the alkylaryl group and the alkylamine group is the same as the aforementioned examples of the alkyl group. In the present disclosure, the heteroaryl in the heteroarylamine group apply to the aforementioned description of the heteroaryl. In the present disclosure, the alkenyl group in the aralkenyl group is the same as the aforementioned examples of the alkenyl group. In the present disclosure, the aforementioned description of the aryl group can be applied except that the arylene is a divalent group. In the present disclosure, the aforementioned description of the heteroaryl can be applied except that the heteroarylene is a divalent group. In the present disclosure, the aforementioned description of the aryl group or cycloalkyl group can be applied except that the hydrocarbon ring is not a monovalent group but formed by combining two substituent groups. In the present disclosure, the aforementioned description of the heteroaryl can be applied, except that the heterocycle is not a monovalent group but formed by combining two substituent groups.

### Compound

Meanwhile, the present disclosure provides the compound of Chemical Formula 1.

Specifically, the compound of Chemical Formula 1 has a structure in which the position 1 of the benzene ring is substituted with carbazol-9-yl, the position 3 is substituted with triazinyl, and the position 5 is substituted with aryl. Also, the compound can have a structure in which carbon at position 8 of the dibenzofuran/dibenzothiophene is unsubstituted or substituted with a deuterium-substituted aryl.

In particular, the compound has a structure in which the carbazol-9-yl substituent group is substituted with at least one deuterium or deuterium-substituted phenyl, and carbons at positions 2, 4, and 6 of the benzene ring are not substituted with deuterium. Such compounds can have increased intramolecular electronic stability as compared to compounds having no deuterium in the molecule and compounds in which at least one of the carbons at positions 2, 4, and 6 of the benzene ring is substituted with deuterium. In addition, as the compound has a carbazol-9-yl structure substituted with deuterium or deuterium-substituted phenyl, the compound has a stronger bond energy in the molecule than a compound having no deuterium because of the characteristic that the bond energy of the C-D bond is greater than the bond energy of the C—H bond, thereby improving the material stability.

Therefore, an organic light emitting device employing the compound can not only exhibit a low driving voltage but also improve lifetime characteristics.

In one embodiment, L can be a single bond, a C₆₋₂₀ arylene that is unsubstituted or substituted with deuterium, or a C₂₋₂₀ heteroarylene containing one heteroatom of O and S which is unsubstituted or substituted with deuterium.

Specifically, L can be a single bond, phenylene, biphenyldiyl, naphthylene, or dibenzofuranylene or dibenzothiophenylene.

For example, L is a single bond or one selected from the group consisting of:

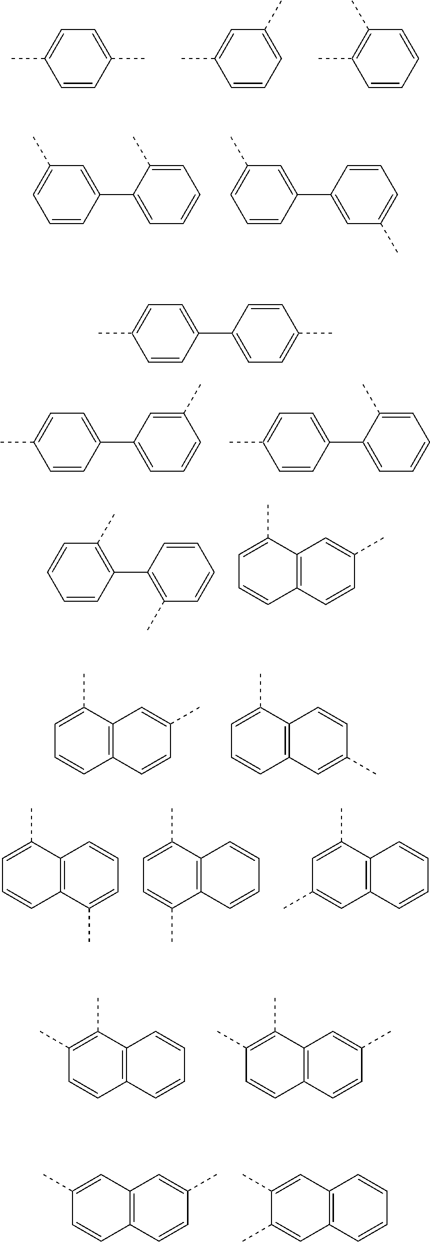

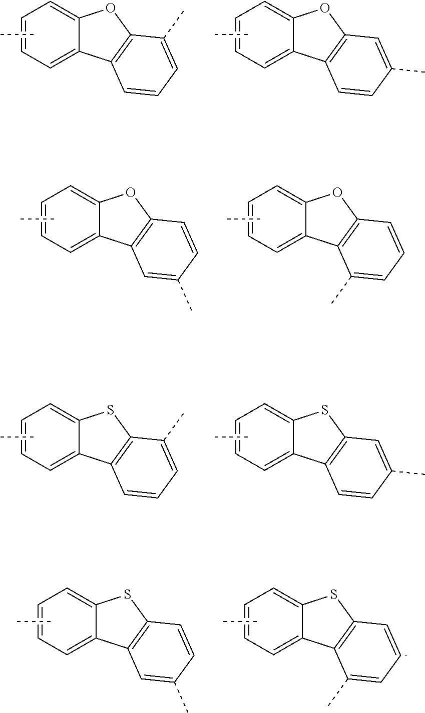

Further, in one embodiment, L₁ and L₂ can be each independently a single bond or a C₆₋₂₀ arylene that is unsubstituted or substituted with deuterium.

Specifically, L₁ and L₂ can be each independently a single bond or phenylene. In other words, L₁ and L₂ can be each independently a single bond, 1,2-phenylene, 1,3-phenylene, or 1,4-phenylene.

For example, both L₁ and L₂ are single bonds; or
one of L₁ and L₂ is a single bond and the rest can be 1,2-phenylene, 1,3-phenylene, or 1,4-phenylene.

Wherein, L₁ and L₂ can be the same as each other. Alternatively, the L₁ and L₂ can be different.

Further, in one embodiment, Ar₁ and Ar₂ are each independently a C₆₋₂₀ aryl which is unsubstituted or substituted with one or more substituent groups selected from the group consisting of deuterium, a C₁₋₁₀ alkyl and a C₆₋₂₀ aryl; or a C₂₋₆₀ heteroaryl containing one or more heteroatoms of N, O and S which is unsubstituted or substituted with one or more substituent groups selected from the group consisting of deuterium, a C₁₋₁₀ alkyl and a C₆₋₂₀ aryl.

Specifically, Ar₁ and Ar₂ are each independently phenyl, biphenylyl, terphenylyl, naphthyl, phenanthryl, triphenylenyl, fluorenyl, dibenzofuranyl, dibenzothiophenyl, or carbazolyl,

wherein the Ar₁ and Ar₂ can be unsubstituted or substituted with one or more substituent groups selected from the group consisting of deuterium, methyl, and phenyl.

Specifically, Ar₁ and Ar₂ are each independently phenyl, biphenylyl, terphenylyl, naphthyl, phenanthryl, triphenylenyl, fluorenyl, dibenzofuranyl, dibenzothiophenyl, or carbazolyl,

wherein the Ar₁ and Ar₂ can be unsubstituted or substituted with 1 to 5 substituent groups selected from the group consisting of deuterium, methyl and phenyl.

More specifically, Ar₁ and Ar₂ can be each independently phenyl, biphenylyl, terphenylyl, naphthyl, phenanthryl, triphenylenyl, 9,9-dimethylfluorenyl, dibenzofuranyl, dibenzothiophenyl, carbazolyl, or 9-phenylcarbazolyl.

For example, Ar₁ and Ar₂ can be each independently any one selected from the group consisting of the following, but is not limited thereto:

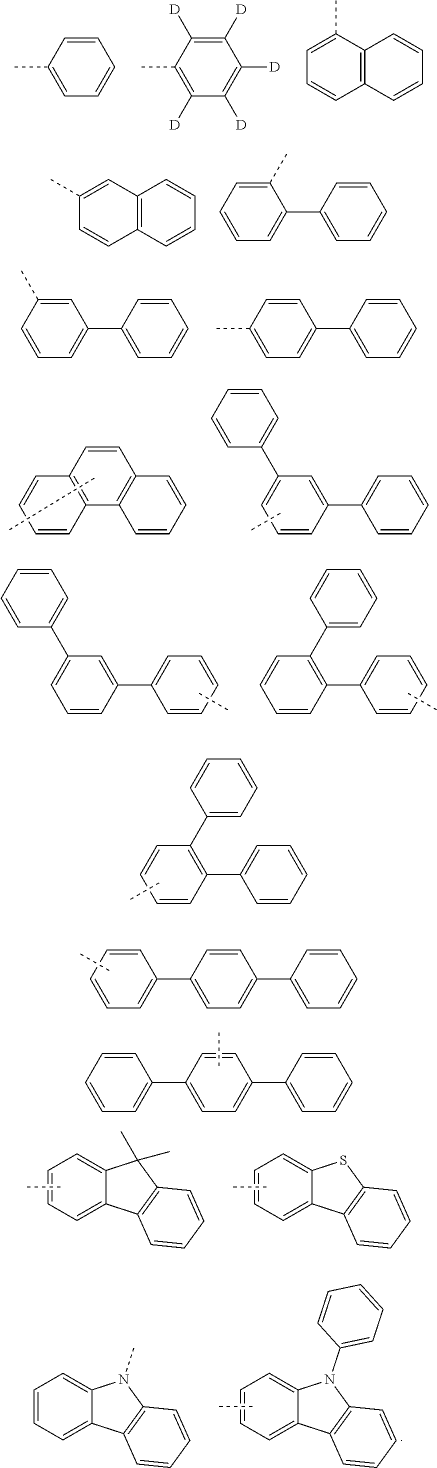

Wherein, Ar₁ and Ar₂ can be equal to each other. Alternatively, Ar₁ and Ar₂ can be different.

Further, in one embodiment, at least one of Ar₁ and Ar₂ can be a C₆₋₁₂ aryl that is unsubstituted or substituted with deuterium.

For example, at least one of Ar₁ and Ar₂ can be

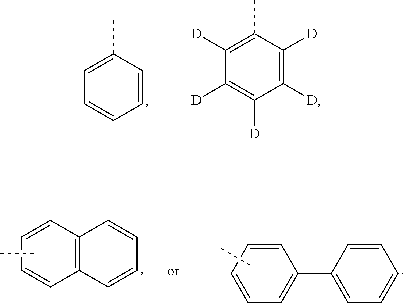

In one embodiment, when L is not a single bond, both Ar₁ and Ar₂ can be phenyl.

More specifically, L is a single bond, and Ar₁ and Ar₂ are each independently phenyl, phenyl substituted with 5 deuteriums, biphenylyl, terphenylyl, naphthyl, phenanthryl, triphenylenyl, 9,9-dimethylfluorenyl, dibenzofuranyl, dibenzothiophenyl, carbazolyl, or 9-phenylcarbazolyl; or

L is a C₆₋₂₀ arylene or a C₂₋₂₀ heteroarylene containing one heteroatom of O and S, and both Ar₁ and Ar₂ can be phenyl.

Further, in one embodiment, L′ can be a single bond, or a C₆₋₂₀ arylene that is unsubstituted or substituted with deuterium.

Specifically, L′ can be a single bond or phenylene. In other words, L′ can be a single bond, 1,2-phenylene, 1,3-phenylene, or 1,4-phenylene.

Further, in one embodiment, Q can be a C₆₋₂₄ aryl which is unsubstituted or substituted with one or more substituent groups selected from the group consisting of deuterium, a C₁₋₁₀ alkyl and a C₆₋₂₀ aryl.

Specifically, Q is phenyl, biphenylyl, naphthyl, phenanthryl, triphenylenyl, benzophenanthryl, or 9,9-dimethylfluorenyl,

wherein Q can be unsubstituted or substituted with deuterium.

Alternatively, wherein Q can be unsubstituted or substituted with at least one deuterium. More specifically, Q can be unsubstituted, or all carbons in the molecule can be substituted with deuterium.

For example, Q can be any one selected from the group consisting of the following, but is not limited thereto:

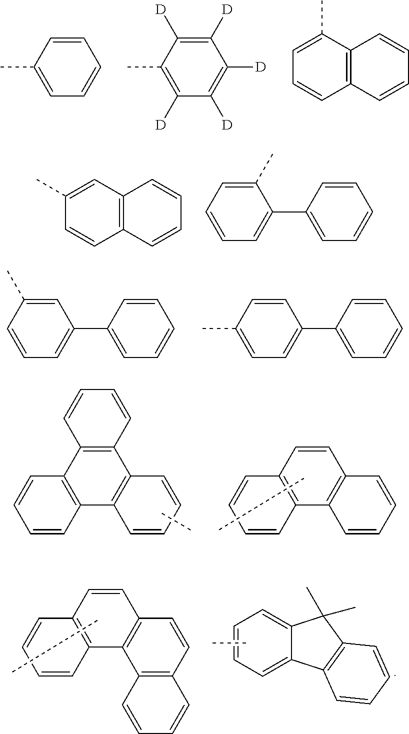

Also, in one embodiment, R can be deuterium or phenyl substituted with 1 to 5 deuteriums.

For example, R can be deuterium, or

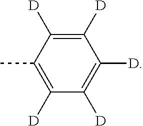

Wherein, n means the number of R, and when n is 2 or more, two or more R can be the same as or different from each other. For example, n can be 1, 2, 3, 4, 5, 6, 7, or 8.

Further, the

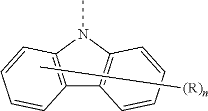

can be any one selected from the group consisting of:

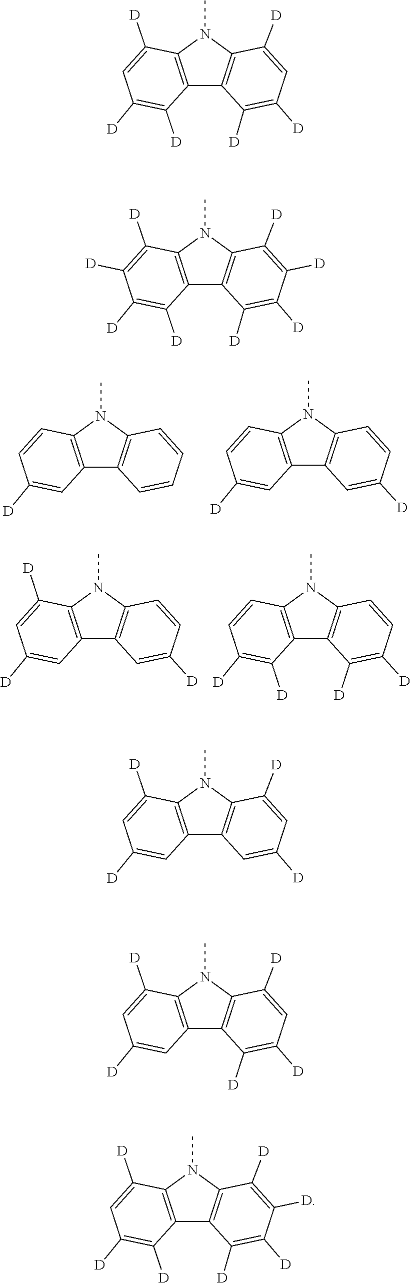

In addition, the compound can be any one of the following Chemical Formulas 1-1 to 1-3:

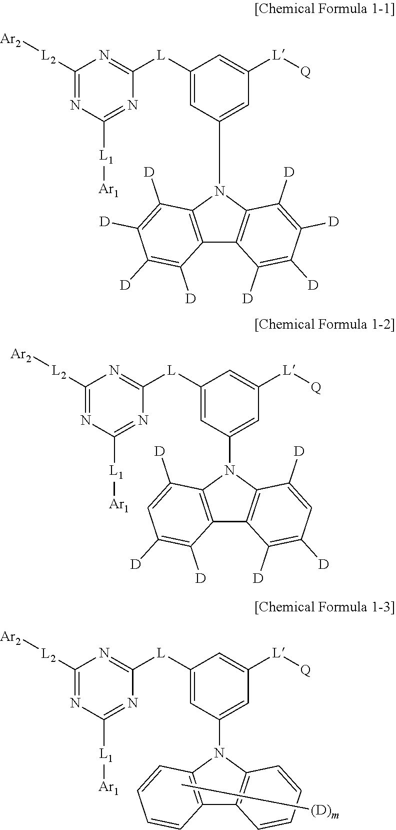

wherein, in Chemical Formulas 1-1 to 1-3:
D is deuterium;
m is 1, 2, 3, 4, 5, or 7; and
L, L₁, L₂, Ar₁, Ar₂, L′ and Q are as defined in Chemical Formulas 1.

Meanwhile, representative examples of the compound of Chemical Formula 1 are as follows:

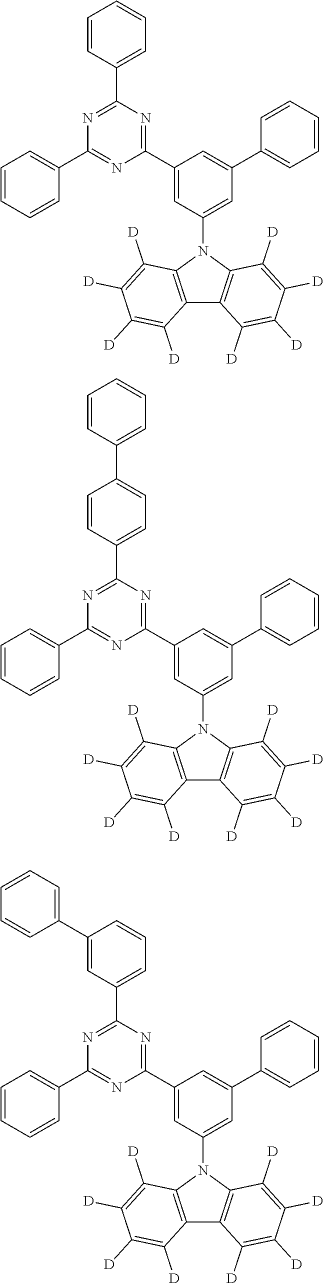

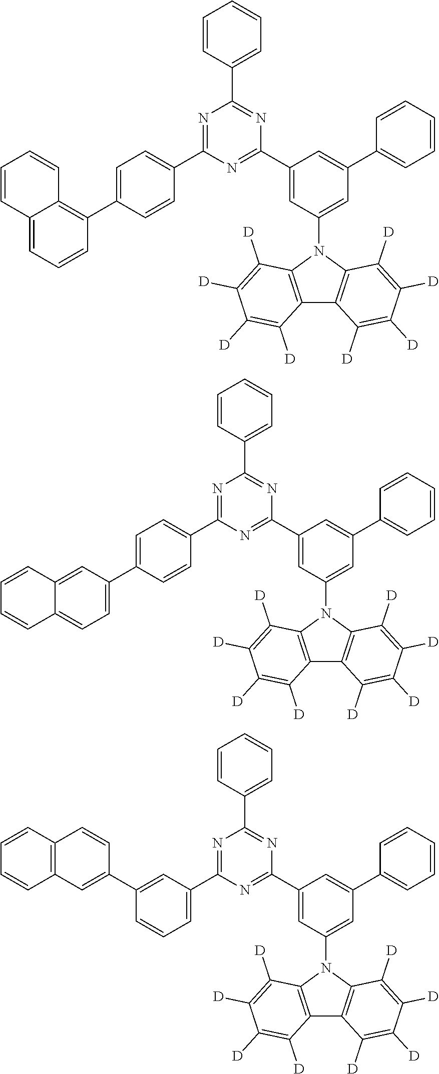

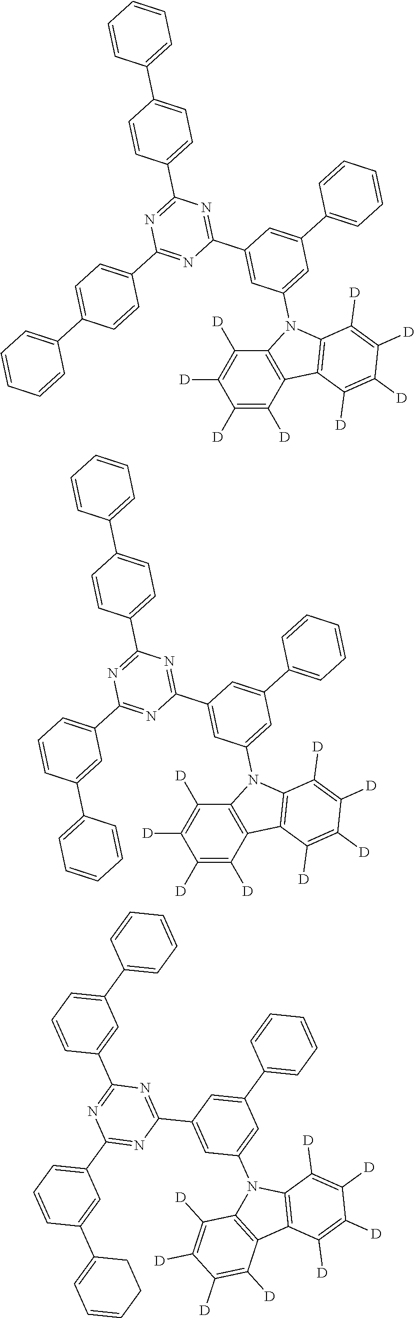

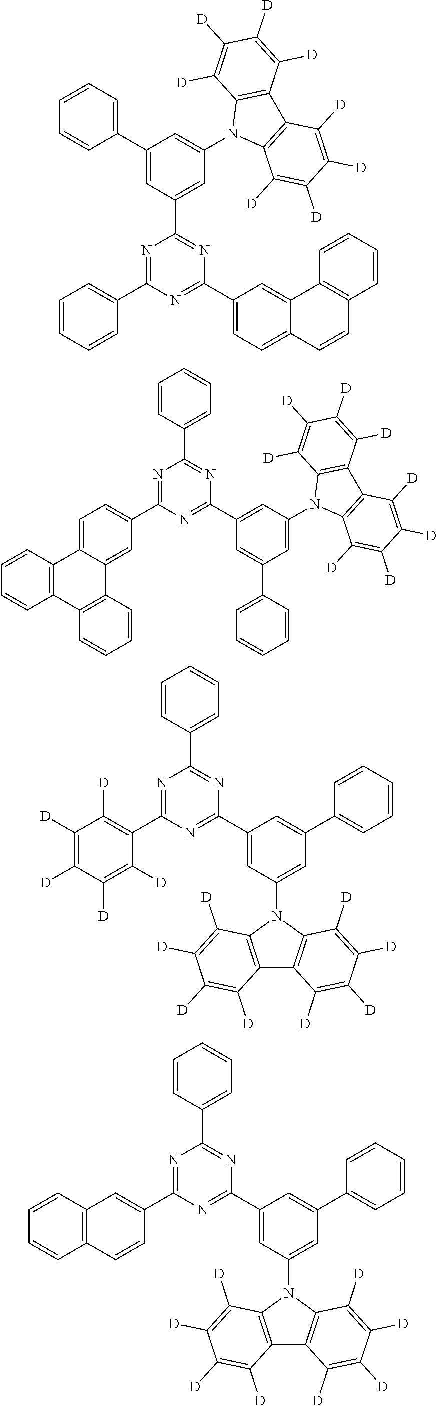

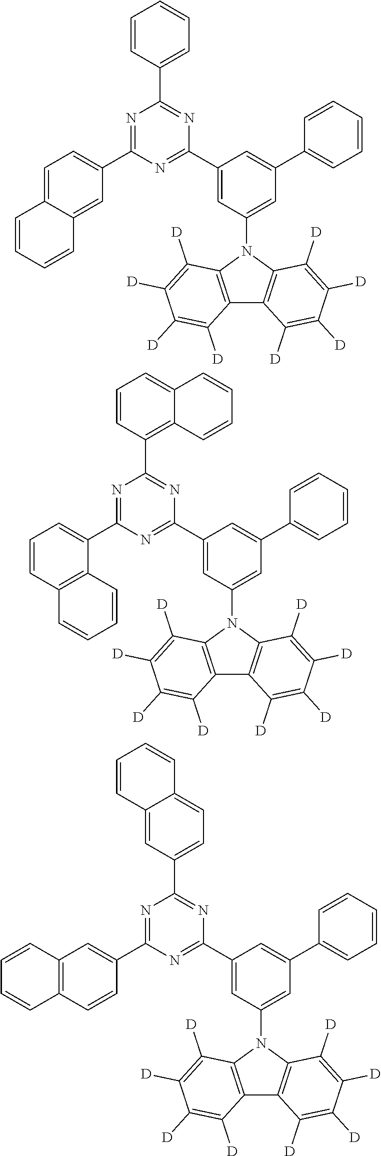

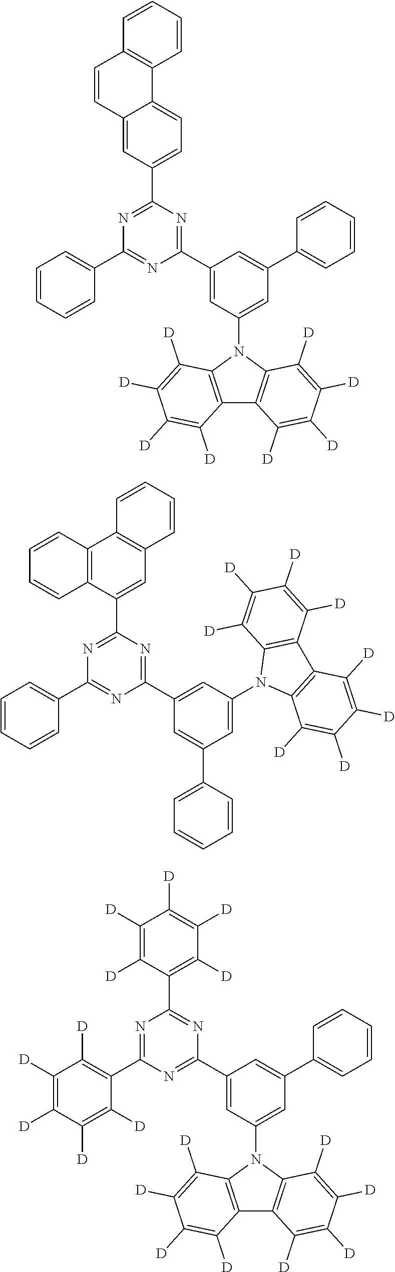

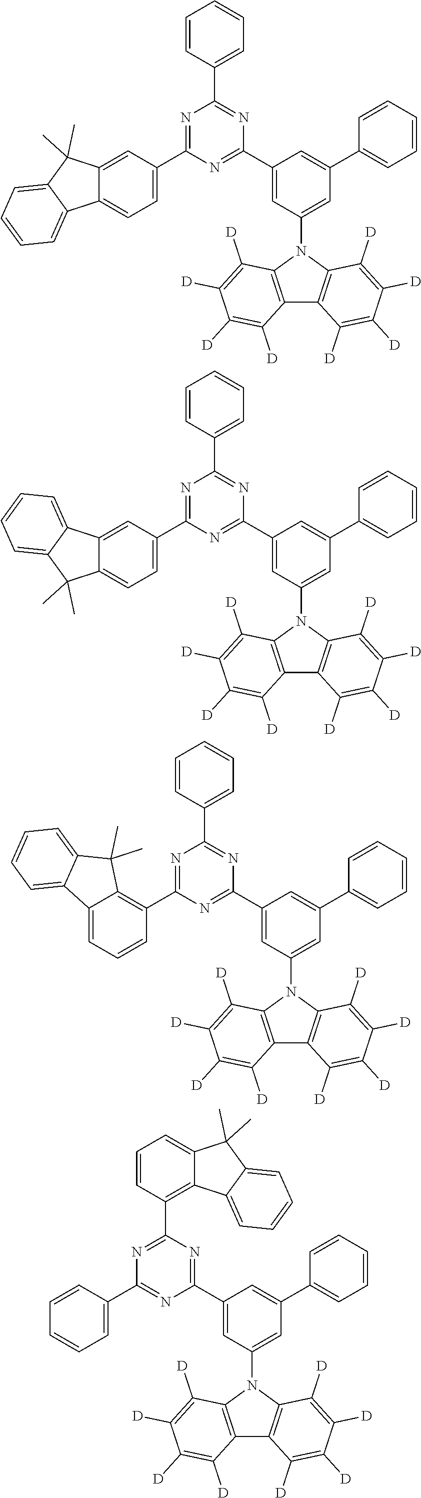

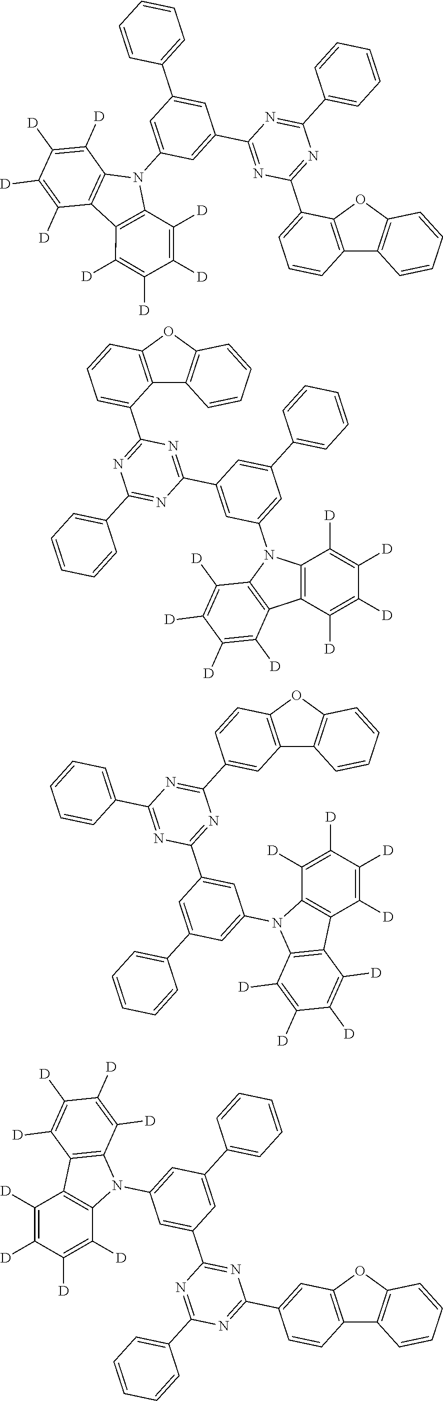

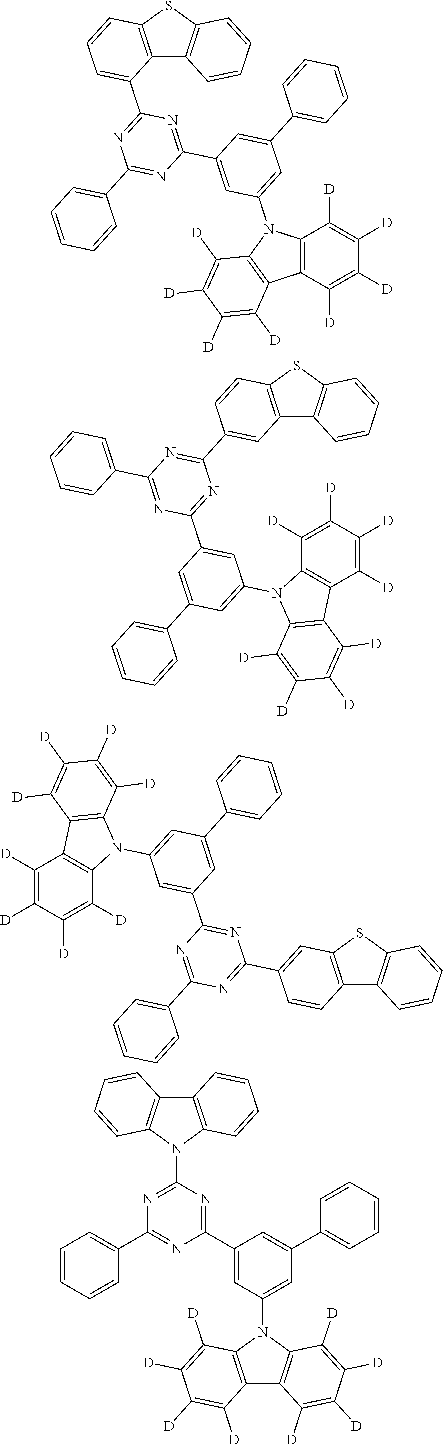

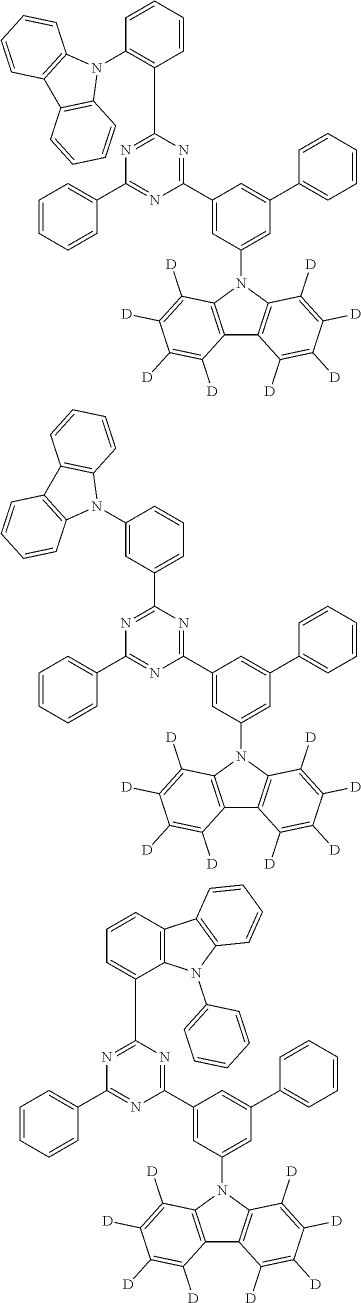

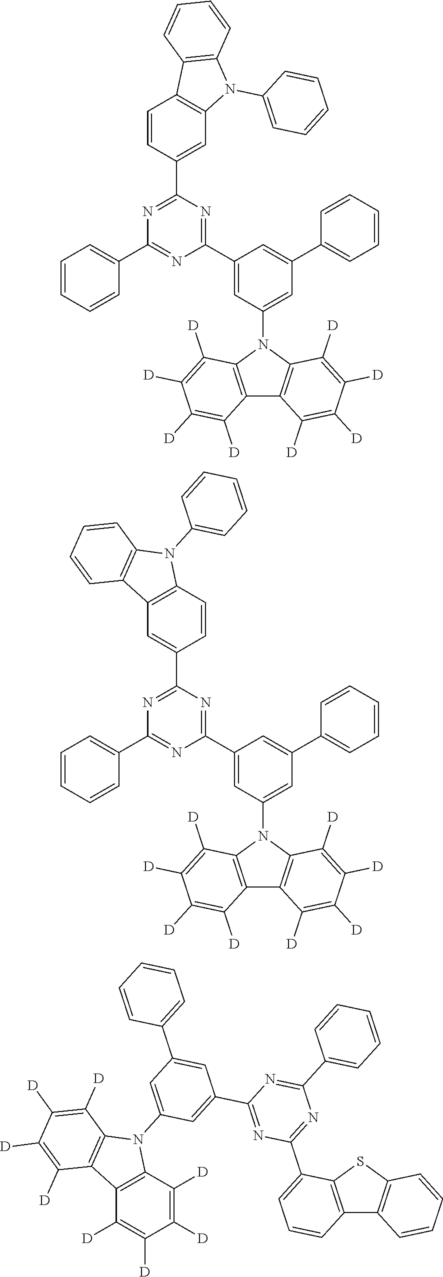

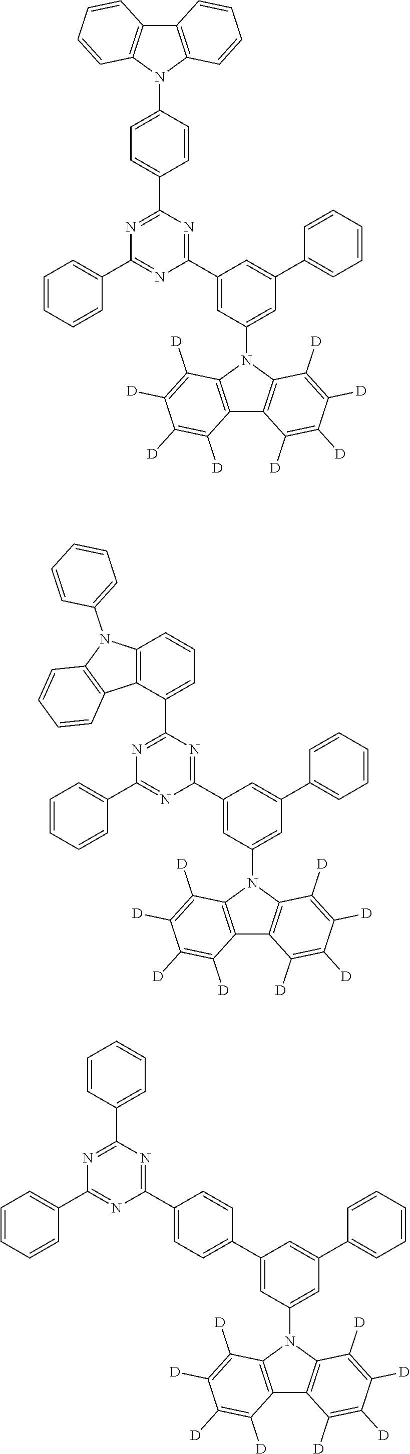 [SMILES: [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3ccc(-n4c5ccccc5c5ccccc54)cc3)n2)c1 ; [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3cccc4c3c3ccccc3n4-c3ccccc3)n2)c1 ; [2H]c1c([2H])c([2H])c2c(c1[2H])c1c([2H])c([2H])c([2H])c([2H])c1n2-c1cc(-c2ccccc2)cc(-c2ccc(-c3nc(-c4ccccc4)nc(-c4ccccc4)n3)cc2)c1]

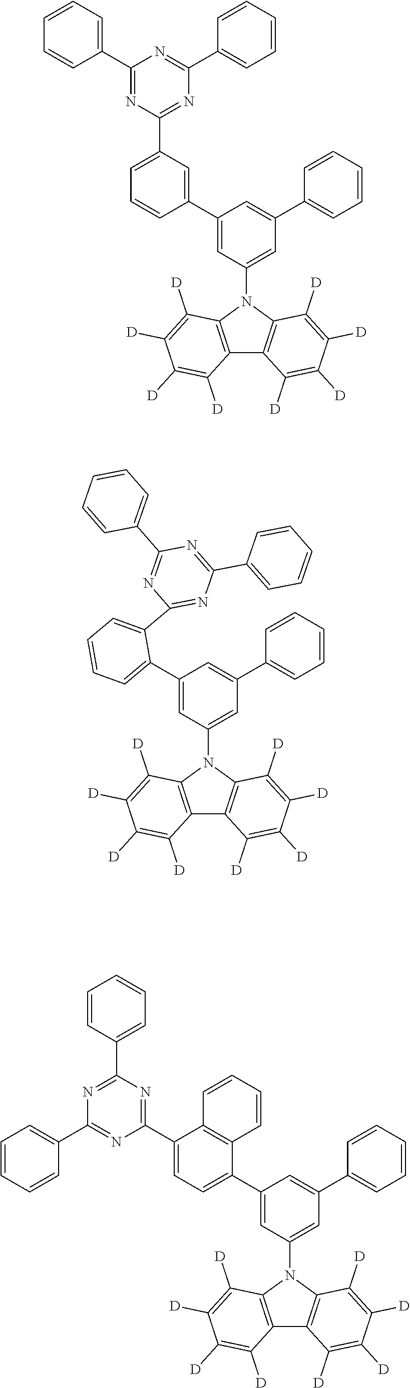 [SMILES: [2H]c1c([2H])c([2H])c2c(c1[2H])c1c([2H])c([2H])c([2H])c([2H])c1n2-c1cc(-c2ccccc2)cc(-c2cccc(-c3nc(-c4ccccc4)nc(-c4ccccc4)n3)c2)c1 ; [2H]c1c([2H])c([2H])c2c(c1[2H])c1c([2H])c([2H])c([2H])c([2H])c1n2-c1cc(-c2ccccc2)cc(-c2ccccc2-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cc(-c2ccccc2)cc(-c2ccc(-c3nc(-c4ccccc4)nc(-c4ccccc4)n3)c3ccccc23)c1]

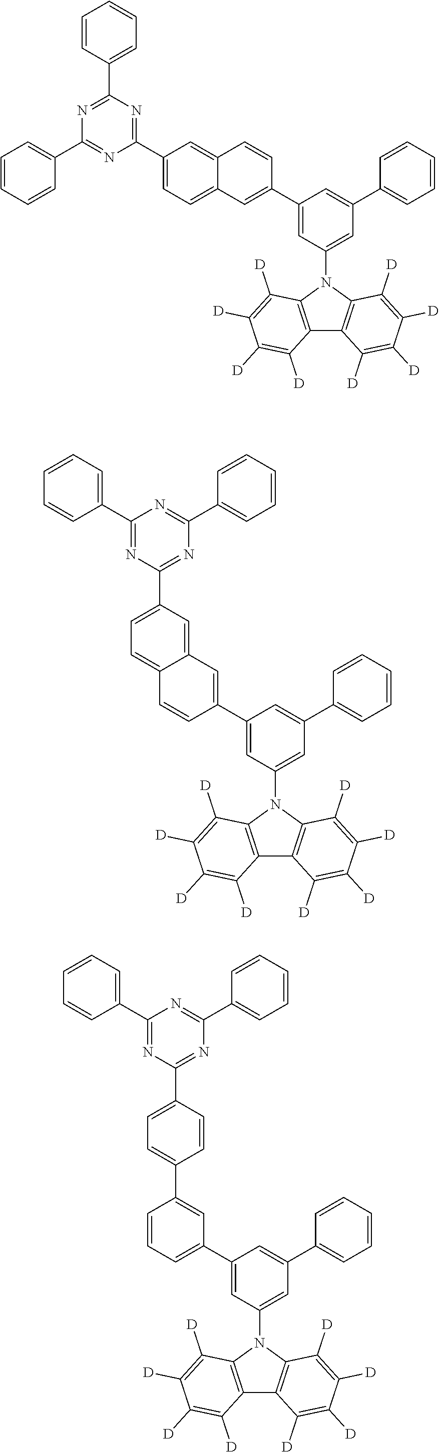

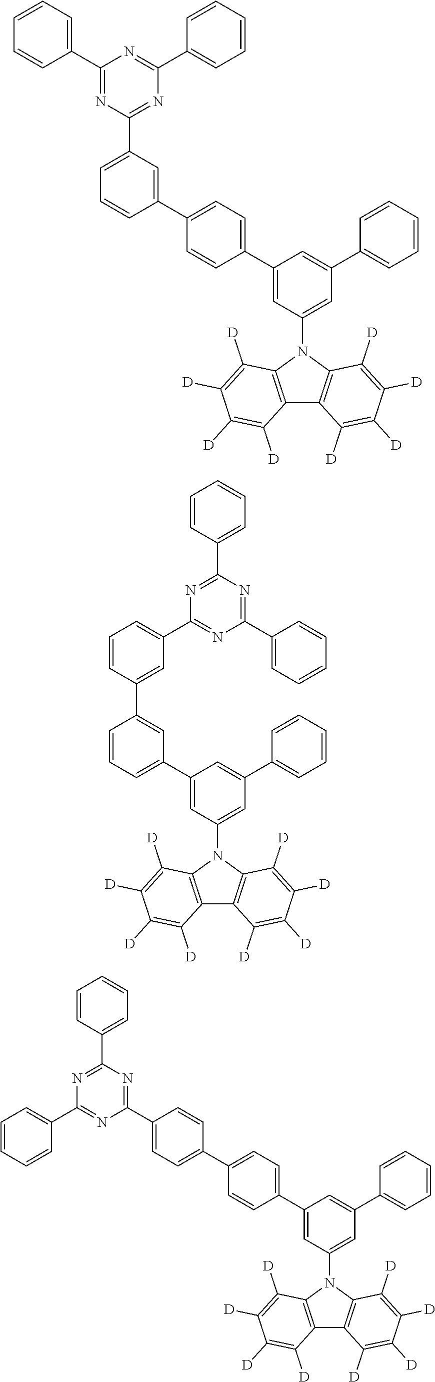

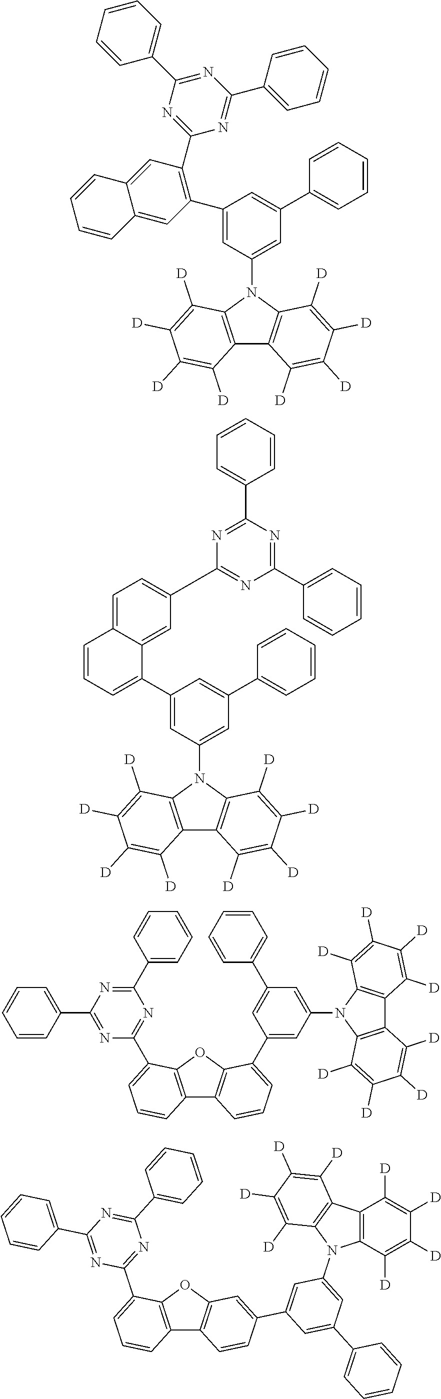

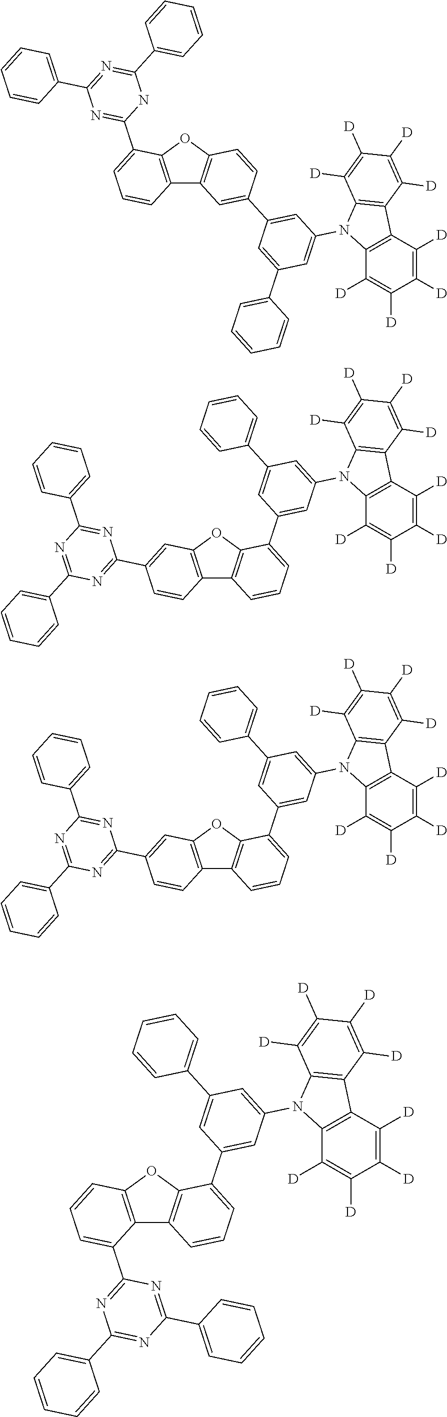

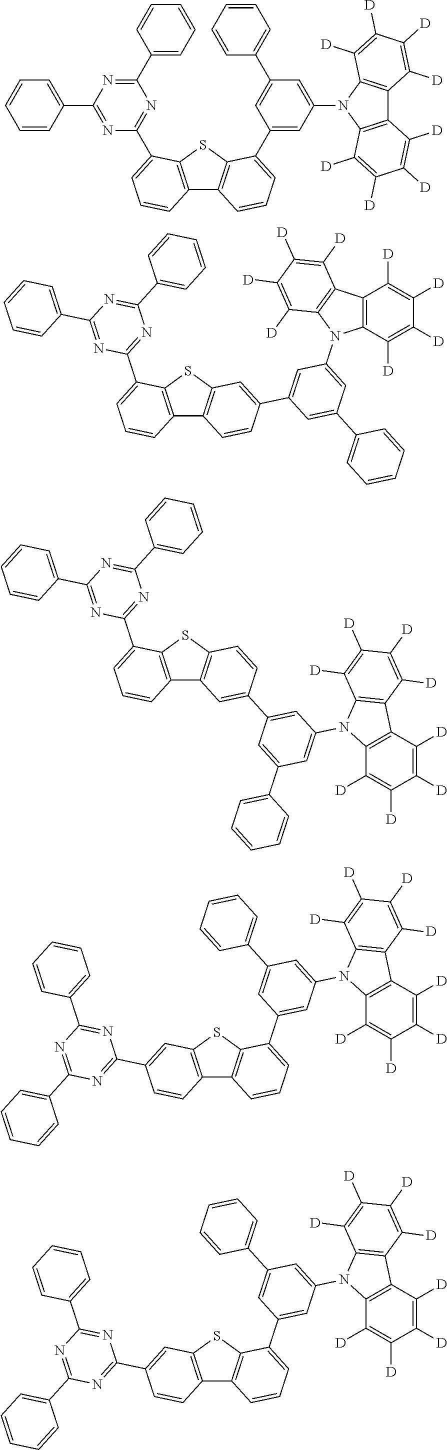

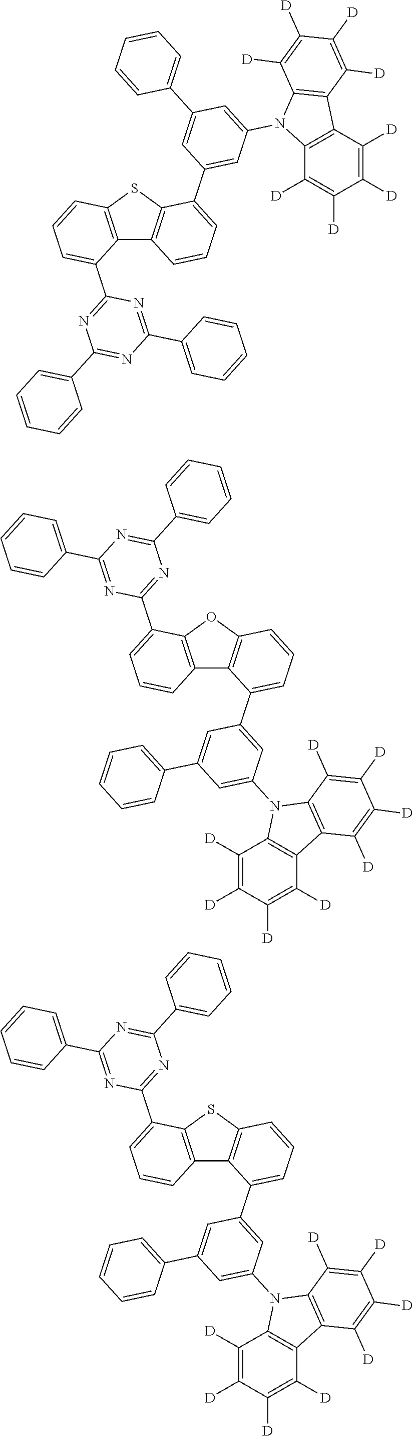 [SMILES: [2H]c1c([2H])c([2H])c2c(c1[2H])c1c([2H])c([2H])c([2H])c([2H])c1n2-c1cc(-c2ccccc2)cc(-c2cccc3c2sc2cccc(-c4nc(-c5ccccc5)nc(-c5ccccc5)n4)c23)c1 ; [2H]c1c([2H])c([2H])c2c(c1[2H])c1c([2H])c([2H])c([2H])c([2H])c1n2-c1cc(-c2ccccc2)cc(-c2cccc3oc4c(-c5nc(-c6ccccc6)nc(-c6ccccc6)n5)cccc4c23)c1 ; [2H]c1c([2H])c([2H])c2c(c1[2H])c1c([2H])c([2H])c([2H])c([2H])c1n2-c1cc(-c2ccccc2)cc(-c2cccc3sc4c(-c5nc(-c6ccccc6)nc(-c6ccccc6)n5)cccc4c23)c1]

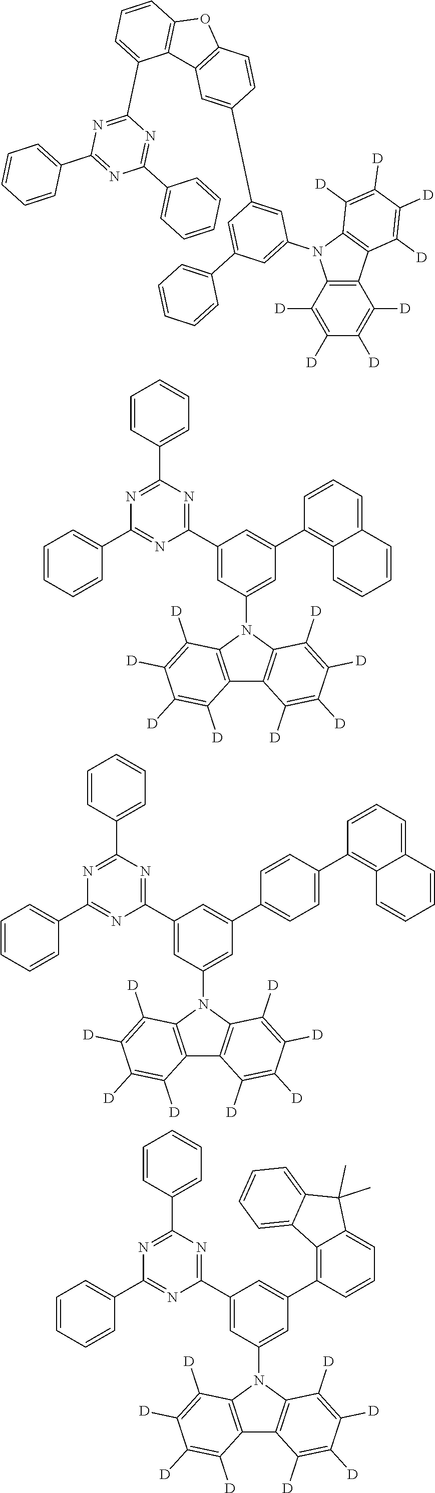

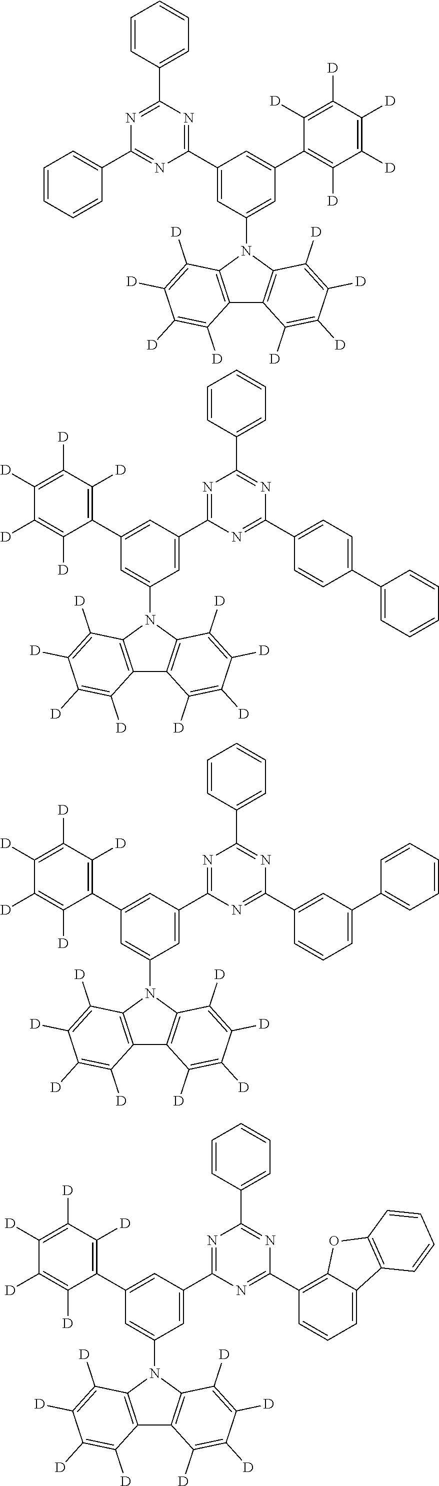

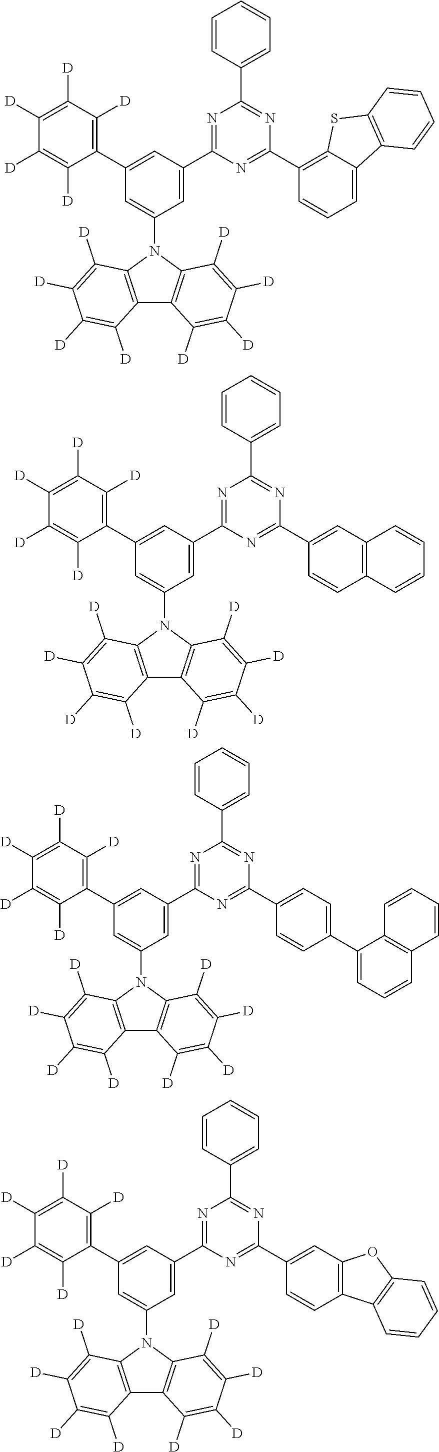

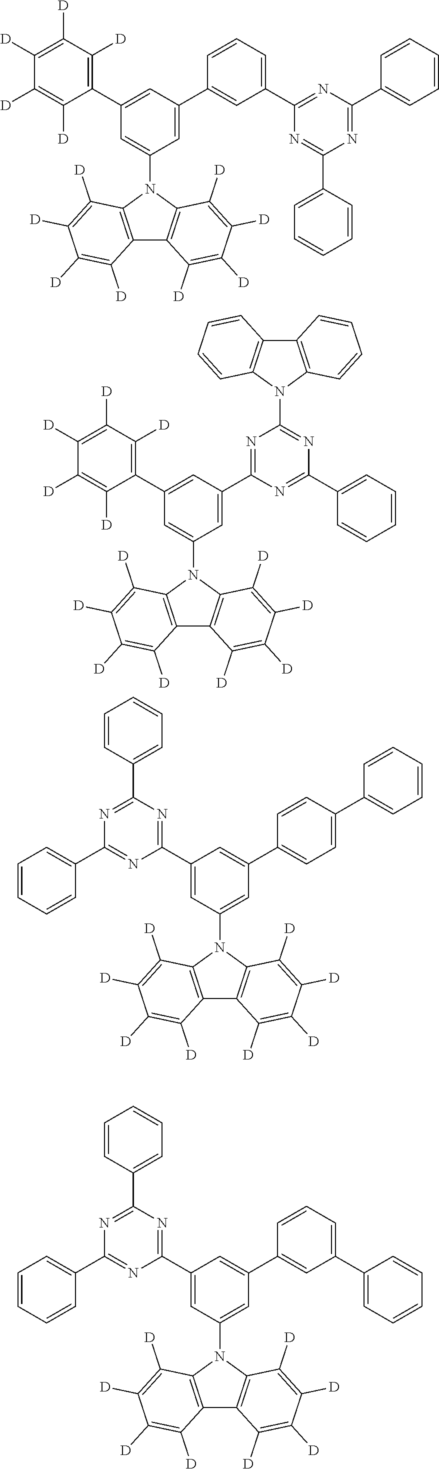

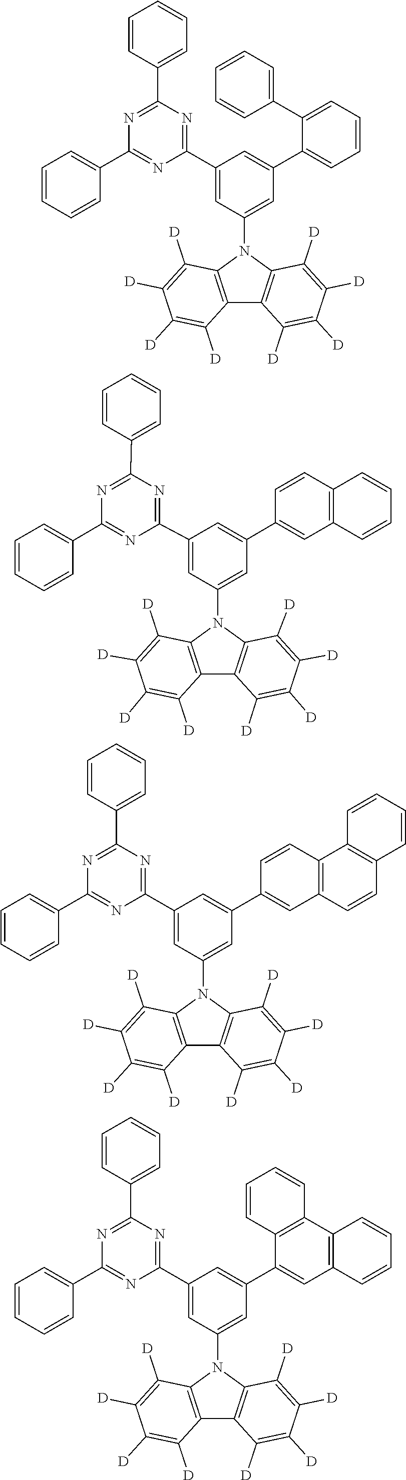

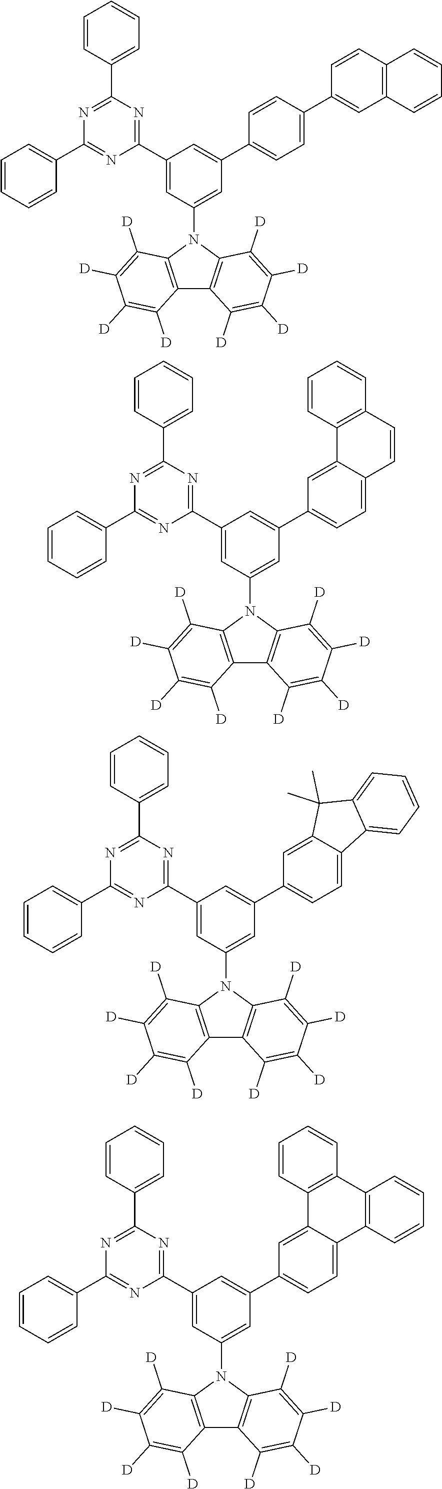

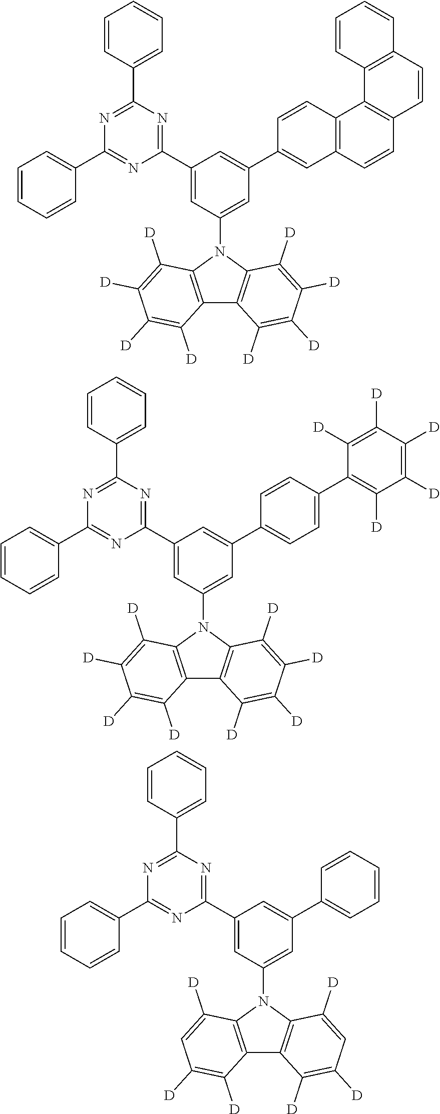

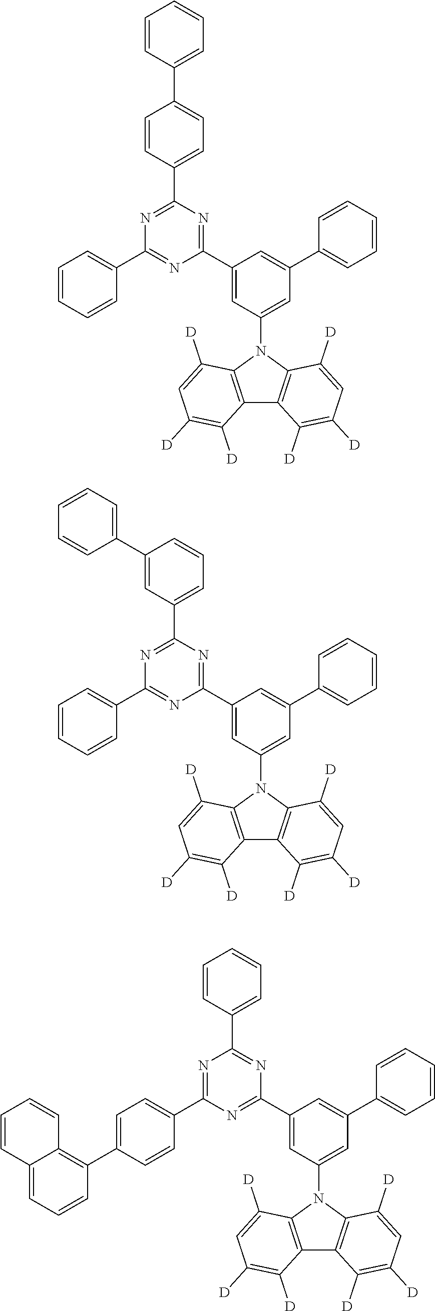 [SMILES: [2H]c1cc([2H])c2c(c1[2H])c1c([2H])c([2H])cc([2H])c1n2-c1cc(-c2ccccc2)cc(-c2nc(-c3ccccc3)nc(-c3ccc(-c4ccccc4)cc3)n2)c1 ; [2H]c1cc([2H])c2c(c1[2H])c1c([2H])c([2H])cc([2H])c1n2-c1cc(-c2ccccc2)cc(-c2nc(-c3ccccc3)nc(-c3cccc(-c4ccccc4)c3)n2)c1 ; [2H]c1cc([2H])c2c(c1[2H])c1c([2H])c([2H])cc([2H])c1n2-c1cc(-c2ccccc2)cc(-c2nc(-c3ccccc3)nc(-c3ccc(-c4cccc5ccccc45)cc3)n2)c1]

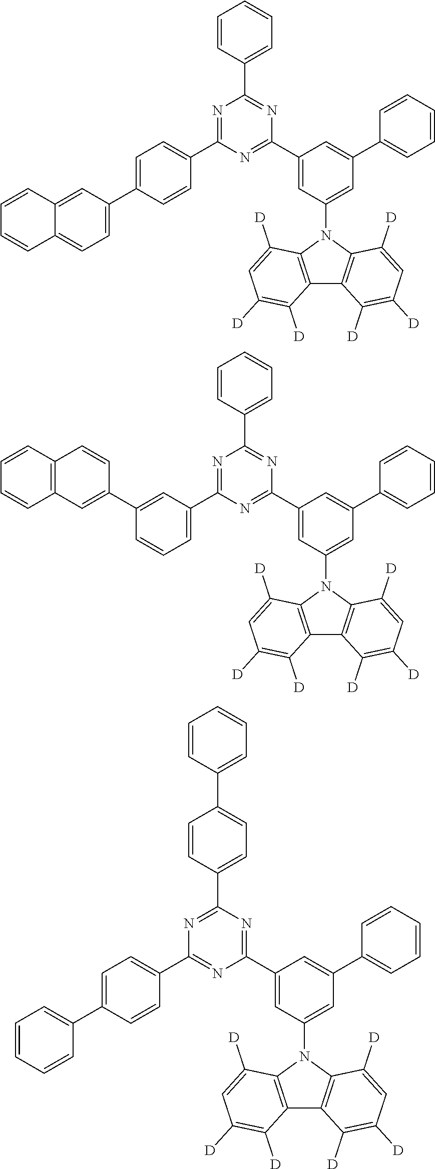

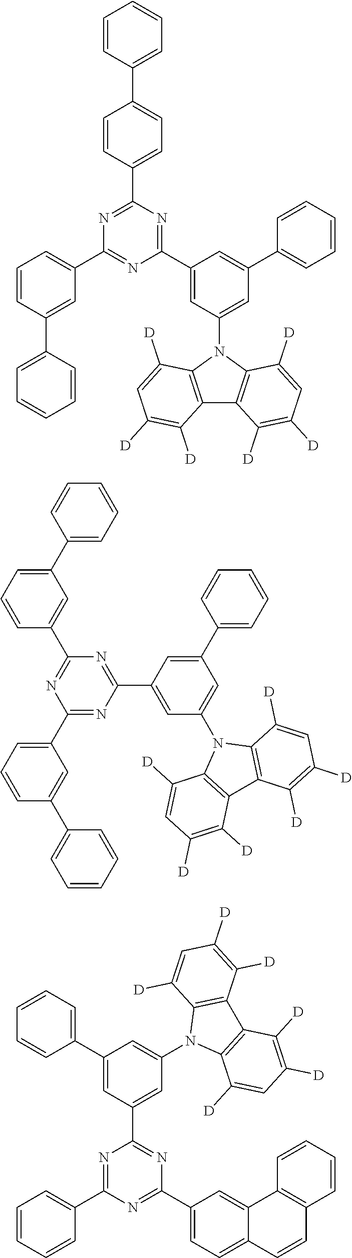

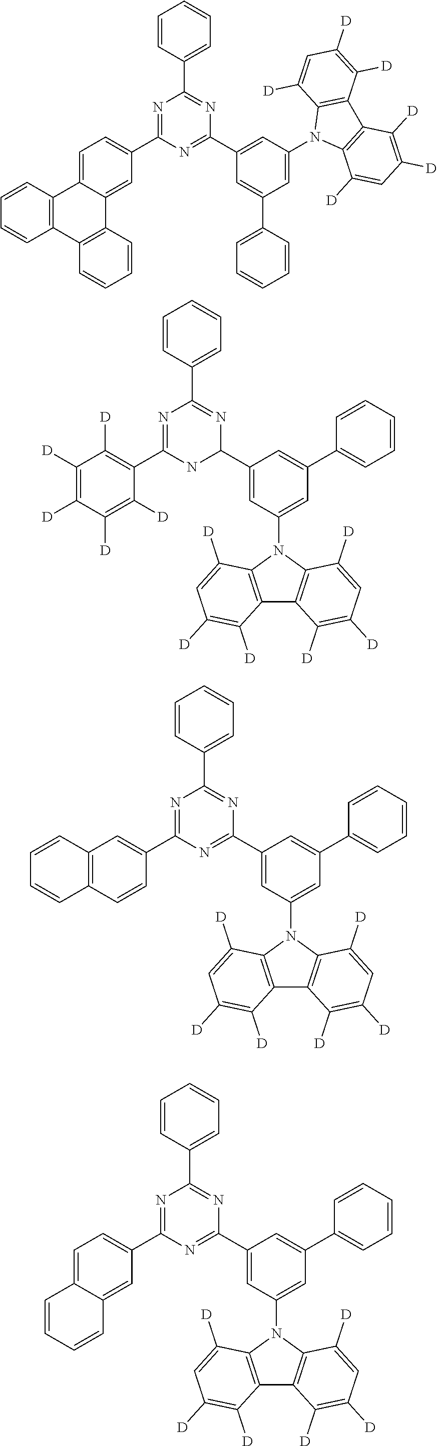

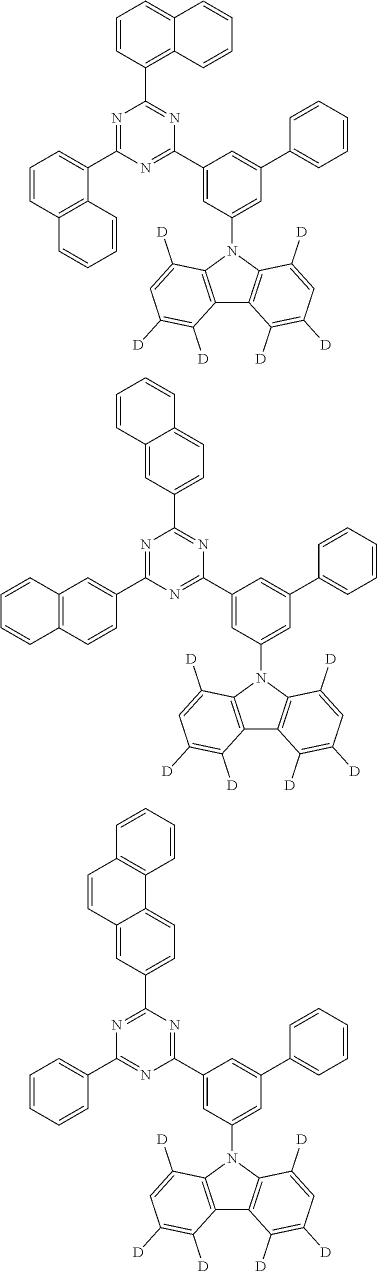

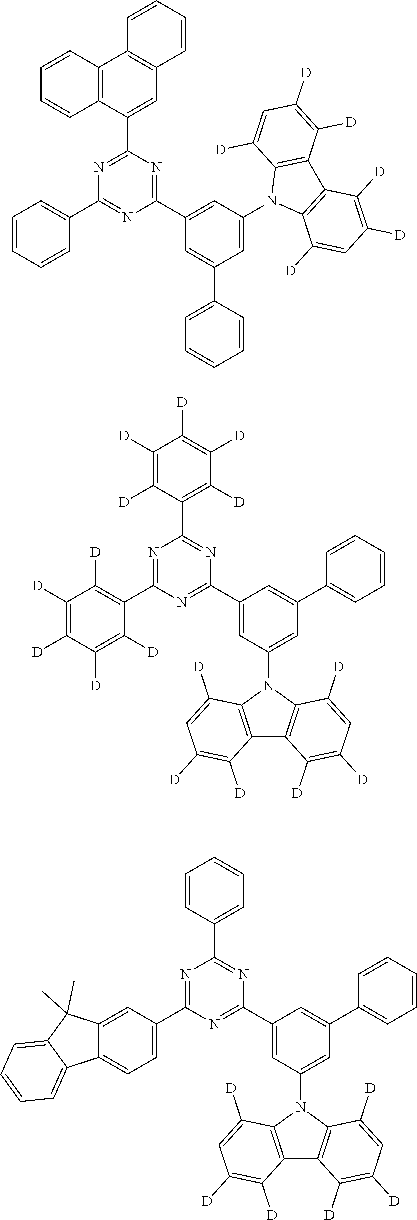 [SMILES: [2H]c1cc([2H])c2c(c1[2H])c1c([2H])c([2H])cc([2H])c1n2-c1cc(-c2ccccc2)cc(-c2nc(-c3ccccc3)nc(-c3cc4ccccc4c4ccccc34)n2)c1 ; [2H]c1cc([2H])c2c(c1[2H])c1c([2H])c([2H])cc([2H])c1n2-c1cc(-c2ccccc2)cc(-c2nc(-c3c([2H])c([2H])c([2H])c([2H])c3[2H])nc(-c3c([2H])c([2H])c([2H])c([2H])c3[2H])n2)c1 ; [2H]c1cc([2H])c2c(c1[2H])c1c([2H])c([2H])cc([2H])c1n2-c1cc(-c2ccccc2)cc(-c2nc(-c3ccccc3)nc(-c3ccc4c(c3)C(C)(C)c3ccccc3-4)n2)c1]

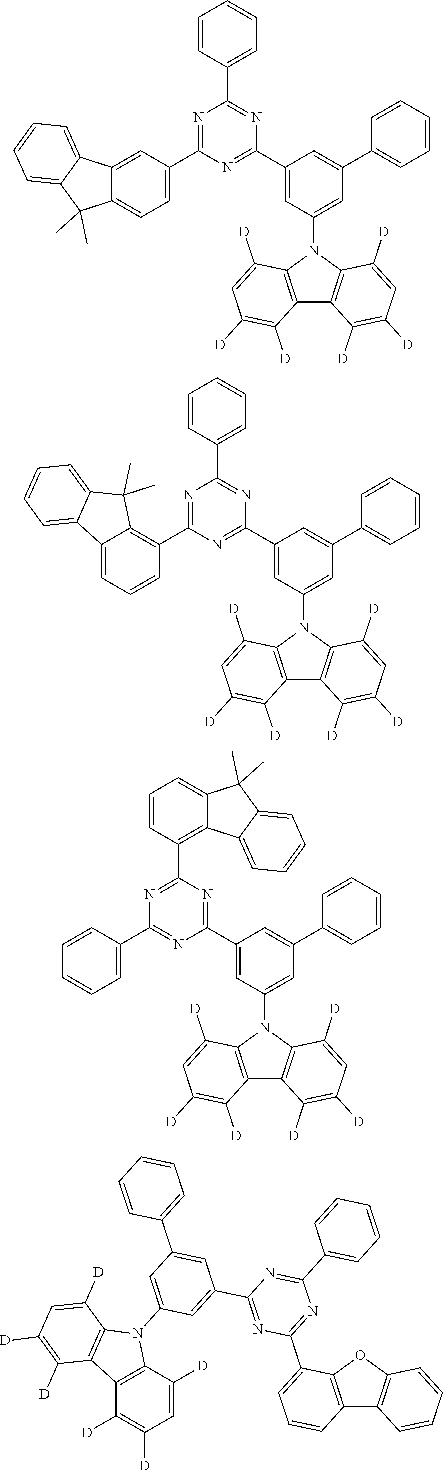

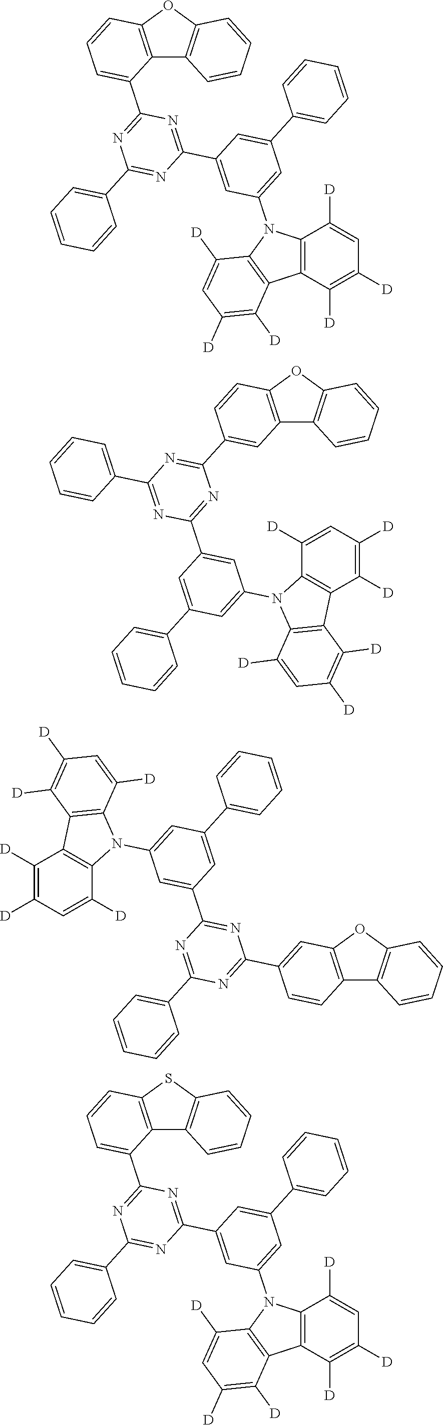

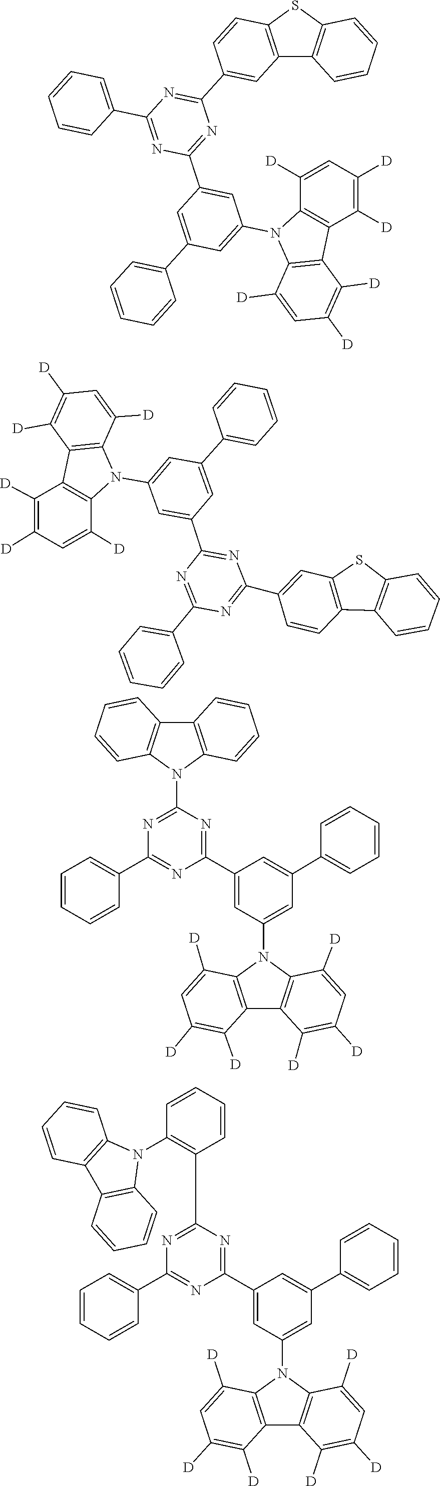

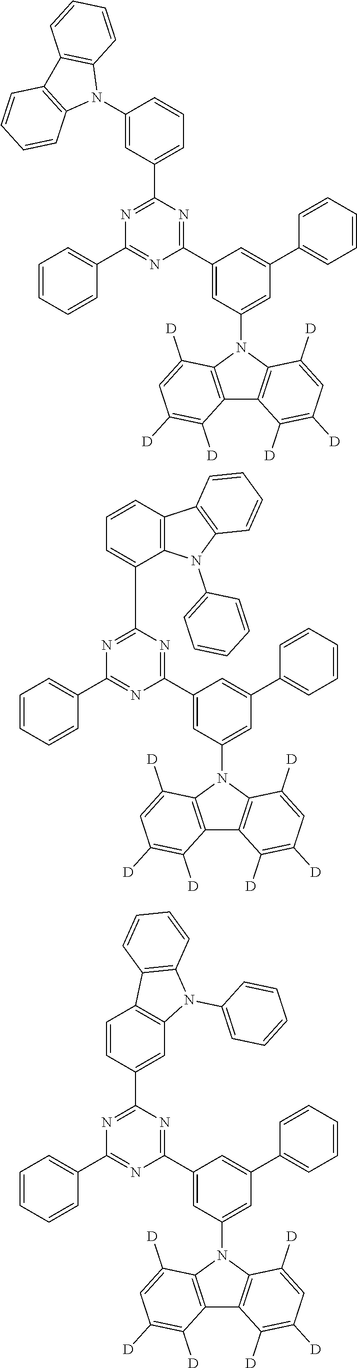

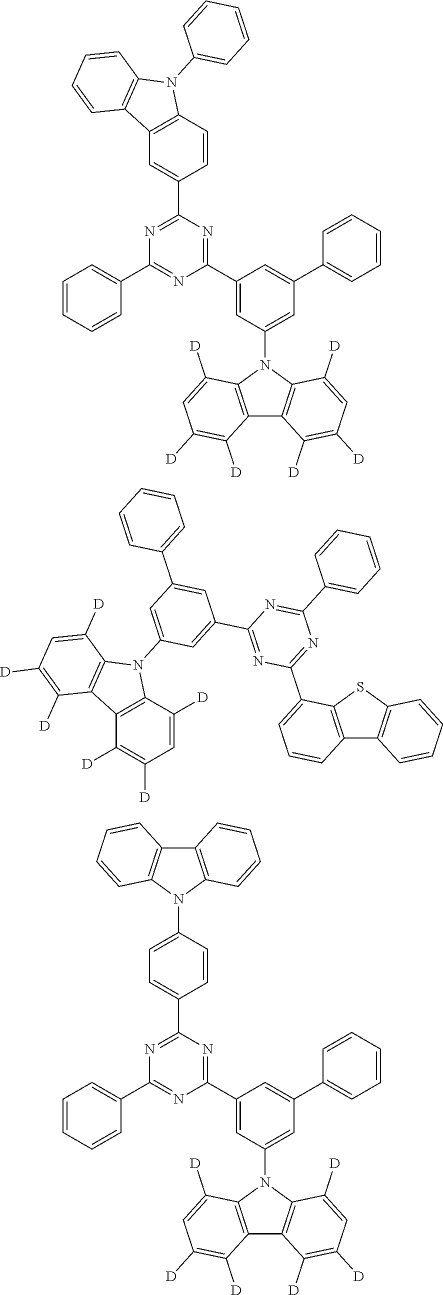

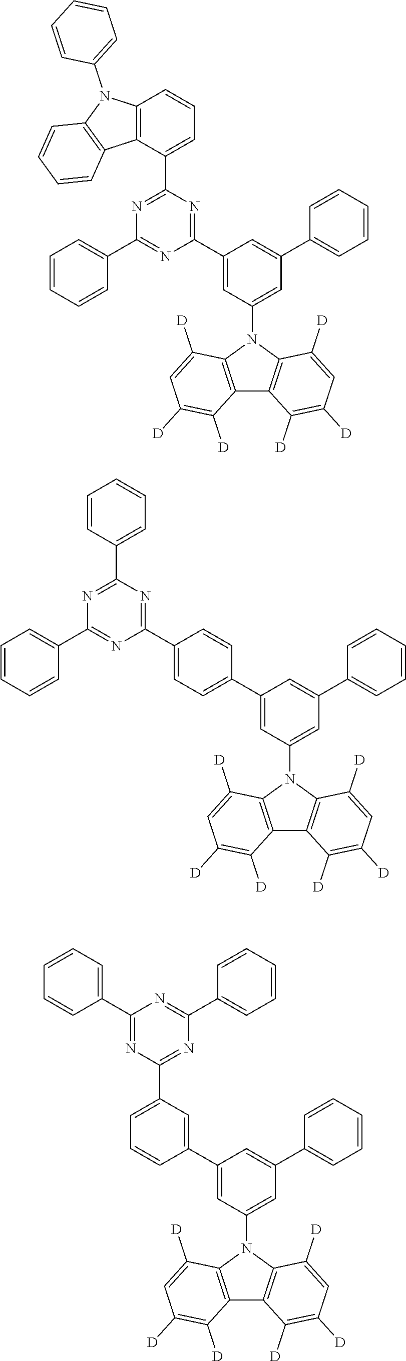 [SMILES: [2H]c1cc([2H])c2c(c1[2H])c1c([2H])c([2H])cc([2H])c1n2-c1cc(-c2ccccc2)cc(-c2nc(-c3ccccc3)nc(-c3cccc4c3c3ccccc3n4-c3ccccc3)n2)c1 ; [2H]c1cc([2H])c2c(c1[2H])c1c([2H])c([2H])cc([2H])c1n2-c1cc(-c2ccccc2)cc(-c2ccc(-c3nc(-c4ccccc4)nc(-c4ccccc4)n3)cc2)c1 ; [2H]c1cc([2H])c2c(c1[2H])c1c([2H])c([2H])cc([2H])c1n2-c1cc(-c2ccccc2)cc(-c2cccc(-c3nc(-c4ccccc4)nc(-c4ccccc4)n3)c2)c1]

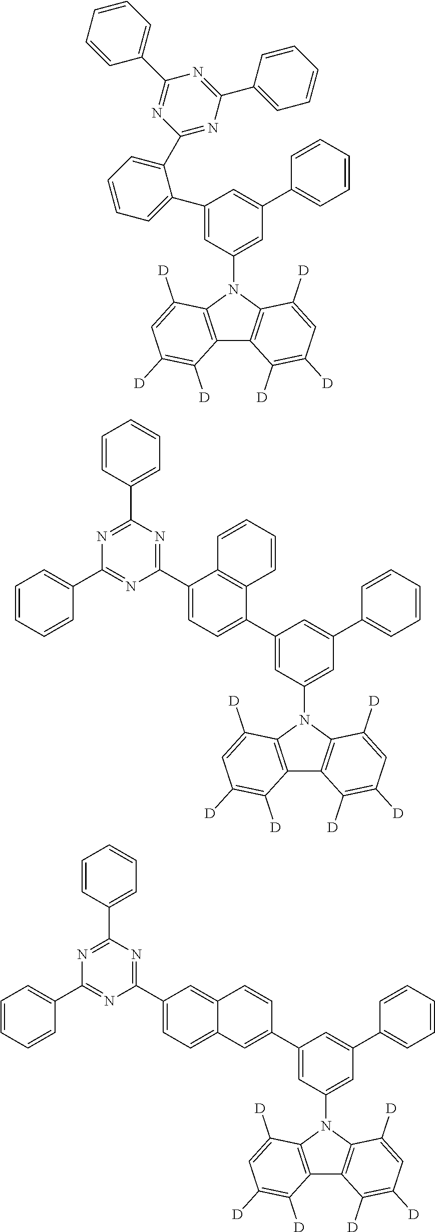

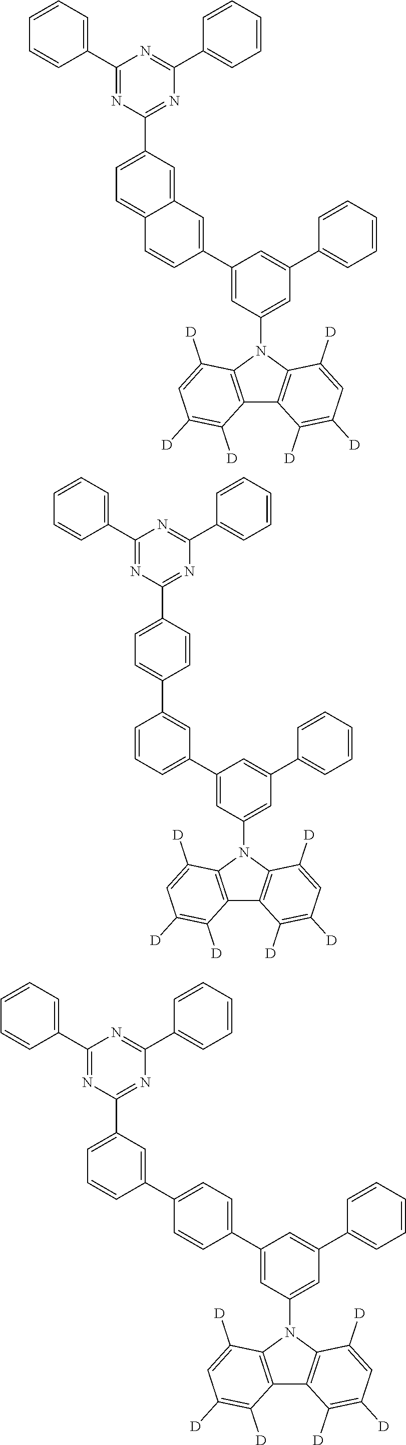

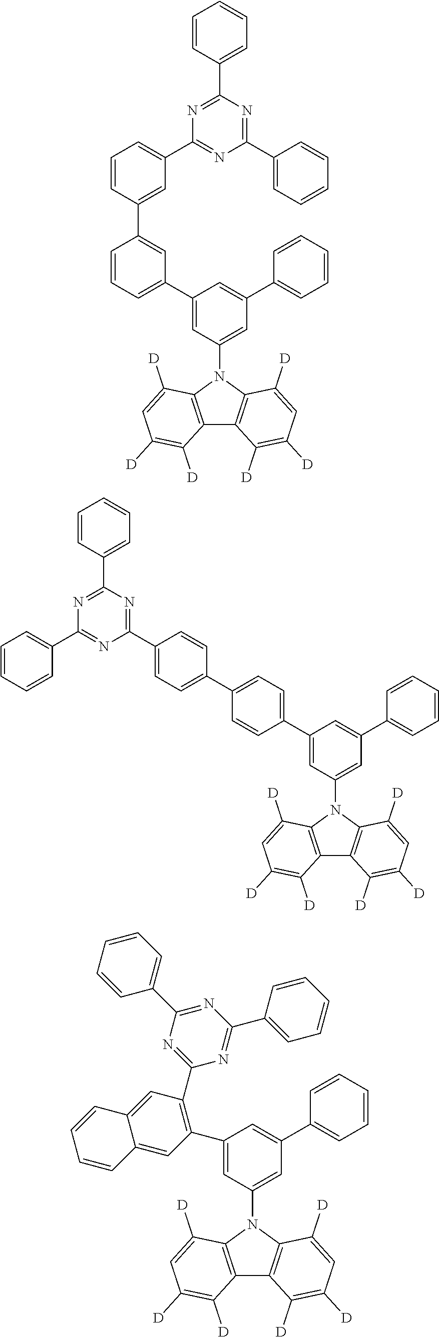

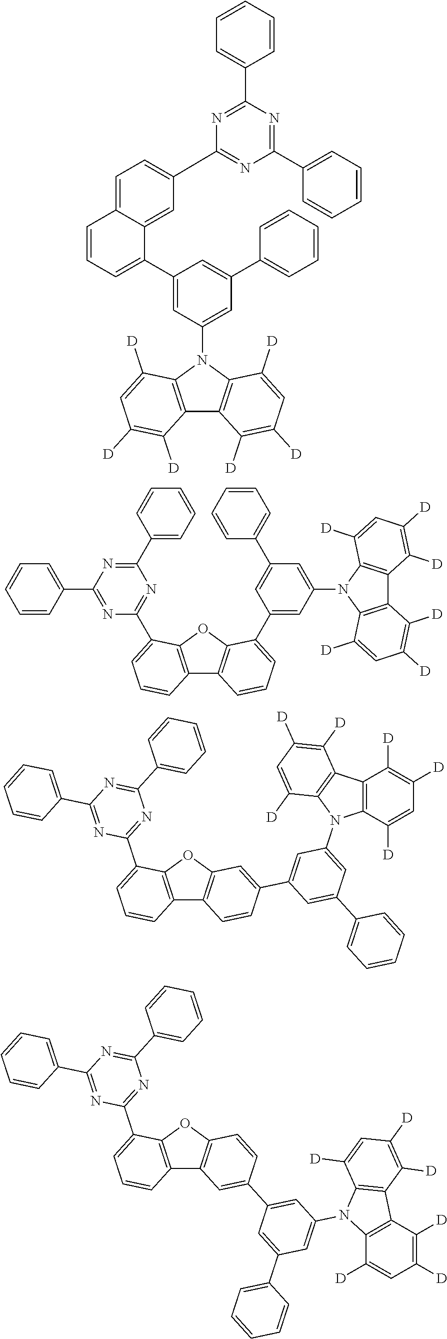

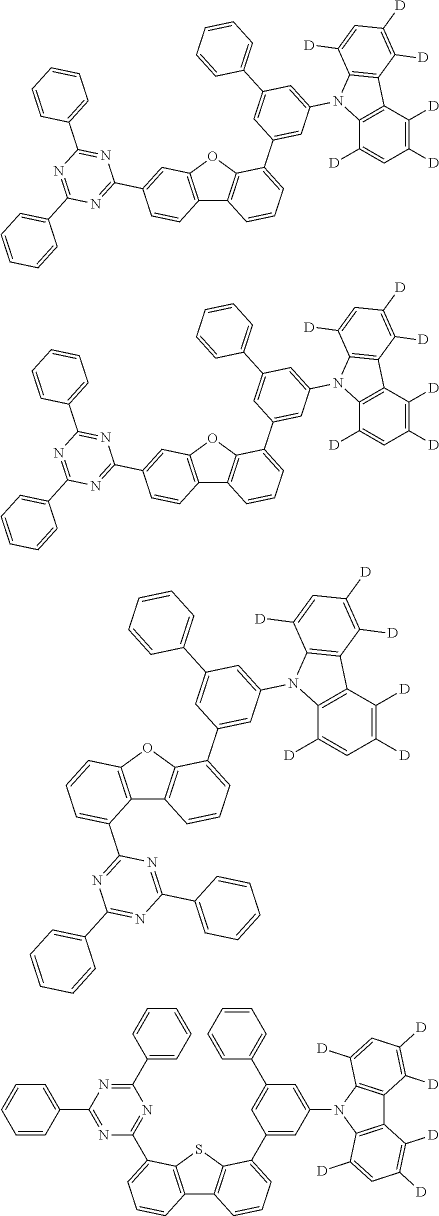

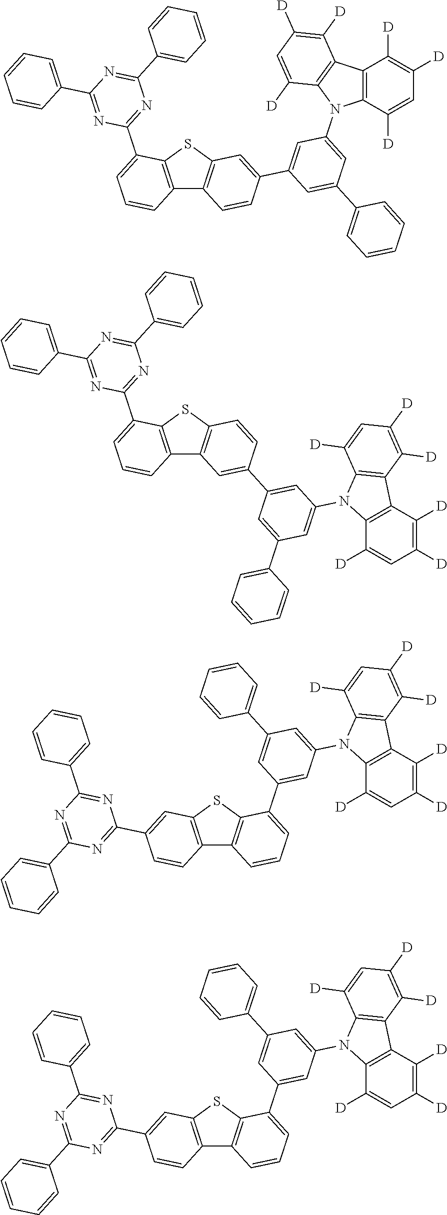

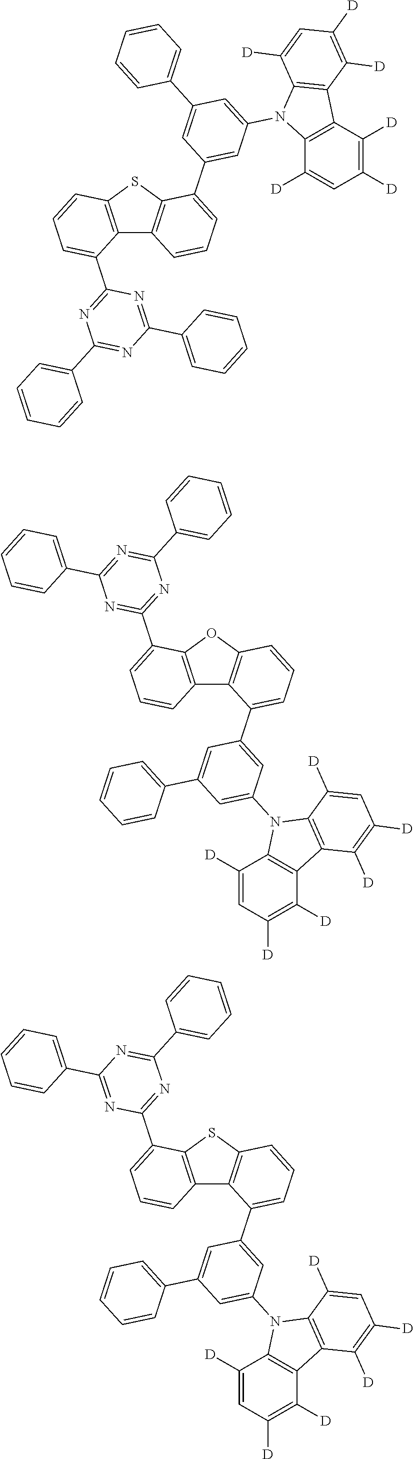

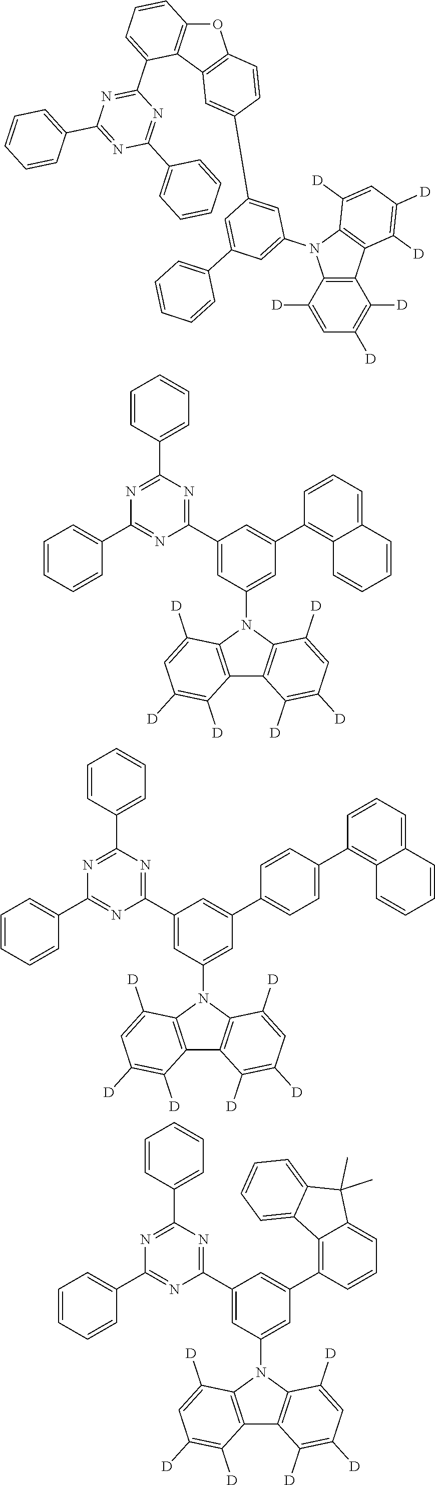

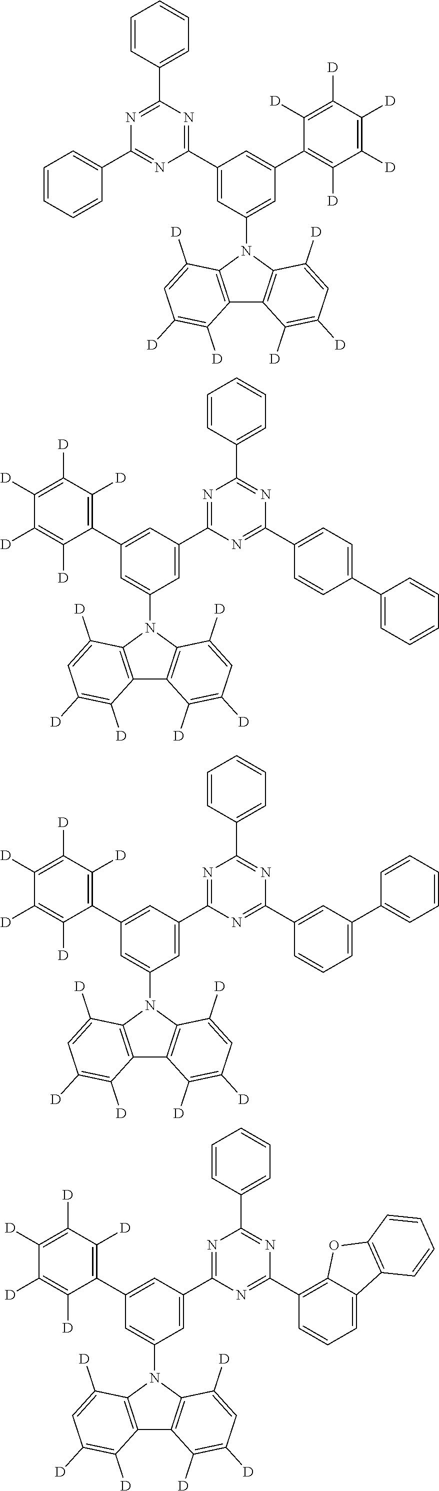

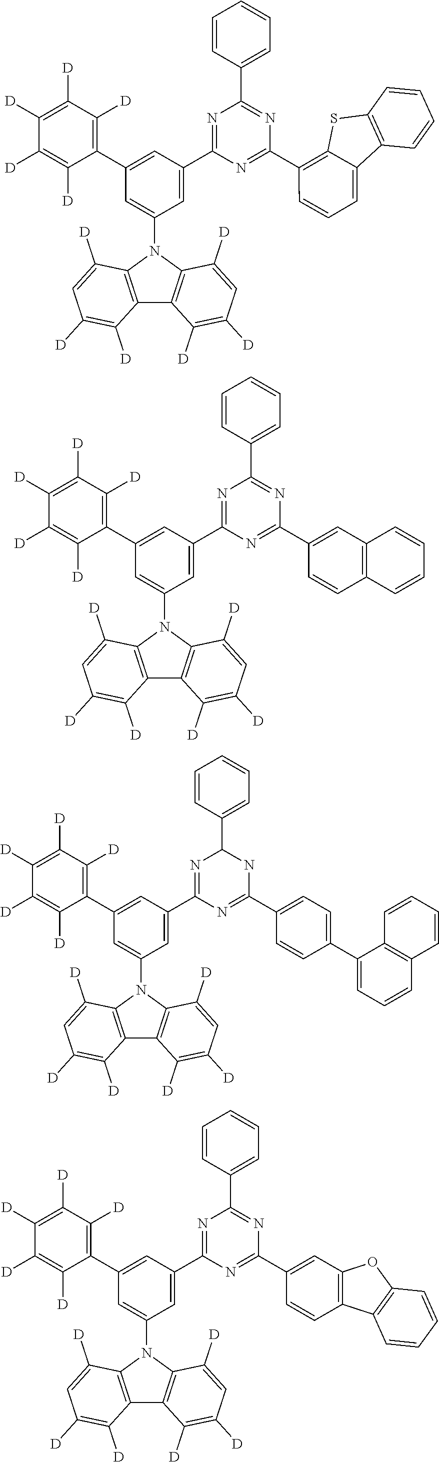

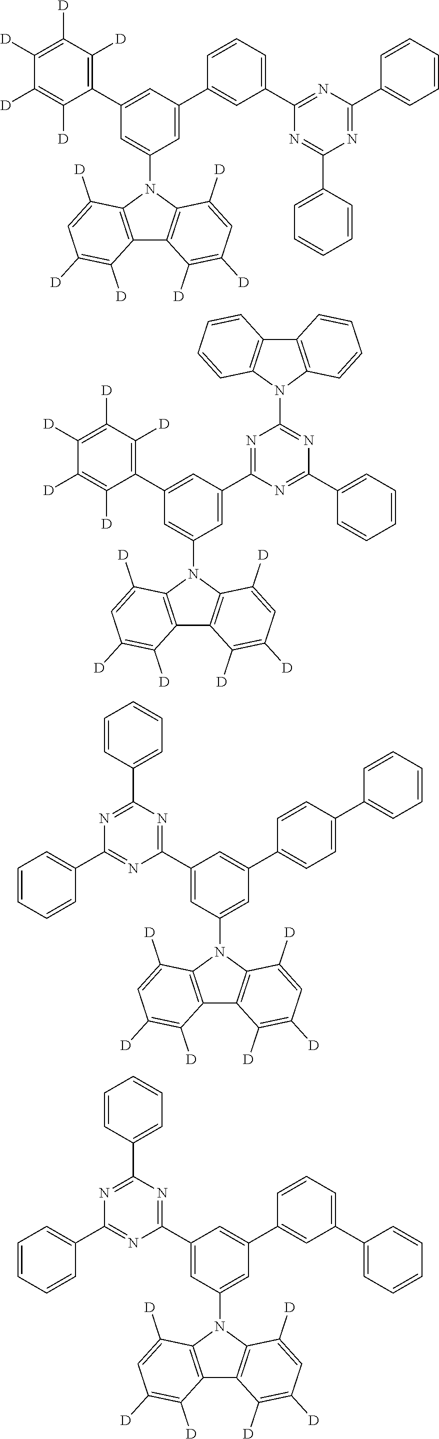

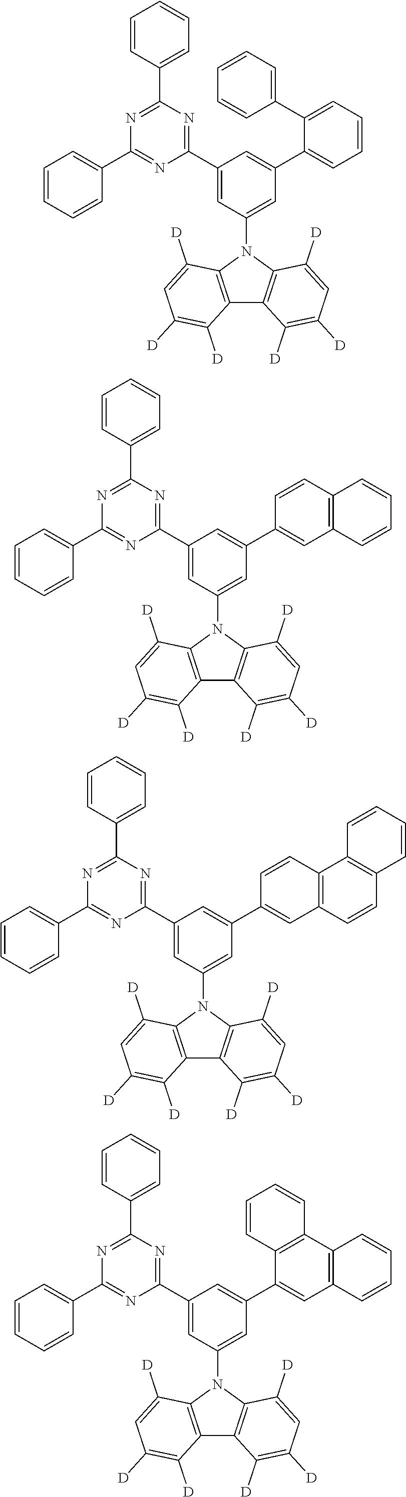

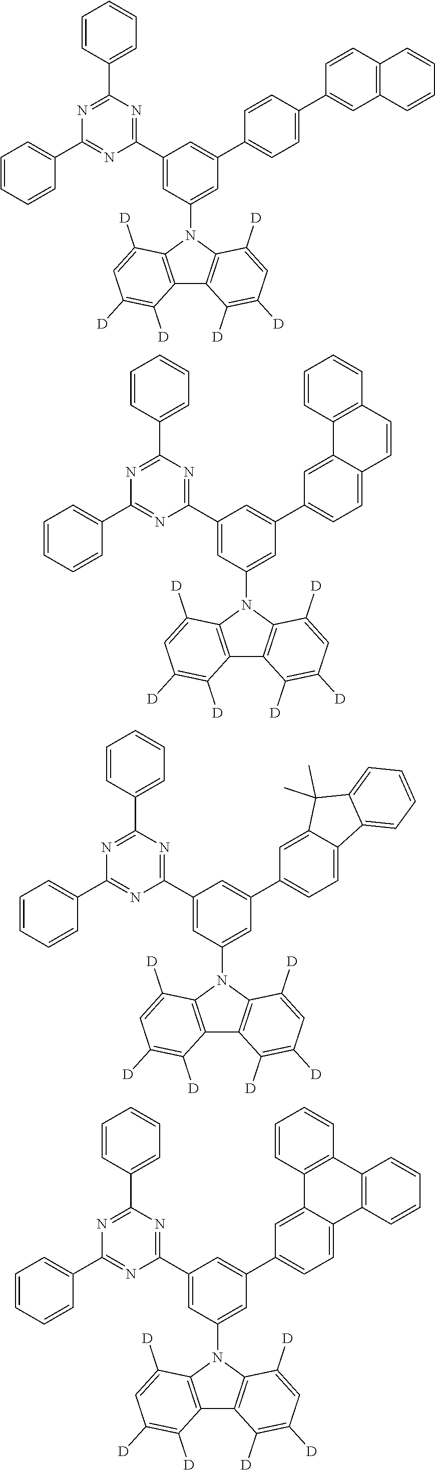

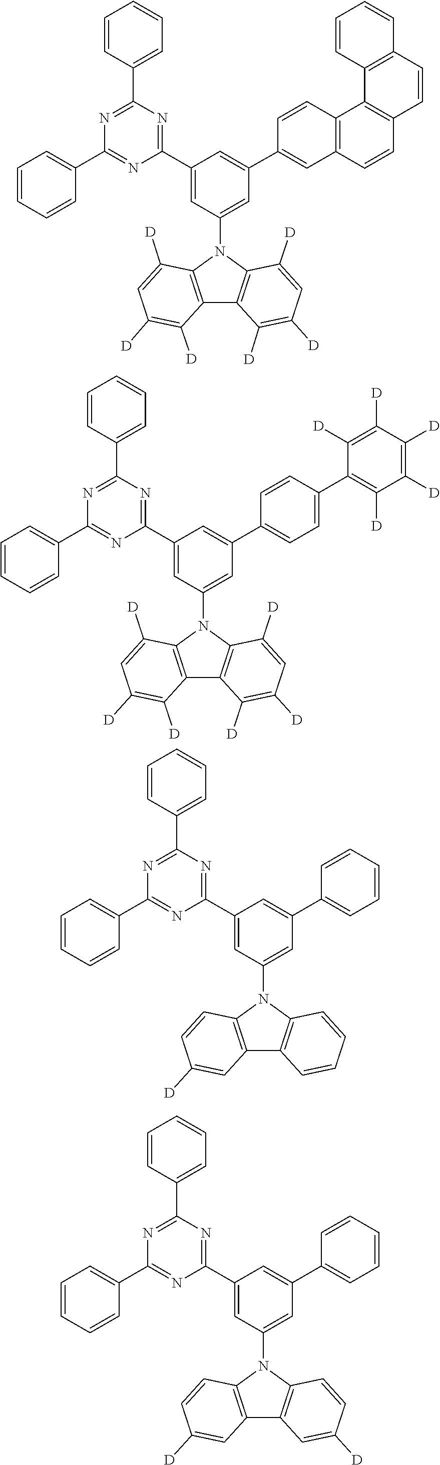

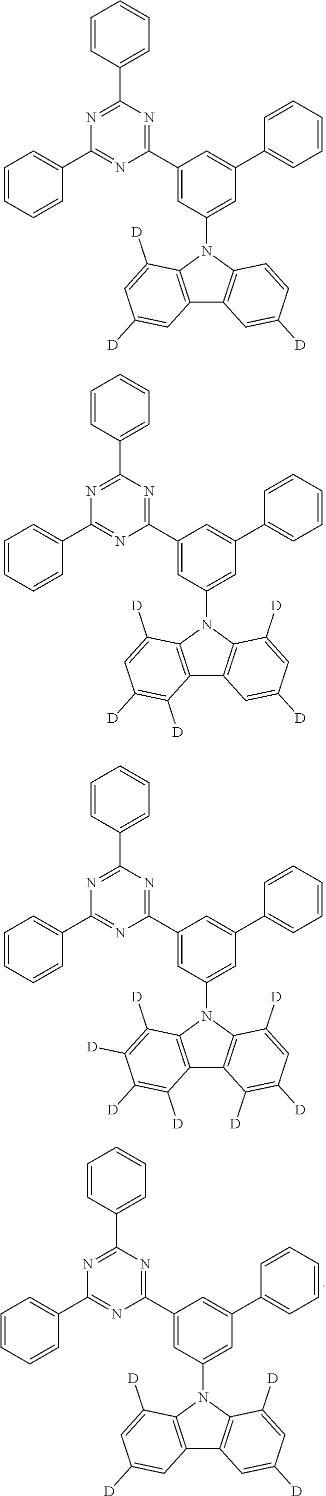

Meanwhile, the compound of Chemical Formula 1 can be prepared by the preparation method as shown in the following Reaction Scheme 1 as an example.

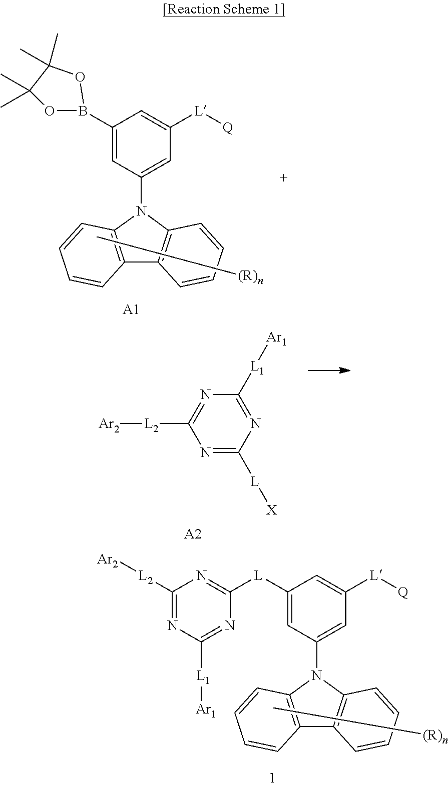

in Reaction Scheme 1, X is halogen, more preferably bromo or chloro, and the remaining substituents are the same as defined above.

Specifically, the compound of Chemical Formula 1 can be prepared from a Suzuki-coupling reaction of the reaction materials A1 and A2. At this time, the Suzuki-coupling reaction is preferably carried out in the presence of a palladium catalyst and a base, and a reactive group for the Suzuki-coupling reaction can be appropriately changed to a reactive group know in the art. Such a preparation method can be further embodied in Preparation Examples described hereinafter.

(Organic Light Emitting Device)

Meanwhile, the present disclosure provides an organic light emitting device comprising a compound of Chemical Formula 1. In one example, the present disclosure provides an organic light emitting device comprising: a first electrode; a second electrode that is opposite to the first electrode; and one or more organic material layers that are between the first electrode and the second electrode, wherein one or more layers of the organic material layers includes the compound of Chemical Formula 1.

The organic material layer of the organic light emitting device of the present disclosure can have a single-layer structure, or it can have a multilayered structure in which two or more organic material layers are stacked. For example, the organic light emitting device of the present disclosure can have a structure comprising a hole injection layer, a hole transport layer, a light emitting layer, an electron transport layer, an electron injection layer and the like as the organic material layer. However, the structure of the organic light emitting device is not limited thereto, and it can include a smaller number of organic layers.

In one embodiment, the organic material layer can include a light emitting layer, wherein the organic material layer including the compound can be a light emitting layer.

In another embodiment, the organic material layer can include a hole injection layer, a hole transport layer, a light emitting layer, an electron transport layer and an electron injection layer, wherein the organic material layer including the compound can be a light emitting layer or an electron transport layer.

In another embodiment, the organic material layer can include a hole injection layer, a hole transport layer, an electron blocking layer, a light emitting layer, an electron transport layer and an electron injection layer, wherein the organic material layer including the compound can be a light emitting layer or an electron transport layer.

In yet another embodiment, the organic material layer can include a hole injection layer, a hole transport layer, an electron blocking layer, a light emitting layer, a hole blocking layer, an electron transport layer and an electron injection layer, wherein the organic material layer including the compound can be a light emitting layer or an electron transport layer.

The organic material layer of the organic light emitting device of the present disclosure can have a single-layer structure, or it can have a multilayered structure in which two or more organic material layers are stacked. For example, the organic light emitting device of the present disclosure can have a structure further comprising a hole injection layer and a hole transport layer between the first electrode and the light emitting layer, and an electron transport layer and an electron injection layer between the light emitting layer and the second electrode, in addition to the light emitting layer, as the organic material layer. However, the structure of the organic light emitting device is not limited thereto, and it can include a smaller number of organic layers or a larger number of organic layers.

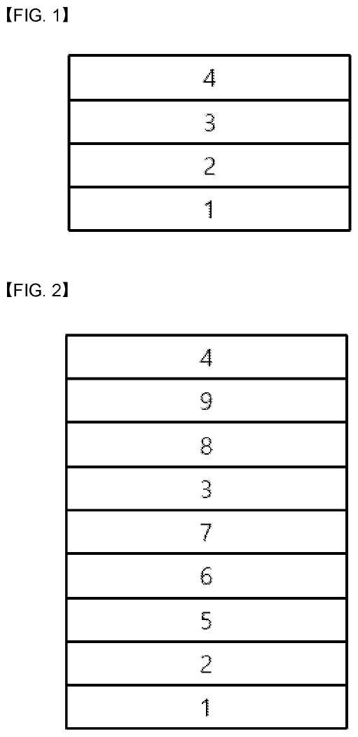

Further, the organic light emitting device according to the present disclosure can be a normal type organic light emitting device in which an anode, one or more organic material layers and a cathode are sequentially stacked on a substrate, wherein the first electrode is an anode, and the second electrode is a cathode. Further, the organic light emitting device according to the present disclosure can be an inverted type organic light emitting device in which a cathode, one or more organic material layers and an anode are sequentially stacked on a substrate, wherein the first electrode is a cathode and the second electrode is an anode. For example, the structure of the organic light emitting device according to one embodiment of the present disclosure is illustrated in FIGS. 1 and 2.

FIG. 1 shows an example of an organic light emitting device comprising a substrate 1, an anode 2, a light emitting layer 3, and a cathode 4. In such a structure, the compound of Chemical Formula 1 can be included in the light emitting layer.

FIG. 2 shows an example of an organic light emitting device comprising a substrate 1, an anode 2, a hole injection layer 5, a hole transport layer 6, an electron blocking layer 7, a light emitting layer 3, an electron transport layer 8, an electron injection layer 9, and a cathode 4. In such a structure, the compound of Chemical Formula 1 can be included in the light emitting layer.

The organic light emitting device according to the present disclosure can be manufactured by materials and methods known in the art, except that at least one of the organic material layers includes the compound of Chemical Formula 1. Further, when the organic light emitting device includes a plurality of organic material layers, the organic material layers can be formed of the same material or different materials.

For example, the organic light emitting device according to the present disclosure can be manufactured by sequentially stacking a first electrode, an organic material layer and a second electrode on a substrate. In this case, the organic light emitting device can be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer and the electron transport layer thereon, and then depositing a material that can be used as the cathode thereon. In addition to such a method, the organic light emitting device can be manufactured by sequentially depositing a cathode material, an organic material layer and an anode material on a substrate.

Further, the compound of Chemical Formula 1 can be formed into an organic layer by a solution coating method as well as a vacuum deposition method at the time of manufacturing an organic light emitting device. Herein, the solution coating method means a spin coating, a dip coating, a doctor blading, an inkjet printing, a screen printing, a spray method, a roll coating, or the like, but is not limited thereto.

In addition to such a method, the organic light emitting device can be manufactured by sequentially depositing a cathode material, an organic material layer and an anode material on a substrate (International Publication WO2003/012890). However, the manufacturing method is not limited thereto.

As an example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive compounds such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

Further, the hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to an electron injection layer or the electron injection material, and further is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

Further, the hole transport layer is a layer that receives holes from a hole injection layer and transports the holes to the light emitting layer. The hole transport material is suitably a material having large mobility to the holes, which can receive holes from the anode or the hole injection layer and transfer the holes to the light emitting layer. The hole transport material includes an arylamine-based organic material, a conductive polymer, a block copolymer in which a conjugate portion and a non-conjugate portion are present together, and the like, but are not limited thereto.

Further, the electron blocking layer refers to a layer which is formed on the hole transport layer, preferably provided in contact with the light emitting layer, and serves to adjust the hole mobility, prevent excessive movement of electrons, and increase the probability of hole-electron coupling, thereby improving the efficiency of the organic light emitting device. The electron blocking layer includes an electron blocking material, and examples of such electron blocking material can include can include an arylamine-based organic material or the like, but is not limited thereto.

Further, the light emitting layer can include a host material and a dopant material. The compound of Chemical Formula 1 can be used as such a host material. Further, the host material can further include a fused aromatic ring derivative, a heterocycle-containing compound or the like in addition to the compound of Chemical Formula 1. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto.

In one embodiment, the light emitting layer can further include a compound of the following Chemical Formula 2, in addition to the compound of Chemical Formula 1:

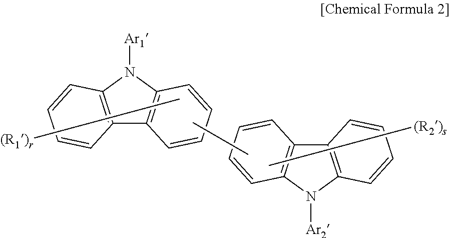

wherein, in Chemical Formula 2:

Ar′₁ and Ar′₂ are each independently a substituted or unsubstituted C₆₋₆₀ aryl or a substituted or unsubstituted C₂₋₆₀ heteroaryl containing one or more heteroatoms of N, O and S;

R′₁ and R′₂ are each independently hydrogen, deuterium, a C₁₋₆₀ alkyl, a C₆₋₆₀ aryl, or a C₂₋₆₀ heteroaryl containing one or more heteroatoms of N, O and S; and

r and s are each independently an integer of 0 to 7.

When the organic light emitting device further includes the compound of Chemical Formula 2, which can efficiently transfer holes to a dopant material from a host material for the light emitting layer, the recombination probability between holes and electrons in the light emitting layer increases with the compound of Chemical Formula 1 having excellent electron transport capability, and thereby is capable of enhancing the efficiency and lifetime of the organic light emitting device.

According to one embodiment, the compound of Chemical Formula 2 can be of the following Chemical Formula 2′:

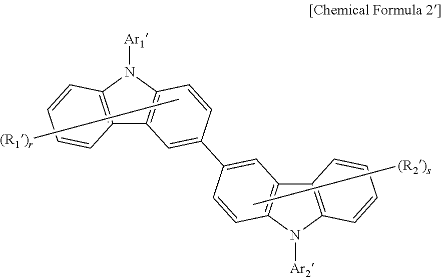

wherein, in Chemical Formula 2′,

Ar′₁, Ar′₂, R′₁, R′₂, r and s are as defined in Chemical Formula 2.

Further, in Chemical Formula 2, Ar′₁ and Ar′₂ are each independently a C₆₋₂₀ aryl, or a C₂₋₂₀ heteroaryl containing one heteroatom of N, O and S,

wherein Ar′₁ can be unsubstituted or substituted with one or more substituents selected from the group consisting of deuterium and a C₆₋₂₀ aryl.

For example, Ar′₁ and Ar′₂ can be each independently phenyl, biphenylyl, terphenylyl, naphthyl, dibenzofuranyl, or dibenzothiophenyl,

wherein Ar′₁ can be unsubstituted or substituted with one or more substituents selected from the group consisting of deuterium and a C₆₋₂₀ aryl.

At this time, at least one of Ar′₁ and Ar′₂ can be phenyl or biphenylyl.

Further, in Chemical Formula 2, R′₁ and R′₂ can be each independently hydrogen, deuterium, or a C₆₋₂₀ aryl.

For example, R′₁ and R′₂ can be each independently hydrogen, deuterium, or phenyl, but are not limited thereto.

Further, r and s, each indicating the number of R′₁ and R′₂, can be each independently 0, 1, 2, 3, 4, 5, 6, or 7.

More specifically, r and s can be each independently 0, 1, or 7.

For example, r+s can be 0 or 1.

Representative examples of the compound of Chemical Formula 2 are as follows:

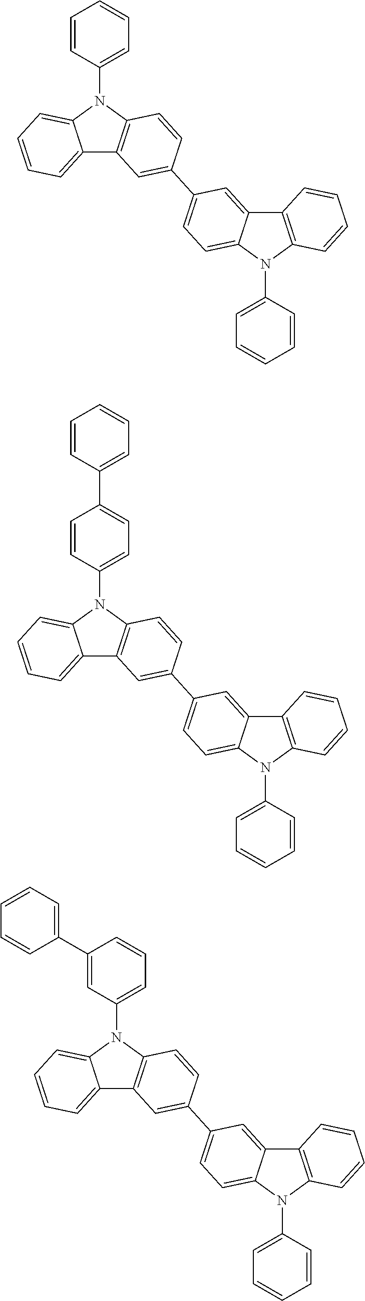 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1]

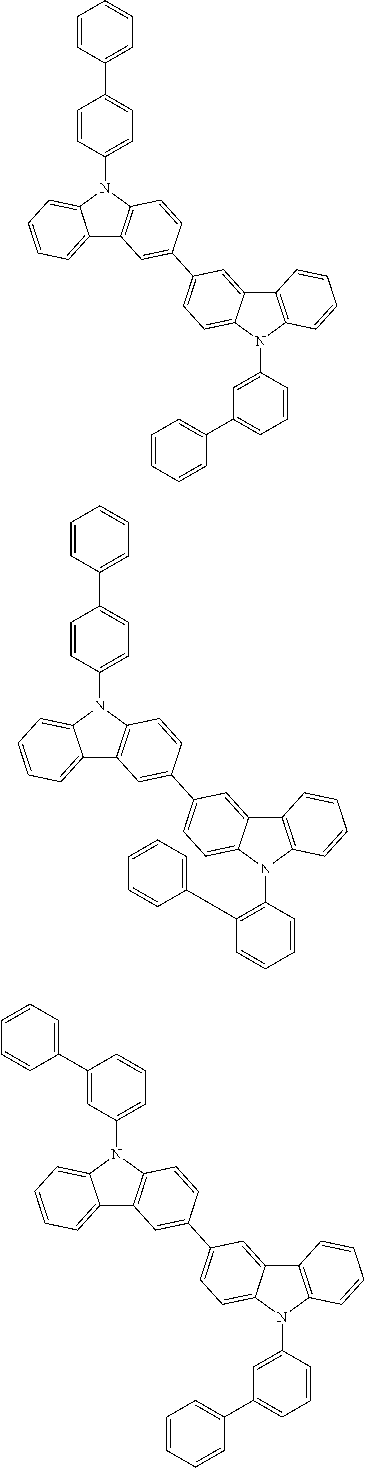 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

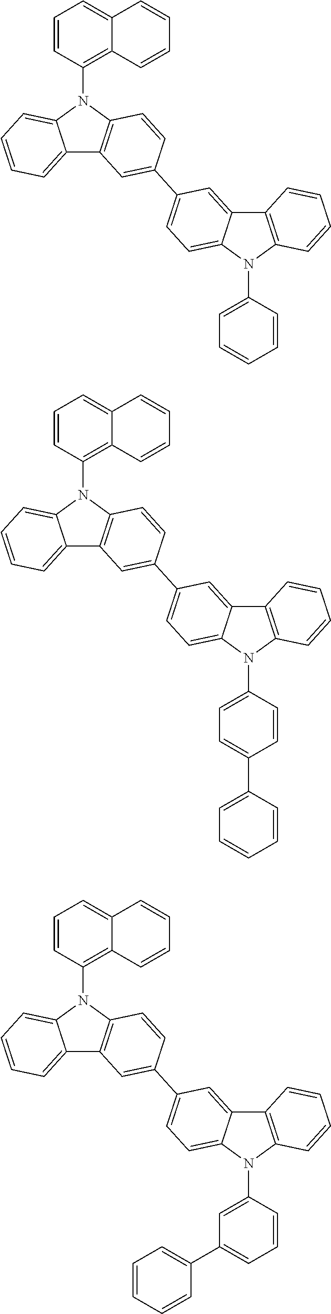 [SMILES: c1ccc(-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2cccc(-n3c4ccccc4c4cc(-c5ccc6c(c5)c5ccccc5n6-c5cccc6ccccc56)ccc43)c2)cc1]

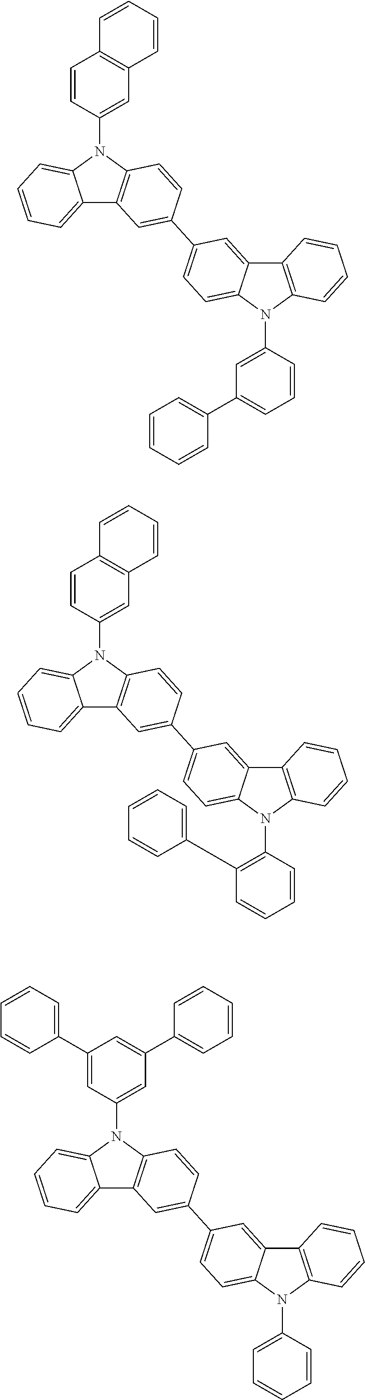 [SMILES: c1ccc(-c2cccc(-n3c4ccccc4c4cc(-c5ccc6c(c5)c5ccccc5n6-c5ccc6ccccc6c5)ccc43)c2)cc1 ; c1ccc(-c2ccccc2-n2c3ccccc3c3cc(-c4ccc5c(c4)c4ccccc4n5-c4ccc5ccccc5c4)ccc32)cc1 ; c1ccc(-c2cc(-c3ccccc3)cc(-n3c4ccccc4c4cc(-c5ccc6c(c5)c5ccccc5n6-c5ccccc5)ccc43)c2)cc1]

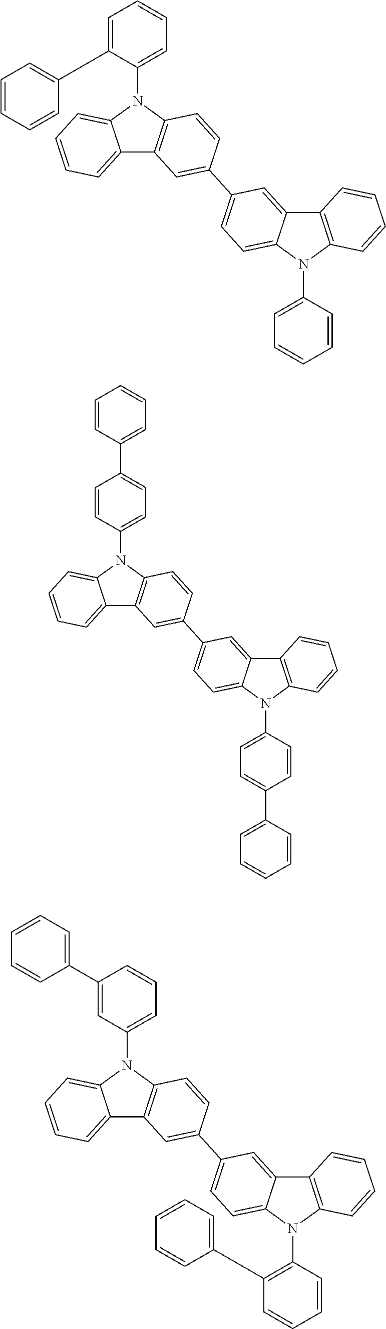 [SMILES: c1ccc(-c2ccccc2-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5ccccc5-c5ccccc5)ccc43)c2)cc1]

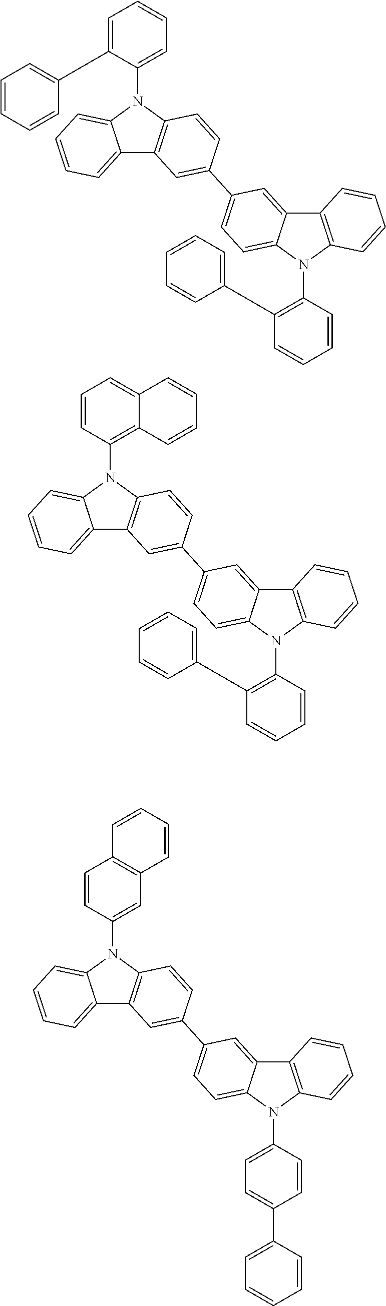 [SMILES: c1ccc(-c2ccccc2-n2c3ccccc3c3cc(-c4ccc5c(c4)c4ccccc4n5-c4ccccc4-c4ccccc4)ccc32)cc1 ; c1ccc(-c2ccccc2-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5ccc6ccccc6c5)ccc43)cc2)cc1]

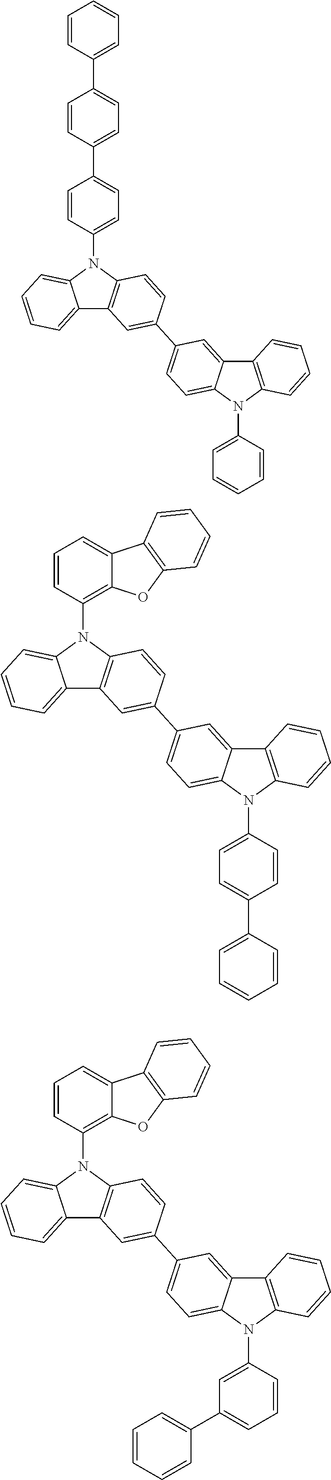

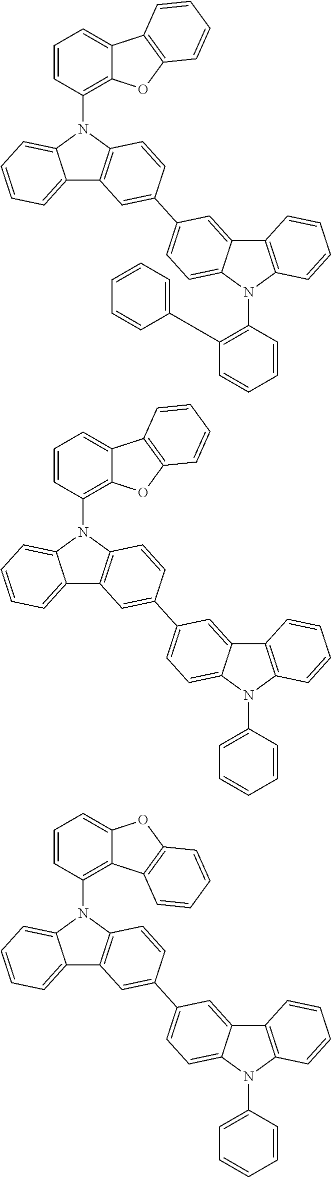

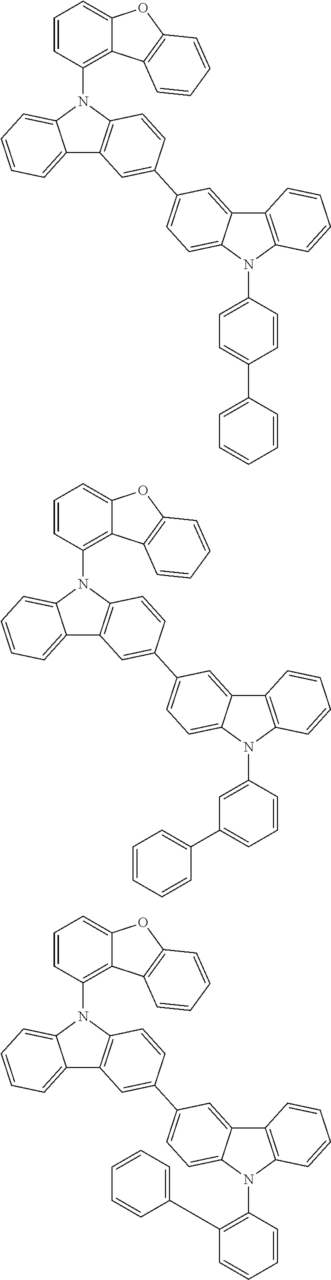

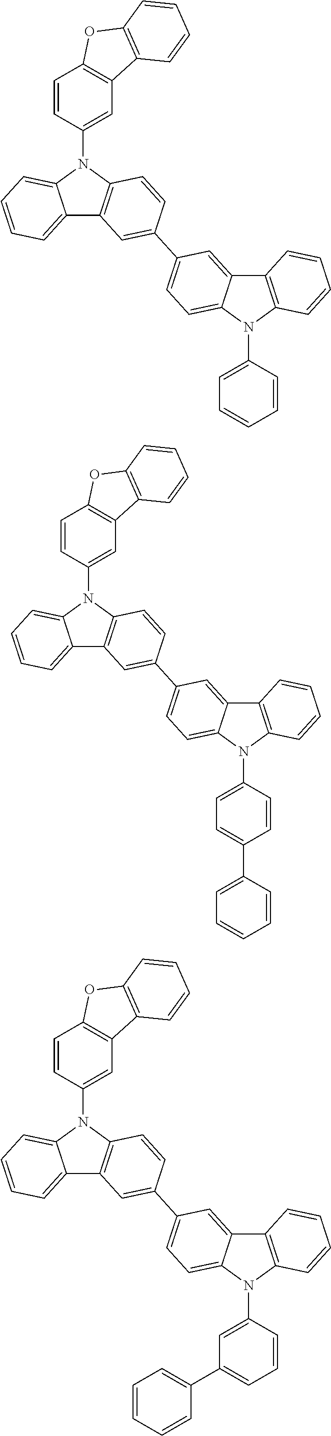 [SMILES: c1ccc(-n2c3ccccc3c3cc(-c4ccc5c(c4)c4ccccc4n5-c4ccc5oc6ccccc6c5c4)ccc32)cc1 ; c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(-c2cccc(-n3c4ccccc4c4cc(-c5ccc6c(c5)c5ccccc5n6-c5ccc6oc7ccccc7c6c5)ccc43)c2)cc1]

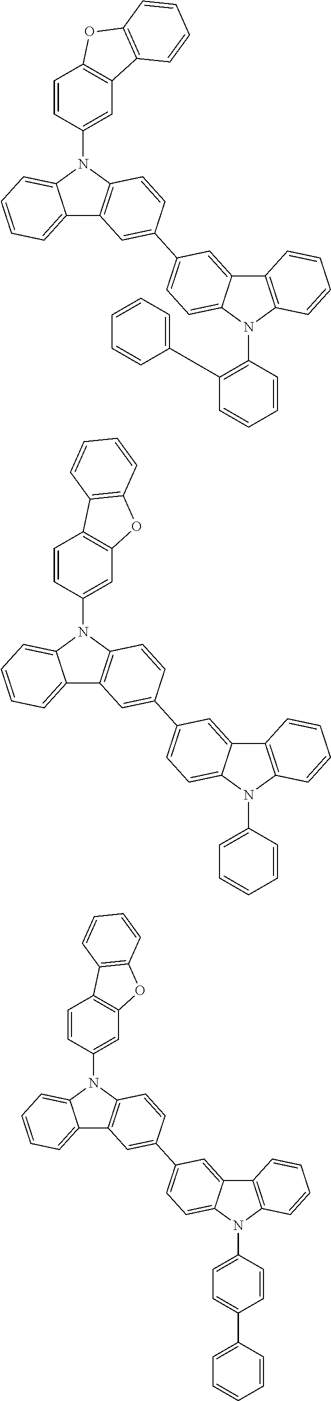 [SMILES: c1ccc(-c2ccccc2-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc5c(c4)oc4ccccc45)ccc32)cc1 ; c1ccc(-c2ccc(-n3c4ccccc4c4cc(-c5ccc6c(c5)c5ccccc5n6-c5ccc6c(c5)oc5ccccc56)ccc43)cc2)cc1]

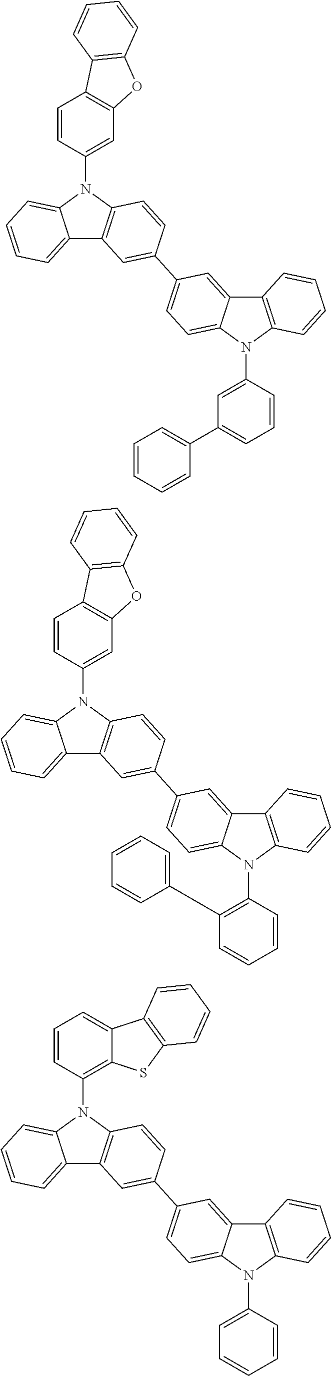

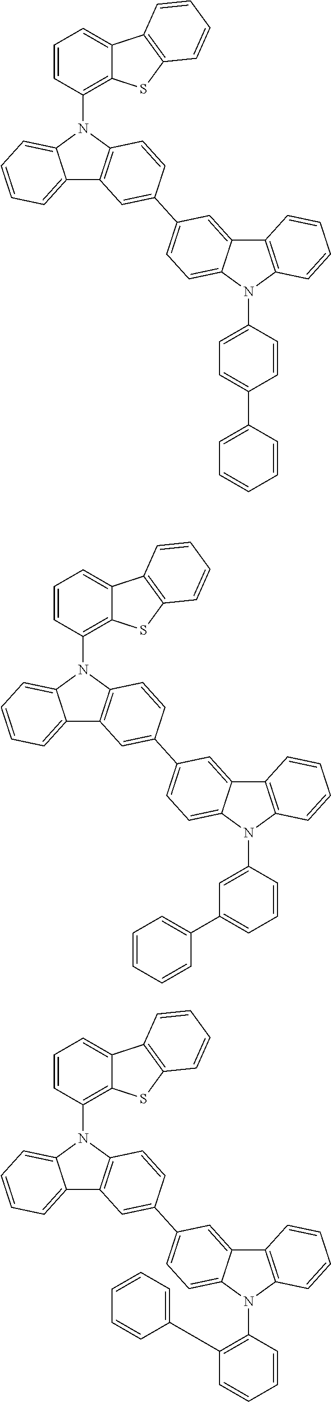

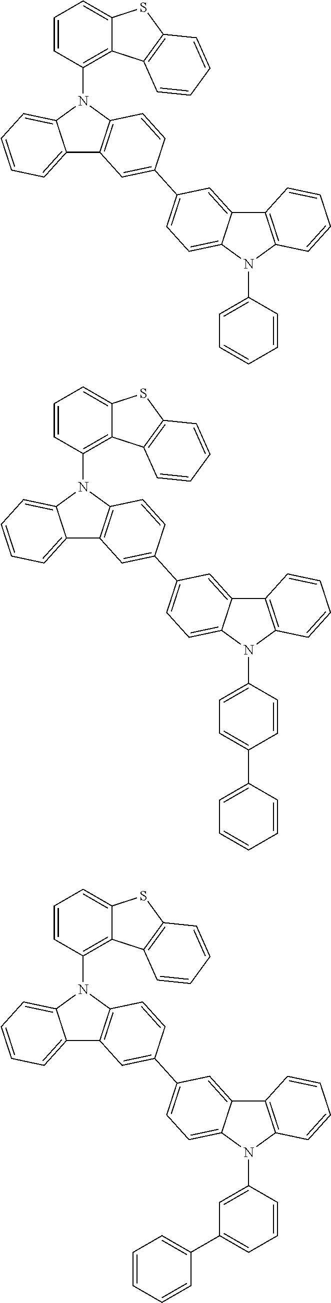 [SMILES: c1ccc(-n2c3ccccc3c3cc(-c4ccc5c(c4)c4ccccc4n5-c4cccc5sc6ccccc6c45)ccc32)cc1 ; c1ccc(-c2ccc(-n3c4ccccc4c4cc(-c5ccc6c(c5)c5ccccc5n6-c5cccc6sc7ccccc7c56)ccc43)cc2)cc1 ; c1ccc(-c2cccc(-n3c4ccccc4c4cc(-c5ccc6c(c5)c5ccccc5n6-c5cccc6sc7ccccc7c56)ccc43)c2)cc1]

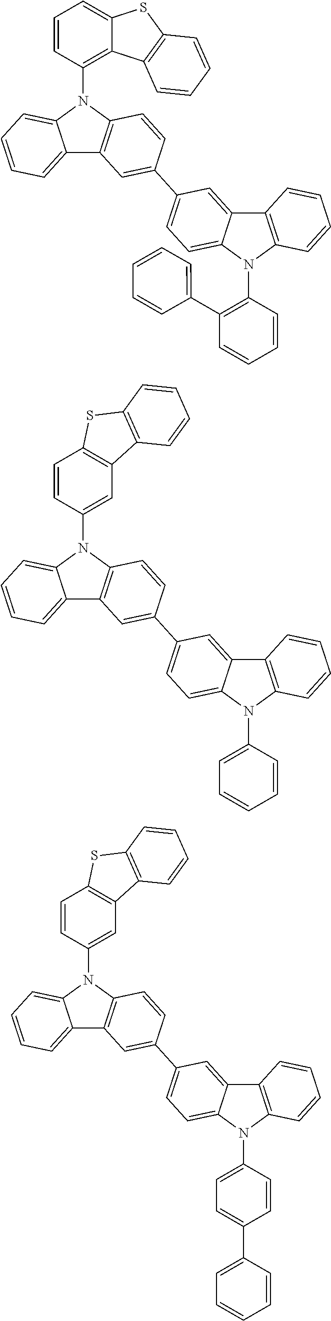 [SMILES: c1ccc(-c2ccccc2-n2c3ccccc3c3cc(-c4ccc5c(c4)c4ccccc4n5-c4cccc5sc6ccccc6c45)ccc32)cc1 ; c1ccc(-n2c3ccccc3c3cc(-c4ccc5c(c4)c4ccccc4n5-c4ccc5sc6ccccc6c5c4)ccc32)cc1 ; c1ccc(-c2ccc(-n3c4ccccc4c4cc(-c5ccc6c(c5)c5ccccc5n6-c5ccc6sc7ccccc7c6c5)ccc43)cc2)cc1]

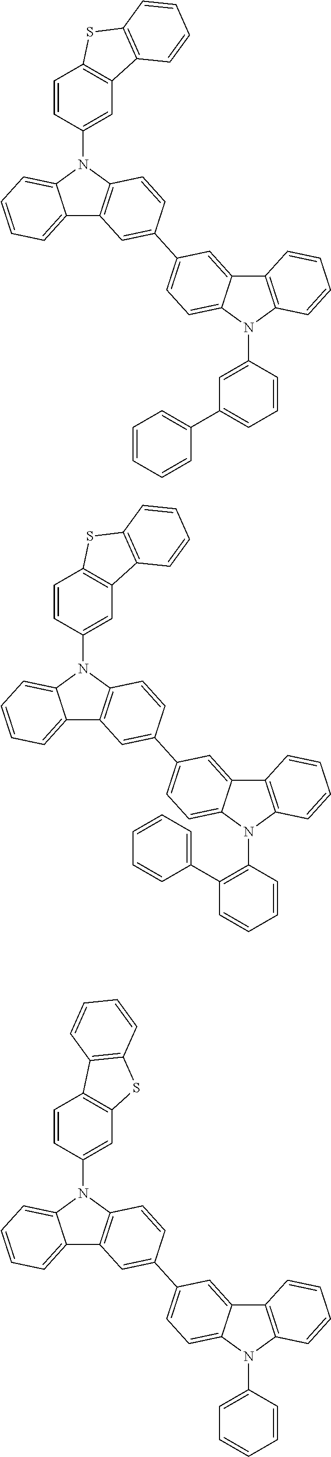

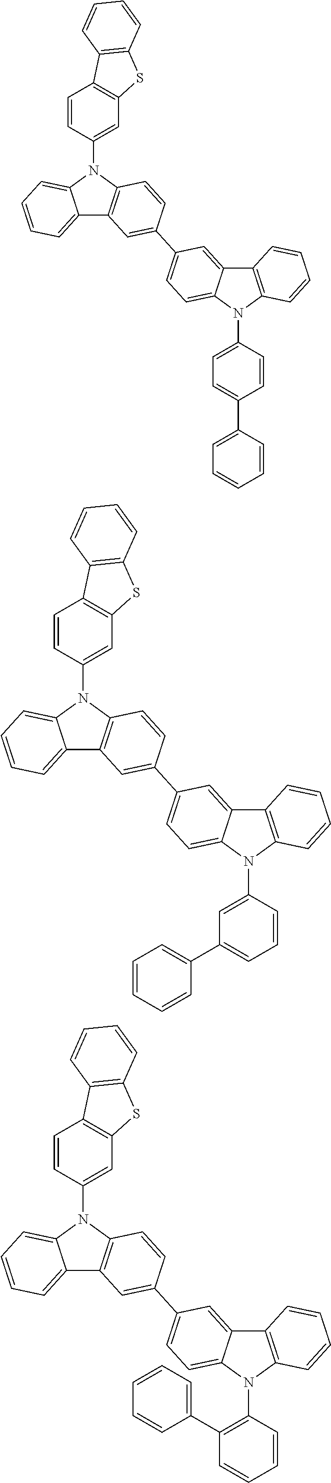

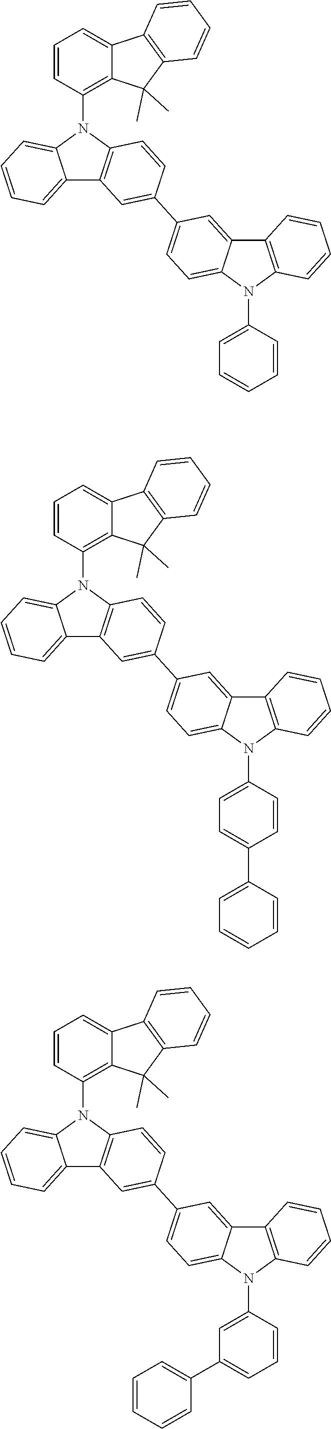 [SMILES: CC1(C)c2ccccc2-c2cccc(-n3c4ccccc4c4cc(-c5ccc6c(c5)c5ccccc5n6-c5ccccc5)ccc43)c21 ; CC1(C)c2ccccc2-c2cccc(-n3c4ccccc4c4cc(-c5ccc6c(c5)c5ccccc5n6-c5ccc(-c6ccccc6)cc5)ccc43)c21 ; CC1(C)c2ccccc2-c2cccc(-n3c4ccccc4c4cc(-c5ccc6c(c5)c5ccccc5n6-c5cccc(-c6ccccc6)c5)ccc43)c21]

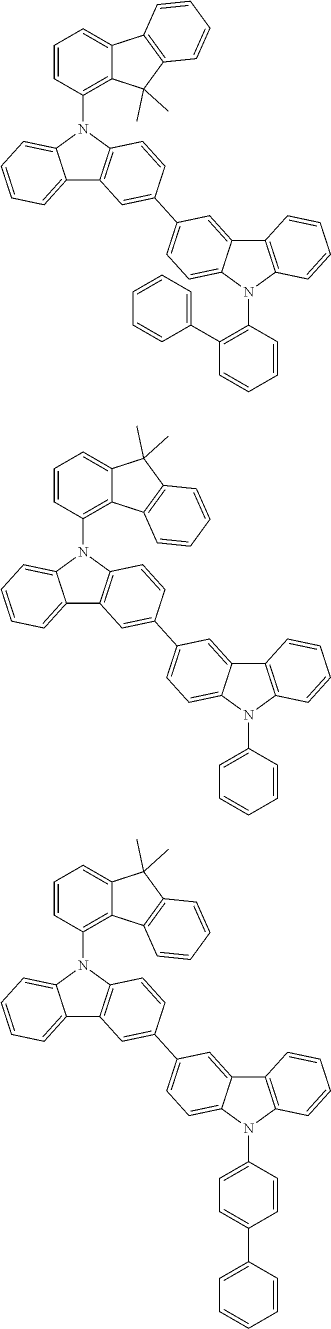 [SMILES: CC1(C)c2ccccc2-c2cccc(-n3c4ccccc4c4cc(-c5ccc6c(c5)c5ccccc5n6-c5ccccc5-c5ccccc5)ccc43)c21 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2c(-n3c4ccccc4c4cc(-c5ccc6c(c5)c5ccccc5n6-c5ccc(-c6ccccc6)cc5)ccc43)cccc21]

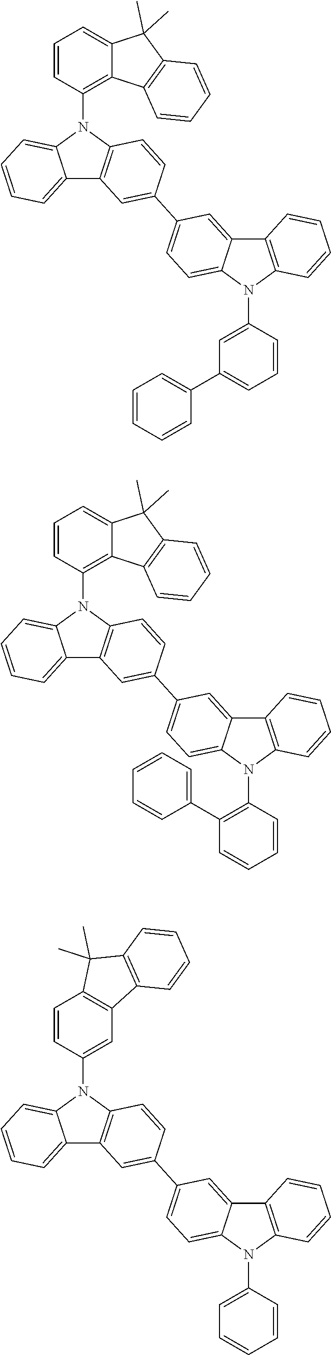 [SMILES: CC1(C)c2ccccc2-c2c(-n3c4ccccc4c4cc(-c5ccc6c(c5)c5ccccc5n6-c5cccc(-c6ccccc6)c5)ccc43)cccc21 ; CC1(C)c2ccccc2-c2c(-n3c4ccccc4c4cc(-c5ccc6c(c5)c5ccccc5n6-c5ccccc5-c5ccccc5)ccc43)cccc21 ; CC1(C)c2ccccc2-c2cc(-n3c4ccccc4c4cc(-c5ccc6c(c5)c5ccccc5n6-c5ccccc5)ccc43)ccc21]

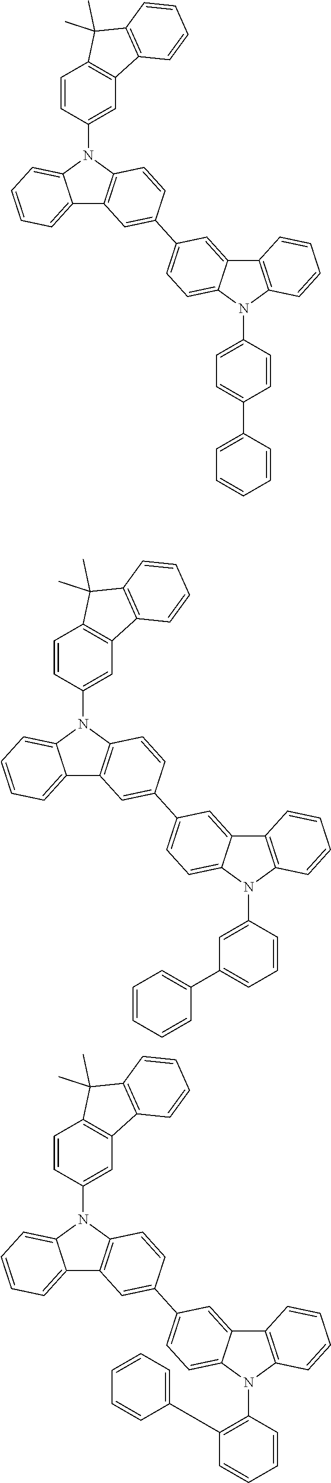

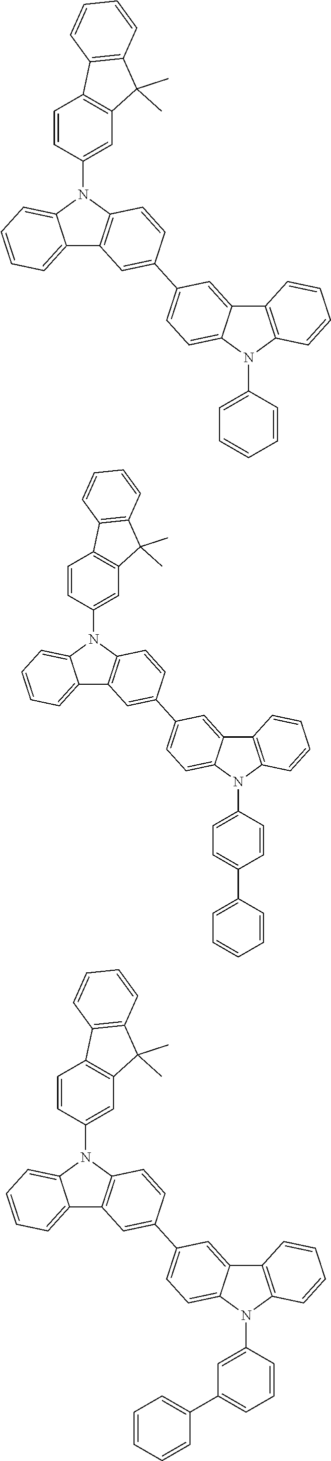 [SMILES: CC1(C)c2ccccc2-c2ccc(-n3c4ccccc4c4cc(-c5ccc6c(c5)c5ccccc5n6-c5ccccc5)ccc43)cc21 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; CC1(C)c2ccccc2-c2ccc(-n3c4ccccc4c4cc(-c5ccc6c(c5)c5ccccc5n6-c5cccc(-c6ccccc6)c5)ccc43)cc21]

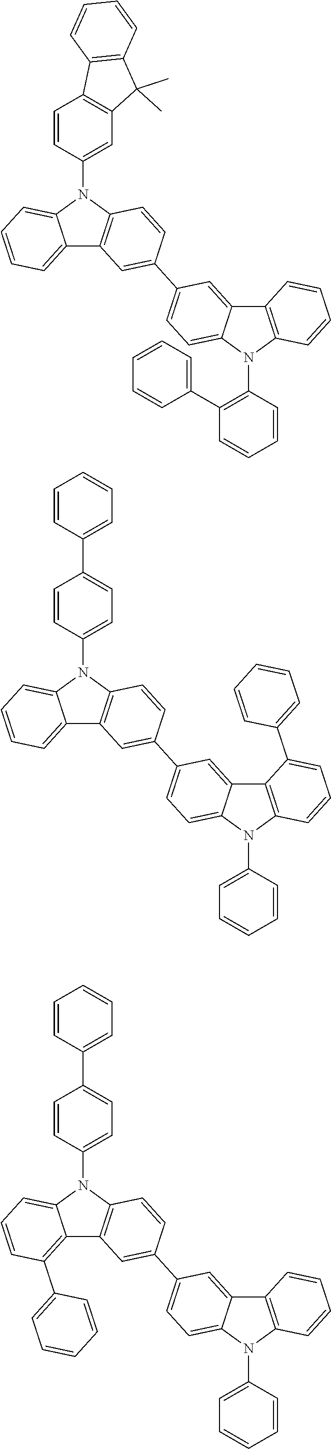 [SMILES: CC1(C)c2ccccc2-c2ccc(-n3c4ccccc4c4cc(-c5ccc6c(c5)c5ccccc5n6-c5ccccc5-c5ccccc5)ccc43)cc21 ; c1ccc(-c2ccc(-n3c4ccccc4c4cc(-c5ccc6c(c5)c5c(-c7ccccc7)cccc5n6-c5ccccc5)ccc43)cc2)cc1 ; c1ccc(-c2ccc(-n3c4ccc(-c5ccc6c(c5)c5ccccc5n6-c5ccccc5)cc4c4c(-c5ccccc5)cccc43)cc2)cc1]

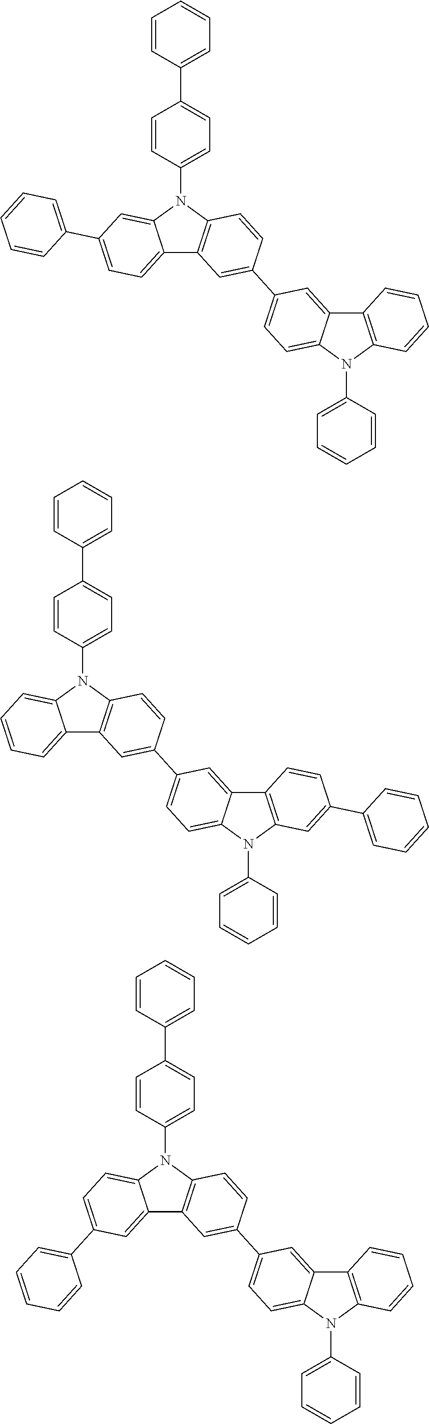 [SMILES: c1ccc(-c2ccc(-n3c4ccc(-c5ccc6c(c5)c5ccccc5n6-c5ccccc5)cc4c4ccc(-c5ccccc5)cc43)cc2)cc1 ; c1ccc(-c2ccc(-n3c4ccccc4c4cc(-c5ccc6c(c5)c5ccc(-c7ccccc7)cc5n6-c5ccccc5)ccc43)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1]

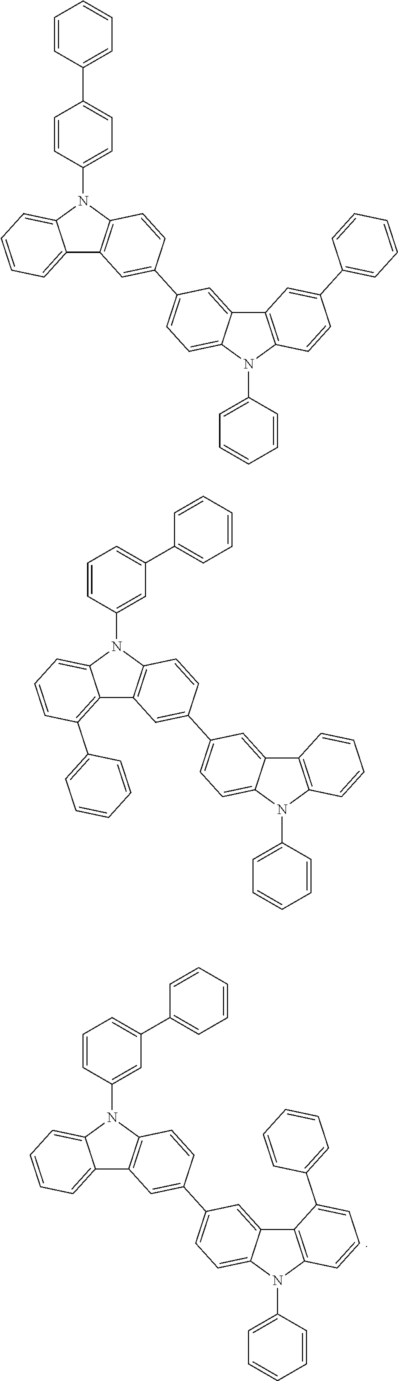 [SMILES: c1ccc(-c2ccc(-n3c4ccccc4c4cc(-c5ccc6c(c5)c5cc(-c7ccccc7)ccc5n6-c5ccccc5)ccc43)cc2)cc1 ; c1ccc(-c2cccc(-n3c4ccc(-c5ccc6c(c5)c5ccccc5n6-c5ccccc5)cc4c4c(-c5ccccc5)cccc43)c2)cc1 ; c1ccc(-c2cccc(-n3c4ccccc4c4cc(-c5ccc6c(c5)c5c(-c7ccccc7)cccc5n6-c5ccccc5)ccc43)c2)cc1]

The compound of Chemical Formula 1 and the compound of Chemical Formula 2, which are two kinds of host materials, can be contained in the light emitting layer at a weight ratio of 10:90 to 90:10, for example, at a weight ratio of 50:50.

Examples of the dopant material include an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

Further, the hole blocking layer refers to a layer which is formed on the light emitting layer, and preferably, is provided in contact with the light emitting layer, and thus severs to control electron mobility, to prevent excessive movement of holes, and to increase the probability of hole-electron bonding, thereby improving the efficiency of the organic light emitting device. The hole blocking layer includes a hole blocking material, and as an example of such hole blocking material, a compound into which an electron-withdrawing group is introduced, such as azine derivatives including triazine; triazole derivatives; oxadiazole derivatives; phenanthroline derivatives; phosphine oxide derivatives can be used, but is not limited thereto.

Further, the electron transport layer is a layer that receives the electrons from the electron injection layer and transports the electrons to the light emitting layer, and is formed on the light emitting layer and the hole blocking layer. The electron transport layer includes an electron transport material, and such an electron transport material is suitably a material which has a large mobility for electrons. Specific examples of the electron transport material include: an Al complex of 8-hydroxyquinoline; a complex including Alq₃; an organic radical compound; a hydroxyflavone-metal complex, and the like, but are not limited thereto. Alternatively, fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like can be used together as the electron transport material, but are not limited thereto.

The electron injection layer is a layer that inject electrons from an electrode and is formed on the electron transport layer. Examples of the electron injection material included in the electron injection layer include LiF, NaCl, CsF, Li₂O, BaO, fluorenone, anthraquinodimethane, diphenoquinone, thiopyran dioxide, oxazole, oxadiazole, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Here, the metal complex compound includes 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but is not limited thereto.

The organic light emitting device according to the present disclosure can be a bottom emission type device, a top emission type device, or a double side emission type device, and in particular, it can be a bottom emission type light emitting device that requires relatively high luminous efficiency.

In addition, the compound of Chemical Formula 1 can be included in an organic solar cell or an organic transistor in addition to an organic light emitting device.

The preparation of the compound of Chemical Formula 1 and the organic light emitting device including the same will be described in detail in the following examples. However, these examples are presented for illustrative purposes only, and are not intended to limit the scope of the present disclosure.

### Preparation Example 1: Preparation of Compound A-3

1) Preparation of Compound A-1

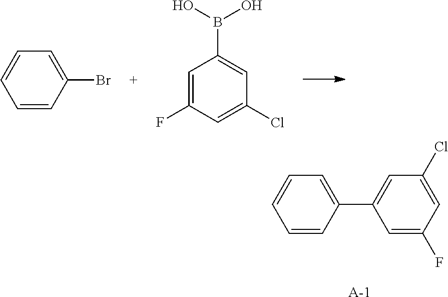 [SMILES: Brc1ccccc1 ; OB(O)c1cc(F)cc(Cl)c1 ; Fc1cc(Cl)cc(-c2ccccc2)c1 ; "A-1"]

Bromobenzene (100 g, 641.2 mmol) and (3-chloro-5-fluorophenyl)boronic acid (111.6 g, 641.2 mmol) were added to 2000 mL of tetrahydrofuran under a nitrogen atmosphere, and the mixture was stirred and refluxed. Then, potassium carbonate (265.9 g, 1923.6 mmol) was dissolved in 266 mL of water and added thereto, and the mixture was sufficiently stirred and then tetrakistriphenyl-phosphinopalladium (22.2 g, 19.2 mmol) was added. After reacting for 1 hour, the reaction mixture was cooled to room temperature, and then the organic layer and the aqueous layer were separated, and the organic layer was distilled. This was again added to 2642 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid compound A-1 (80.6 g, 61%, MS: [M+H]⁺=207).

2) Preparation of Compound A-2

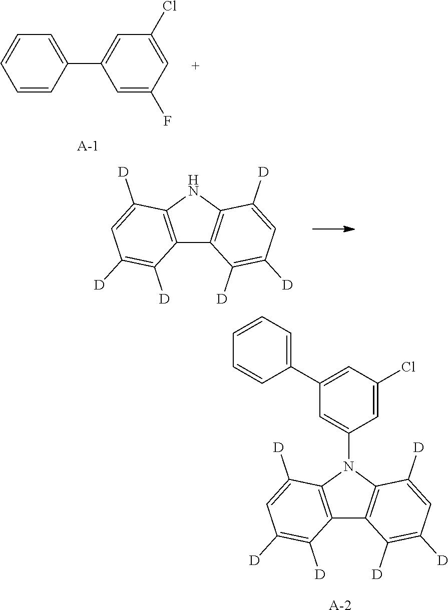

A-1 (100 g, 485.4 mmol) and 9H-carbazole-1,3,4,5,6,8-d6 (84 g, 485.4 mmol) were added to 2000 mL of xylene under a nitrogen atmosphere, and the mixture was stirred and refluxed. Then, sodium tert-butoxide (140 g, 1456.1 mmol) was added thereto, and the mixture was sufficiently stirred, and then bis(tri-tert-butylphosphine)palladium (7.4 g, 14.6 mmol) was added. After reacting for 4 hours, the reaction mixture was cooled to room temperature and the resulting solid was filtered. The solid was added to 5229 mL of chloroform, dissolved, washed twice with water, the organic layer was separated, anhydrous magnesium sulfate was added, stirred, filtered, and the filtrate was distilled under reduced pressure. The concentrated compound was purified through a silica column using chloroform and ethyl acetate to prepare a white solid compound A-2 (111.6 g, 64%, MS: [M+H]⁺=360.1).

3) Preparation of Compound A-3

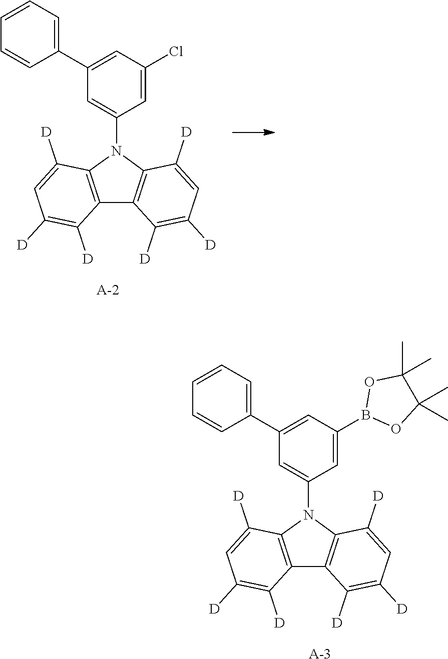

A-2 (50 g, 139.2 mmol) and bis(pinacolato)diboron (38.9 g, 153.1 mmol) were added to 1000 mL of 1,4-dioxane under a nitrogen atmosphere, and the mixture was stirred and refluxed. Then, potassium acetate (40.1 g, 417.7 mmol) was added thereto, and the mixture was sufficiently stirred and then palladium dibenzylideneacetone palladium (2.4 g, 4.2 mmol) and tricyclohexylphosphine (2.3 g, 8.4 mmol) were added. After reacting for 5 hours, the reaction mixture was subjected to filtration treatment to remove a salt, and then the filtered organic layer was distilled. This was added again to 628 mL of chloroform, dissolved and washed twice with water. The organic layer was then separated, anhydrous magnesium sulfate was added, stirred and then filtered. The filtrate was distilled under reduced pressure. The concentrated compound was recrystallized from chloroform and ethanol to prepare a gray solid compound A-3 (51.5 g, 82%, MS: [M+H]⁺=452.3).

### Preparation Example 2: Preparation of Compound B-3

1) Preparation of Compound B-1

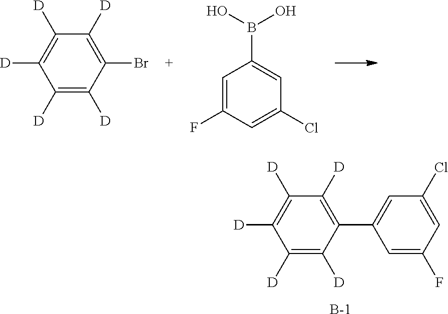

1-Bromobenzene-2,3,4,5,6-d5 (100 g, 621.2 mmol) and (3-chloro-5-fluorophenyl)boronic acid (108.1 g, 621.2 mmol) were added to 2000 mL of tetrahydrofuran under a nitrogen atmosphere, and the mixture was stirred and refluxed. Then, potassium carbonate (257.6 g, 1863.5 mmol) was dissolved in 258 mL of water and added thereto, and the mixture was sufficiently stirred and then tetrakistriphenyl-phosphinopalladium (21.5 g, 18.6 mmol) was added. After reacting for 1 hour, the reaction mixture was cooled to room temperature, and then the organic layer and the aqueous layer were separated, and the organic layer was distilled. This was again added to 2622 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid compound B-1 (80 g, 61%, MS: [M+H]⁺=212.1).

2) Preparation of Compound B-2

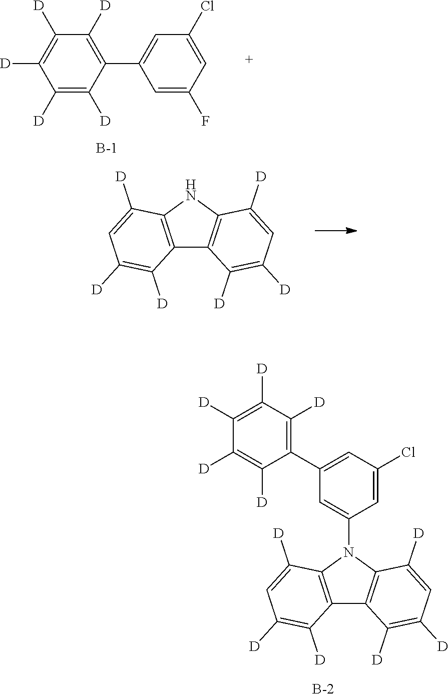

B-1 (100 g, 473.8 mmol) and 9H-carbazole-1,3,4,5,6,8-d6 (82 g, 473.8 mmol) were added to 2000 mL of xylene under a nitrogen atmosphere, and the mixture was stirred and refluxed. Then, sodium tert-butoxide (136.6 g, 1421.4 mmol) was added thereto, and the mixture was sufficiently stirred, and then bis(tri-tert-butylphosphine)palladium (7.3 g, 14.2 mmol) was added. After reacting for 1 hours, the reaction mixture was cooled to room temperature and the resulting solid was filtered. The solid was added to 5176 mL of chloroform, dissolved, washed twice with water, the organic layer was separated, anhydrous magnesium sulfate was added, stirred, filtered, and the filtrate was distilled under reduced pressure. The concentrated compound was purified through a silica column using chloroform and ethyl acetate to prepare a white solid compound B-2 (103.5 g, 60%, MS: [M+H]⁺=365.2).

3) Preparation of Compound B-3

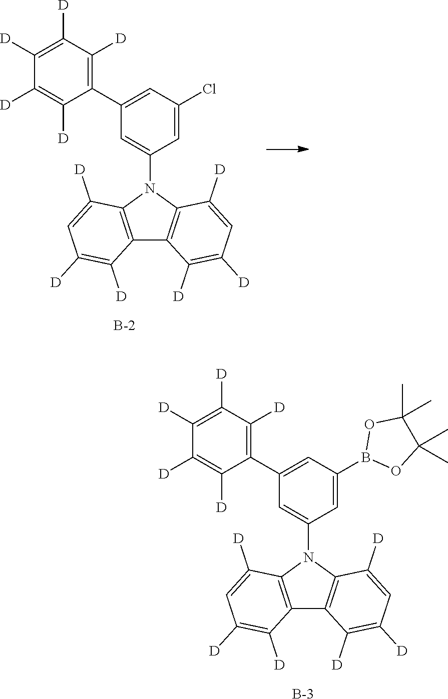

B-2 (50 g, 137.3 mmol) and bis(pinacolato)diboron (38.4 g, 151 mmol) were added to 1000 mL of 1,4-dioxane under a nitrogen atmosphere, and the mixture was stirred and refluxed. Then, potassium acetate (39.6 g, 411.9 mmol) was added thereto, and the mixture was sufficiently stirred and then palladium dibenzylideneacetone palladium (2.4 g, 4.1 mmol) and tricyclohexylphosphine (2.3 g, 8.2 mmol) were added. After reacting for 4 hours, the reaction mixture was subjected to filtration treatment to remove a salt, and then the filtered organic layer was distilled. This was added again to 626 mL of chloroform, dissolved and washed twice with water. The organic layer was then separated, anhydrous magnesium sulfate was added, stirred and then filtered. The filtrate was distilled under reduced pressure. The concentrated compound was recrystallized from chloroform and ethanol to prepare a gray solid compound B-3 (32 g, 51%, MS: [M+H]⁺=457.3).

### Preparation Example 3: Preparation of Compound C-3

1) Preparation of Compound C-2

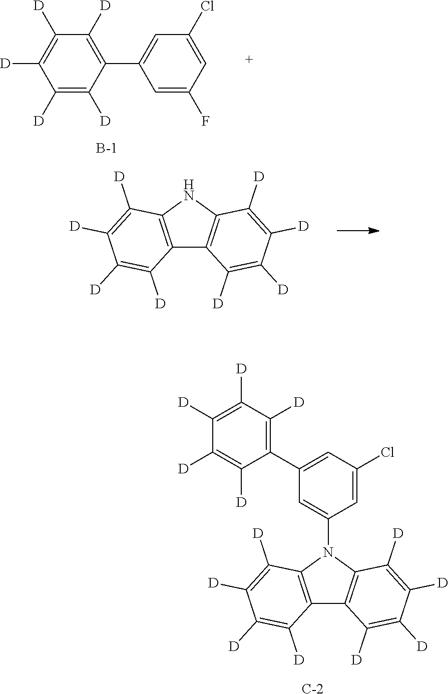

B-1 (100 g, 473.8 mmol) and 9H-carbazole-1,2,3,4,5,6,7,8-d8 (83 g, 473.8 mmol) were added to 2000 mL of xylene under a nitrogen atmosphere, and the mixture was stirred and refluxed. Then, sodium tert-butoxide (136.6 g, 1421.4 mmol) was added thereto, and the mixture was sufficiently stirred, and then bis(tri-tert-butylphosphine)palladium (7.3 g, 14.2 mmol) was added. After reacting for 5 hours, the reaction mixture was cooled to room temperature and the resulting solid was filtered. The solid was added to 5205 mL of chloroform, dissolved, washed twice with water, the organic layer was separated, anhydrous magnesium sulfate was added, stirred, filtered, and the filtrate was distilled under reduced pressure. The concentrated compound was purified through a silica column using chloroform and ethyl acetate to prepare a white solid compound C-2 (131.9 g, 76%, MS: [M+H]⁺=367.2).

2) Preparation of Compound C-3

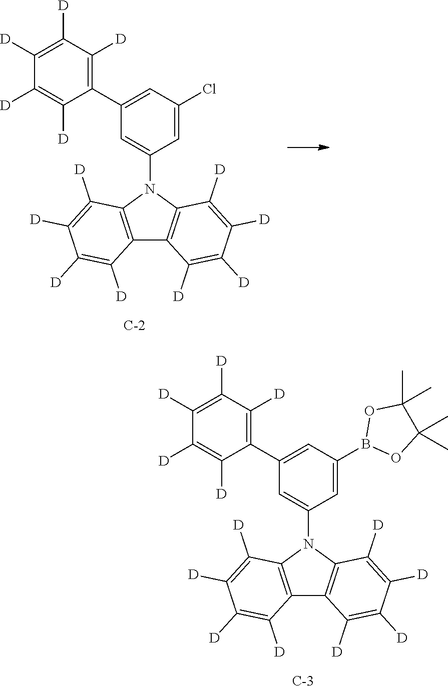

C-2 (50 g, 137.3 mmol) and bis(pinacolato)diboron (38.4 g, 151 mmol) were added to 1000 mL of 1,4-dioxane under a nitrogen atmosphere, and the mixture was stirred and refluxed. Then, potassium acetate (39.6 g, 411.9 mmol) was added thereto, and the mixture was sufficiently stirred and then palladium dibenzylideneacetone palladium (2.4 g, 4.1 mmol) and tricyclohexylphosphine (2.3 g, 8.2 mmol) were added. After reacting for 5 hours, the reaction mixture was subjected to filtration treatment to remove a salt, and then the filtered organic layer was distilled. This was added again to 626 mL of chloroform, dissolved and washed twice with water. The organic layer was then separated, anhydrous magnesium sulfate was added, stirred and then filtered. The filtrate was distilled under reduced pressure. The concentrated compound was recrystallized from chloroform and ethanol to prepare a gray solid compound C-3 (48.9 g, 78%, MS: [M+H]⁺=457.3).

### Preparation Example 4: Preparation of Compound D-3

1) Preparation of Compound D-1

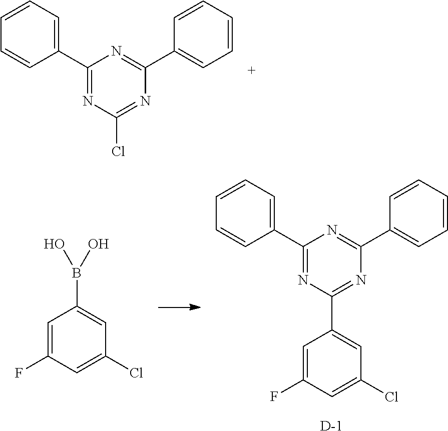

2-Chloro-4,6-diphenyl-1,3,5-triazine (100 g, 200.3 mmol) and (3-chloro-5-fluorophenyl)boronic acid (68.7 g, 200.3 mmol) were added to 2000 mL of tetrahydrofuran under a nitrogen atmosphere, and the mixture was stirred and refluxed. Then, potassium carbonate (83.1 g, 601 mmol) was dissolved in 83 mL of water and added thereto, and the mixture was sufficiently stirred and then tetrakistriphenyl-phosphinopalladium (6.9 g, 6 mmol) was added. After reacting for 2 hours, the reaction mixture was cooled to room temperature, and then the organic layer and the aqueous layer were separated, and the organic layer was distilled. This was again added to 1447 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid compound D-1 (41.2 g, 57%, MS: [M+H]⁺=362.1).

2) Preparation of Compound D-2

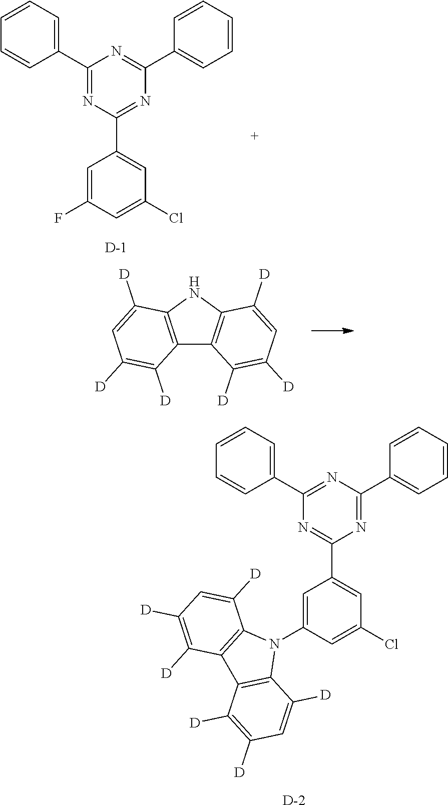

D-1 (30 g, 83.1 mmol) and 9H-carbazole-1,3,4,5,6,8-d6 (14.4 g, 83.1 mmol) were added to 600 mL of dimethylformamide under a nitrogen atmosphere, and the mixture was stirred and refluxed. Then, potassium triphosphate (52.9 g, 249.3 mmol) was added thereto and the mixture was sufficiently stirred. After reacting for 5 hours, the reaction mixture was cooled to room temperature, and the organic layer was subjected to filtration treatment to remove a salt, and then the filtered organic layer was distilled. This was added again to 427 mL of chloroform, dissolved and washed twice with water. The organic layer was then separated, anhydrous magnesium sulfate was added, stirred and then filtered. The filtrate was distilled under reduced pressure. The concentrated compound was recrystallized from chloroform and ethyl acetate to prepare a gray solid compound D-2 (25.6 g, 60%, MS: [M+H]⁺=515.2).

3) Preparation of Compound D-3

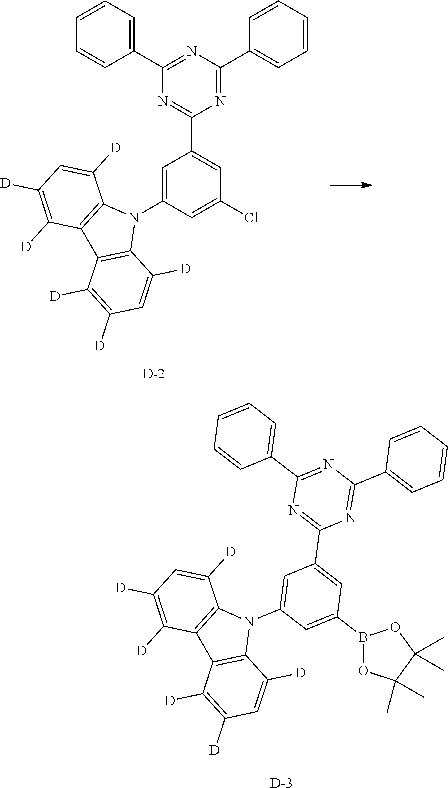

D-2 (50 g, 97.2 mmol) and bis(pinacolato)diboron (27.2 g, 107 mmol) were added to 1000 mL of 1,4-dioxane under a nitrogen atmosphere, and the mixture was stirred and refluxed. Then, potassium acetate (28 g, 291.7 mmol) was added thereto, and the mixture was sufficiently stirred and then palladium dibenzylideneacetone palladium (1.7 g, 2.9 mmol) and tricyclohexylphosphine (1.6 g, 5.8 mmol) were added. After reacting for 7 hours, the reaction mixture was subjected to filtration treatment to remove a salt, and then the filtered organic layer was distilled. This was added again to 590 mL of chloroform, dissolved and washed twice with water. The organic layer was then separated, anhydrous magnesium sulfate was added, stirred and then filtered. The filtrate was distilled under reduced pressure. The concentrated compound was recrystallized from chloroform and ethanol to prepare a gray solid compound D-3 (35.4 g, 60%, MS: [M+H]⁺=607.3).

### Synthesis Example 1: Preparation of Compound 1

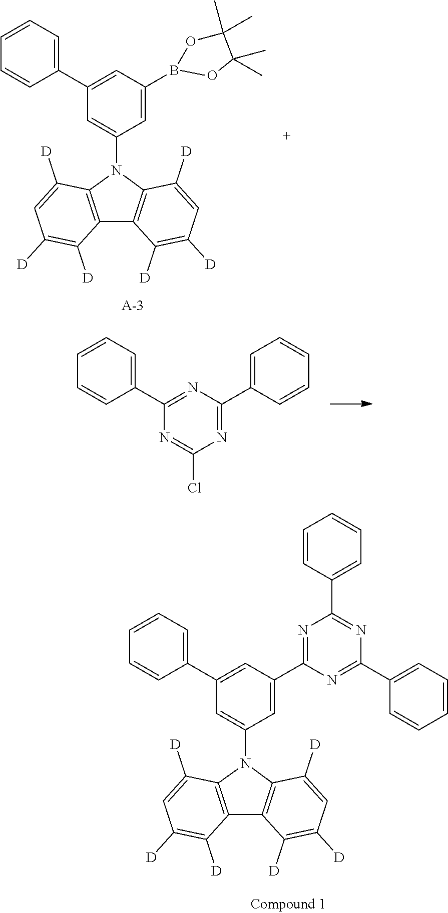

A-3 (20 g, 44.3 mmol) and 2-chloro-4,6-diphenyl-1,3,5-triazine (11.8 g, 44.3 mmol) were added to 400 mL of tetrahydrofuran under a nitrogen atmosphere, and the mixture was stirred and refluxed. Then, potassium carbonate (18.4 g, 133 mmol) was dissolved in 18 mL of water and added thereto, and the mixture was sufficiently stirred and then tetrakistriphenyl-phosphinopalladium (1.5 g, 1.3 mmol) was added. After reacting for 3 hour, the reaction mixture was cooled to room temperature, and then the organic layer and the aqueous layer were separated, and the organic layer was distilled. This was again added to 1233 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 1 (19.2 g, 78%, MS: [M+H]⁺=557.3).

### Synthesis Example 2: Preparation of Compound 2

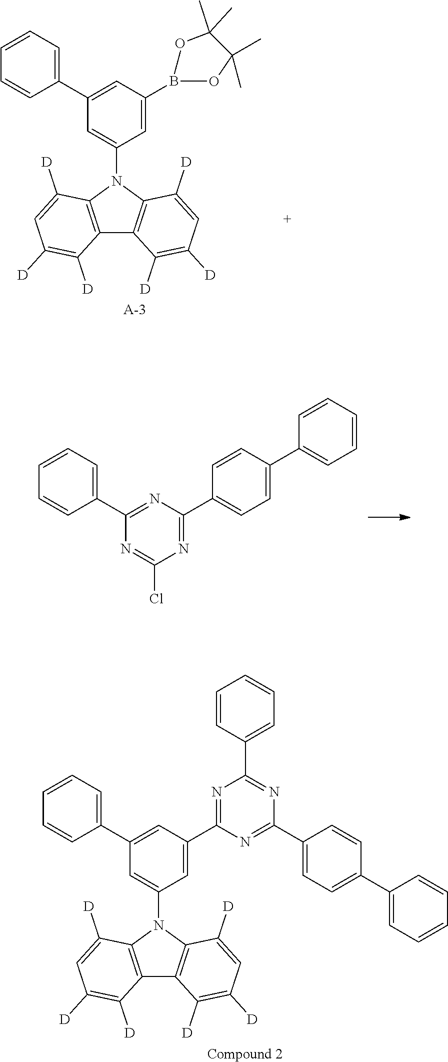

A-3 (20 g, 44.3 mmol) and 2-([1,1′-biphenyl]-4-yl)-4-chloro-6-phenyl-1,3,5-triazine (15.2 g, 44.3 mmol) were added to 400 mL of tetrahydrofuran under a nitrogen atmosphere, and the mixture was stirred and refluxed. Then, potassium carbonate (18.4 g, 133 mmol) was dissolved in 18 mL of water and added thereto, and the mixture was sufficiently stirred and then tetrakistriphenyl-phosphinopalladium (1.5 g, 1.3 mmol) was added. After reacting for 2 hours, the reaction mixture was cooled to room temperature, and then the organic layer and the aqueous layer were separated, and the organic layer was distilled. This was again added to 1401 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 2 (17.1 g, 61%, MS: [M+H]⁺=633.3).

### Synthesis Example 3: Preparation of Compound 3

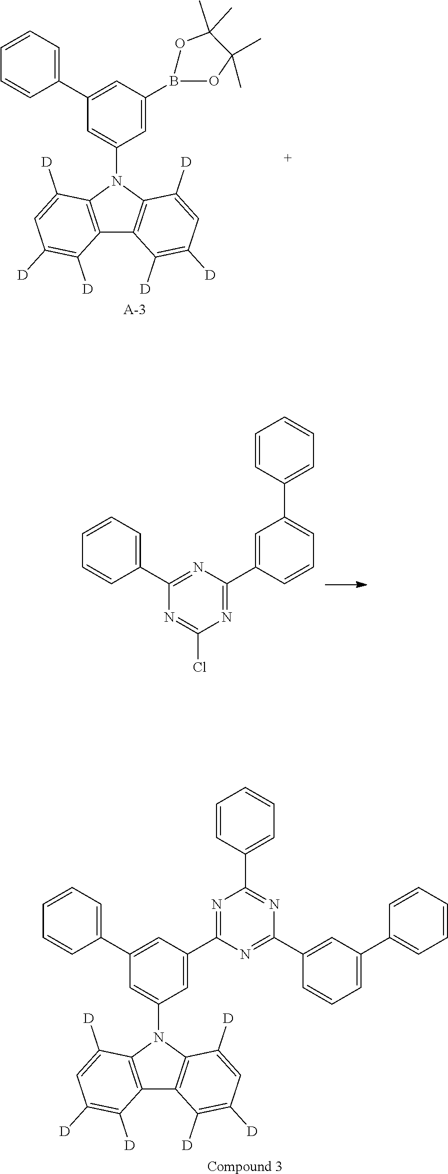

A-3 (20 g, 44.3 mmol) and 2-([1,1′-biphenyl]-4-yl)-3-chloro-6-phenyl-1,3,5-triazine (15.2 g, 44.3 mmol) were added to 400 mL of tetrahydrofuran under a nitrogen atmosphere, and the mixture was stirred and refluxed. Then, potassium carbonate (18.4 g, 133 mmol) was dissolved in 18 mL of water and added thereto, and the mixture was sufficiently stirred and then tetrakistriphenyl-phosphinopalladium (1.5 g, 1.3 mmol) was added. After reacting for 3 hour, the reaction mixture was cooled to room temperature, and then the organic layer and the aqueous layer were separated, and the organic layer was distilled. This was again added to 1401 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 3 (15.7 g, 56%, MS: [M+H]⁺=633.3).

### Synthesis Example 4: Preparation of Compound 4

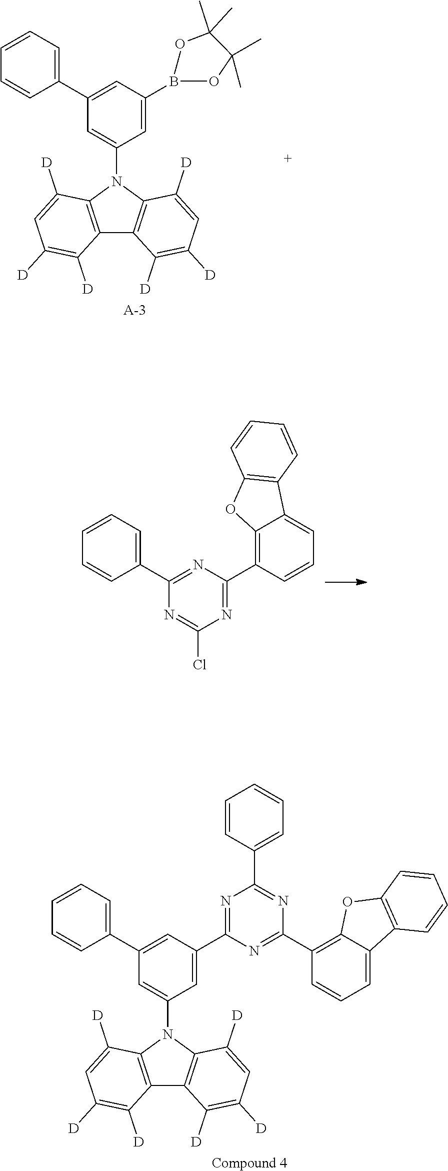

A-3 (20 g, 44.3 mmol) and 2-chloro-4-(dibenzo[b,d]furan-4-yl)-6-phenyl-1,3,5-triazine (15.8 g, 44.3 mmol) were added to 400 mL of tetrahydrofuran under a nitrogen atmosphere, and the mixture was stirred and refluxed. Then, potassium carbonate (18.4 g, 133 mmol) was dissolved in 18 mL of water and added thereto, and the mixture was sufficiently stirred and then tetrakistriphenyl-phosphinopalladium (1.5 g, 1.3 mmol) was added. After reacting for 3 hour, the reaction mixture was cooled to room temperature, and then the organic layer and the aqueous layer were separated, and the organic layer was distilled. This was again added to 1432 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 4 (14.9 g, 52%, MS: [M+H]⁺=647.3).

### Synthesis Example 5: Preparation of Compound 5

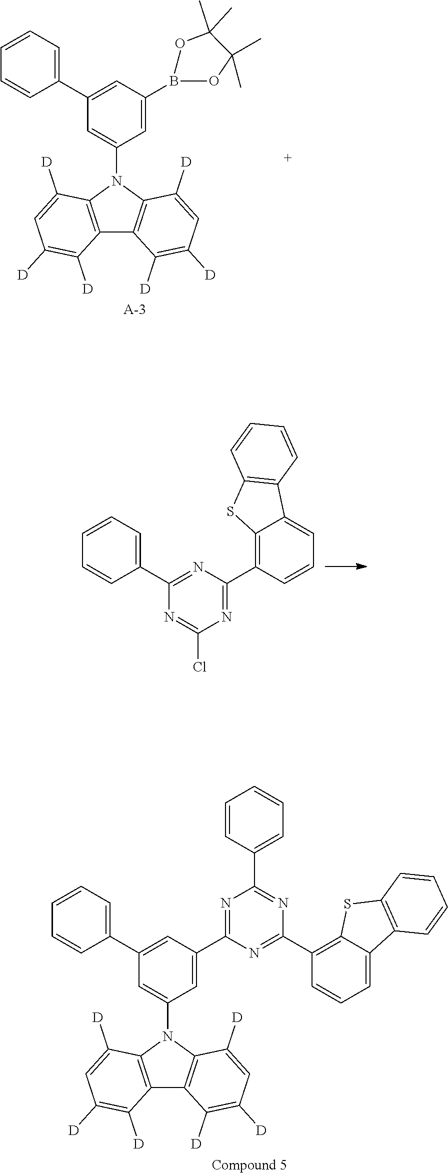

A-3 (20 g, 44.3 mmol) and 2-chloro-4-(dibenzo[b,d]thiophen-4-yl)-6-phenyl-1,3,5-triazine (16.5 g, 44.3 mmol) were added to 400 mL of tetrahydrofuran under a nitrogen atmosphere, and the mixture was stirred and refluxed. Then, potassium carbonate (18.4 g, 133 mmol) was dissolved in 18 mL of water and added thereto, and the mixture was sufficiently stirred and then tetrakistriphenyl-phosphinopalladium (1.5 g, 1.3 mmol) was added. After reacting for 3 hour, the reaction mixture was cooled to room temperature, and then the organic layer and the aqueous layer were separated, and the organic layer was distilled. This was again added to 1468 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 5 (21.1 g, 72%, MS: [M+H]⁺=663.2).

### Synthesis Example 6: Preparation of Compound 6

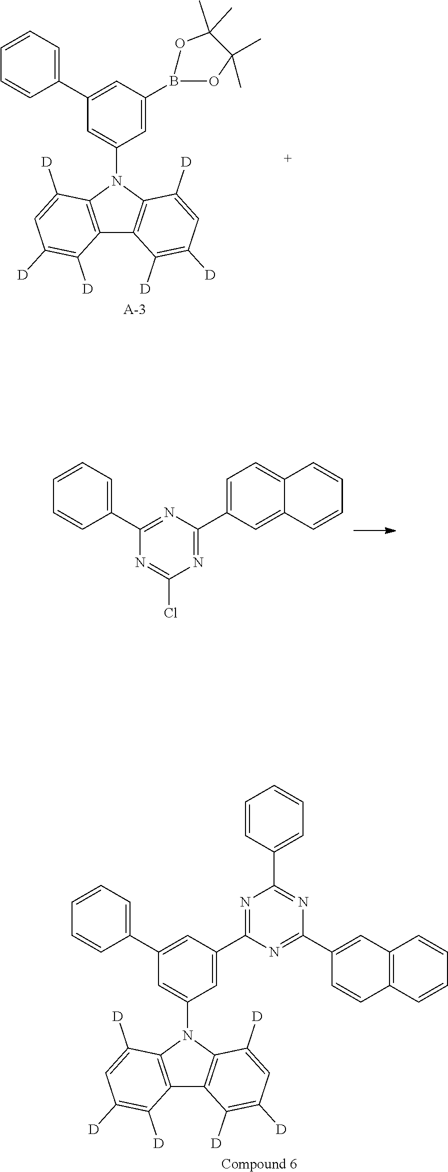

A-3 (20 g, 44.3 mmol) and 2-chloro-4-(naphthalen-2-yl)-6-phenyl-1,3,5-triazine (14.1 g, 44.3 mmol) were added to 400 mL of tetrahydrofuran under a nitrogen atmosphere, and the mixture was stirred and refluxed. Then, potassium carbonate (18.4 g, 133 mmol) was dissolved in 18 mL of water and added thereto, and the mixture was sufficiently stirred and then tetrakistriphenyl-phosphinopalladium (1.5 g, 1.3 mmol) was added. After reacting for 3 hour, the reaction mixture was cooled to room temperature, and then the organic layer and the aqueous layer were separated, and the organic layer was distilled. This was again added to 1344 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 6 (19.1 g, 71%, MS: [M+H]⁺=607.3).

### Synthesis Example 7: Preparation of Compound 7

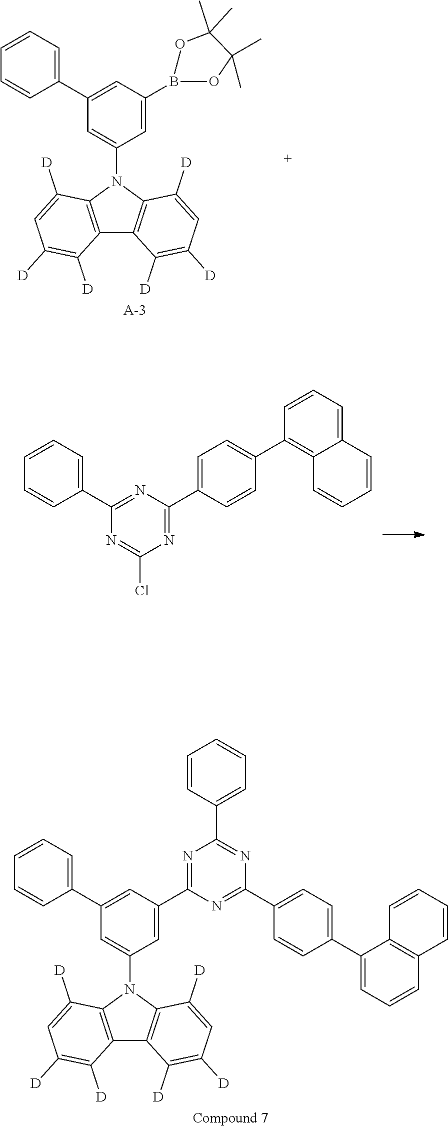

A-3 (20 g, 44.3 mmol) and 2-chloro-4-(4-(naphthalen-1-yl)phenyl)-6-phenyl-1,3,5-triazine (17.4 g, 44.3 mmol) were added to 400 mL of tetrahydrofuran under a nitrogen atmosphere, and the mixture was stirred and refluxed. Then, potassium carbonate (18.4 g, 133 mmol) was dissolved in 18 mL of water and added thereto, and the mixture was sufficiently stirred and then tetrakistriphenyl-phosphinopalladium (1.5 g, 1.3 mmol) was added. After reacting for 2 hours, the reaction mixture was cooled to room temperature, and then the organic layer and the aqueous layer were separated, and the organic layer was distilled. This was again added to 1512 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 7 (23.6 g, 78%, MS: [M+H]⁺=683.3).

### Synthesis Example 8: Preparation of Compound 8

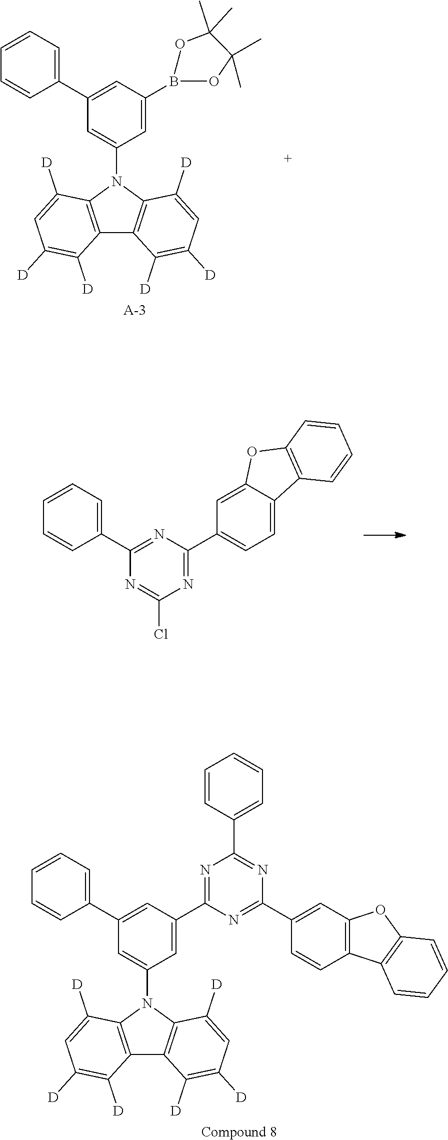

A-3 (20 g, 44.3 mmol) and 2-chloro-4-(dibenzo[b,d]furan-3-yl)-6-phenyl-1,3,5-triazine (15.8 g, 44.3 mmol) were added to 400 mL of tetrahydrofuran under a nitrogen atmosphere, and the mixture was stirred and refluxed. Then, potassium carbonate (18.4 g, 133 mmol) was dissolved in 18 mL of water and added thereto, and the mixture was sufficiently stirred and then tetrakistriphenyl-phosphinopalladium (1.5 g, 1.3 mmol) was added. After reacting for 3 hour, the reaction mixture was cooled to room temperature, and then the organic layer and the aqueous layer were separated, and the organic layer was distilled. This was again added to 1432 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 8 (18.9 g, 66%, MS: [M+H]⁺=647.3).

### Synthesis Example 9: Preparation of Compound 9

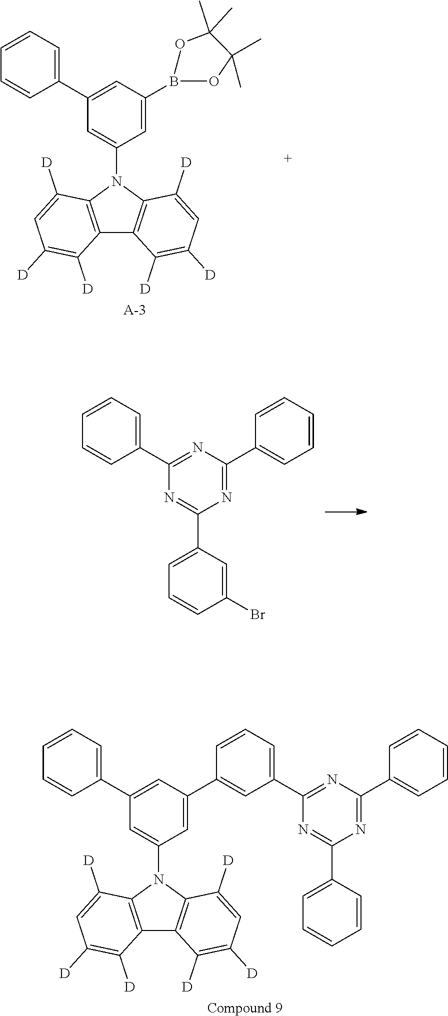

A-3 (20 g, 44.3 mmol) and 2-(3-bromophenyl)-4,6-diphenyl-1,3,5-triazine (17.2 g, 44.3 mmol) were added to 400 mL of tetrahydrofuran under a nitrogen atmosphere, and the mixture was stirred and refluxed. Then, potassium carbonate (18.4 g, 133 mmol) was dissolved in 18 mL of water and added thereto, and the mixture was sufficiently stirred and then tetrakistriphenyl-phosphinopalladium (1.5 g, 1.3 mmol) was added. After reacting for 3 hour, the reaction mixture was cooled to room temperature, and then the organic layer and the aqueous layer were separated, and the organic layer was distilled. This was again added to 1401 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 9 (15.4 g, 55%, MS: [M+H]⁺=633.3).

### Synthesis Example 10: Preparation of Compound 10

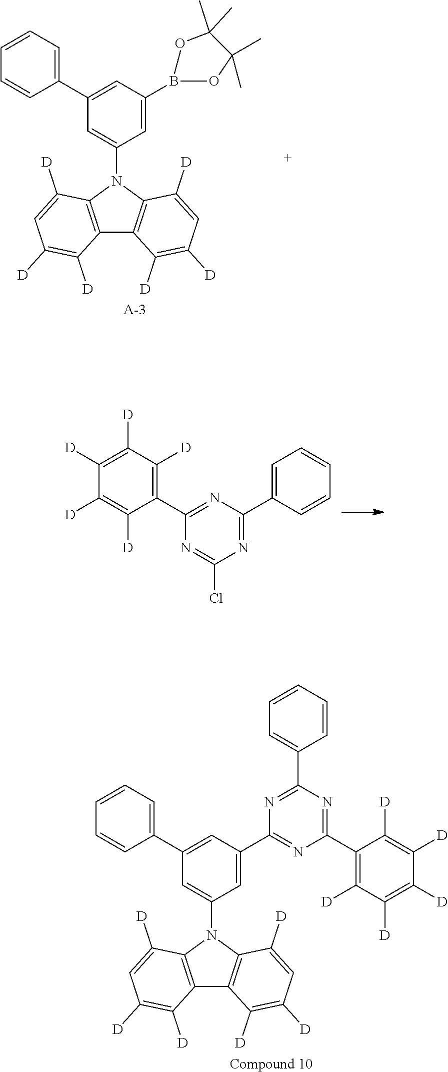

A-3 (20 g, 44.3 mmol) and 2-chloro-4-phenyl-6-(phenyl-d5)-1,3,5-triazine (12.3 g, 44.3 mmol) were added to 400 mL of tetrahydrofuran under a nitrogen atmosphere, and the mixture was stirred and refluxed. Then, potassium carbonate (18.4 g, 133 mmol) was dissolved in 18 mL of water and added thereto, and the mixture was sufficiently stirred and then tetrakistriphenyl-phosphinopalladium (1.5 g, 1.3 mmol) was added. After reacting for 1 hour, the reaction mixture was cooled to room temperature, and then the organic layer and the aqueous layer were separated, and the organic layer was distilled. This was again added to 1244 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 10 (15.7 g, 63%, MS: [M+H]⁺=562.3).

### Synthesis Example 11: Preparation of Compound 11

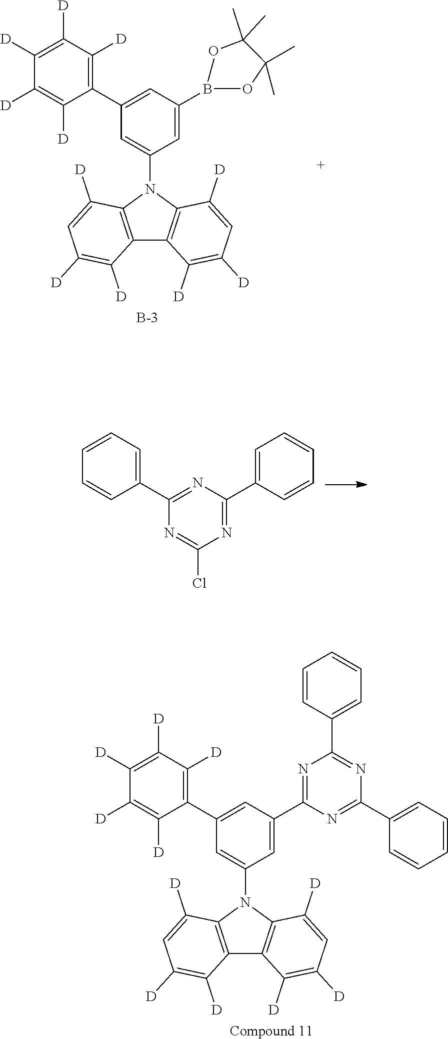

B-3 (20 g, 43.8 mmol) and 2-chloro-4,6-diphenyl-1,3,5-triazine (11.7 g, 43.8 mmol) were added to 400 mL of tetrahydrofuran under a nitrogen atmosphere, and the mixture was stirred and refluxed. Then, potassium carbonate (18.2 g, 131.5 mmol) was dissolved in 18 mL of water and added thereto, and the mixture was sufficiently stirred and then tetrakistriphenyl-phosphinopalladium (1.5 g, 1.3 mmol) was added. After reacting for 3 hour, the reaction mixture was cooled to room temperature, and then the organic layer and the aqueous layer were separated, and the organic layer was distilled. This was again added to 1230 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 11 (18.2 g, 74%, MS: [M+H]⁺=562.3).

### Synthesis Example 12: Preparation of Compound 12

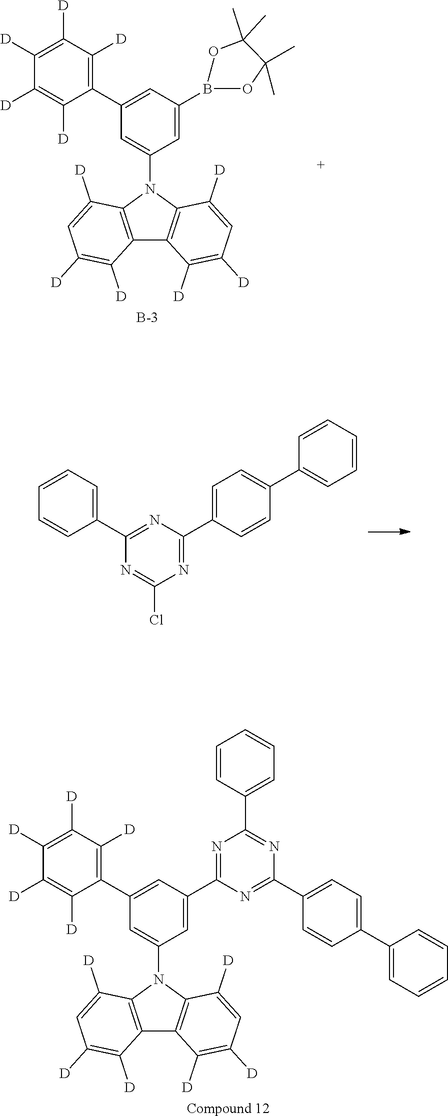

B-3 (20 g, 43.8 mmol) and 2-([1,1′-biphenyl]-4-yl)-4-chloro-6-phenyl-1,3,5-triazine (15 g, 43.8 mmol) were added to 400 mL of tetrahydrofuran under a nitrogen atmosphere, and the mixture was stirred and refluxed. Then, potassium carbonate (18.2 g, 131.5 mmol) was dissolved in 18 mL of water and added thereto, and the mixture was sufficiently stirred and then tetrakistriphenyl-phosphinopalladium (1.5 g, 1.3 mmol) was added. After reacting for 2 hours, the reaction mixture was cooled to room temperature, and then the organic layer and the aqueous layer were separated, and the organic layer was distilled. This was again added to 1397 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 12 (15.4 g, 55%, MS: [M+H]⁺=638.3).

### Synthesis Example 13: Preparation of Compound 13

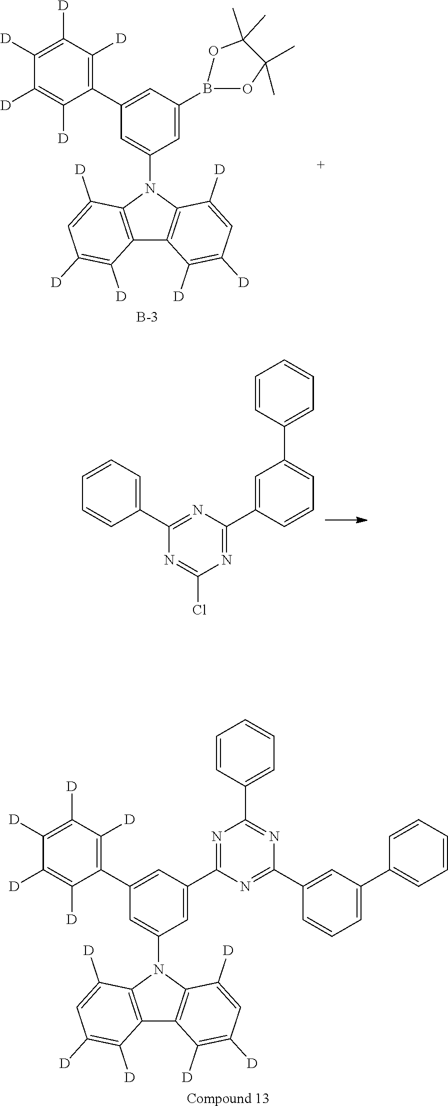

B-3 (20 g, 43.8 mmol) and 2-([1,1′-biphenyl]-4-yl)-3-chloro-6-phenyl-1,3,5-triazine (15 g, 43.8 mmol) were added to 400 mL of tetrahydrofuran under a nitrogen atmosphere, and the mixture was stirred and refluxed. Then, potassium carbonate (18.2 g, 131.5 mmol) was dissolved in 18 mL of water and added thereto, and the mixture was sufficiently stirred and then tetrakistriphenyl-phosphinopalladium (1.5 g, 1.3 mmol) was added. After reacting for 2 hours, the reaction mixture was cooled to room temperature, and then the organic layer and the aqueous layer were separated, and the organic layer was distilled. This was again added to 1397 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 13 (14.8 g, 53%, MS: [M+H]⁺=638.3).

### Synthesis Example 14: Preparation of Compound 14

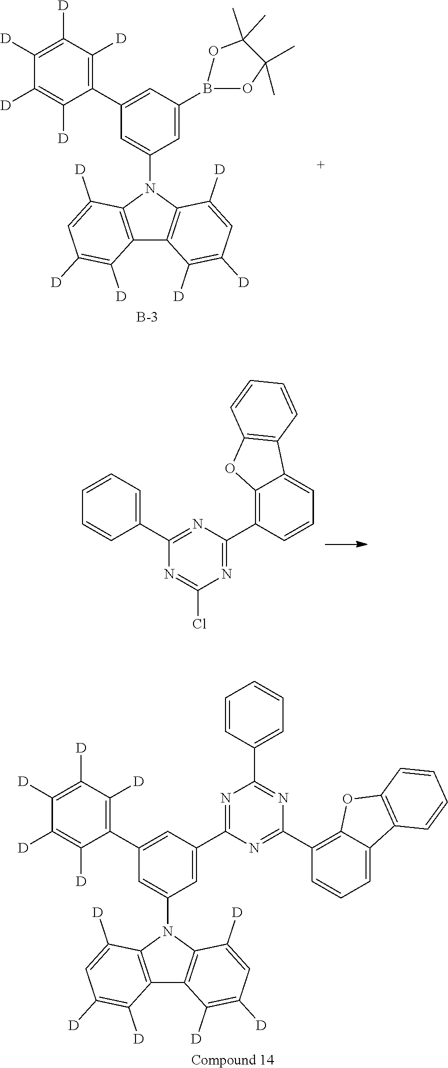

B-3 (20 g, 43.8 mmol) and 2-chloro-4-(dibenzo[b,d]furan-4-yl)-6-phenyl-1,3,5-triazine (15.7 g, 43.8 mmol) were added to 400 mL of tetrahydrofuran under a nitrogen atmosphere, and the mixture was stirred and refluxed. Then, potassium carbonate (18.2 g, 131.5 mmol) was dissolved in 18 mL of water and added thereto, and the mixture was sufficiently stirred and then tetrakistriphenyl-phosphinopalladium (1.5 g, 1.3 mmol) was added. After reacting for 1 hour, the reaction mixture was cooled to room temperature, and then the organic layer and the aqueous layer were separated, and the organic layer was distilled. This was again added to 1427 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 14 (22.3 g, 78%, MS: [M+H]⁺=652.3).

### Synthesis Example 15: Preparation of Compound 15

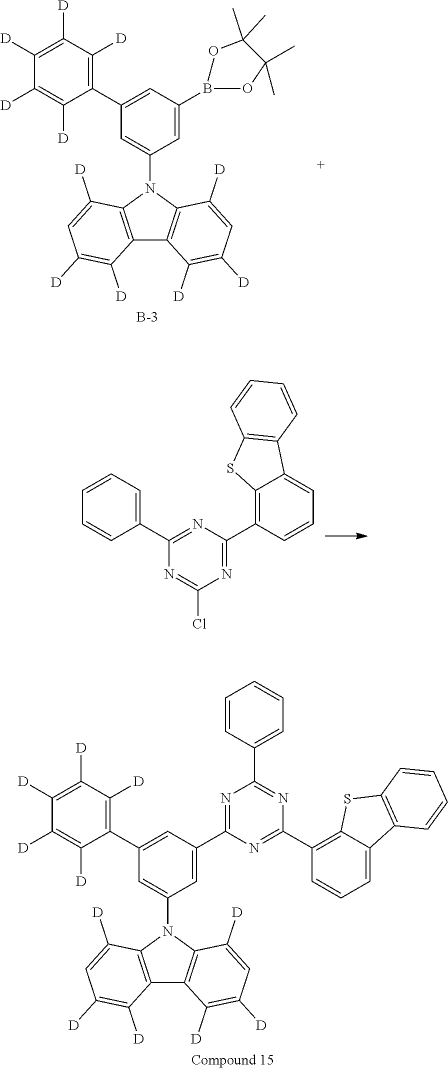

B-3 (20 g, 43.8 mmol) and 2-chloro-4-(dibenzo[b,d]thiophen-4-yl)-6-phenyl-1,3,5-triazine (16.4 g, 43.8 mmol) were added to 400 mL of tetrahydrofuran under a nitrogen atmosphere, and the mixture was stirred and refluxed. Then, potassium carbonate (18.2 g, 131.5 mmol) was dissolved in 18 mL of water and added thereto, and the mixture was sufficiently stirred and then tetrakistriphenyl-phosphinopalladium (1.5 g, 1.3 mmol) was added. After reacting for 1 hour, the reaction mixture was cooled to room temperature, and then the organic layer and the aqueous layer were separated, and the organic layer was distilled. This was again added to 1462 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 15 (17.8 g, 61%, MS: [M+H]⁺=668.2).

### Synthesis Example 16: Preparation of Compound 16

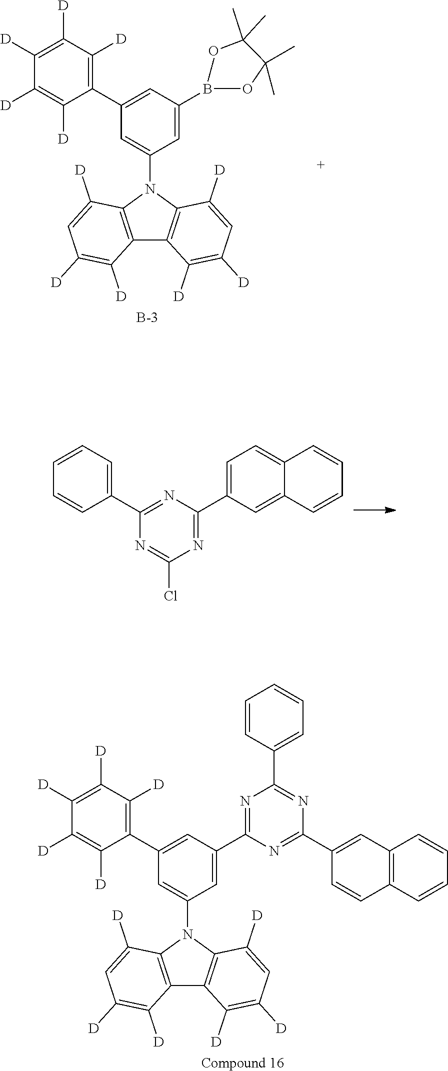

B-3 (20 g, 43.8 mmol) and 2-chloro-4-(naphthalen-2-yl)-6-phenyl-1,3,5-triazine (13.9 g, 43.8 mmol) were added to 400 mL of tetrahydrofuran under a nitrogen atmosphere, and the mixture was stirred and refluxed. Then, potassium carbonate (18.2 g, 131.5 mmol) was dissolved in 18 mL of water and added thereto, and the mixture was sufficiently stirred and then tetrakistriphenyl-phosphinopalladium (1.5 g, 1.3 mmol) was added. After reacting for 2 hours, the reaction mixture was cooled to room temperature, and then the organic layer and the aqueous layer were separated, and the organic layer was distilled. This was again added to 1340 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 16 (13.9 g, 52%, MS: [M+H]⁺=612.3).

### Synthesis Example 17: Preparation of Compound 17

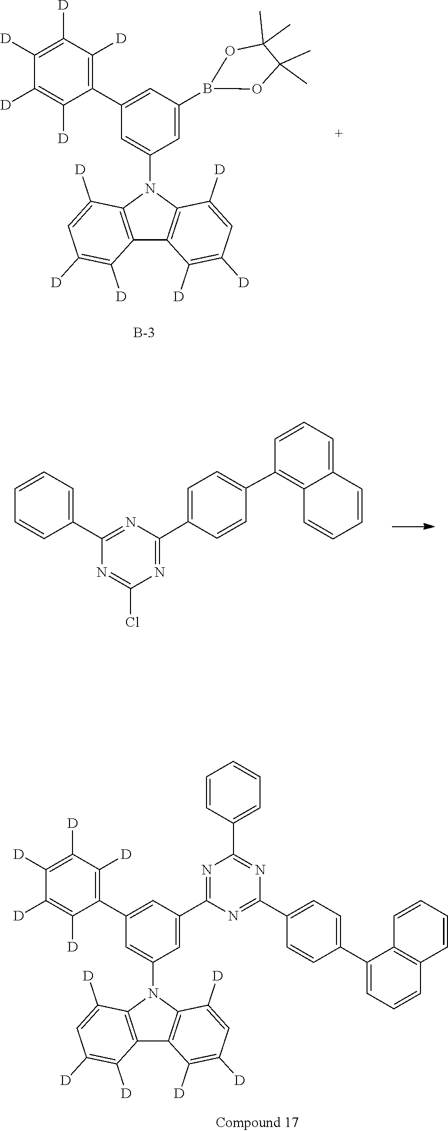

B-3 (20 g, 43.8 mmol) and 2-chloro-4-(4-(naphthalen-1-yl)phenyl)-6-phenyl-1,3,5-triazine (17.2 g, 43.8 mmol) were added to 400 mL of tetrahydrofuran under a nitrogen atmosphere, and the mixture was stirred and refluxed. Then, potassium carbonate (18.2 g, 131.5 mmol) was dissolved in 18 mL of water and added thereto, and the mixture was sufficiently stirred and then tetrakistriphenyl-phosphinopalladium (1.5 g, 1.3 mmol) was added. After reacting for 3 hour, the reaction mixture was cooled to room temperature, and then the organic layer and the aqueous layer were separated, and the organic layer was distilled. This was again added to 1506 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 17 (16.3 g, 54%, MS: [M+H]⁺=688.3).

### Synthesis Example 18: Preparation of Compound 18

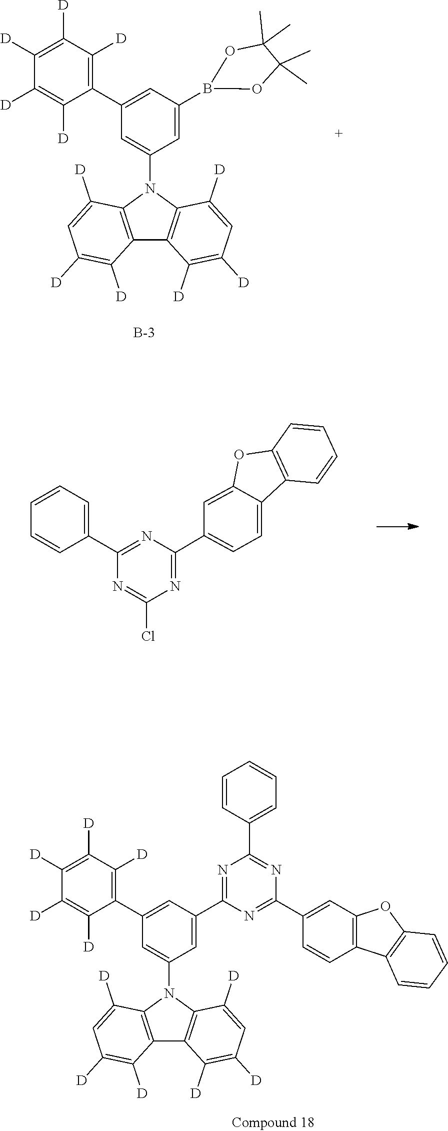

B-3 (20 g, 43.8 mmol) and 2-chloro-4-(dibenzo[b,d]furan-3-yl)-6-phenyl-1,3,5-triazine (15.7 g, 43.8 mmol) were added to 400 mL of tetrahydrofuran under a nitrogen atmosphere, and the mixture was stirred and refluxed. Then, potassium carbonate (18.2 g, 131.5 mmol) was dissolved in 18 mL of water and added thereto, and the mixture was sufficiently stirred and then tetrakistriphenyl-phosphinopalladium (1.5 g, 1.3 mmol) was added. After reacting for 2 hours, the reaction mixture was cooled to room temperature, and then the organic layer and the aqueous layer were separated, and the organic layer was distilled. This was again added to 1427 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 18 (15.1 g, 53%, MS: [M+H]⁺=652.3).

### Synthesis Example 19: Preparation of Compound 19

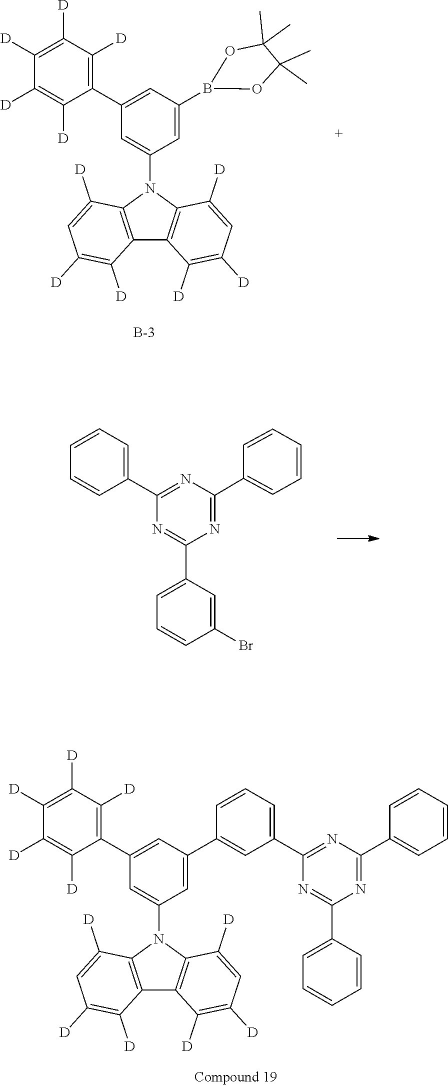

B-3 (20 g, 43.8 mmol) and 2-(3-bromophenyl)-4,6-diphenyl-1,3,5-triazine (17 g, 43.8 mmol) were added to 400 mL of tetrahydrofuran under a nitrogen atmosphere, and the mixture was stirred and refluxed. Then, potassium carbonate (18.2 g, 131.5 mmol) was dissolved in 18 mL of water and added thereto, and the mixture was sufficiently stirred and then tetrakistriphenyl-phosphinopalladium (1.5 g, 1.3 mmol) was added. After reacting for 3 hour, the reaction mixture was cooled to room temperature, and then the organic layer and the aqueous layer were separated, and the organic layer was distilled. This was again added to 1408 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 19 (22.5 g, 80%, MS: [M+H]⁺=643.3).

### Synthesis Example 20: Preparation of Compound 20

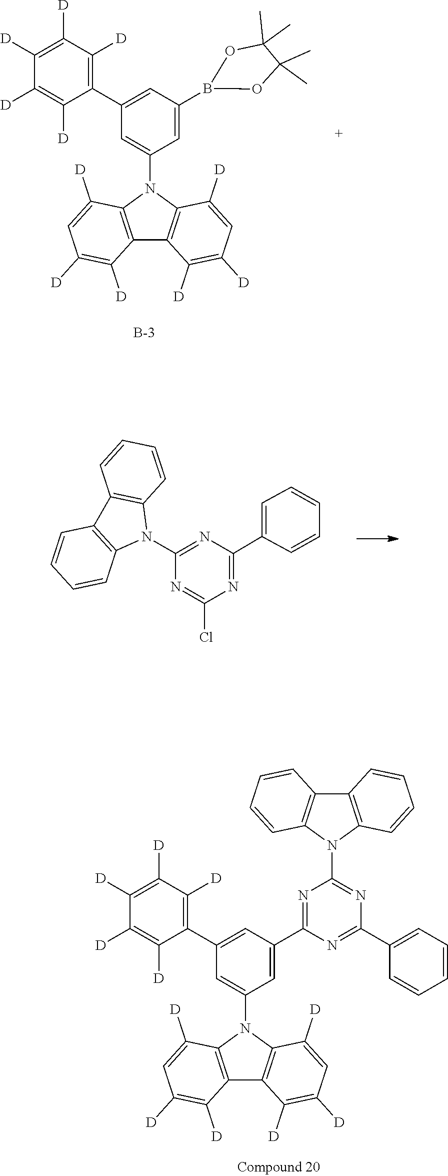

B-3 (100 g, 219.2 mmol) and 9-(4-chloro-6-phenyl-1,3,5-triazin-2-yl)-9H-carbazole (78 g, 219.2 mmol) were added to 2000 mL of tetrahydrofuran under a nitrogen atmosphere, and the mixture was stirred and refluxed. Then, potassium carbonate (90.9 g, 657.5 mmol) was dissolved in 91 mL of water and added thereto, and the mixture was sufficiently stirred and then tetrakistriphenyl-phosphinopalladium (7.6 g, 6.6 mmol) was added. After reacting for 2 hours, the reaction mixture was cooled to room temperature, and then the organic layer and the aqueous layer were separated, and the organic layer was distilled. This was again added to 2850 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 20 (102.6 g, 72%, MS: [M+H]⁺=651.3).

### Synthesis Example 21: Preparation of Compound 21

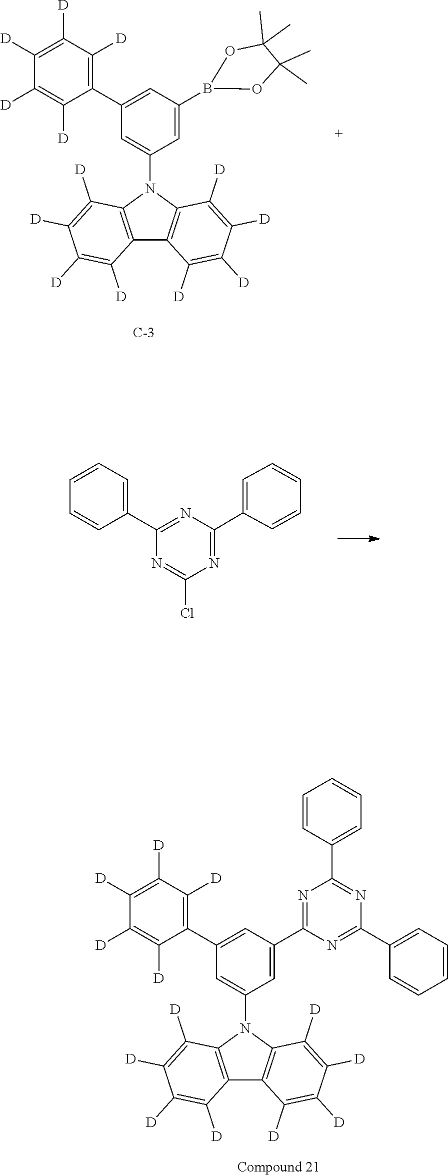

C-3 (20 g, 43.6 mmol) and 2-chloro-4,6-diphenyl-1,3,5-triazine (11.7 g, 43.6 mmol) were added to 400 mL of tetrahydrofuran under a nitrogen atmosphere, and the mixture was stirred and refluxed. Then, potassium carbonate (18.1 g, 130.9 mmol) was dissolved in 18 mL of water and added thereto, and the mixture was sufficiently stirred and then tetrakistriphenyl-phosphinopalladium (1.5 g, 1.3 mmol) was added. After reacting for 2 hours, the reaction mixture was cooled to room temperature, and then the organic layer and the aqueous layer were separated, and the organic layer was distilled. This was again added to 1229 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 21 (13 g, 53%, MS: [M+H]⁺=564.3).

### Synthesis Example 22: Preparation of Compound 22

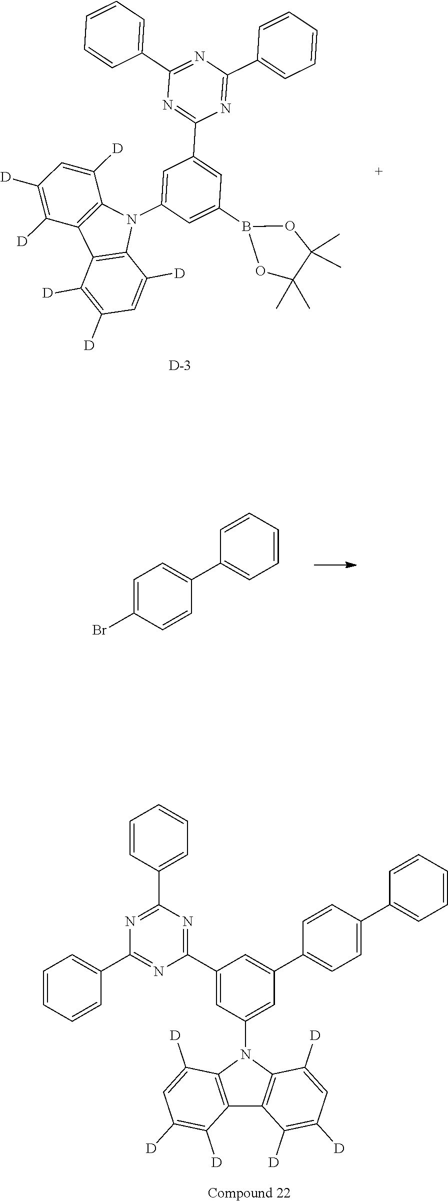

D-3 (20 g, 32.9 mmol) and 4-bromo-1,1′-biphenyl (7.6 g, 32.9 mmol) were added to 400 mL of tetrahydrofuran under a nitrogen atmosphere, and the mixture was stirred and refluxed. Then, potassium carbonate (13.6 g, 98.6 mmol) was dissolved in 14 mL of water and added thereto, and the mixture was sufficiently stirred and then tetrakistriphenyl-phosphinopalladium (1.1 g, 1 mmol) was added. After reacting for 1 hour, the reaction mixture was cooled to room temperature, and then the organic layer and the aqueous layer were separated, and the organic layer was distilled. This was again added to 1040 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 22 (13.3 g, 64%, MS: [M+H]⁺=633.3).

### Synthesis Example 23: Preparation of Compound 23

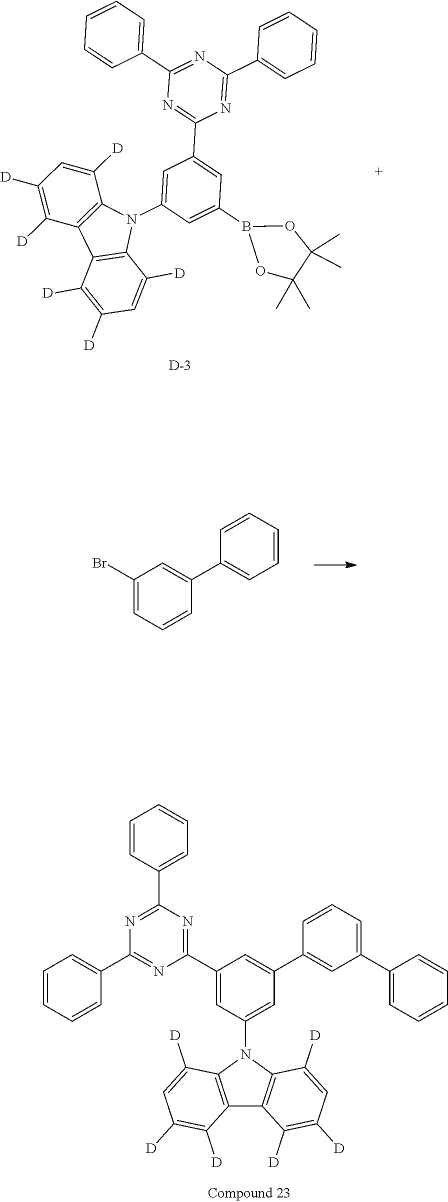

D-3 (20 g, 32.9 mmol) and 3-bromo-1,1′-biphenyl (7.6 g, 32.9 mmol) were added to 400 mL of tetrahydrofuran under a nitrogen atmosphere, and the mixture was stirred and refluxed. Then, potassium carbonate (13.6 g, 98.6 mmol) was dissolved in 14 mL of water and added thereto, and the mixture was sufficiently stirred and then tetrakistriphenyl-phosphinopalladium (1.1 g, 1 mmol) was added. After reacting for 2 hours, the reaction mixture was cooled to room temperature, and then the organic layer and the aqueous layer were separated, and the organic layer was distilled. This was again added to 1040 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 23 (16.2 g, 78%, MS: [M+H]⁺=633.3).

### Synthesis Example 24: Preparation of Compound 24

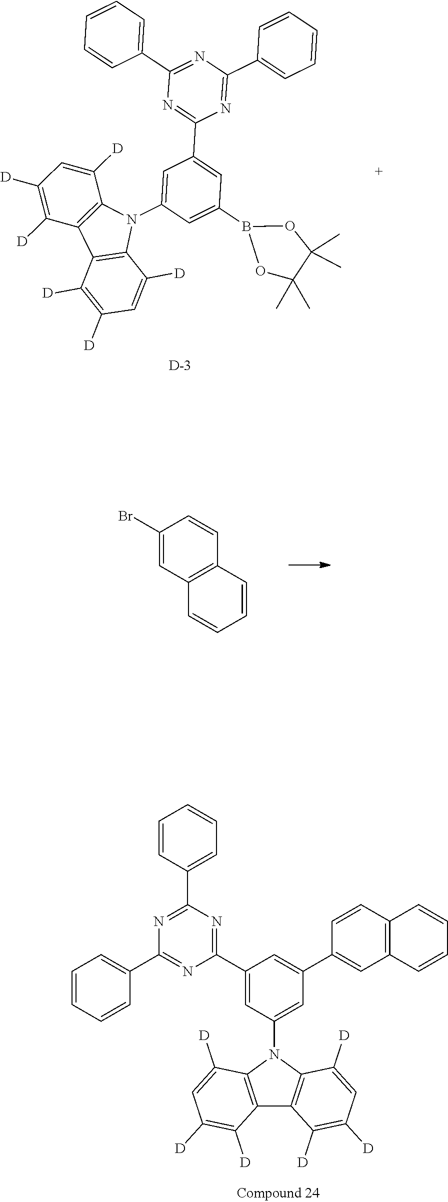

D-3 (20 g, 32.9 mmol) and 2-bromonaphthalene (6.8 g, 32.9 mmol) were added to 400 mL of tetrahydrofuran under a nitrogen atmosphere, and the mixture was stirred and refluxed. Then, potassium carbonate (13.6 g, 98.6 mmol) was dissolved in 14 mL of water and added thereto, and the mixture was sufficiently stirred and then tetrakistriphenyl-phosphinopalladium (1.1 g, 1 mmol) was added. After reacting for 3 hour, the reaction mixture was cooled to room temperature, and then the organic layer and the aqueous layer were separated, and the organic layer was distilled. This was again added to 997 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 24 (13 g, 65%, MS: [M+H]⁺=607.3).

### Synthesis Example 25: Preparation of Compound 25

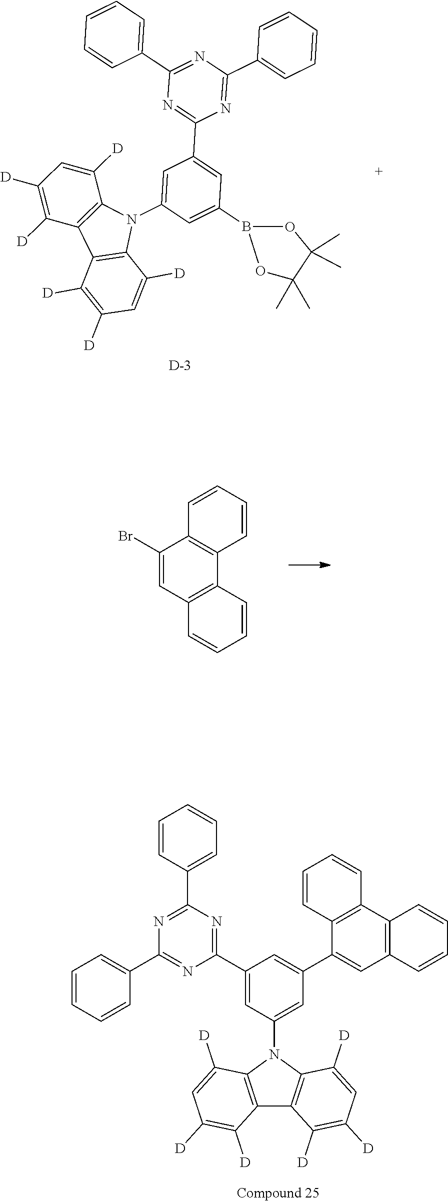

D-3 (20 g, 32.9 mmol) and 9-bromophenanthrene (8.4 g, 32.9 mmol) were added to 400 mL of tetrahydrofuran under a nitrogen atmosphere, and the mixture was stirred and refluxed. Then, potassium carbonate (13.6 g, 98.6 mmol) was dissolved in 14 mL of water and added thereto, and the mixture was sufficiently stirred and then tetrakistriphenyl-phosphinopalladium (1.1 g, 1 mmol) was added. After reacting for 3 hour, the reaction mixture was cooled to room temperature, and then the organic layer and the aqueous layer were separated, and the organic layer was distilled. This was again added to 1079 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 25 (13.6 g, 63%, MS: [M+H]⁺=657.3).

### Synthesis Example 26: Preparation of Compound 26

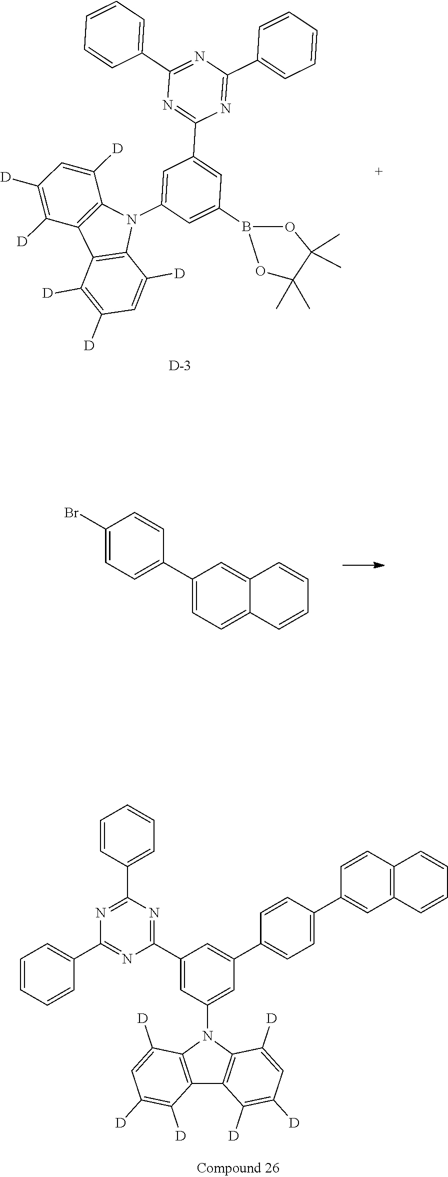

D-3 (20 g, 32.9 mmol) and 2-(4-bromophenyl)naphthalene (9.3 g, 32.9 mmol) were added to 400 mL of tetrahydrofuran under a nitrogen atmosphere, and the mixture was stirred and refluxed. Then, potassium carbonate (13.6 g, 98.6 mmol) was dissolved in 14 mL of water and added thereto, and the mixture was sufficiently stirred and then tetrakistriphenyl-phosphinopalladium (1.1 g, 1 mmol) was added. After reacting for 3 hour, the reaction mixture was cooled to room temperature, and then the organic layer and the aqueous layer were separated, and the organic layer was distilled. This was again added to 1122 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 26 (15 g, 67%, MS: [M+H]⁺=683.3).

### Synthesis Example 27: Preparation of Compound 27

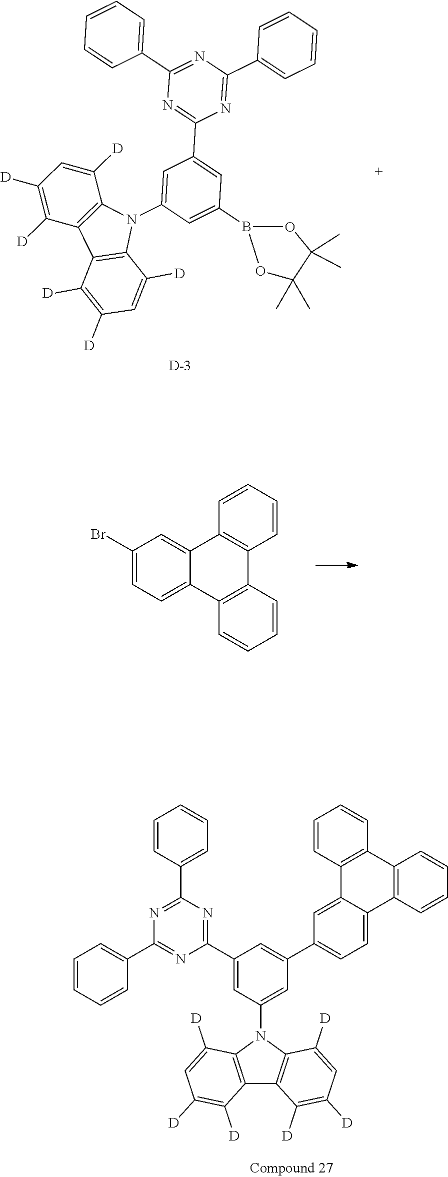

D-3 (20 g, 32.9 mmol) and 2-bromotriphenylene (11.7 g, 32.9 mmol) were added to 400 mL of tetrahydrofuran under a nitrogen atmosphere, and the mixture was stirred and refluxed. Then, potassium carbonate (13.6 g, 98.6 mmol) was dissolved in 14 mL of water and added thereto, and the mixture was sufficiently stirred and then tetrakistriphenyl-phosphinopalladium (1.1 g, 1 mmol) was added. After reacting for 1 hour, the reaction mixture was cooled to room temperature, and then the organic layer and the aqueous layer were separated, and the organic layer was distilled. This was again added to 1161 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 27 (16.7 g, 72%, MS: [M+H]⁺=707.3).

### Synthesis Example 28: Preparation of Compound 28

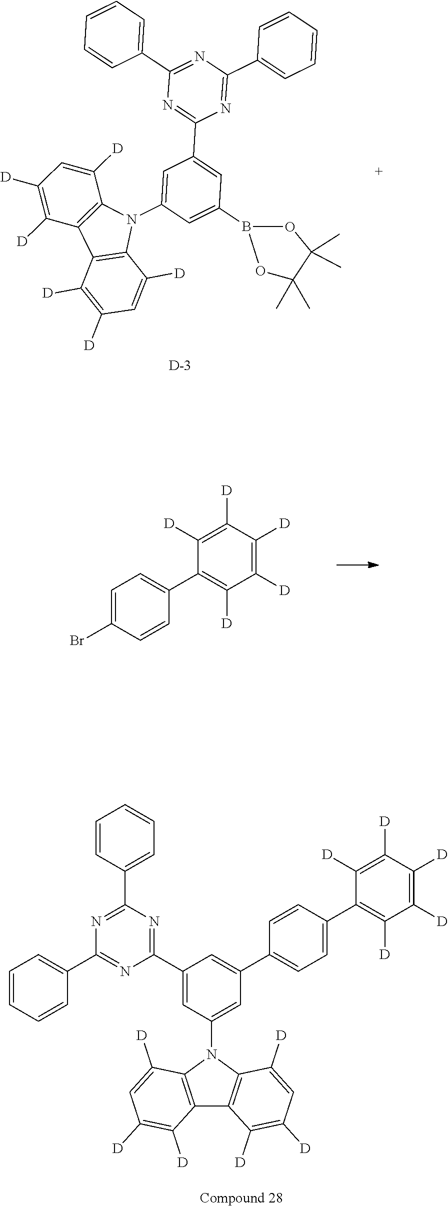

D-3 (20 g, 32.9 mmol) and 4-bromo-1,1′-biphenyl-2′,3′,4′,5′,6′-d5 (7.8 g, 32.9 mmol) were added to 400 mL of tetrahydrofuran under a nitrogen atmosphere, and the mixture was stirred and refluxed. Then, potassium carbonate (13.6 g, 98.6 mmol) was dissolved in 14 mL of water and added thereto, and the mixture was sufficiently stirred and then tetrakistriphenyl-phosphinopalladium (1.1 g, 1 mmol) was added. After reacting for 3 hour, the reaction mixture was cooled to room temperature, and then the organic layer and the aqueous layer were separated, and the organic layer was distilled. This was again added to 1048 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 28 (11.1 g, 53%, MS: [M+H]⁺=638.3).

### Synthesis Example 29: Preparation of Compound 29

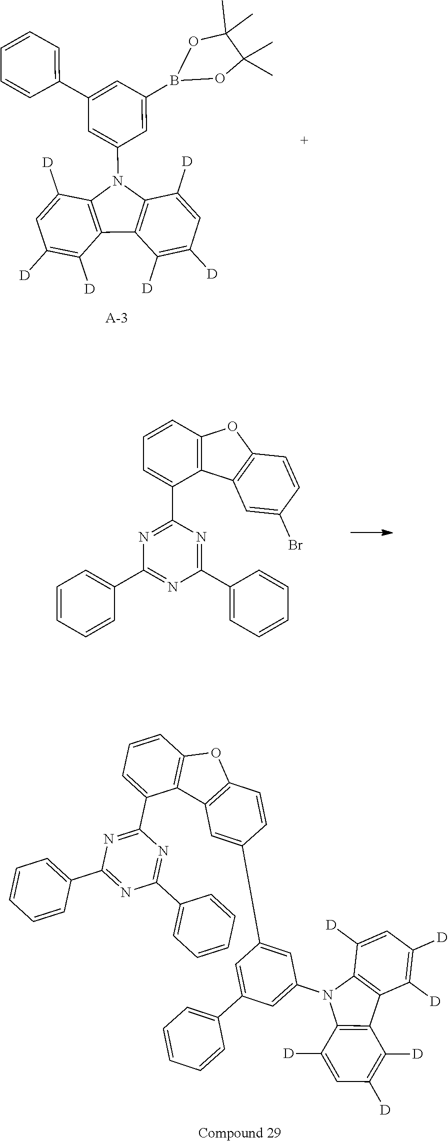

A-3 (20 g, 44.3 mmol) and 2-(8-bromodibenzo[b,d]furan-1-yl)-4,6-diphenyl-1,3,5-triazine (21.1 g, 44.3 mmol) were added to 400 mL of tetrahydrofuran under a nitrogen atmosphere, and the mixture was stirred and refluxed. Then, potassium carbonate (18.4 g, 133 mmol) was dissolved in 18 mL of water and added thereto, and the mixture was sufficiently stirred and then tetrakistriphenyl-phosphinopalladium (1.5 g, 1.3 mmol) was added. After reacting for 3 hour, the reaction mixture was cooled to room temperature, and then the organic layer and the aqueous layer were separated, and the organic layer was distilled. This was again added to 1601 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a gray solid Compound 29 (23.4 g, 73%, MS: [M+H]⁺=723.3).

### Example 1: Manufacture of Organic Light Emitting Device

A glass substrate on which a thin film of ITO (indium tin oxide) was coated in a thickness of 1,300 Å was placed in distilled water containing a detergent dissolved therein and ultrasonically cleaned. In this case, the detergent used was a product commercially available from Fischer Co. and the distilled water was one which had been twice filtered by using a filter commercially available from Millipore Co. The ITO was cleaned for 30 minutes, and ultrasonic cleaning was then repeated twice for 10 minutes using distilled water. After the cleaning with distilled water was completed, the substrate was ultrasonically washed with the solvents of isopropyl alcohol, acetone, and methanol, and dried, after which it was transported to a plasma cleaner. Then, the substrate was cleaned with oxygen plasma for 5 minutes, and then transferred to a vacuum evaporator.

On the ITO transparent electrode thus prepared, the following Compound HI-1 was thermally vacuum-deposited to a thickness of 50 Å to form a hole injection layer. The following Compound HT-1 was thermally vacuum-deposited to a thickness of 250 Å on the hole injection layer to form hole transport layer, and the following Compound HT-2 was vacuum deposited on the HT-1 deposited layer to a thickness of 50 Å to form a hole blocking layer.

Then, Compound 1 prepared in the previous Synthesis Example 1 as a light emitting layer, the following Compound YGH-1, and the following phosphorescence dopant YGD-1 were vacuum deposited at a weight ratio of 44:44:12 on the HT-2 deposited layer to form a light emitting layer with a thickness of 400 Å.

The following Compound ET-1 was vacuum deposited on the light emitting layer to a thickness of 250 Å to form an electron transport layer, and the following Compound ET-2 and LiF were vacuum deposited at a weight ratio of 8:2 on the electron transport layer to form an electron injection layer with a film thickness of 100 Å. Aluminum was deposited on the electron injection layer to a thickness of 1,000 Å to form a cathode.

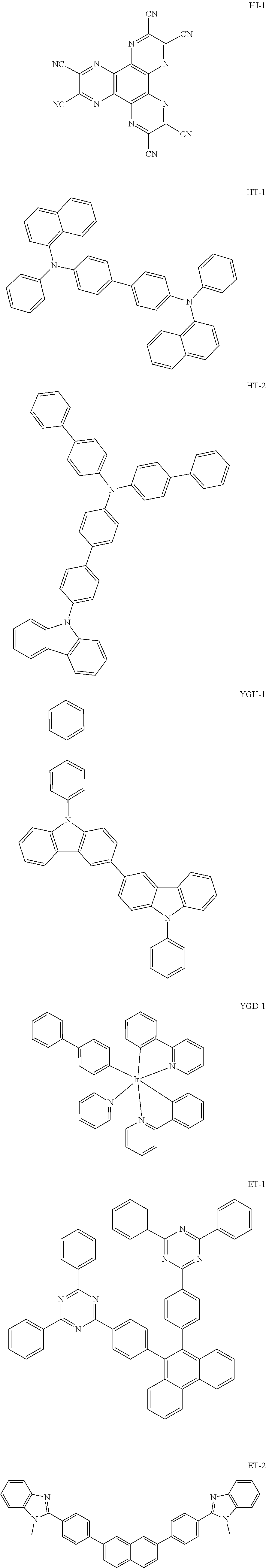

In the above-mentioned processes, the deposition rate of the organic material was maintained at 0.4˜ 0.7 Å/sec, the deposition rate of aluminum was maintained at 2 Å/sec, and the degree of vacuum during deposition was maintained at 1×10⁻⁷˜ 5×10⁻⁸ torr.

### Examples 2 to 29

The organic light emitting devices were manufactured in the same manner as in Example 1, except that as one of the host materials for the light emitting layer in Example 1, the Compound shown in Table 1 below was used instead of the Compound 1 of Synthetic Example 1.

At this time, the structures of the compounds used in Examples 1 to 29 are as follows.

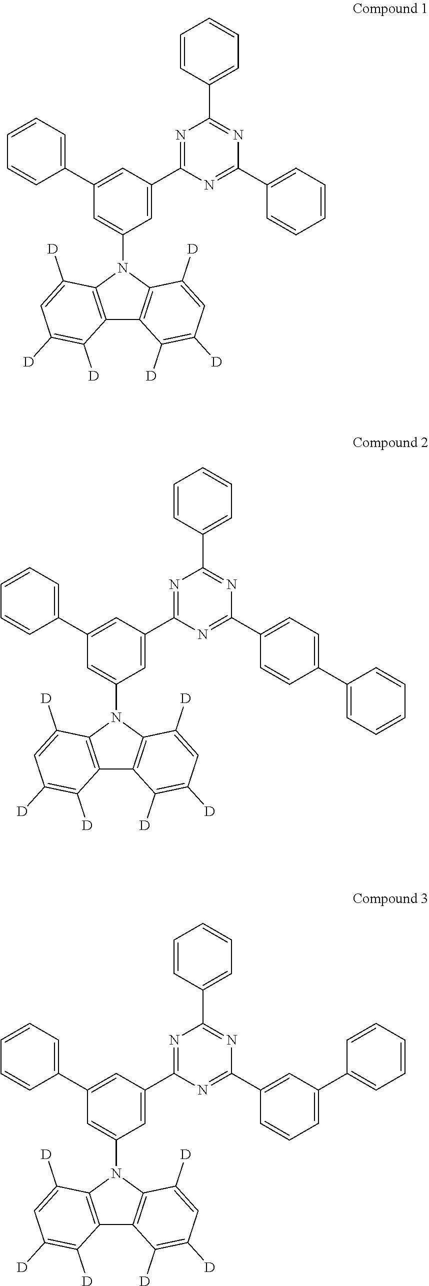 [SMILES: [2H]c1cc([2H])c2c(c1[2H])c1c([2H])c([2H])cc([2H])c1n2-c1cc(-c2ccccc2)cc(-c2nc(-c3ccccc3)nc(-c3ccccc3)n2)c1 ; [2H]c1cc([2H])c2c(c1[2H])c1c([2H])c([2H])cc([2H])c1n2-c1cc(-c2ccccc2)cc(-c2nc(-c3ccccc3)nc(-c3ccc(-c4ccccc4)cc3)n2)c1 ; [2H]c1cc([2H])c2c(c1[2H])c1c([2H])c([2H])cc([2H])c1n2-c1cc(-c2ccccc2)cc(-c2nc(-c3ccccc3)nc(-c3cccc(-c4ccccc4)c3)n2)c1]

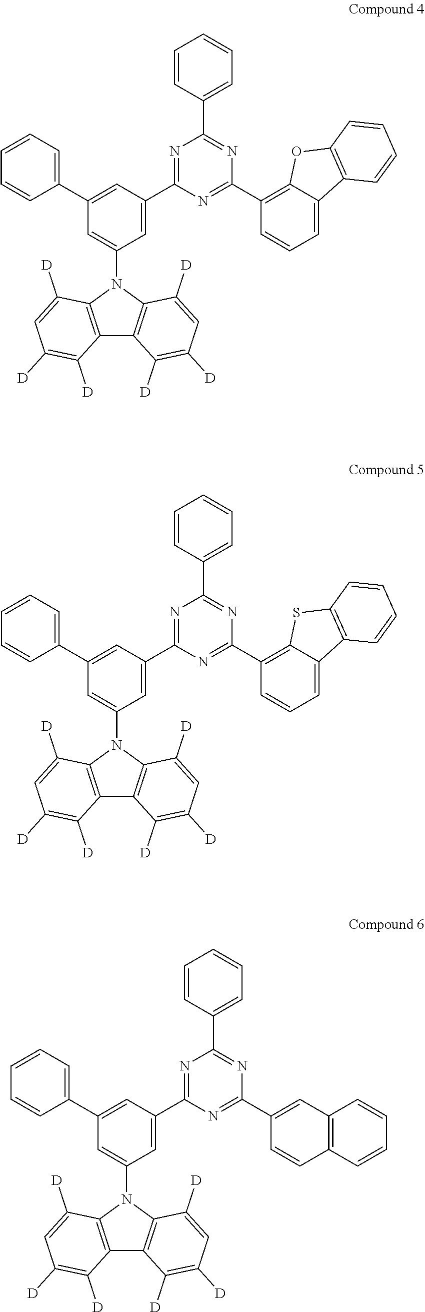 [SMILES: [2H]c1cc([2H])c2c(c1[2H])c1c([2H])c([2H])cc([2H])c1n2-c1cc(-c2ccccc2)cc(-c2nc(-c3ccccc3)nc(-c3cccc4c3oc3ccccc34)n2)c1 ; [2H]c1cc([2H])c2c(c1[2H])c1c([2H])c([2H])cc([2H])c1n2-c1cc(-c2ccccc2)cc(-c2nc(-c3ccccc3)nc(-c3cccc4c3sc3ccccc34)n2)c1 ; [2H]c1cc([2H])c2c(c1[2H])c1c([2H])c([2H])cc([2H])c1n2-c1cc(-c2ccccc2)cc(-c2nc(-c3ccccc3)nc(-c3ccc4ccccc4c3)n2)c1]

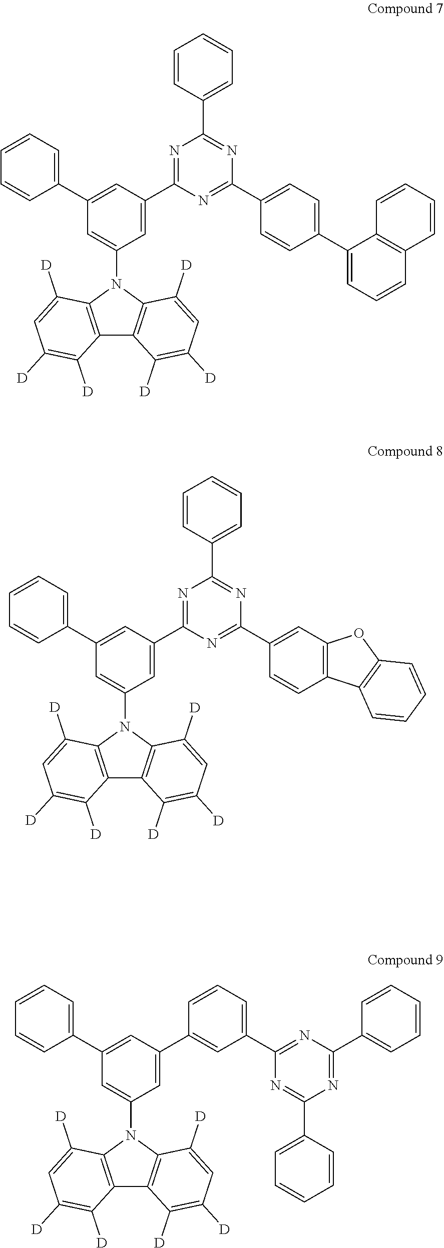 [SMILES: [2H]c1cc([2H])c2c(c1[2H])c1c([2H])c([2H])cc([2H])c1n2-c1cc(-c2ccccc2)cc(-c2nc(-c3ccccc3)nc(-c3ccc(-c4cccc5ccccc45)cc3)n2)c1 ; [2H]c1cc([2H])c2c(c1[2H])c1c([2H])c([2H])cc([2H])c1n2-c1cc(-c2ccccc2)cc(-c2nc(-c3ccccc3)nc(-c3ccc4c(c3)oc3ccccc34)n2)c1 ; [2H]c1cc([2H])c2c(c1[2H])c1c([2H])c([2H])cc([2H])c1n2-c1cc(-c2ccccc2)cc(-c2cccc(-c3nc(-c4ccccc4)nc(-c4ccccc4)n3)c2)c1]

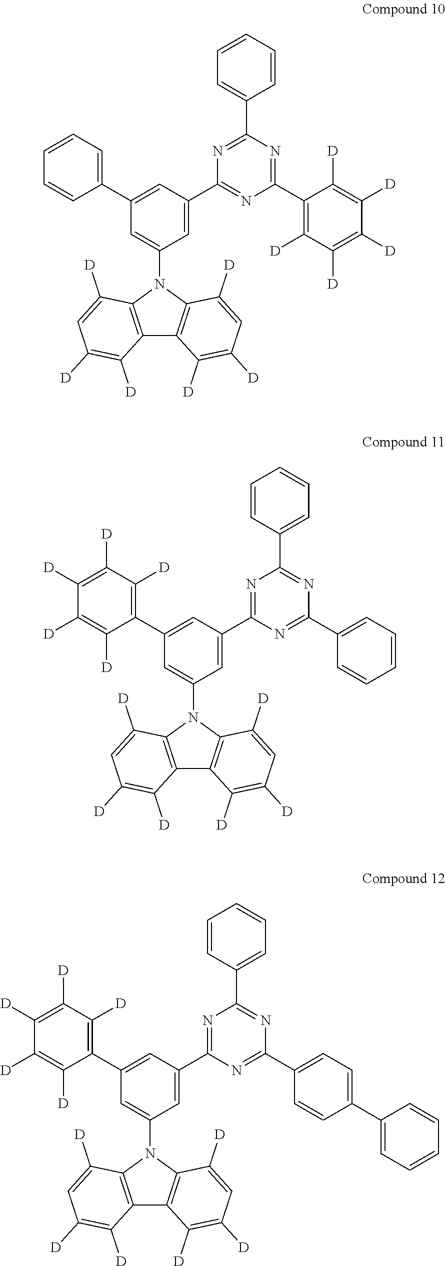 [SMILES: [2H]c1cc([2H])c2c(c1[2H])c1c([2H])c([2H])cc([2H])c1n2-c1cc(-c2ccccc2)cc(-c2nc(-c3ccccc3)nc(-c3c([2H])c([2H])c([2H])c([2H])c3[2H])n2)c1 ; [2H]c1cc([2H])c2c(c1[2H])c1c([2H])c([2H])cc([2H])c1n2-c1cc(-c2nc(-c3ccccc3)nc(-c3ccccc3)n2)cc(-c2c([2H])c([2H])c([2H])c([2H])c2[2H])c1 ; [2H]c1cc([2H])c2c(c1[2H])c1c([2H])c([2H])cc([2H])c1n2-c1cc(-c2nc(-c3ccccc3)nc(-c3ccc(-c4ccccc4)cc3)n2)cc(-c2c([2H])c([2H])c([2H])c([2H])c2[2H])c1]

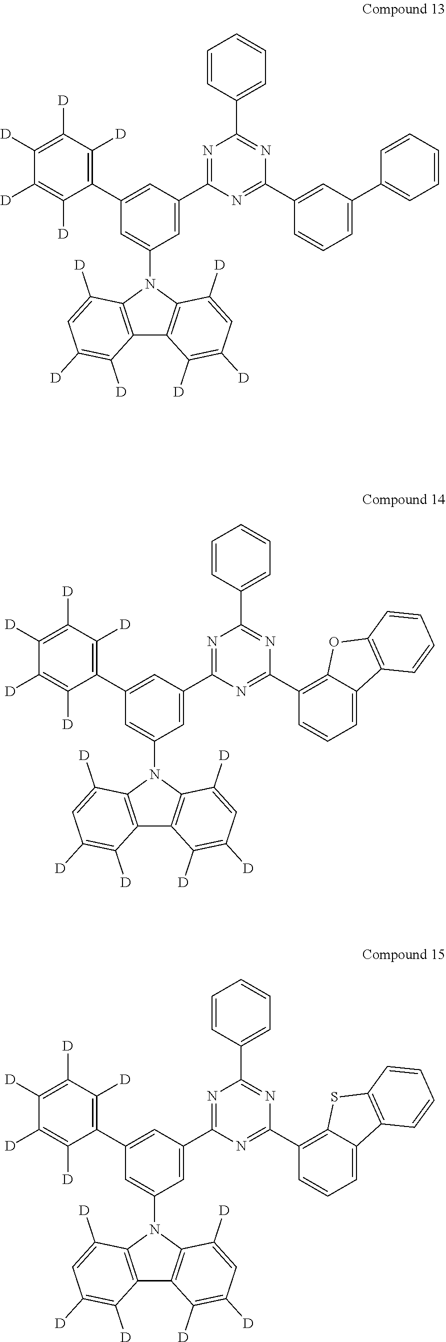 [SMILES: [2H]c1cc([2H])c2c(c1[2H])c1c([2H])c([2H])cc([2H])c1n2-c1cc(-c2nc(-c3ccccc3)nc(-c3cccc(-c4ccccc4)c3)n2)cc(-c2c([2H])c([2H])c([2H])c([2H])c2[2H])c1 ; [2H]c1cc([2H])c2c(c1[2H])c1c([2H])c([2H])cc([2H])c1n2-c1cc(-c2nc(-c3ccccc3)nc(-c3cccc4c3oc3ccccc34)n2)cc(-c2c([2H])c([2H])c([2H])c([2H])c2[2H])c1 ; [2H]c1cc([2H])c2c(c1[2H])c1c([2H])c([2H])cc([2H])c1n2-c1cc(-c2nc(-c3ccccc3)nc(-c3cccc4c3sc3ccccc34)n2)cc(-c2c([2H])c([2H])c([2H])c([2H])c2[2H])c1]

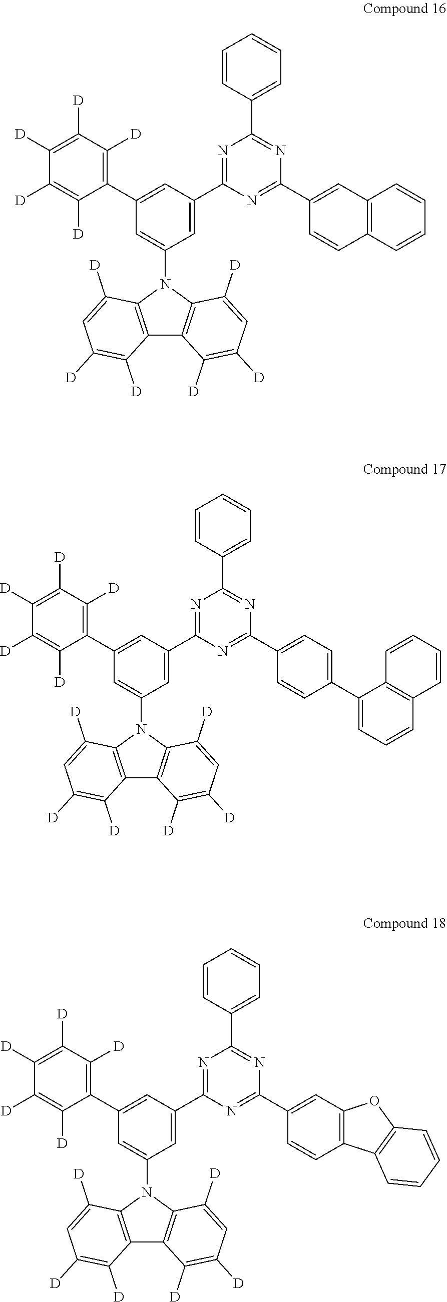 [SMILES: [2H]c1cc([2H])c2c(c1[2H])c1c([2H])c([2H])cc([2H])c1n2-c1cc(-c2nc(-c3ccccc3)nc(-c3ccc4ccccc4c3)n2)cc(-c2c([2H])c([2H])c([2H])c([2H])c2[2H])c1 ; [2H]c1cc([2H])c2c(c1[2H])c1c([2H])c([2H])cc([2H])c1n2-c1cc(-c2nc(-c3ccccc3)nc(-c3ccc(-c4cccc5ccccc45)cc3)n2)cc(-c2c([2H])c([2H])c([2H])c([2H])c2[2H])c1 ; [2H]c1cc([2H])c2c(c1[2H])c1c([2H])c([2H])cc([2H])c1n2-c1cc(-c2nc(-c3ccccc3)nc(-c3ccc4c(c3)oc3ccccc34)n2)cc(-c2c([2H])c([2H])c([2H])c([2H])c2[2H])c1]

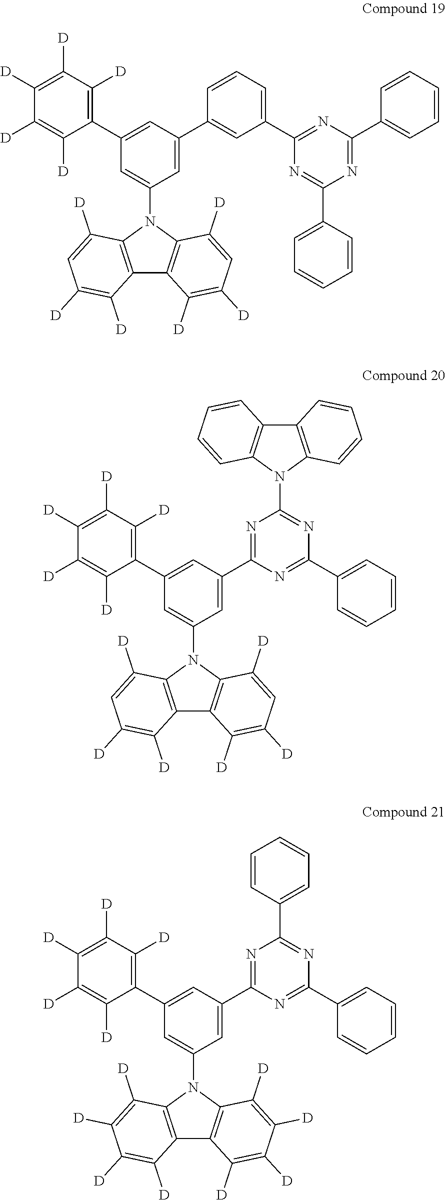 [SMILES: [2H]c1cc([2H])c2c(c1[2H])c1c([2H])c([2H])cc([2H])c1n2-c1cc(-c2cccc(-c3nc(-c4ccccc4)nc(-c4ccccc4)n3)c2)cc(-c2c([2H])c([2H])c([2H])c([2H])c2[2H])c1 ; [2H]c1cc([2H])c2c(c1[2H])c1c([2H])c([2H])cc([2H])c1n2-c1cc(-c2nc(-c3ccccc3)nc(-n3c4ccccc4c4ccccc43)n2)cc(-c2c([2H])c([2H])c([2H])c([2H])c2[2H])c1 ; [2H]c1c([2H])c([2H])c(-c2cc(-c3nc(-c4ccccc4)nc(-c4ccccc4)n3)cc(-n3c4c([2H])c([2H])c([2H])c([2H])c4c4c([2H])c([2H])c([2H])c([2H])c43)c2)c([2H])c1[2H]]

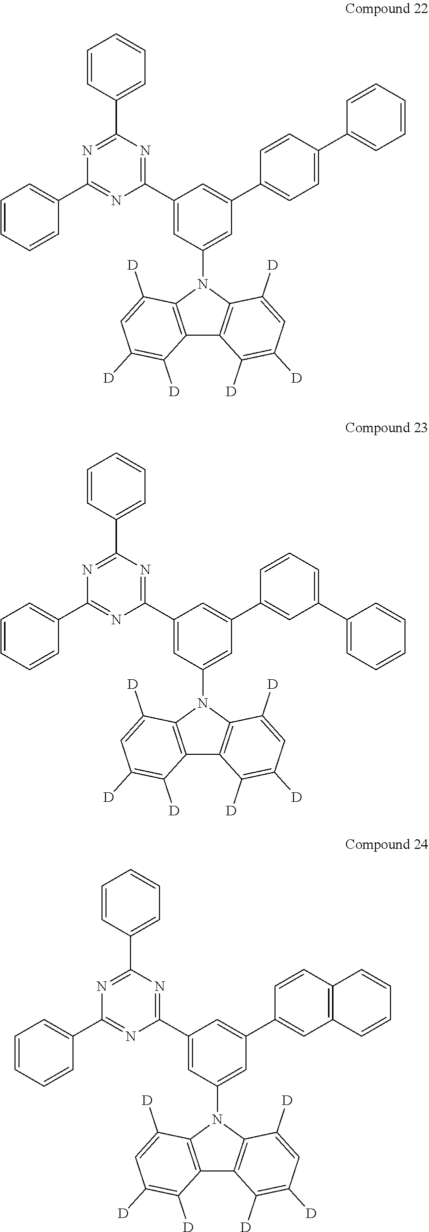 [SMILES: [2H]c1cc([2H])c2c(c1[2H])c1c([2H])c([2H])cc([2H])c1n2-c1cc(-c2ccc(-c3ccccc3)cc2)cc(-c2nc(-c3ccccc3)nc(-c3ccccc3)n2)c1 ; [2H]c1cc([2H])c2c(c1[2H])c1c([2H])c([2H])cc([2H])c1n2-c1cc(-c2cccc(-c3ccccc3)c2)cc(-c2nc(-c3ccccc3)nc(-c3ccccc3)n2)c1 ; [2H]c1cc([2H])c2c(c1[2H])c1c([2H])c([2H])cc([2H])c1n2-c1cc(-c2ccc3ccccc3c2)cc(-c2nc(-c3ccccc3)nc(-c3ccccc3)n2)c1]

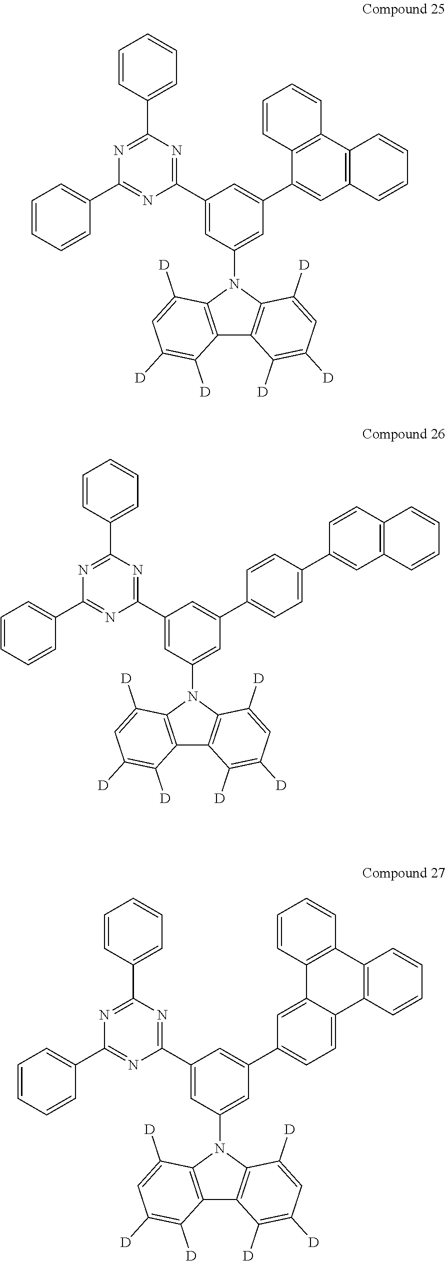 [SMILES: [2H]c1cc([2H])c2c(c1[2H])c1c([2H])c([2H])cc([2H])c1n2-c1cc(-c2nc(-c3ccccc3)nc(-c3ccccc3)n2)cc(-c2cc3ccccc3c3ccccc23)c1 ; [2H]c1cc([2H])c2c(c1[2H])c1c([2H])c([2H])cc([2H])c1n2-c1cc(-c2ccc(-c3ccc4ccccc4c3)cc2)cc(-c2nc(-c3ccccc3)nc(-c3ccccc3)n2)c1 ; [2H]c1cc([2H])c2c(c1[2H])c1c([2H])c([2H])cc([2H])c1n2-c1cc(-c2ccc3c4ccccc4c4ccccc4c3c2)cc(-c2nc(-c3ccccc3)nc(-c3ccccc3)n2)c1]

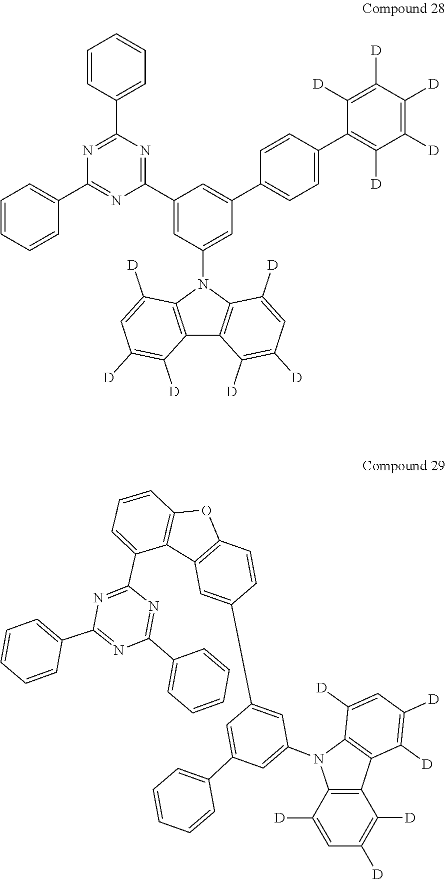 [SMILES: [2H]c1cc([2H])c2c(c1[2H])c1c([2H])c([2H])cc([2H])c1n2-c1cc(-c2ccc(-c3c([2H])c([2H])c([2H])c([2H])c3[2H])cc2)cc(-c2nc(-c3ccccc3)nc(-c3ccccc3)n2)c1 ; [2H]c1cc([2H])c2c(c1[2H])c1c([2H])c([2H])cc([2H])c1n2-c1cc(-c2ccccc2)cc(-c2ccc3oc4cccc(-c5nc(-c6ccccc6)nc(-c6ccccc6)n5)c4c3c2)c1]

### Comparative Examples 1 to 4

The organic light emitting devices were manufactured in the same manner as in Example 1, except that as one of the host materials for the light emitting layer in Example 1, the Compound shown in Table 1 below was used instead of the Compound 1 of Synthetic Example 1. At this time, the Compounds CE1 to CE4 of Table 1 below are as follows.

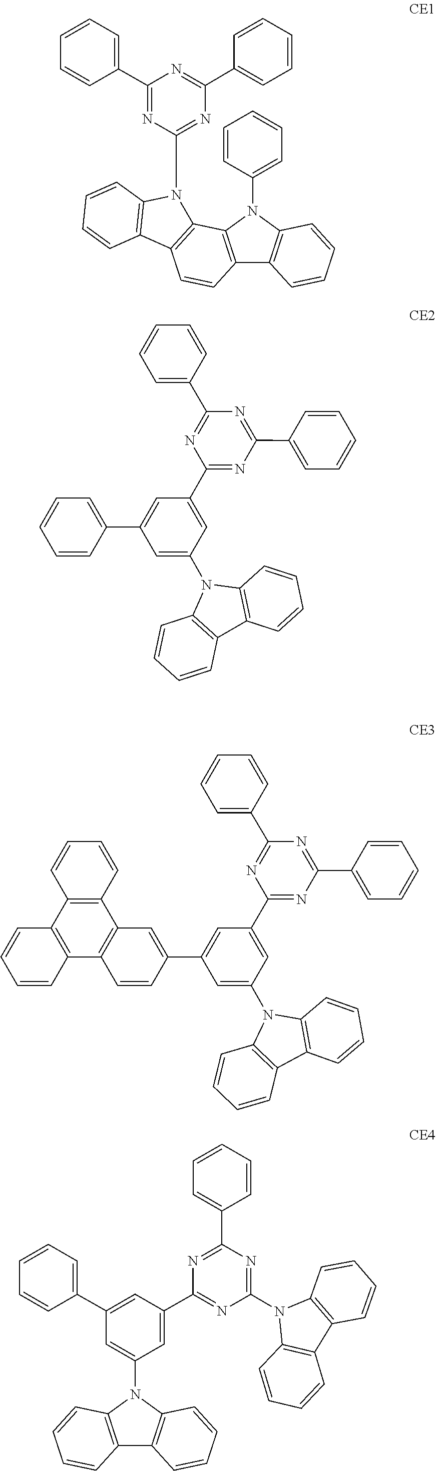

### Experimental Example 1: Evaluation of Device Characteristics

The voltage and efficiency of the organic light emitting devices manufactured in Examples and Comparative Examples were measured at a current density of 10 mA/cm², and the lifetime was measured at current density of 50 mA/cm². The results are shown in Table 1 below. At this time, LT₉₅ means the time required for the luminance to be reduced to 95% of the initial luminance.

TABLE 1

| | | | Efficiency | Color | Lifetime(h) |
|---|---|---|---|---|---|
| | | Voltage (V) | (Cd/A) | coordinate | (LT₉₅ at |
| | Compound | (@10 mA/cm²) | (@10 mA/cm²) | (x, y) | 50 mA/cm²) |
| Example 1 | Compound 1 | 4 | 82 | 0.45, 0.54 | 160 |
| Example 2 | Compound 2 | 4.1 | 83 | 0.45, 0.54 | 180 |
| Example 3 | Compound 3 | 4.2 | 81 | 0.45, 0.54 | 200 |
| Example 4 | Compound 4 | 4.2 | 82 | 0.45, 0.53 | 170 |
| Example 5 | Compound 5 | 4.3 | 80 | 0.45, 0.54 | 220 |
| Example 6 | Compound 6 | 4.2 | 81 | 0.45, 0.54 | 150 |
| Example 7 | Compound 7 | 4.3 | 80 | 0.45, 0.53 | 150 |
| Example 8 | Compound 8 | 4.2 | 81 | 0.45, 0.54 | 230 |
| Example 9 | Compound 9 | 4.2 | 84 | 0.45, 0.53 | 140 |
| Example 10 | Compound | 4 | 82 | 0.45, 0.54 | 180 |
| | 10 | | | | |
| Example 11 | Compound | 4 | 82 | 0.45, 0.54 | 180 |
| | 11 | | | | |
| Example 12 | Compound | 4.1 | 83 | 0.45, 0.54 | 200 |
| | 12 | | | | |
| Example 13 | Compound | 4.2 | 81 | 0.45, 0.54 | 220 |
| | 13 | | | | |
| Example 14 | Compound | 4.2 | 82 | 0.45, 0.54 | 190 |
| | 14 | | | | |
| Example 15 | Compound | 4.3 | 80 | 0.45, 0.53 | 240 |
| | 15 | | | | |
| Example 16 | Compound | 4.2 | 81 | 0.45, 0.54 | 160 |
| | 16 | | | | |
| Example 17 | Compound | 4.3 | 80 | 0.45, 0.54 | 160 |
| | 17 | | | | |
| Example 18 | Compound | 4.2 | 81 | 0.45, 0.53 | 250 |
| | 18 | | | | |
| Example 19 | Compound | 4.2 | 84 | 0.45, 0.54 | 150 |
| | 19 | | | | |
| Example 20 | Compound | 4.3 | 77 | 0.45, 0.53 | 190 |
| | 20 | | | | |
| Example 21 | Compound | 4 | 82 | 0.45, 0.53 | 190 |
| | 21 | | | | |
| Example 22 | Compound | 4.1 | 82 | 0.45, 0.53 | 160 |
| | 22 | | | | |
| Example 23 | Compound | 4.2 | 79 | 0.45, 0.53 | 170 |
| | 23 | | | | |
| Example 24 | Compound | 4.2 | 79 | 0.45, 0.53 | 150 |
| | 24 | | | | |
| Example 25 | Compound | 4.3 | 80 | 0.45, 0.53 | 160 |
| | 25 | | | | |
| Example 26 | Compound | 4.2 | 80 | 0.45, 0.53 | 160 |
| | 26 | | | | |
| Example 27 | Compound | 4.3 | 79 | 0.45, 0.53 | 200 |
| | 27 | | | | |
| Example 28 | Compound | 4.1 | 82 | 0.45, 0.53 | 170 |
| | 28 | | | | |
| Example 29 | Compound | 4.3 | 79 | 0.45, 0.53 | 230 |
| | 29 | | | | |
| Comparative | CE1 | 4.1 | 75 | 0.45, 0.53 | 100 |
| Example 1 | | | | | |
| Comparative | CE2 | 4 | 82 | 0.45, 0.54 | 100 |
| Example 2 | | | | | |
| Comparative | CE3 | 4.3 | 79 | 0.45, 0.53 | 120 |
| Example 3 | | | | | |
| Comparative | CE4 | 4.3 | 77 | 0.45, 0.53 | 110 |
| Example 4 | | | | | |

As shown in Table 1, it can be confirmed that the organic light emitting devices of Examples using the compound of Chemical Formula 1 as a host material for the light emitting layer exhibit remarkably improved lifetime characteristics without reducing the efficiency as compared to the organic light emitting device of Comparative Example using a compound having a structure different therefrom. Therefore, considering that the luminous efficiency and lifetime characteristics of organic light emitting devices generally have a trade-off relationship, it can be confirmed that the compound of the present disclosure can improve the characteristics of the organic light emitting devices as compared to the compounds of Comparative Examples.

### DESCRIPTION OF THE SYMBOLS

| | |
|---|---|
| 1: substrate | 2: anode |
| 3: light emitting layer | 4: cathode |
| 5: hole injection layer | 6: hole transport layer |
| 7: electron blocking layer | 8: electron transport layer |
| 9: electron injection layer | |

1. A compound of Chemical Formula 1: 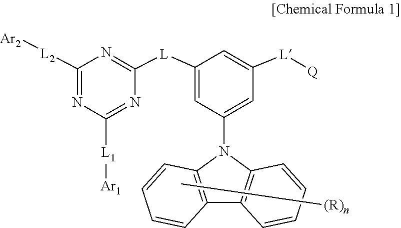
wherein, in Chemical Formula 1:
L is a single bond, a substituted or unsubstituted C₆₋₆₀ arylene, or a substituted or unsubstituted C₂₋₆₀ heteroarylene containing one or more heteroatoms of N, O and S;
L₁ and L₂ are each independently a single bond or a substituted or unsubstituted C₆₋₆₀ arylene;
Ar₁ and Ar₂ are each independently a substituted or unsubstituted C₆₋₆₀ aryl or a substituted or unsubstituted C₂₋₆₀ heteroaryl containing one or more heteroatoms of N, O and S;
L′ is a single bond or a substituted or unsubstituted C₆₋₆₀ arylene;
Q is a substituted or unsubstituted C₆₋₆₀ aryl;
R is deuterium or phenyl substituted with deuterium; and
n is an integer of 1 to 8.

2. The compound according to claim 1, wherein:
L is a single bond, phenylene, biphenyldiyl, naphthylene, dibenzofuranylene, or dibenzothiophenylene.

3. The compound according to claim 1, wherein:
L₁ and L₂ are each independently a single bond or phenylene.

4. The compound according to claim 1, wherein:
Ar₁ and Ar₂ are each independently phenyl, biphenylyl, terphenylyl, naphthyl, phenanthryl, triphenylenyl, fluorenyl, dibenzofuranyl, dibenzothiophenyl, or carbazolyl,
wherein the Ar₁ and Ar₂ are unsubstituted, or substituted with one or more substituent groups selected from the group consisting of deuterium, methyl and phenyl.

5. The compound according to claim 4, wherein:
Ar₁ and Ar₂ are each independently any one substituent selected from the group consisting of:

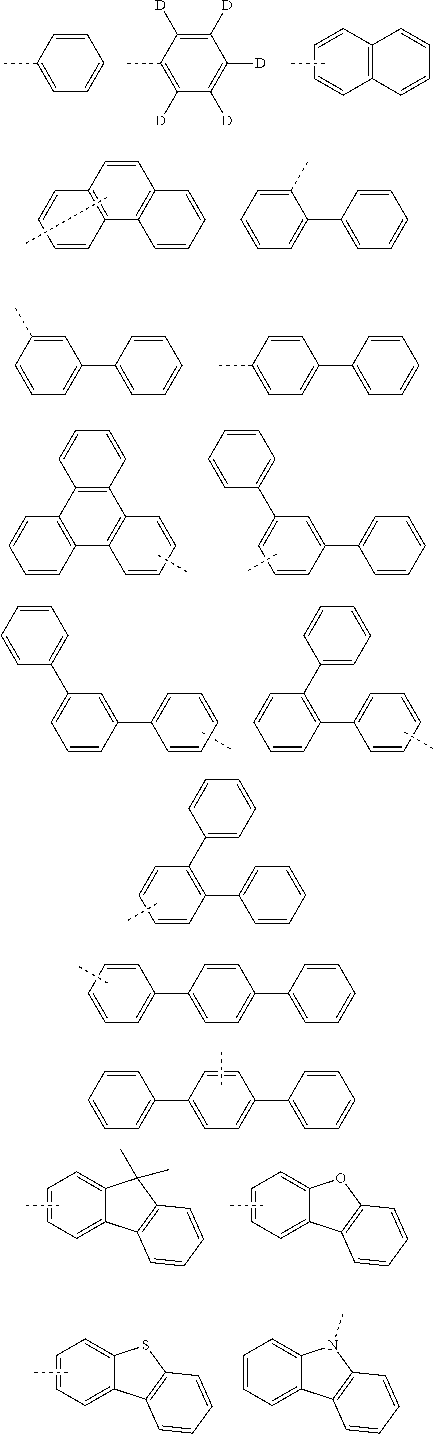

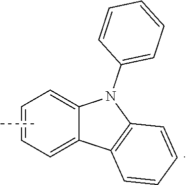

6. The compound according to claim 1, wherein:
at least one of Ar₁ and Ar₂ is a C₆₋₁₂ aryl that is unsubstituted or substituted with deuterium.

7. The compound according to claim 1, wherein:
L is a single bond, and Ar₁ and Ar₂ are each independently phenyl, phenyl substituted with 5 deuteriums, biphenylyl, terphenylyl, naphthyl, phenanthryl, triphenylenyl, 9,9-dimethylfluorenyl, dibenzofuranyl, dibenzothiophenyl, carbazolyl, or 9-phenylcarbazolyl; or
L is a C₆₋₂₀ arylene or a C₂₋₂₀ heteroarylene containing one heteroatom of O and S, and both Ar₁ and Ar₂ are phenyl.

8. The compound according to claim 1, wherein:
L′ is a single bond or phenylene.

9. The compound according to claim 1, wherein:
Q is phenyl, biphenylyl, naphthyl, phenanthryl, triphenylenyl, benzophenanthryl, or 9,9-dimethylfluorenyl,
wherein Q is unsubstituted or substituted with deuterium.

10. The compound according to claim 9, wherein:
Q is any one substituent selected from the group consisting of:

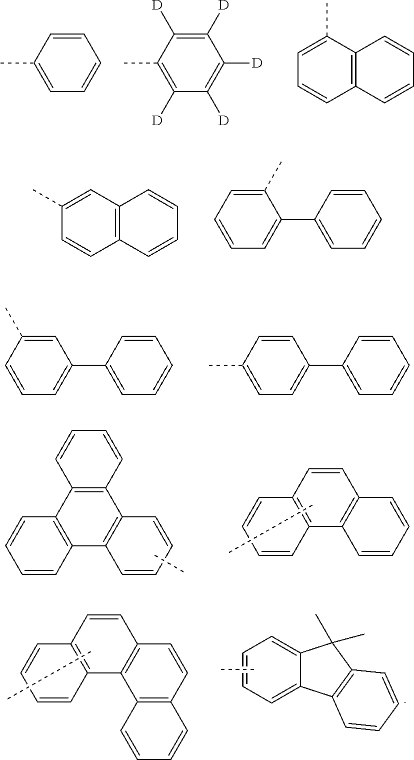

11. The compound according to claim 1, wherein:
R is deuterium or

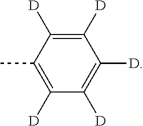

12. The compound according to claim 1, wherein:
the 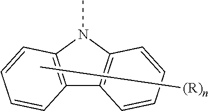
group is any one group selected from the group consisting of:

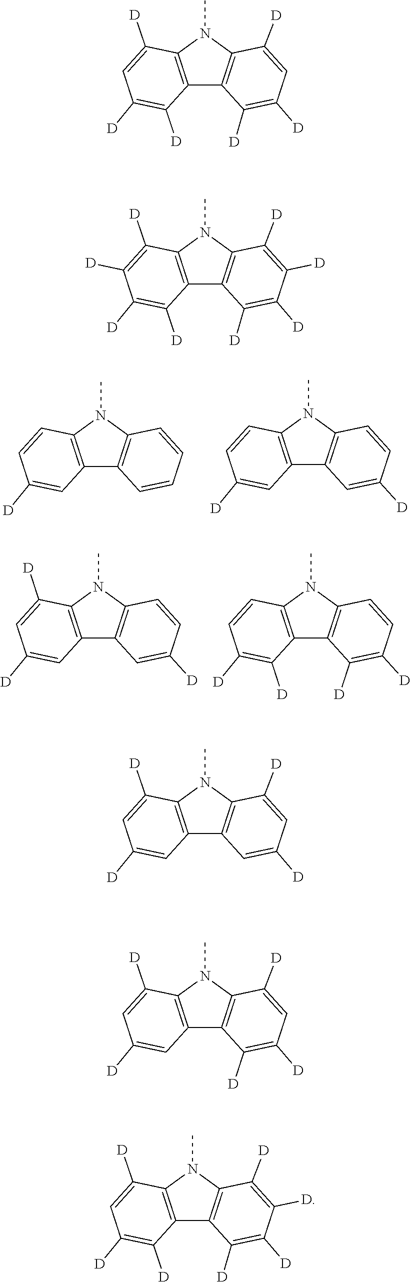

13. The compound according to claim 1, wherein:
the compound is any one of the following Chemical Formulas 1-1 to 1-3: 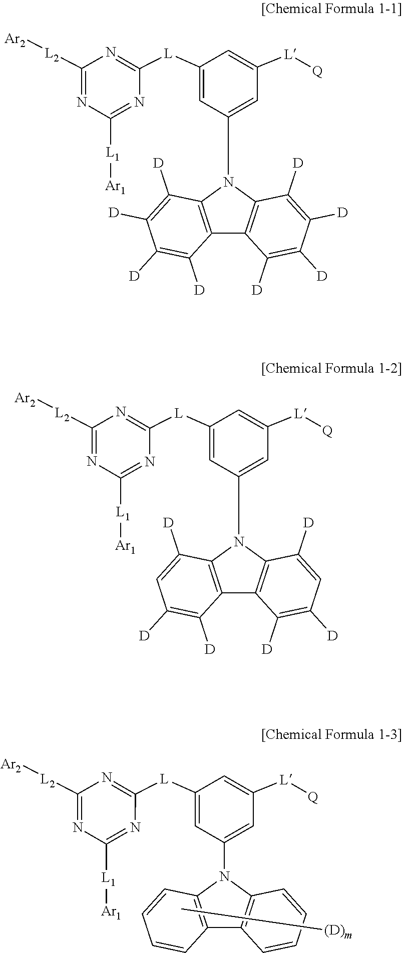
wherein, in Chemical Formulas 1-1 to 1-3:
D is deuterium;
m is 1, 2, 3, 4, 5, or 7; and
L, L₁, L₂, Ar₁, Ar₂, L′ and Q are as defined in claim 1.

14. The compound according to claim 1, wherein:
the compound is any one compound selected from the group consisting of the following compounds:

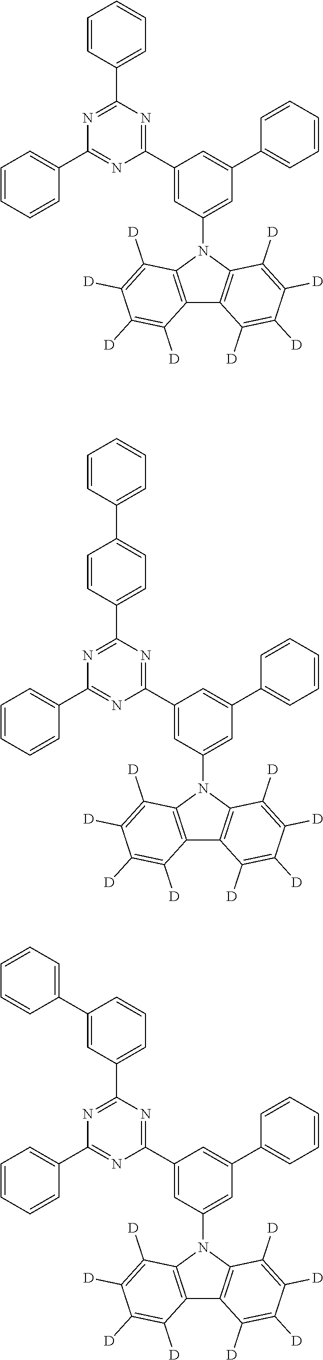 [SMILES: [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3ccc(-c4ccccc4)cc3)n2)c1 ; [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3cccc(-c4ccccc4)c3)n2)c1]

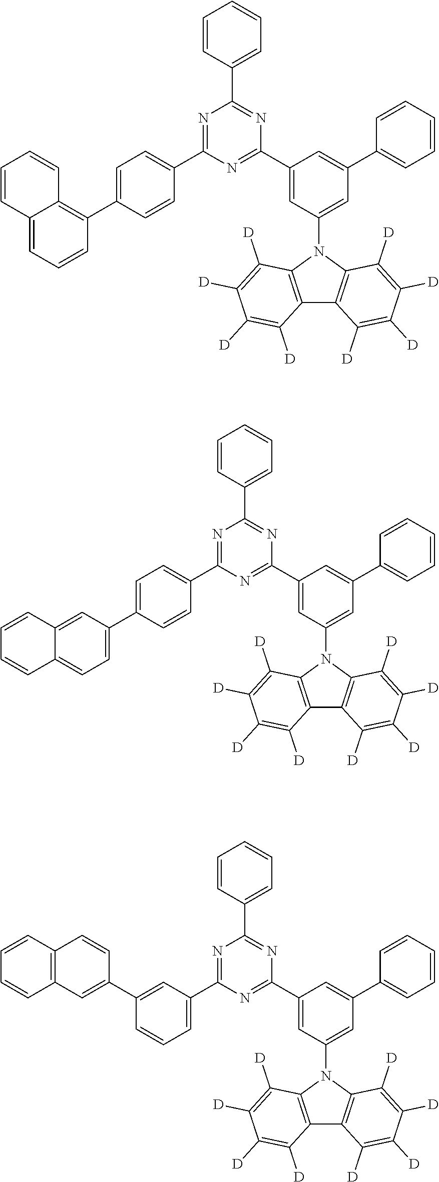 [SMILES: [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3ccc(-c4cccc5ccccc45)cc3)n2)c1 ; [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3ccc(-c4ccc5ccccc5c4)cc3)n2)c1 ; [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3cccc(-c4ccc5ccccc5c4)c3)n2)c1]

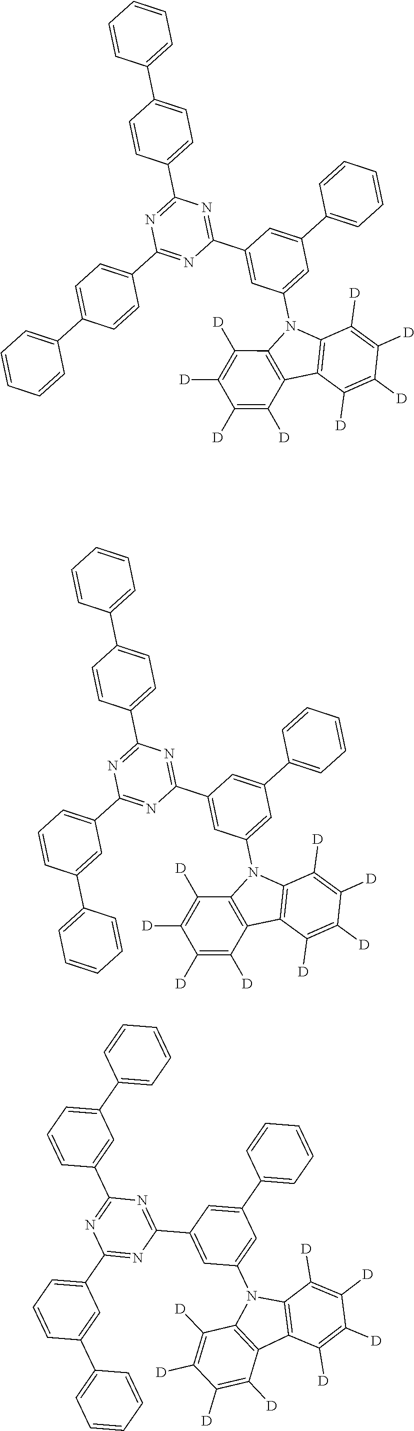 [SMILES: [2H]c1c([2H])c([2H])c2c(c1[2H])c1c([2H])c([2H])c([2H])c([2H])c1n2-c1cc(-c2ccccc2)cc(-c2nc(-c3ccc(-c4ccccc4)cc3)nc(-c3ccc(-c4ccccc4)cc3)n2)c1 ; [2H]c1c([2H])c([2H])c2c(c1[2H])c1c([2H])c([2H])c([2H])c([2H])c1n2-c1cc(-c2ccccc2)cc(-c2nc(-c3ccc(-c4ccccc4)cc3)nc(-c3cccc(-c4ccccc4)c3)n2)c1 ; [2H]c1c([2H])c([2H])c2c(c1[2H])c1c([2H])c([2H])c([2H])c([2H])c1n2-c1cc(-c2ccccc2)cc(-c2nc(-c3cccc(-c4ccccc4)c3)nc(-c3cccc(-c4ccccc4)c3)n2)c1]

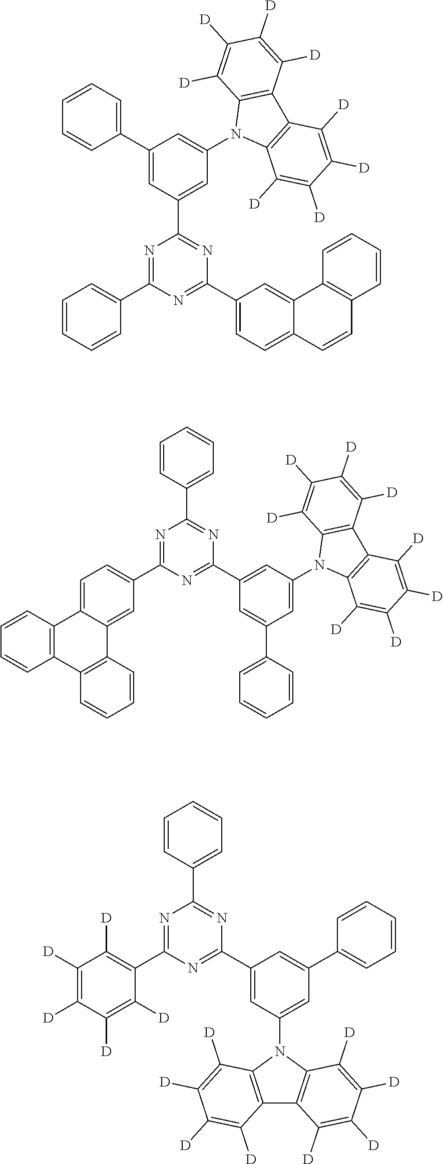 [SMILES: [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3ccc4ccc5ccccc5c4c3)n2)c1 ; [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3ccc4c5ccccc5c5ccccc5c4c3)n2)c1 ; [2H]c1c([2H])c([2H])c(-c2nc(-c3ccccc3)nc(-c3cc(-c4ccccc4)cc(-n4c5c([2H])c([2H])c([2H])c([2H])c5c5c([2H])c([2H])c([2H])c([2H])c54)c3)n2)c([2H])c1[2H]]

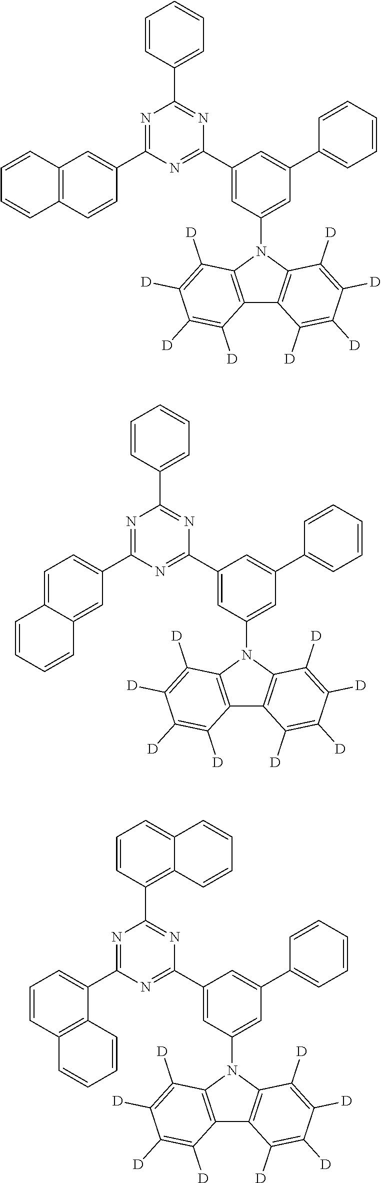 [SMILES: [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3ccc4ccccc4c3)n2)c1 ; [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3ccc4ccccc4c3)n2)c1 ; [2H]c1c([2H])c([2H])c2c(c1[2H])c1c([2H])c([2H])c([2H])c([2H])c1n2-c1cc(-c2ccccc2)cc(-c2nc(-c3cccc4ccccc34)nc(-c3cccc4ccccc34)n2)c1]

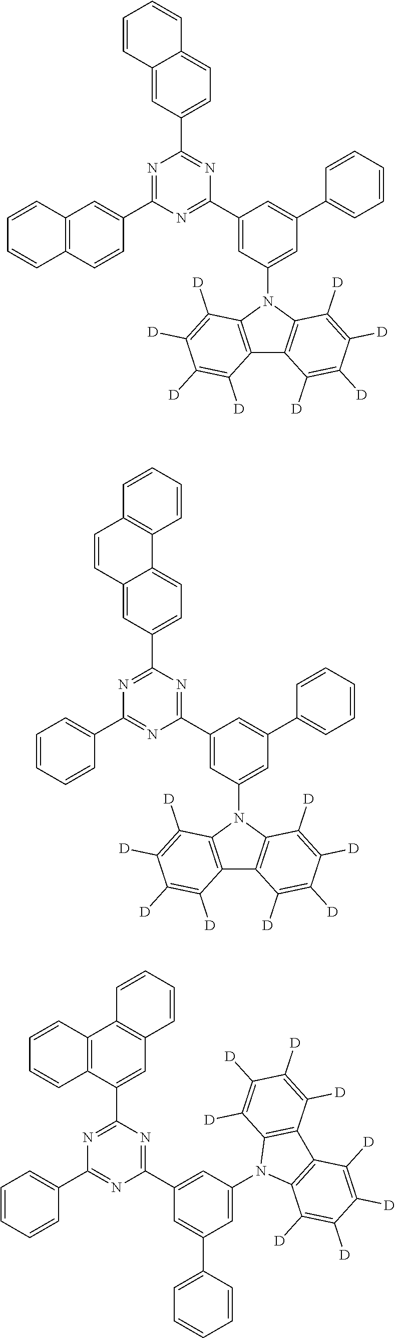 [SMILES: [2H]c1c([2H])c([2H])c2c(c1[2H])c1c([2H])c([2H])c([2H])c([2H])c1n2-c1cc(-c2ccccc2)cc(-c2nc(-c3ccc4ccccc4c3)nc(-c3ccc4ccccc4c3)n2)c1 ; [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3ccc4c(ccc5ccccc54)c3)n2)c1 ; [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3cc4ccccc4c4ccccc34)n2)c1]

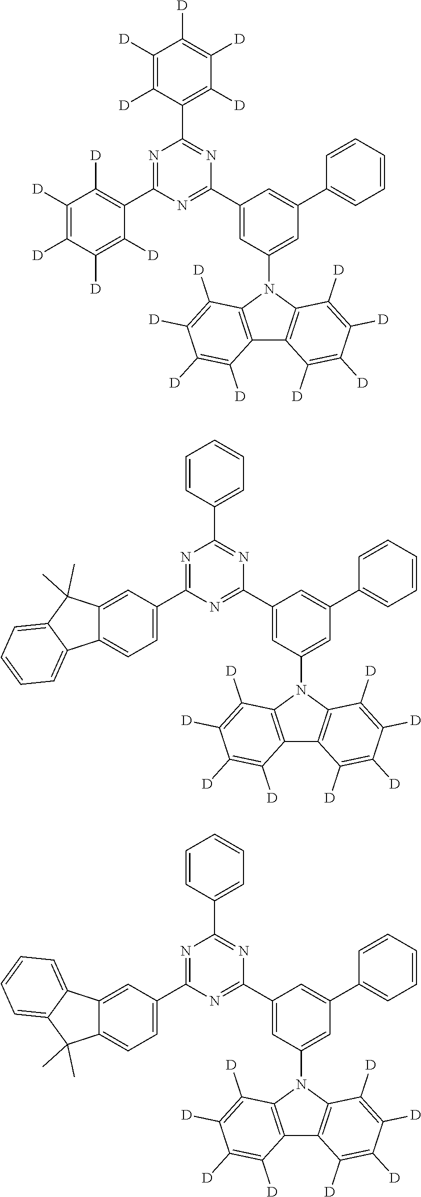 [SMILES: [2H]c1c([2H])c([2H])c(-c2nc(-c3cc(-c4ccccc4)cc(-n4c5c([2H])c([2H])c([2H])c([2H])c5c5c([2H])c([2H])c([2H])c([2H])c54)c3)nc(-c3c([2H])c([2H])c([2H])c([2H])c3[2H])n2)c([2H])c1[2H] ; [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3ccc4c(c3)C(C)(C)c3ccccc3-4)n2)c1 ; [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3ccc4c(c3)-c3ccccc3C4(C)C)n2)c1]

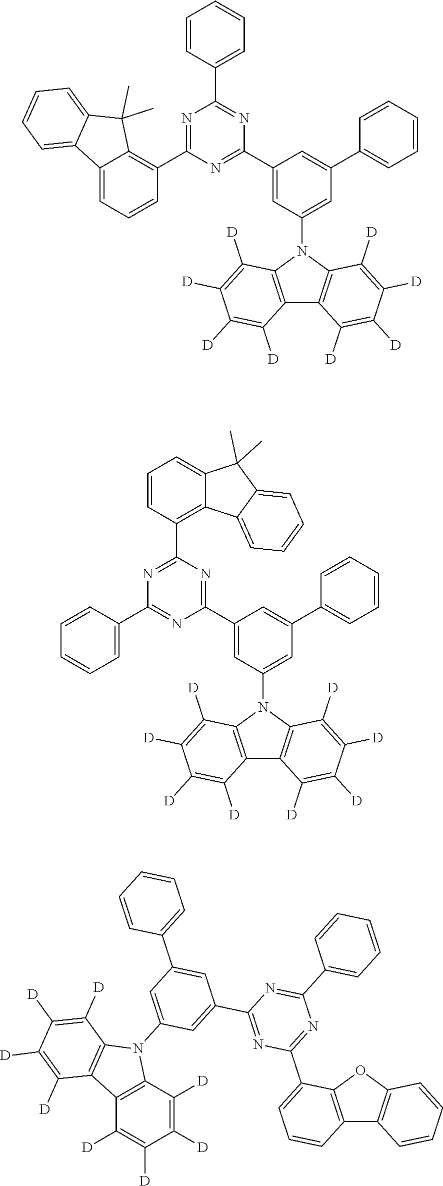 [SMILES: [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3cccc4c3C(C)(C)c3ccccc3-4)n2)c1 ; [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3cccc4c3-c3ccccc3C4(C)C)n2)c1 ; [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3cccc4c3oc3ccccc34)n2)c1]

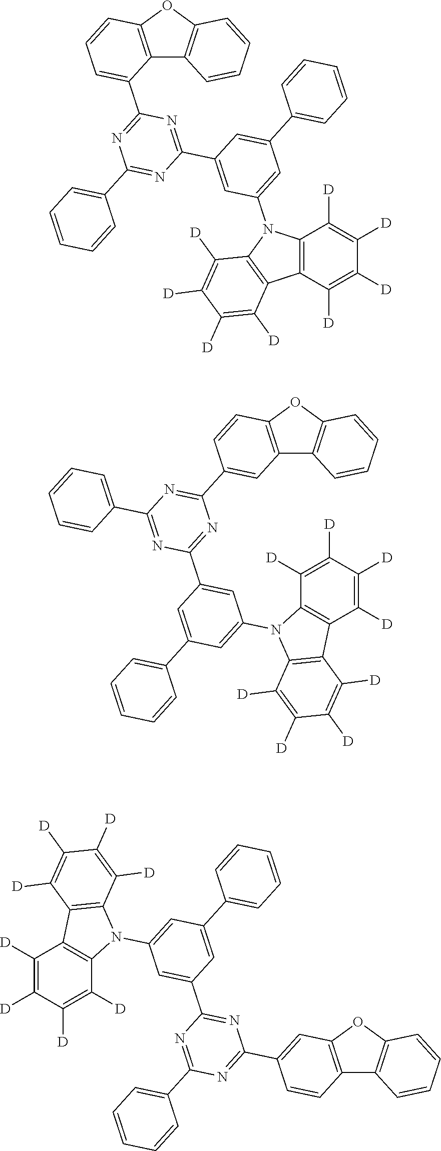 [SMILES: [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3cccc4oc5ccccc5c34)n2)c1 ; [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3ccc4oc5ccccc5c4c3)n2)c1 ; [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3ccc4c(c3)oc3ccccc34)n2)c1]

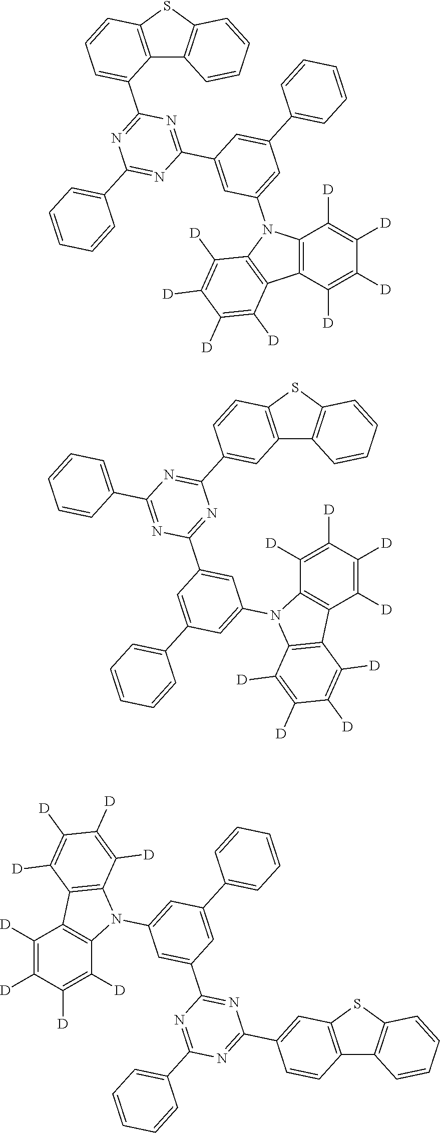 [SMILES: [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3cccc4sc5ccccc5c34)n2)c1 ; [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3ccc4sc5ccccc5c4c3)n2)c1 ; [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3ccc4c(c3)sc3ccccc34)n2)c1]

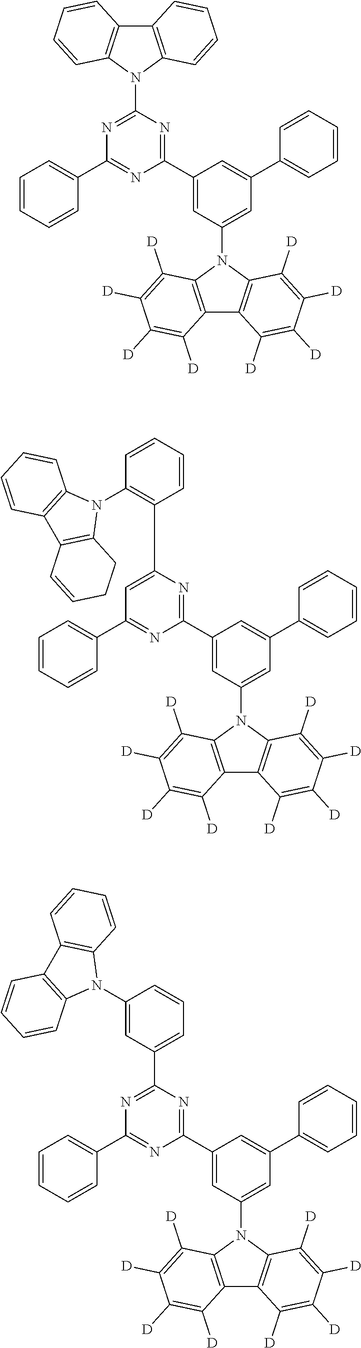

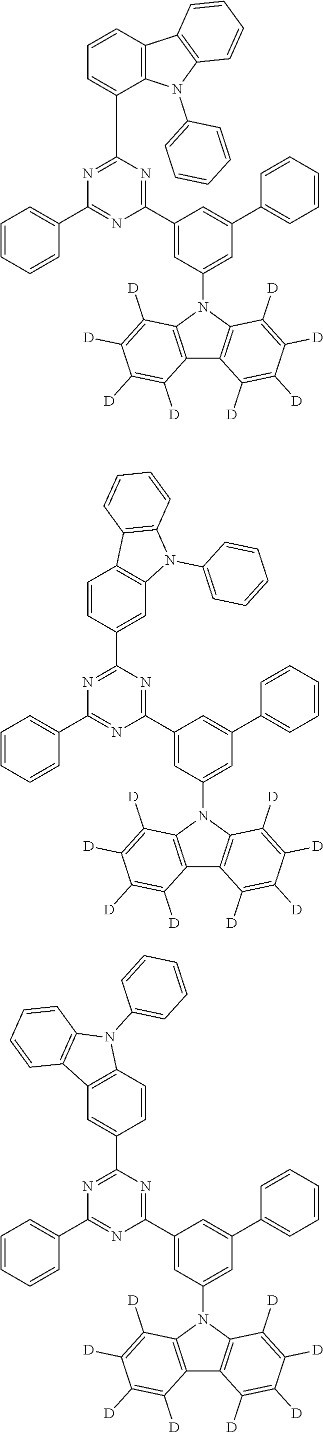

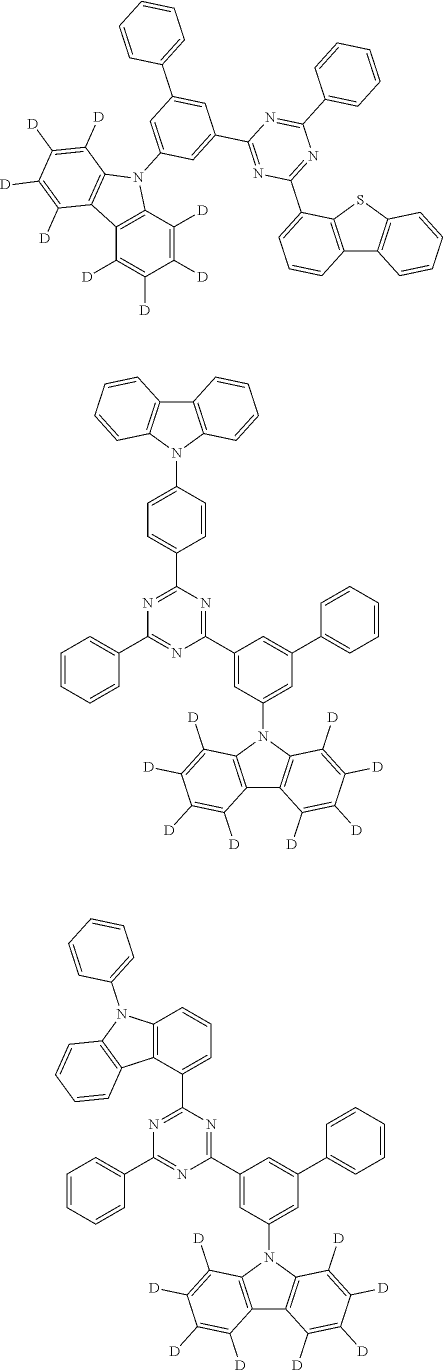 [SMILES: [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3cccc4c3sc3ccccc34)n2)c1 ; [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3ccc(-n4c5ccccc5c5ccccc54)cc3)n2)c1 ; [2H]c1c([2H])c([2H])c2c(c1[2H])c1c([2H])c([2H])c([2H])c([2H])c1n2-c1cc(-c2ccccc2)cc(-c2nc(-c3ccccc3)nc(-c3cccc4c3c3ccccc3n4-c3ccccc3)n2)c1]

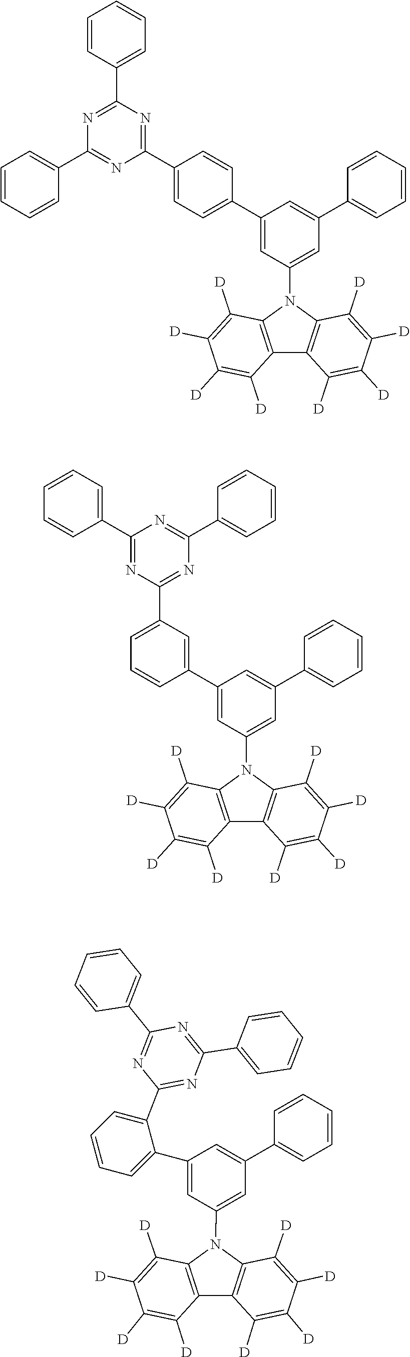 [SMILES: [2H]c1c([2H])c([2H])c2c(c1[2H])c1c([2H])c([2H])c([2H])c([2H])c1n2-c1cc(-c2ccccc2)cc(-c2ccc(-c3nc(-c4ccccc4)nc(-c4ccccc4)n3)cc2)c1 ; [2H]c1c([2H])c([2H])c2c(c1[2H])c1c([2H])c([2H])c([2H])c([2H])c1n2-c1cc(-c2ccccc2)cc(-c2cccc(-c3nc(-c4ccccc4)nc(-c4ccccc4)n3)c2)c1 ; [2H]c1c([2H])c([2H])c2c(c1[2H])c1c([2H])c([2H])c([2H])c([2H])c1n2-c1cc(-c2ccccc2)cc(-c2ccccc2-c2nc(-c3ccccc3)nc(-c3ccccc3)n2)c1]

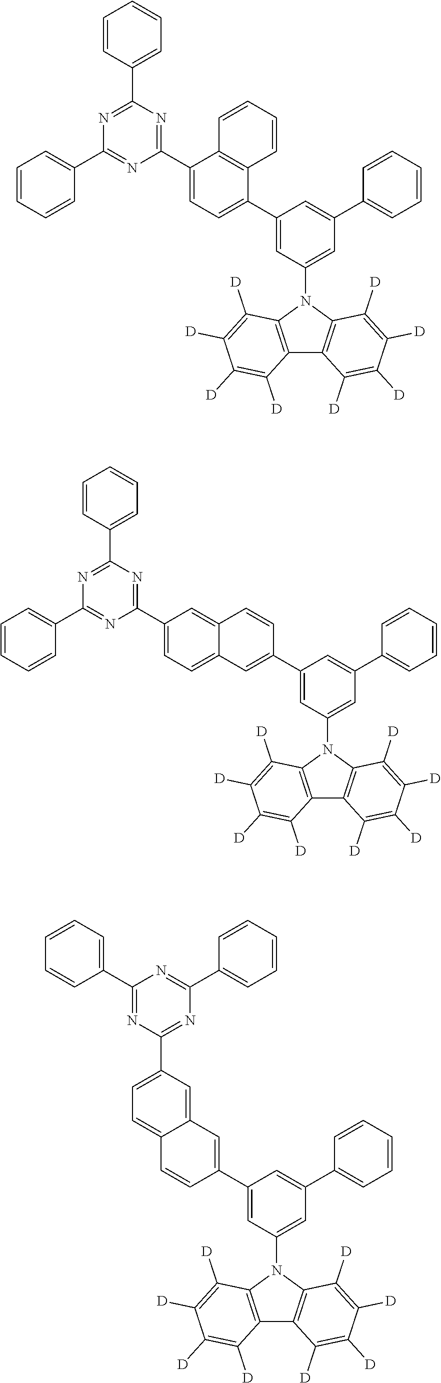 [SMILES: [2H]c1c([2H])c([2H])c2c(c1[2H])c1c([2H])c([2H])c([2H])c([2H])c1n2-c1cc(-c2ccccc2)cc(-c2ccc(-c3nc(-c4ccccc4)nc(-c4ccccc4)n3)c3ccccc23)c1 ; [2H]c1c([2H])c([2H])c2c(c1[2H])c1c([2H])c([2H])c([2H])c([2H])c1n2-c1cc(-c2ccccc2)cc(-c2ccc3cc(-c4nc(-c5ccccc5)nc(-c5ccccc5)n4)ccc3c2)c1 ; [2H]c1c([2H])c([2H])c2c(c1[2H])c1c([2H])c([2H])c([2H])c([2H])c1n2-c1cc(-c2ccccc2)cc(-c2ccc3ccc(-c4nc(-c5ccccc5)nc(-c5ccccc5)n4)cc3c2)c1]

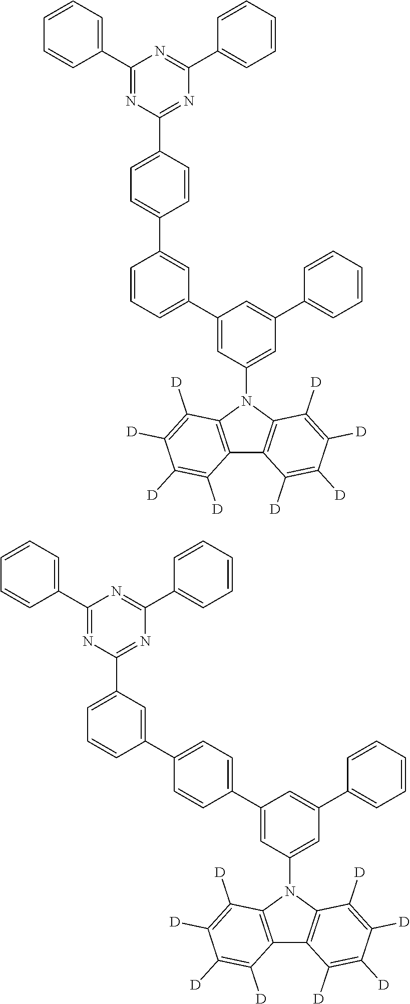

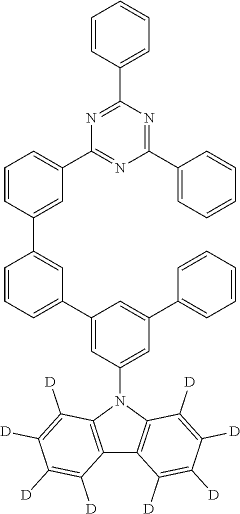 [SMILES: [2H]c1c([2H])c([2H])c2c(c1[2H])c1c([2H])c([2H])c([2H])c([2H])c1n2-c1cc(-c2ccccc2)cc(-c2cccc(-c3cccc(-c4nc(-c5ccccc5)nc(-c5ccccc5)n4)c3)c2)c1]

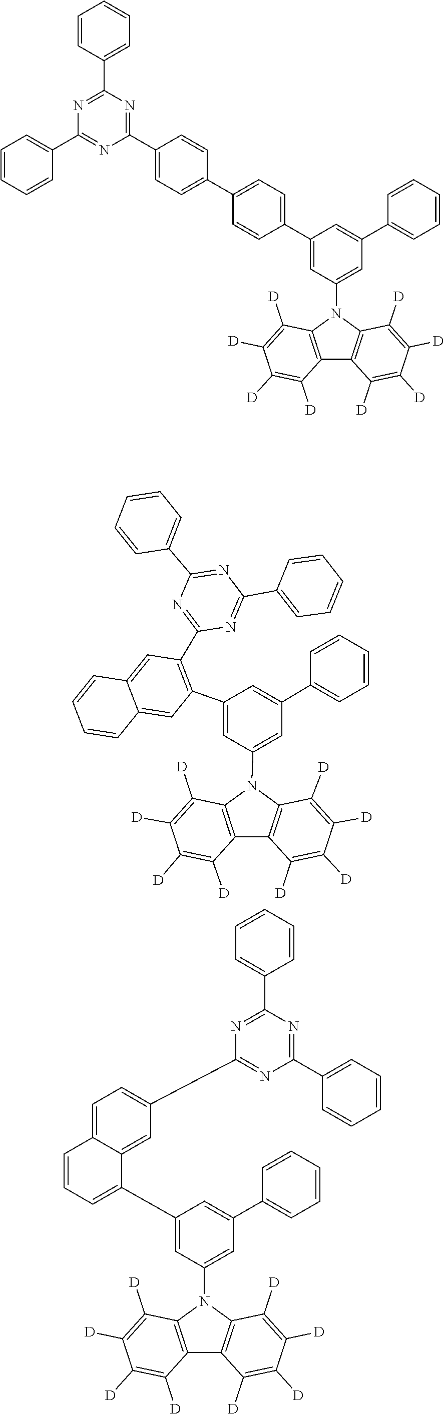

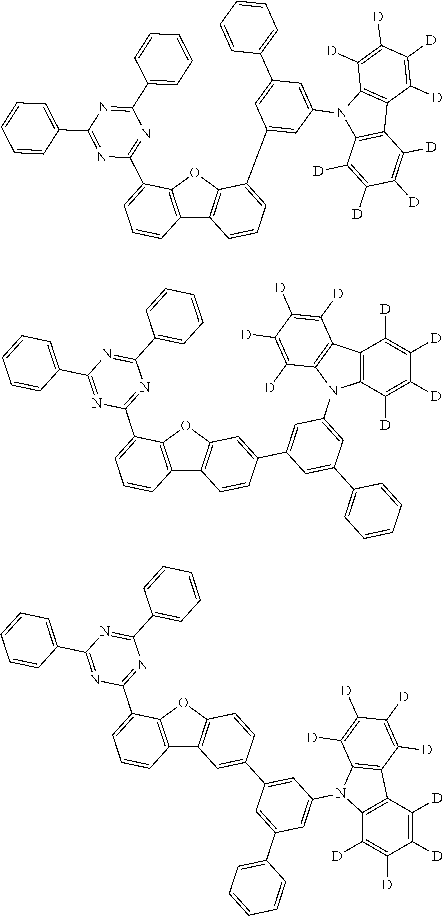 [SMILES: [2H]c1c([2H])c([2H])c2c(c1[2H])c1c([2H])c([2H])c([2H])c([2H])c1n2-c1cc(-c2ccccc2)cc(-c2cccc3c2oc2c(-c4nc(-c5ccccc5)nc(-c5ccccc5)n4)cccc23)c1 ; [2H]c1c([2H])c([2H])c2c(c1[2H])c1c([2H])c([2H])c([2H])c([2H])c1n2-c1cc(-c2ccccc2)cc(-c2ccc3c(c2)oc2c(-c4nc(-c5ccccc5)nc(-c5ccccc5)n4)cccc23)c1 ; [2H]c1c([2H])c([2H])c2c(c1[2H])c1c([2H])c([2H])c([2H])c([2H])c1n2-c1cc(-c2ccccc2)cc(-c2ccc3oc4c(-c5nc(-c6ccccc6)nc(-c6ccccc6)n5)cccc4c3c2)c1]

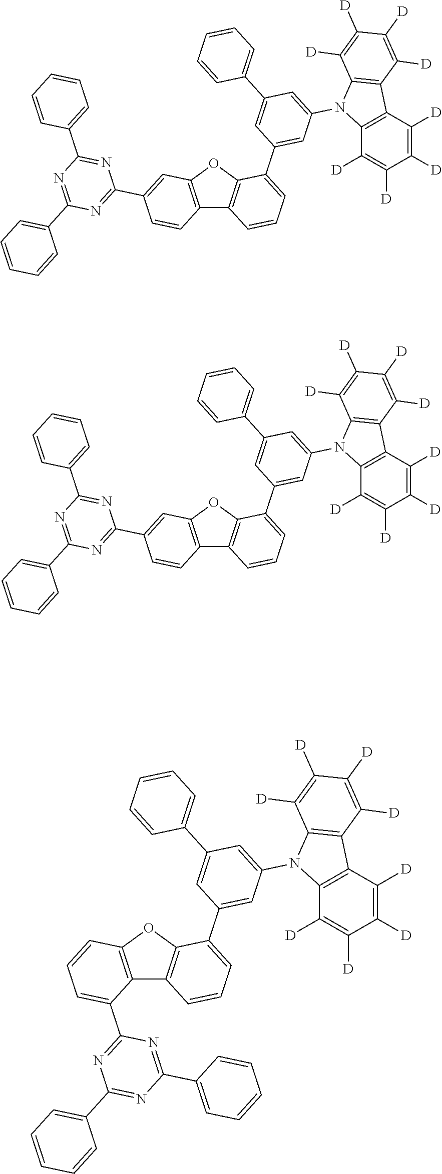 [SMILES: [2H]c1c([2H])c([2H])c2c(c1[2H])c1c([2H])c([2H])c([2H])c([2H])c1n2-c1cc(-c2ccccc2)cc(-c2cccc3c2oc2cc(-c4nc(-c5ccccc5)nc(-c5ccccc5)n4)ccc23)c1 ; [2H]c1c([2H])c([2H])c2c(c1[2H])c1c([2H])c([2H])c([2H])c([2H])c1n2-c1cc(-c2ccccc2)cc(-c2cccc3c2oc2cc(-c4nc(-c5ccccc5)nc(-c5ccccc5)n4)ccc23)c1 ; [2H]c1c([2H])c([2H])c2c(c1[2H])c1c([2H])c([2H])c([2H])c([2H])c1n2-c1cc(-c2ccccc2)cc(-c2cccc3c2oc2cccc(-c4nc(-c5ccccc5)nc(-c5ccccc5)n4)c23)c1]

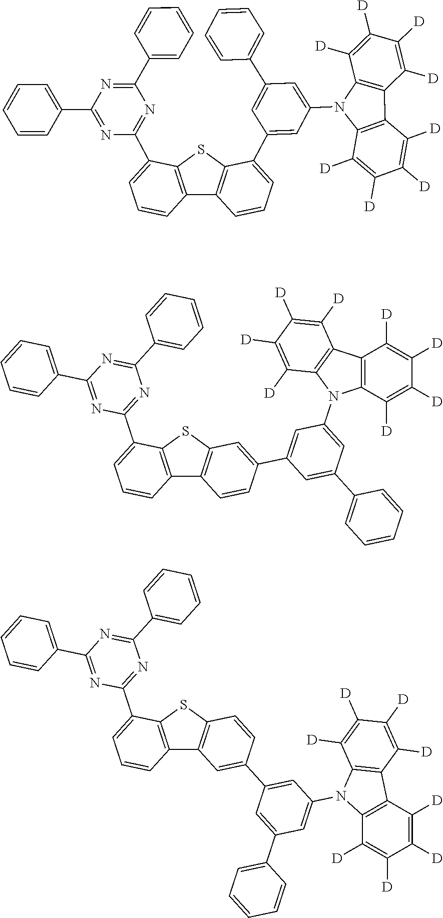 [SMILES: [2H]c1c([2H])c([2H])c2c(c1[2H])c1c([2H])c([2H])c([2H])c([2H])c1n2-c1cc(-c2ccccc2)cc(-c2cccc3c2sc2c(-c4nc(-c5ccccc5)nc(-c5ccccc5)n4)cccc23)c1 ; [2H]c1c([2H])c([2H])c2c(c1[2H])c1c([2H])c([2H])c([2H])c([2H])c1n2-c1cc(-c2ccccc2)cc(-c2ccc3c(c2)sc2c(-c4nc(-c5ccccc5)nc(-c5ccccc5)n4)cccc23)c1 ; [2H]c1c([2H])c([2H])c2c(c1[2H])c1c([2H])c([2H])c([2H])c([2H])c1n2-c1cc(-c2ccccc2)cc(-c2ccc3sc4c(-c5nc(-c6ccccc6)nc(-c6ccccc6)n5)cccc4c3c2)c1]

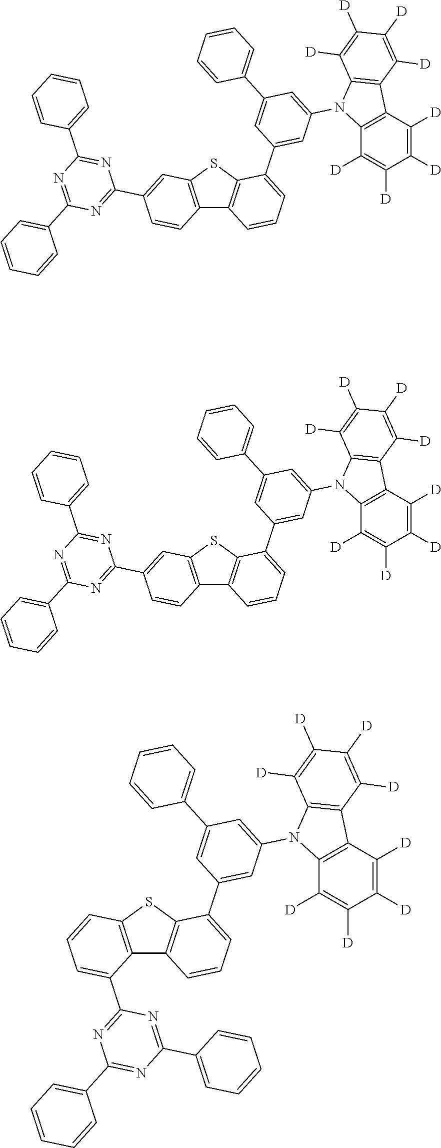 [SMILES: [2H]c1c([2H])c([2H])c2c(c1[2H])c1c([2H])c([2H])c([2H])c([2H])c1n2-c1cc(-c2ccccc2)cc(-c2cccc3c2sc2cc(-c4nc(-c5ccccc5)nc(-c5ccccc5)n4)ccc23)c1 ; [2H]c1c([2H])c([2H])c2c(c1[2H])c1c([2H])c([2H])c([2H])c([2H])c1n2-c1cc(-c2ccccc2)cc(-c2cccc3c2sc2cc(-c4nc(-c5ccccc5)nc(-c5ccccc5)n4)ccc23)c1 ; [2H]c1c([2H])c([2H])c2c(c1[2H])c1c([2H])c([2H])c([2H])c([2H])c1n2-c1cc(-c2ccccc2)cc(-c2cccc3c2sc2cccc(-c4nc(-c5ccccc5)nc(-c5ccccc5)n4)c23)c1]

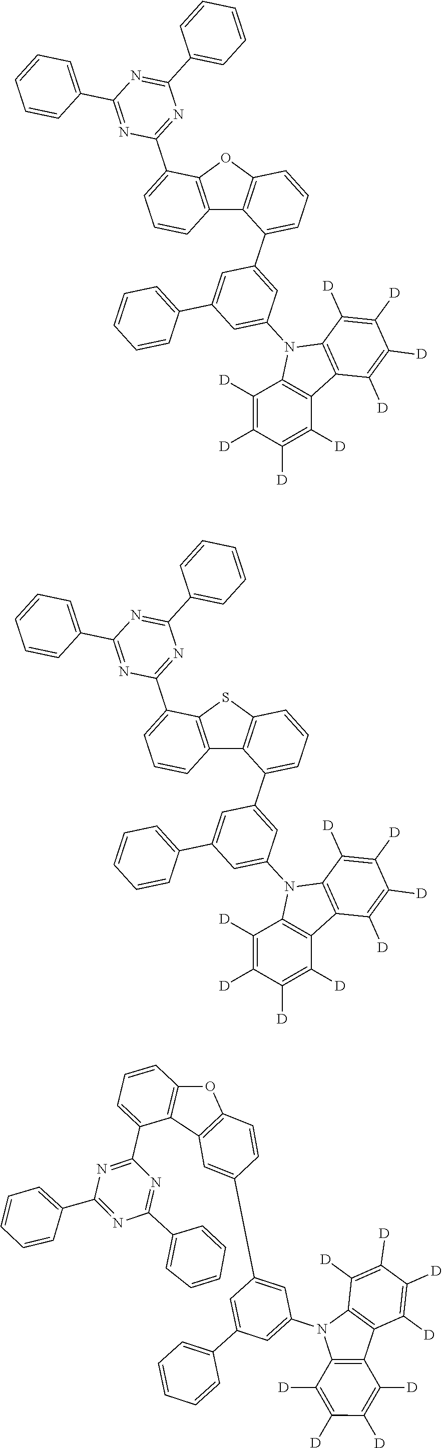 [SMILES: [2H]c1c([2H])c([2H])c2c(c1[2H])c1c([2H])c([2H])c([2H])c([2H])c1n2-c1cc(-c2ccccc2)cc(-c2cccc3oc4c(-c5nc(-c6ccccc6)nc(-c6ccccc6)n5)cccc4c23)c1 ; [2H]c1c([2H])c([2H])c2c(c1[2H])c1c([2H])c([2H])c([2H])c([2H])c1n2-c1cc(-c2ccccc2)cc(-c2cccc3sc4c(-c5nc(-c6ccccc6)nc(-c6ccccc6)n5)cccc4c23)c1 ; [2H]c1c([2H])c([2H])c2c(c1[2H])c1c([2H])c([2H])c([2H])c([2H])c1n2-c1cc(-c2ccccc2)cc(-c2ccc3oc4cccc(-c5nc(-c6ccccc6)nc(-c6ccccc6)n5)c4c3c2)c1]

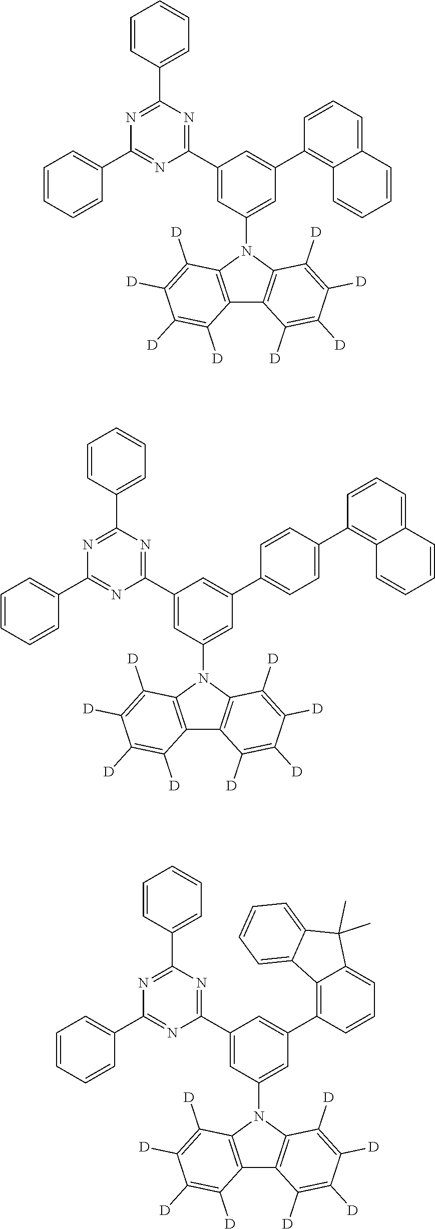 [SMILES: [2H]c1c([2H])c([2H])c2c(c1[2H])c1c([2H])c([2H])c([2H])c([2H])c1n2-c1cc(-c2nc(-c3ccccc3)nc(-c3ccccc3)n2)cc(-c2cccc3ccccc23)c1 ; [2H]c1c([2H])c([2H])c2c(c1[2H])c1c([2H])c([2H])c([2H])c([2H])c1n2-c1cc(-c2ccc(-c3cccc4ccccc34)cc2)cc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cc(-c2nc(-c3ccccc3)nc(-c3ccccc3)n2)cc(-c2cccc3c2-c2ccccc2C3(C)C)c1]

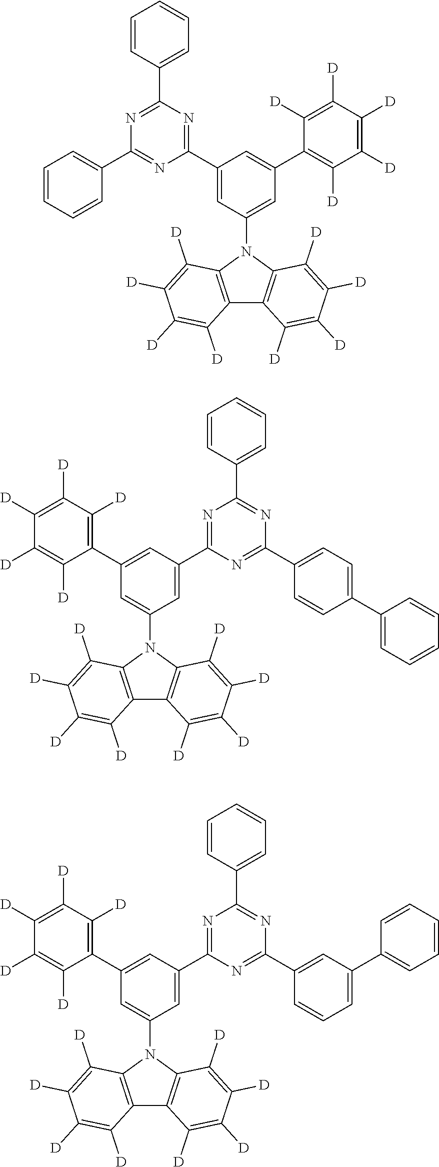

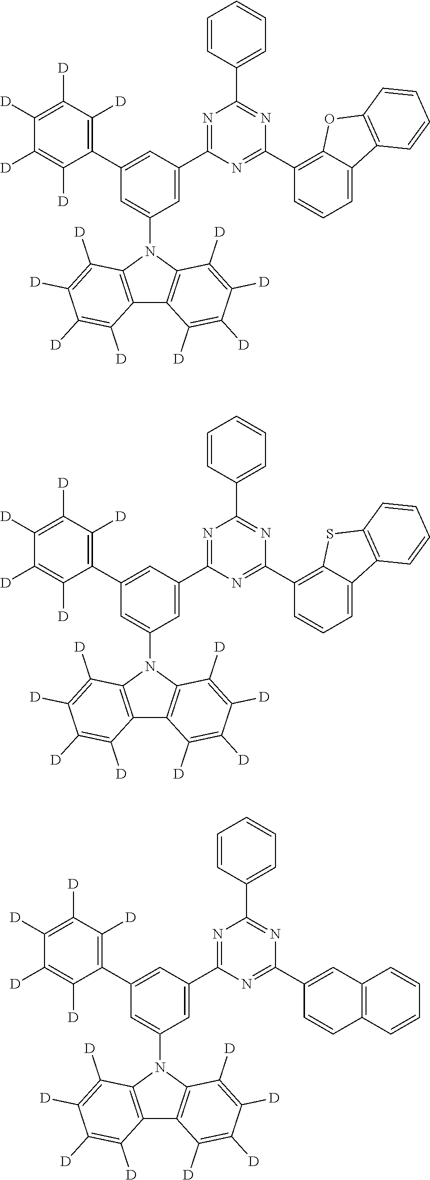 [SMILES: [2H]c1c([2H])c([2H])c(-c2cc(-c3nc(-c4ccccc4)nc(-c4cccc5c4oc4ccccc45)n3)cc(-n3c4c([2H])c([2H])c([2H])c([2H])c4c4c([2H])c([2H])c([2H])c([2H])c43)c2)c([2H])c1[2H] ; [2H]c1c([2H])c([2H])c(-c2cc(-c3nc(-c4ccccc4)nc(-c4cccc5c4sc4ccccc45)n3)cc(-n3c4c([2H])c([2H])c([2H])c([2H])c4c4c([2H])c([2H])c([2H])c([2H])c43)c2)c([2H])c1[2H] ; [2H]c1c([2H])c([2H])c(-c2cc(-c3nc(-c4ccccc4)nc(-c4ccc5ccccc5c4)n3)cc(-n3c4c([2H])c([2H])c([2H])c([2H])c4c4c([2H])c([2H])c([2H])c([2H])c43)c2)c([2H])c1[2H]]

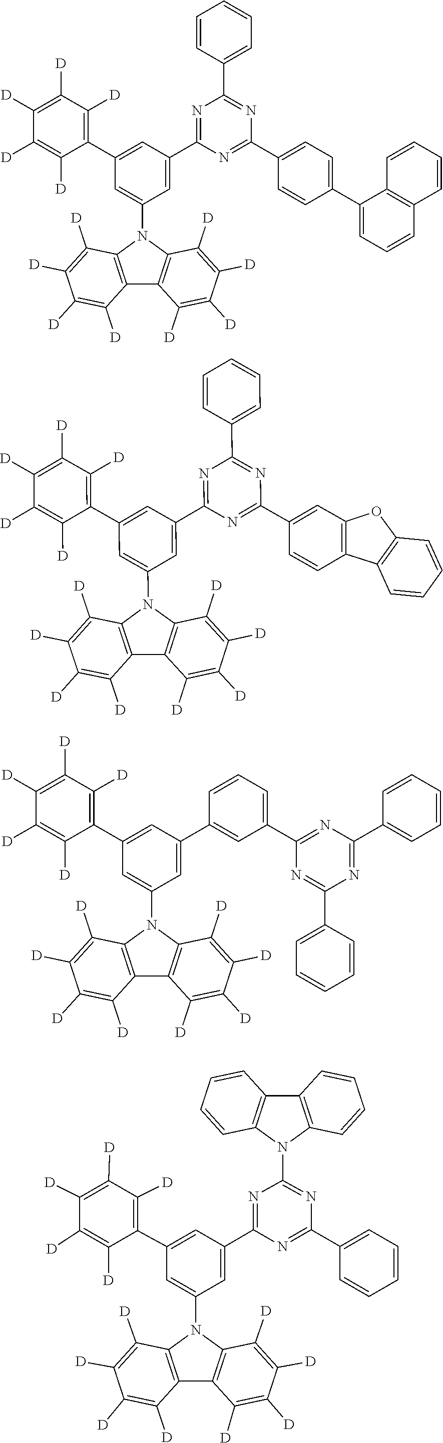

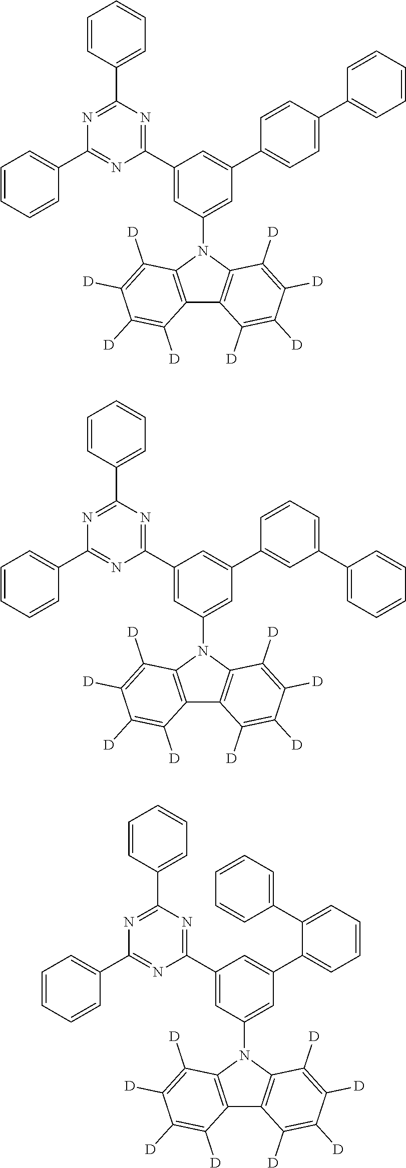 [SMILES: [2H]c1c([2H])c([2H])c2c(c1[2H])c1c([2H])c([2H])c([2H])c([2H])c1n2-c1cc(-c2ccc(-c3ccccc3)cc2)cc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cc(-c2cccc(-c3ccccc3)c2)cc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cc(-c2nc(-c3ccccc3)nc(-c3ccccc3)n2)cc(-c2ccccc2-c2ccccc2)c1]

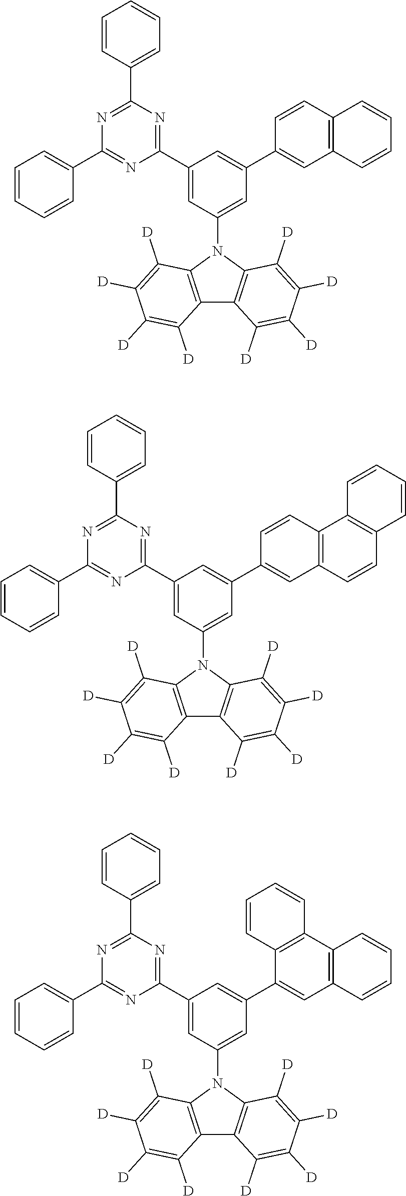 [SMILES: [2H]c1c([2H])c([2H])c2c(c1[2H])c1c([2H])c([2H])c([2H])c([2H])c1n2-c1cc(-c2ccc3ccccc3c2)cc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cc(-c2ccc3c(ccc4ccccc43)c2)cc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cc(-c2nc(-c3ccccc3)nc(-c3ccccc3)n2)cc(-c2cc3ccccc3c3ccccc23)c1]

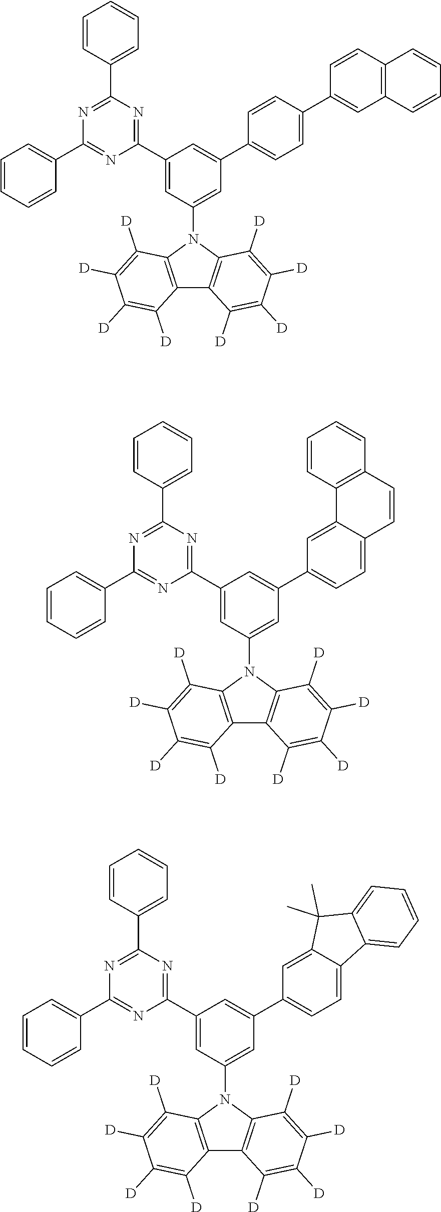 [SMILES: [2H]c1c([2H])c([2H])c2c(c1[2H])c1c([2H])c([2H])c([2H])c([2H])c1n2-c1cc(-c2ccc(-c3ccc4ccccc4c3)cc2)cc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cc(-c2ccc3ccc4ccccc4c3c2)cc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cc(-c2ccc3c(c2)C(C)(C)c2ccccc2-3)cc(-c2nc(-c3ccccc3)nc(-c3ccccc3)n2)c1]

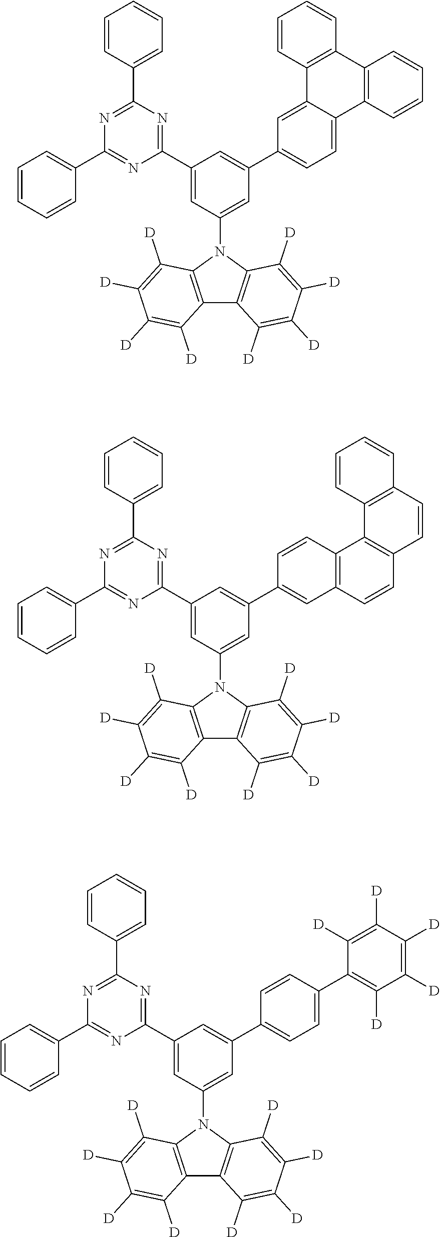 [SMILES: [2H]c1c([2H])c([2H])c2c(c1[2H])c1c([2H])c([2H])c([2H])c([2H])c1n2-c1cc(-c2ccc3c4ccccc4c4ccccc4c3c2)cc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cc(-c2ccc3c(ccc4ccc5ccccc5c43)c2)cc(-c2nc(-c3ccccc3)nc(-c3ccccc3)n2)c1 ; [2H]c1c([2H])c([2H])c(-c2ccc(-c3cc(-c4nc(-c5ccccc5)nc(-c5ccccc5)n4)cc(-n4c5c([2H])c([2H])c([2H])c([2H])c5c5c([2H])c([2H])c([2H])c([2H])c54)c3)cc2)c([2H])c1[2H]]

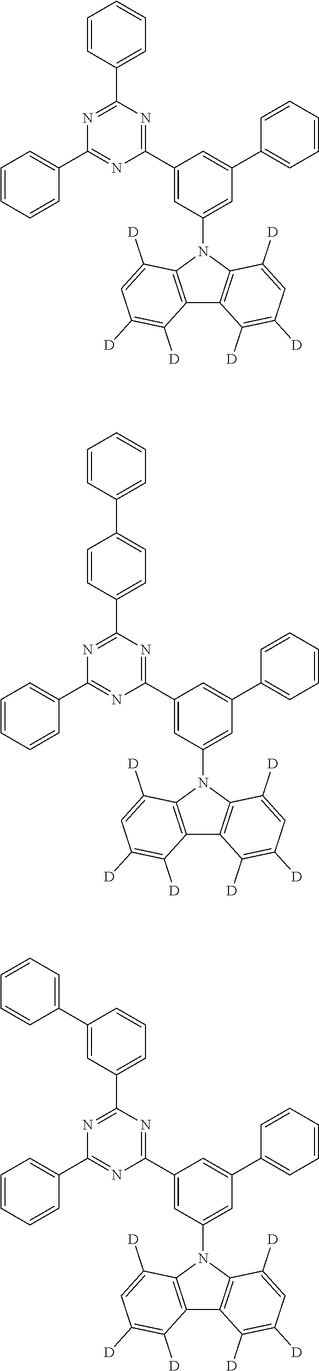 [SMILES: [2H]c1cc([2H])c2c(c1[2H])c1c([2H])c([2H])cc([2H])c1n2-c1cc(-c2ccccc2)cc(-c2nc(-c3ccccc3)nc(-c3ccccc3)n2)c1 ; [2H]c1cc([2H])c2c(c1[2H])c1c([2H])c([2H])cc([2H])c1n2-c1cc(-c2ccccc2)cc(-c2nc(-c3ccccc3)nc(-c3ccc(-c4ccccc4)cc3)n2)c1 ; [2H]c1cc([2H])c2c(c1[2H])c1c([2H])c([2H])cc([2H])c1n2-c1cc(-c2ccccc2)cc(-c2nc(-c3ccccc3)nc(-c3cccc(-c4ccccc4)c3)n2)c1]

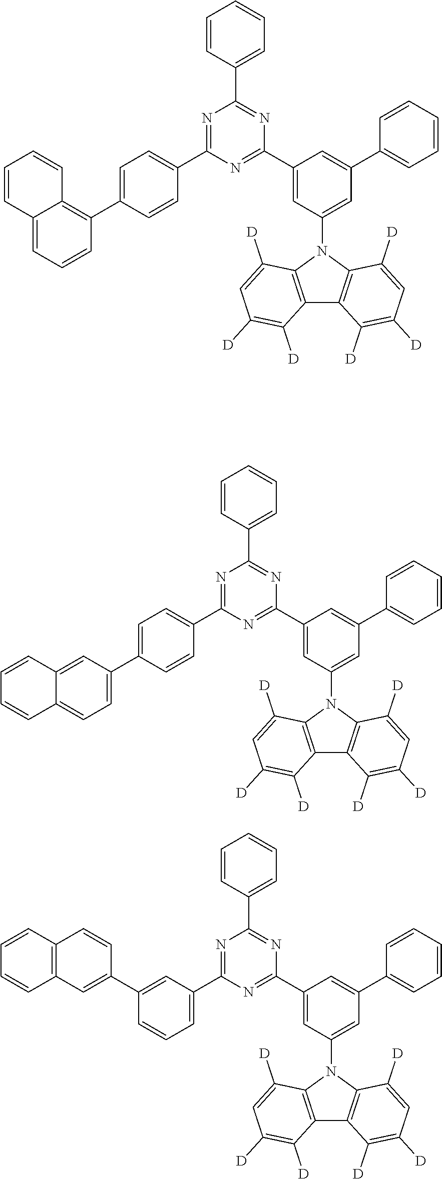

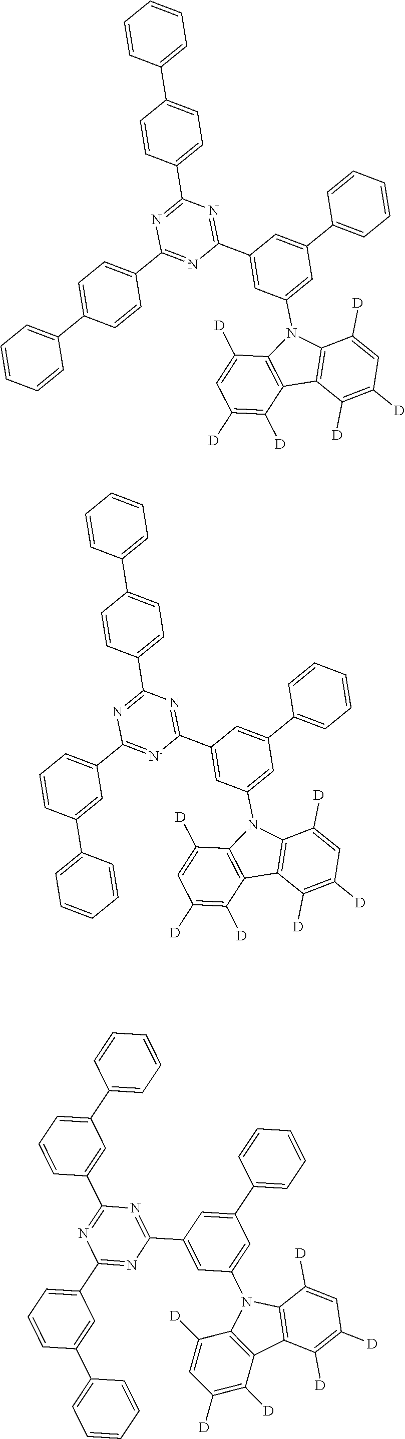

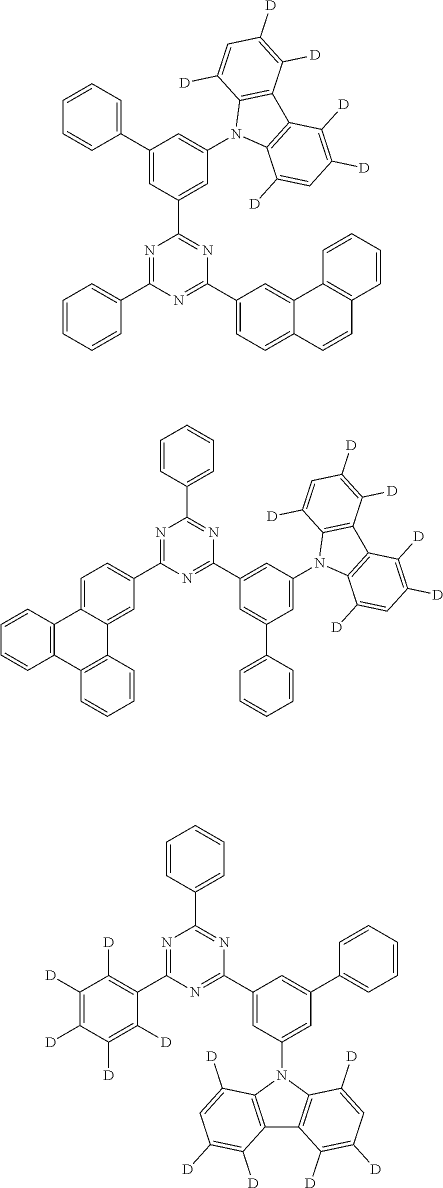 [SMILES: [2H]c1cc([2H])c2c(c1[2H])c1c([2H])c([2H])cc([2H])c1n2-c1cc(-c2ccccc2)cc(-c2nc(-c3ccccc3)nc(-c3ccc4ccc5ccccc5c4c3)n2)c1 ; [2H]c1cc([2H])c2c(c1[2H])c1c([2H])c([2H])cc([2H])c1n2-c1cc(-c2ccccc2)cc(-c2nc(-c3ccccc3)nc(-c3ccc4c5ccccc5c5ccccc5c4c3)n2)c1 ; [2H]c1cc([2H])c2c(c1[2H])c1c([2H])c([2H])cc([2H])c1n2-c1cc(-c2ccccc2)cc(-c2nc(-c3ccccc3)nc(-c3c([2H])c([2H])c([2H])c([2H])c3[2H])n2)c1]

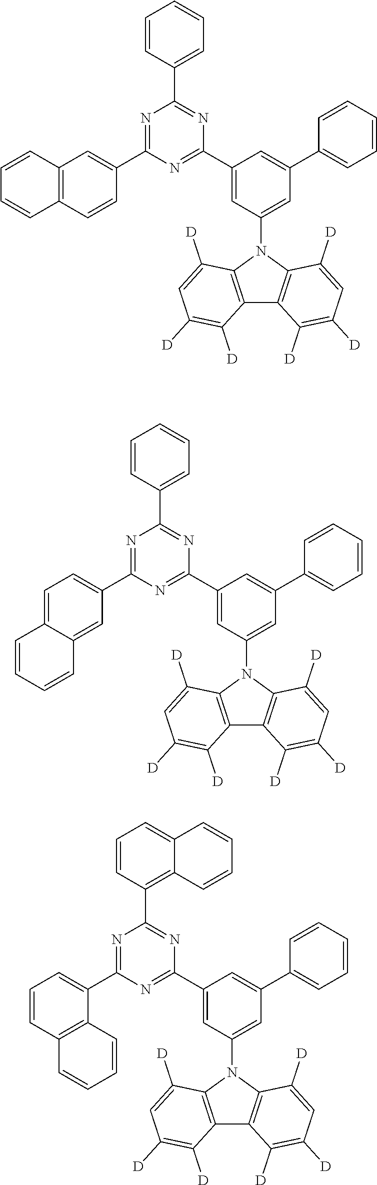

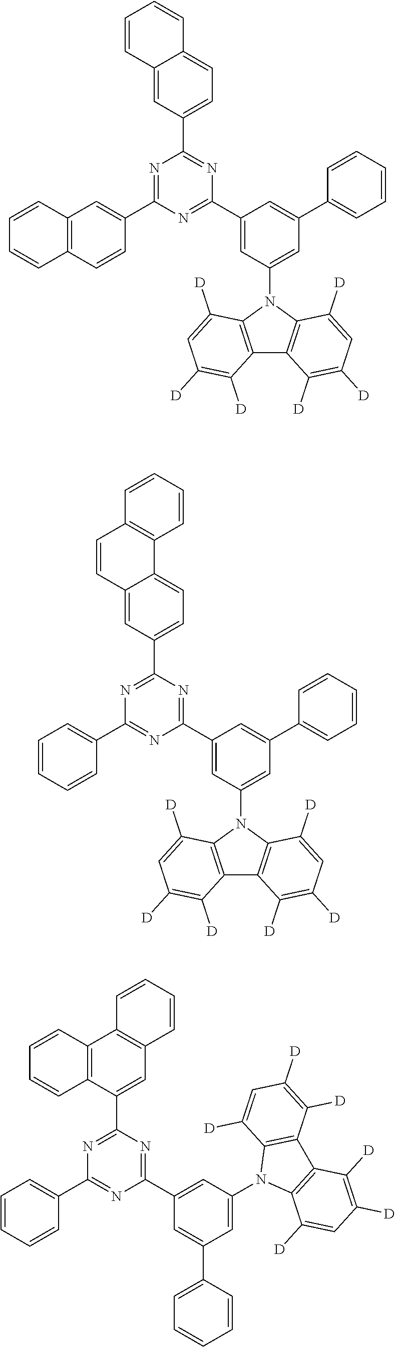 [SMILES: [2H]c1cc([2H])c2c(c1[2H])c1c([2H])c([2H])cc([2H])c1n2-c1cc(-c2ccccc2)cc(-c2nc(-c3ccc4ccccc4c3)nc(-c3ccc4ccccc4c3)n2)c1 ; [2H]c1cc([2H])c2c(c1[2H])c1c([2H])c([2H])cc([2H])c1n2-c1cc(-c2ccccc2)cc(-c2nc(-c3ccccc3)nc(-c3ccc4c(ccc5ccccc54)c3)n2)c1 ; [2H]c1cc([2H])c2c(c1[2H])c1c([2H])c([2H])cc([2H])c1n2-c1cc(-c2ccccc2)cc(-c2nc(-c3ccccc3)nc(-c3cc4ccccc4c4ccccc34)n2)c1]

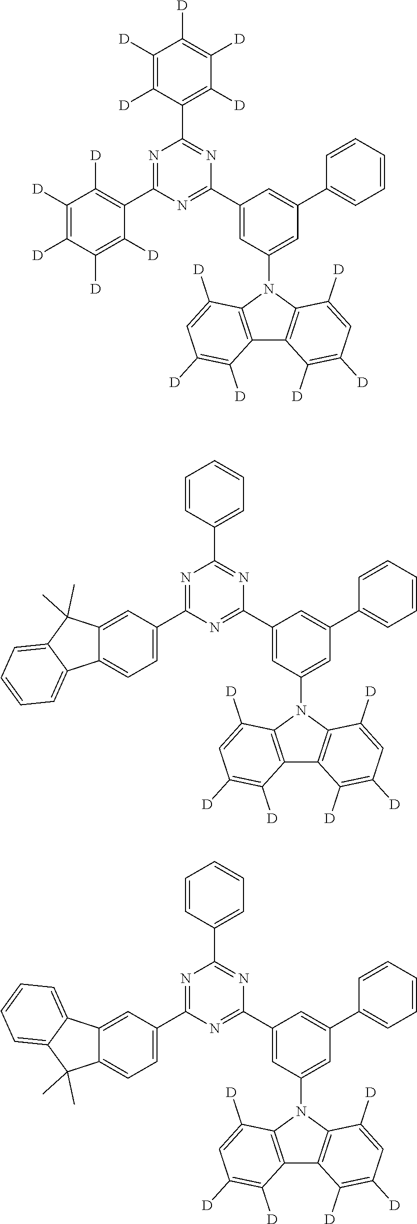 [SMILES: [2H]c1cc([2H])c2c(c1[2H])c1c([2H])c([2H])cc([2H])c1n2-c1cc(-c2ccccc2)cc(-c2nc(-c3c([2H])c([2H])c([2H])c([2H])c3[2H])nc(-c3c([2H])c([2H])c([2H])c([2H])c3[2H])n2)c1 ; [2H]c1cc([2H])c2c(c1[2H])c1c([2H])c([2H])cc([2H])c1n2-c1cc(-c2ccccc2)cc(-c2nc(-c3ccccc3)nc(-c3ccc4c(c3)C(C)(C)c3ccccc3-4)n2)c1 ; [2H]c1cc([2H])c2c(c1[2H])c1c([2H])c([2H])cc([2H])c1n2-c1cc(-c2ccccc2)cc(-c2nc(-c3ccccc3)nc(-c3ccc4c(c3)-c3ccccc3C4(C)C)n2)c1]

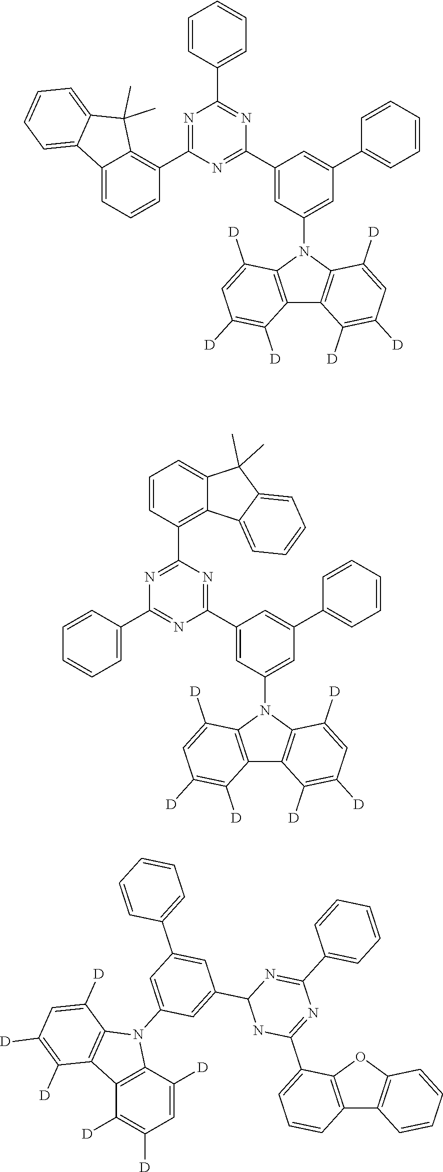

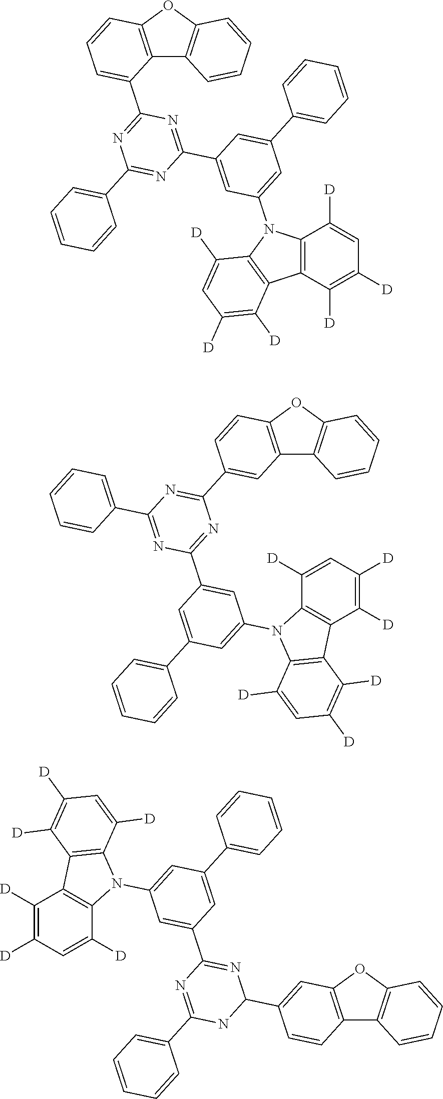

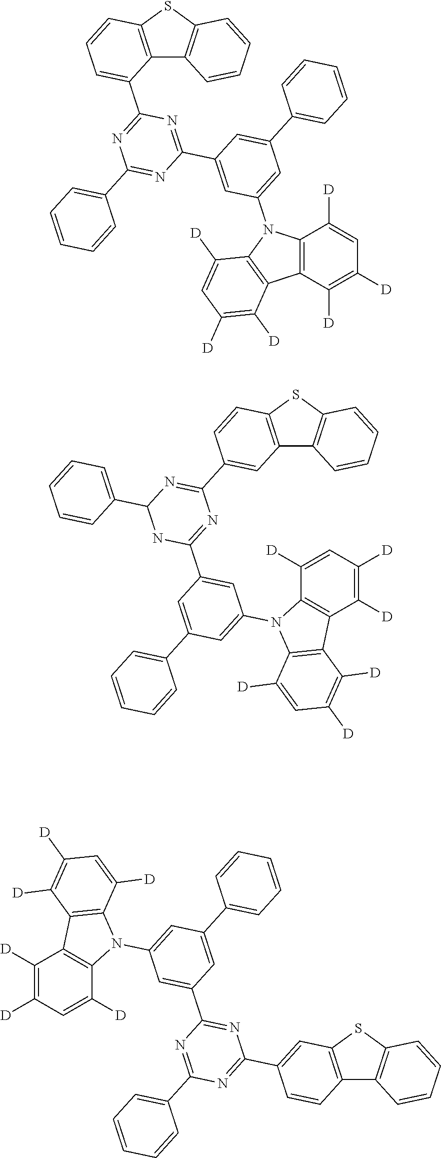

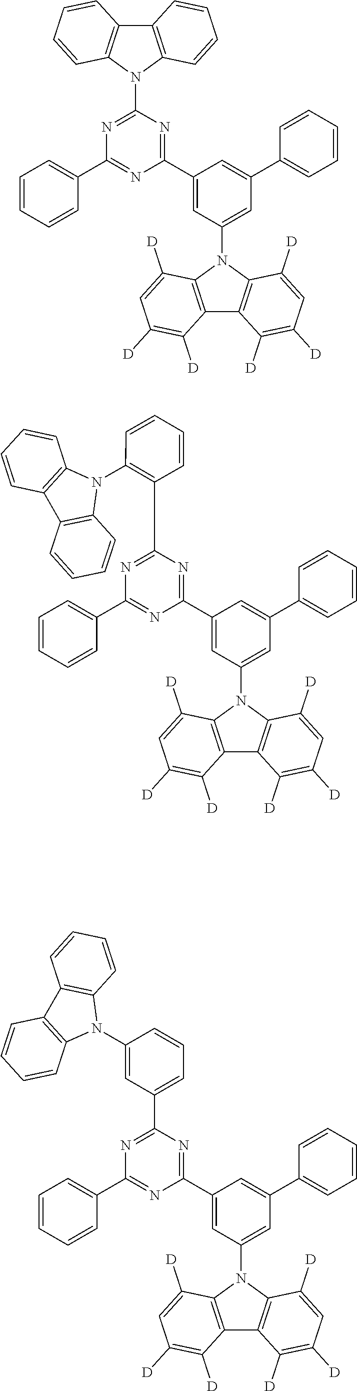

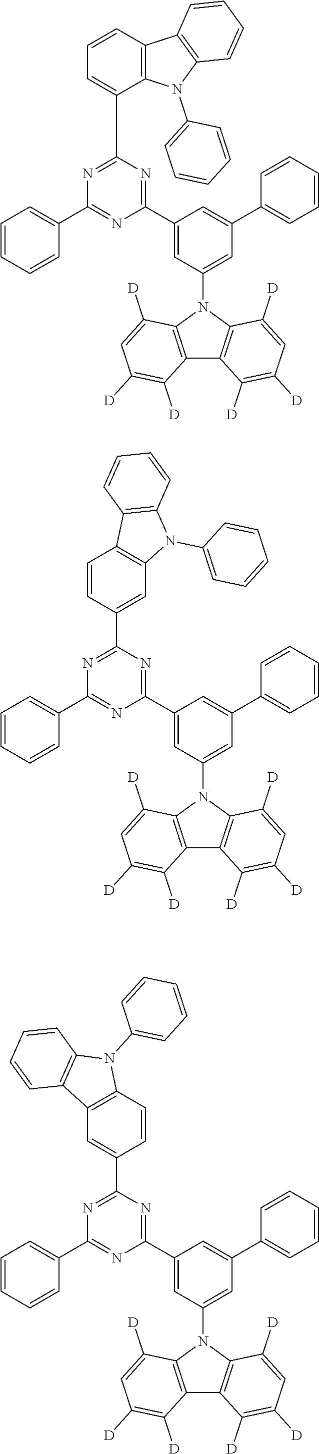

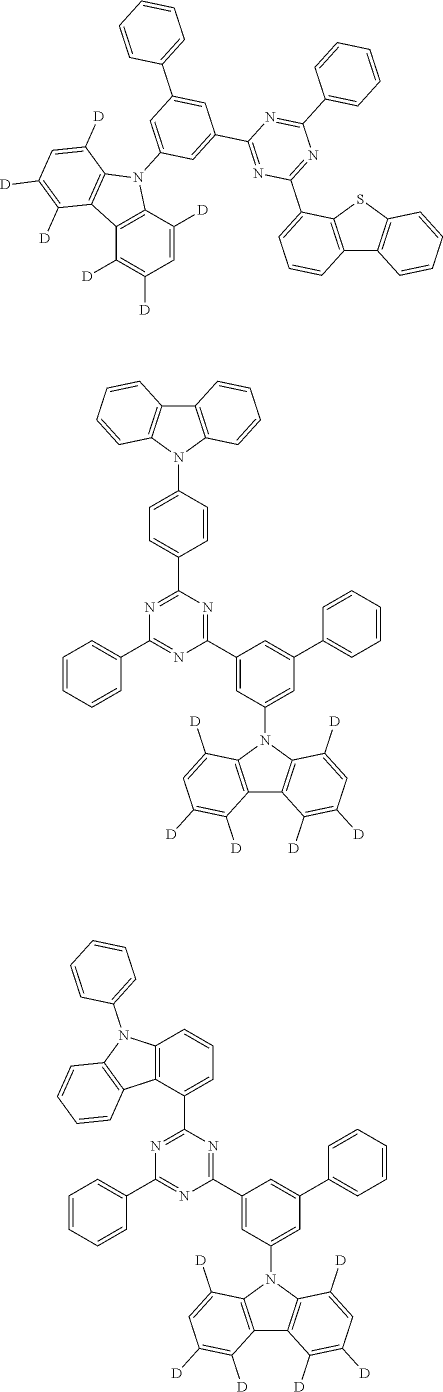 [SMILES: [2H]c1cc([2H])c2c(c1[2H])c1c([2H])c([2H])cc([2H])c1n2-c1cc(-c2ccccc2)cc(-c2nc(-c3ccccc3)nc(-c3cccc4c3sc3ccccc34)n2)c1 ; [2H]c1cc([2H])c2c(c1[2H])c1c([2H])c([2H])cc([2H])c1n2-c1cc(-c2ccccc2)cc(-c2nc(-c3ccccc3)nc(-c3ccc(-n4c5ccccc5c5ccccc54)cc3)n2)c1 ; [2H]c1cc([2H])c2c(c1[2H])c1c([2H])c([2H])cc([2H])c1n2-c1cc(-c2ccccc2)cc(-c2nc(-c3ccccc3)nc(-c3cccc4c3c3ccccc3n4-c3ccccc3)n2)c1]

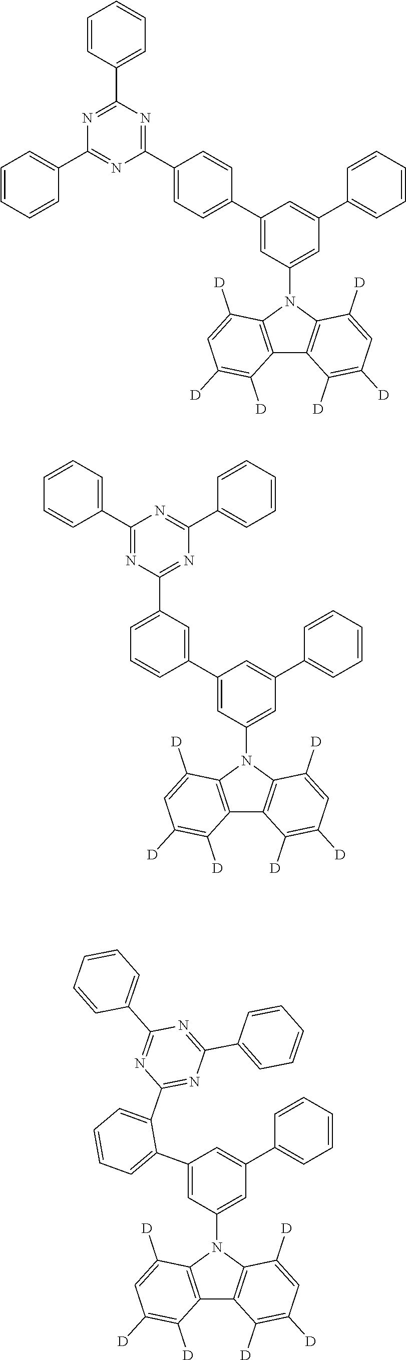 [SMILES: [2H]c1cc([2H])c2c(c1[2H])c1c([2H])c([2H])cc([2H])c1n2-c1cc(-c2ccccc2)cc(-c2ccc(-c3nc(-c4ccccc4)nc(-c4ccccc4)n3)cc2)c1 ; [2H]c1cc([2H])c2c(c1[2H])c1c([2H])c([2H])cc([2H])c1n2-c1cc(-c2ccccc2)cc(-c2cccc(-c3nc(-c4ccccc4)nc(-c4ccccc4)n3)c2)c1 ; [2H]c1cc([2H])c2c(c1[2H])c1c([2H])c([2H])cc([2H])c1n2-c1cc(-c2ccccc2)cc(-c2ccccc2-c2nc(-c3ccccc3)nc(-c3ccccc3)n2)c1]

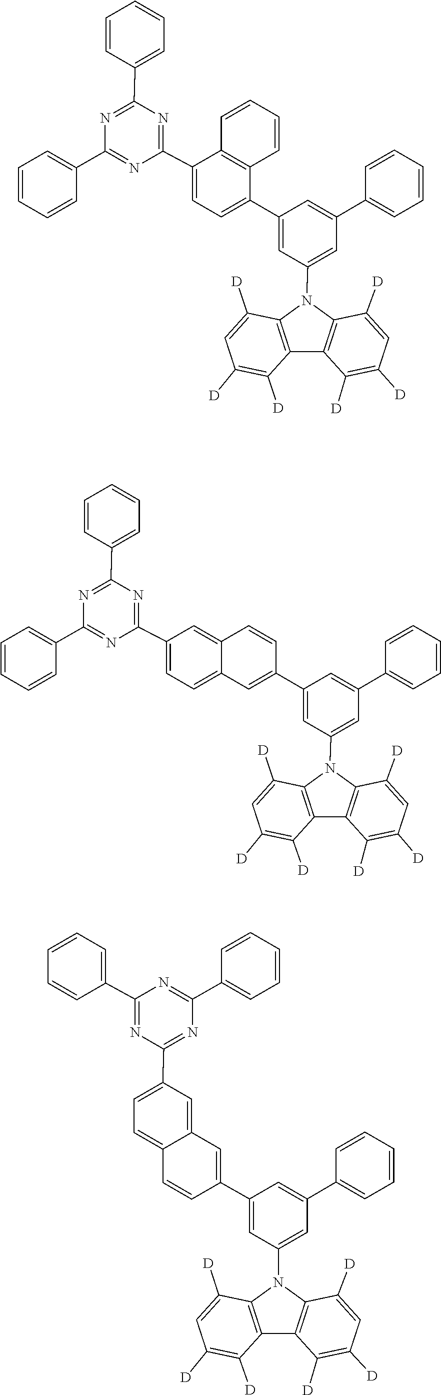 [SMILES: [2H]c1cc([2H])c2c(c1[2H])c1c([2H])c([2H])cc([2H])c1n2-c1cc(-c2ccccc2)cc(-c2ccc(-c3nc(-c4ccccc4)nc(-c4ccccc4)n3)c3ccccc23)c1 ; [2H]c1cc([2H])c2c(c1[2H])c1c([2H])c([2H])cc([2H])c1n2-c1cc(-c2ccccc2)cc(-c2ccc3cc(-c4nc(-c5ccccc5)nc(-c5ccccc5)n4)ccc3c2)c1 ; [2H]c1cc([2H])c2c(c1[2H])c1c([2H])c([2H])cc([2H])c1n2-c1cc(-c2ccccc2)cc(-c2ccc3ccc(-c4nc(-c5ccccc5)nc(-c5ccccc5)n4)cc3c2)c1]

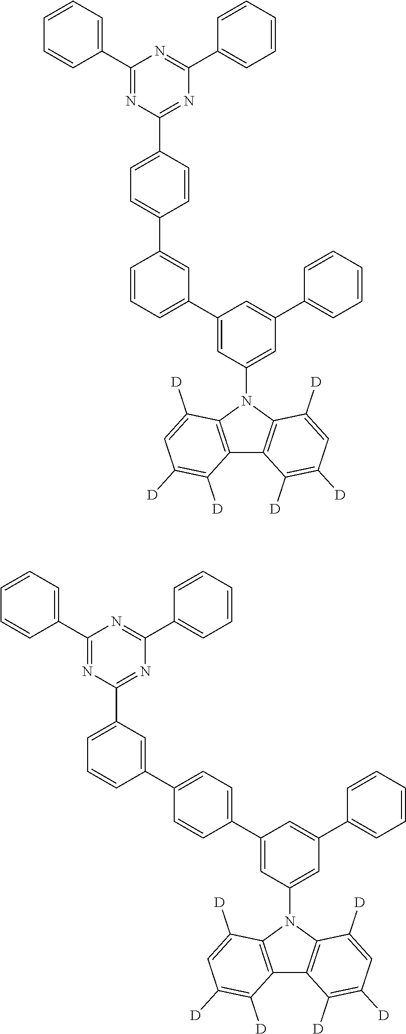 [SMILES: [2H]c1cc([2H])c2c(c1[2H])c1c([2H])c([2H])cc([2H])c1n2-c1cc(-c2ccccc2)cc(-c2cccc(-c3ccc(-c4nc(-c5ccccc5)nc(-c5ccccc5)n4)cc3)c2)c1 ; [2H]c1cc([2H])c2c(c1[2H])c1c([2H])c([2H])cc([2H])c1n2-c1cc(-c2ccccc2)cc(-c2ccc(-c3cccc(-c4nc(-c5ccccc5)nc(-c5ccccc5)n4)c3)cc2)c1]

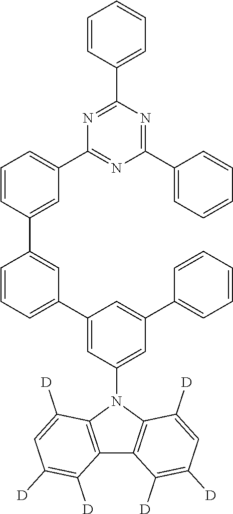 [SMILES: [2H]c1cc([2H])c2c(c1[2H])c1c([2H])c([2H])cc([2H])c1n2-c1cc(-c2ccccc2)cc(-c2cccc(-c3cccc(-c4nc(-c5ccccc5)nc(-c5ccccc5)n4)c3)c2)c1]

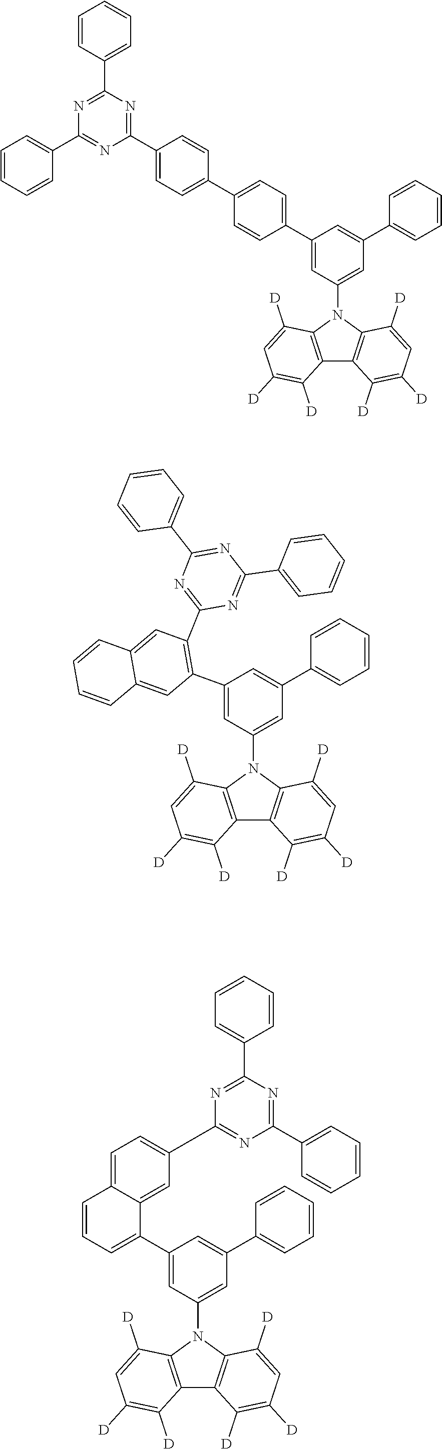 [SMILES: [2H]c1cc([2H])c2c(c1[2H])c1c([2H])c([2H])cc([2H])c1n2-c1cc(-c2ccccc2)cc(-c2ccc(-c3ccc(-c4nc(-c5ccccc5)nc(-c5ccccc5)n4)cc3)cc2)c1 ; [2H]c1cc([2H])c2c(c1[2H])c1c([2H])c([2H])cc([2H])c1n2-c1cc(-c2ccccc2)cc(-c2cc3ccccc3cc2-c2nc(-c3ccccc3)nc(-c3ccccc3)n2)c1 ; [2H]c1cc([2H])c2c(c1[2H])c1c([2H])c([2H])cc([2H])c1n2-c1cc(-c2ccccc2)cc(-c2cccc3ccc(-c4nc(-c5ccccc5)nc(-c5ccccc5)n4)cc23)c1]

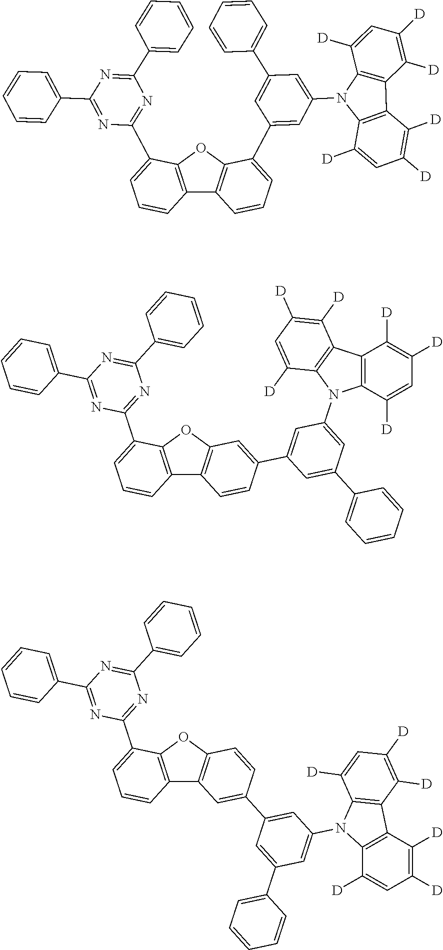 [SMILES: [2H]c1cc([2H])c2c(c1[2H])c1c([2H])c([2H])cc([2H])c1n2-c1cc(-c2ccccc2)cc(-c2cccc3c2oc2c(-c4nc(-c5ccccc5)nc(-c5ccccc5)n4)cccc23)c1 ; [2H]c1cc([2H])c2c(c1[2H])c1c([2H])c([2H])cc([2H])c1n2-c1cc(-c2ccccc2)cc(-c2ccc3c(c2)oc2c(-c4nc(-c5ccccc5)nc(-c5ccccc5)n4)cccc23)c1 ; [2H]c1cc([2H])c2c(c1[2H])c1c([2H])c([2H])cc([2H])c1n2-c1cc(-c2ccccc2)cc(-c2ccc3oc4c(-c5nc(-c6ccccc6)nc(-c6ccccc6)n5)cccc4c3c2)c1]

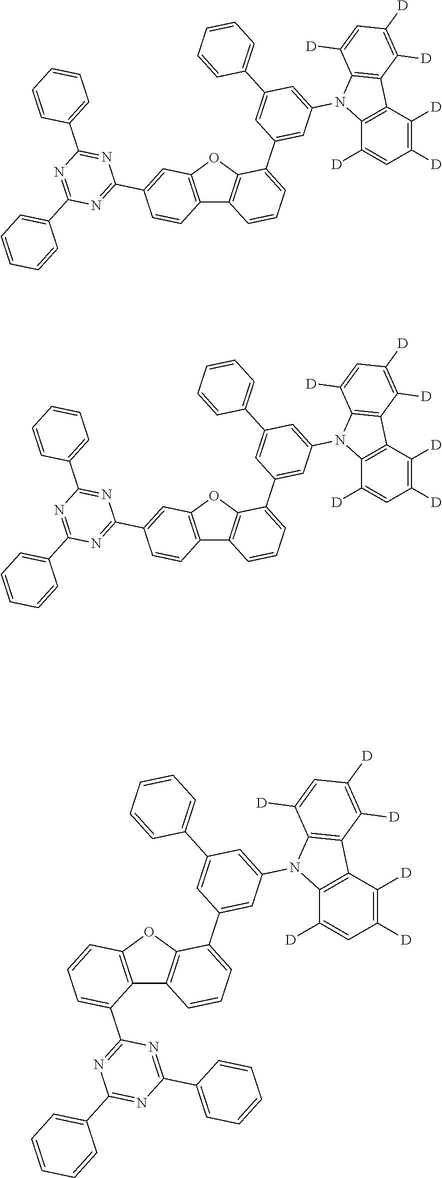 [SMILES: [2H]c1cc([2H])c2c(c1[2H])c1c([2H])c([2H])cc([2H])c1n2-c1cc(-c2ccccc2)cc(-c2cccc3c2oc2cc(-c4nc(-c5ccccc5)nc(-c5ccccc5)n4)ccc23)c1 ; [2H]c1cc([2H])c2c(c1[2H])c1c([2H])c([2H])cc([2H])c1n2-c1cc(-c2ccccc2)cc(-c2cccc3c2oc2cc(-c4nc(-c5ccccc5)nc(-c5ccccc5)n4)ccc23)c1 ; [2H]c1cc([2H])c2c(c1[2H])c1c([2H])c([2H])cc([2H])c1n2-c1cc(-c2ccccc2)cc(-c2cccc3c2oc2cccc(-c4nc(-c5ccccc5)nc(-c5ccccc5)n4)c23)c1]

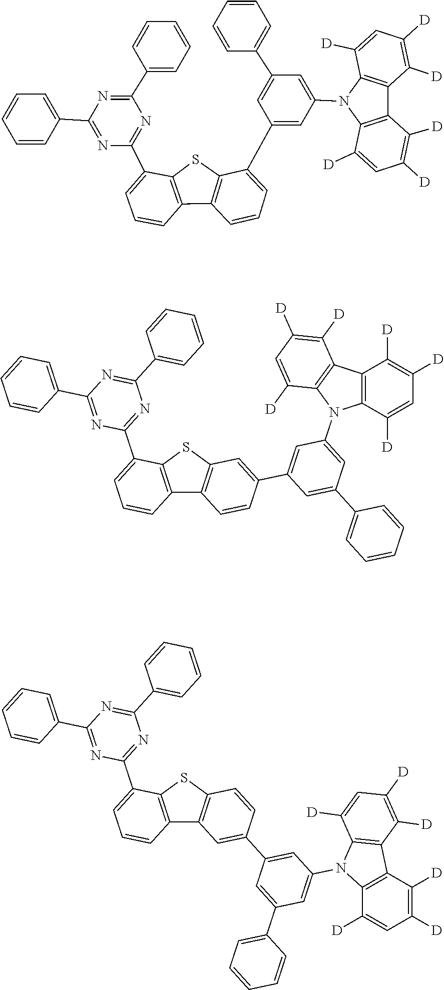 [SMILES: [2H]c1cc([2H])c2c(c1[2H])c1c([2H])c([2H])cc([2H])c1n2-c1cc(-c2ccccc2)cc(-c2cccc3c2sc2c(-c4nc(-c5ccccc5)nc(-c5ccccc5)n4)cccc23)c1 ; [2H]c1cc([2H])c2c(c1[2H])c1c([2H])c([2H])cc([2H])c1n2-c1cc(-c2ccccc2)cc(-c2ccc3c(c2)sc2c(-c4nc(-c5ccccc5)nc(-c5ccccc5)n4)cccc23)c1 ; [2H]c1cc([2H])c2c(c1[2H])c1c([2H])c([2H])cc([2H])c1n2-c1cc(-c2ccccc2)cc(-c2ccc3sc4c(-c5nc(-c6ccccc6)nc(-c6ccccc6)n5)cccc4c3c2)c1]

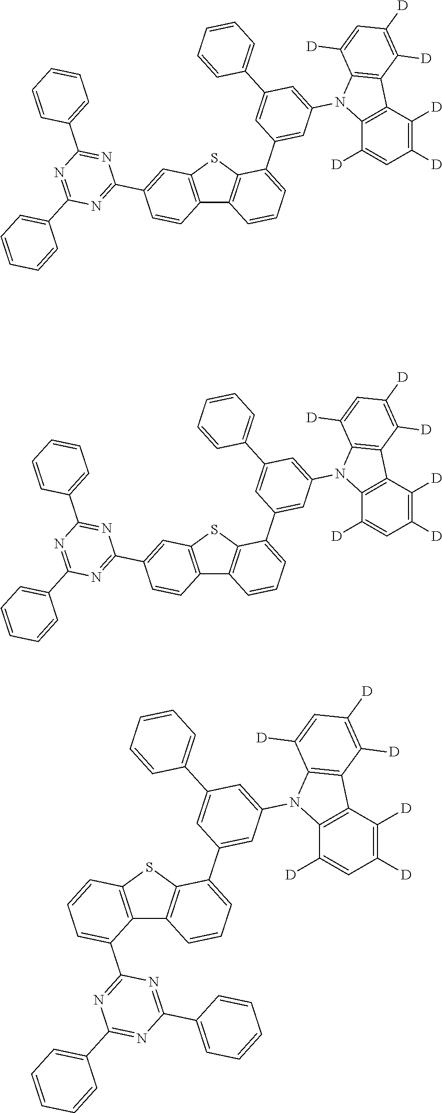 [SMILES: [2H]c1cc([2H])c2c(c1[2H])c1c([2H])c([2H])cc([2H])c1n2-c1cc(-c2ccccc2)cc(-c2cccc3c2sc2cc(-c4nc(-c5ccccc5)nc(-c5ccccc5)n4)ccc23)c1 ; [2H]c1cc([2H])c2c(c1[2H])c1c([2H])c([2H])cc([2H])c1n2-c1cc(-c2ccccc2)cc(-c2cccc3c2sc2cc(-c4nc(-c5ccccc5)nc(-c5ccccc5)n4)ccc23)c1 ; [2H]c1cc([2H])c2c(c1[2H])c1c([2H])c([2H])cc([2H])c1n2-c1cc(-c2ccccc2)cc(-c2cccc3c2sc2cccc(-c4nc(-c5ccccc5)nc(-c5ccccc5)n4)c23)c1]

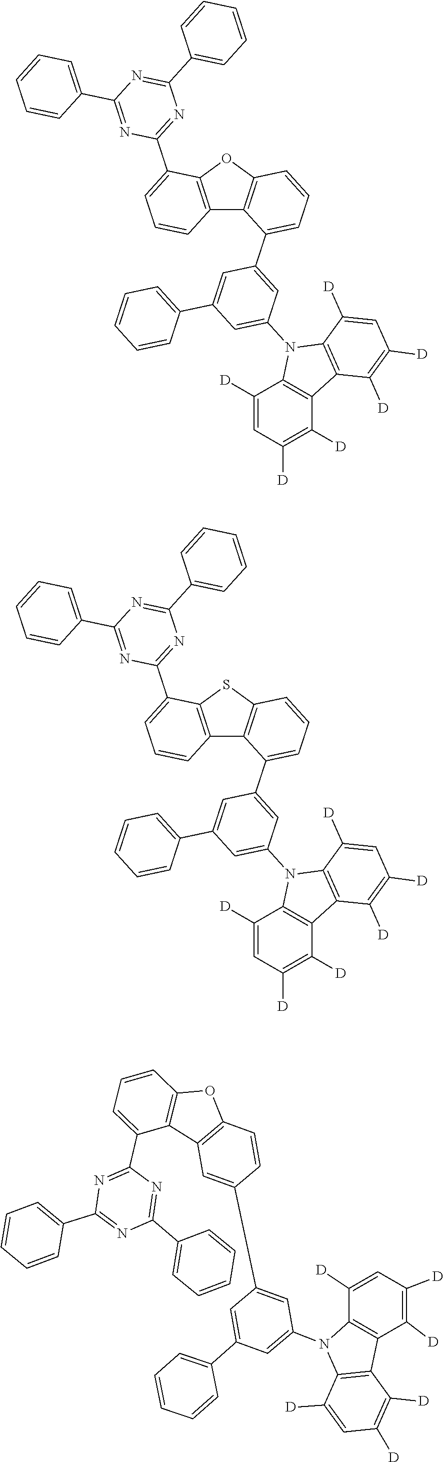 [SMILES: [2H]c1cc([2H])c2c(c1[2H])c1c([2H])c([2H])cc([2H])c1n2-c1cc(-c2ccccc2)cc(-c2cccc3oc4c(-c5nc(-c6ccccc6)nc(-c6ccccc6)n5)cccc4c23)c1 ; [2H]c1cc([2H])c2c(c1[2H])c1c([2H])c([2H])cc([2H])c1n2-c1cc(-c2ccccc2)cc(-c2cccc3sc4c(-c5nc(-c6ccccc6)nc(-c6ccccc6)n5)cccc4c23)c1 ; [2H]c1cc([2H])c2c(c1[2H])c1c([2H])c([2H])cc([2H])c1n2-c1cc(-c2ccccc2)cc(-c2ccc3oc4cccc(-c5nc(-c6ccccc6)nc(-c6ccccc6)n5)c4c3c2)c1]

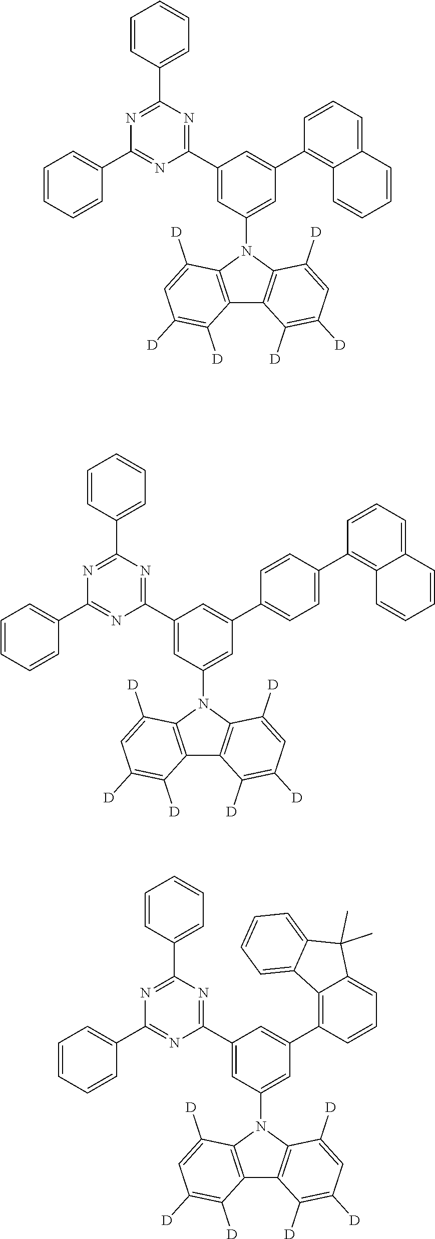 [SMILES: [2H]c1cc([2H])c2c(c1[2H])c1c([2H])c([2H])cc([2H])c1n2-c1cc(-c2nc(-c3ccccc3)nc(-c3ccccc3)n2)cc(-c2cccc3ccccc23)c1 ; [2H]c1cc([2H])c2c(c1[2H])c1c([2H])c([2H])cc([2H])c1n2-c1cc(-c2ccc(-c3cccc4ccccc34)cc2)cc(-c2nc(-c3ccccc3)nc(-c3ccccc3)n2)c1 ; [2H]c1cc([2H])c2c(c1[2H])c1c([2H])c([2H])cc([2H])c1n2-c1cc(-c2nc(-c3ccccc3)nc(-c3ccccc3)n2)cc(-c2cccc3c2-c2ccccc2C3(C)C)c1]

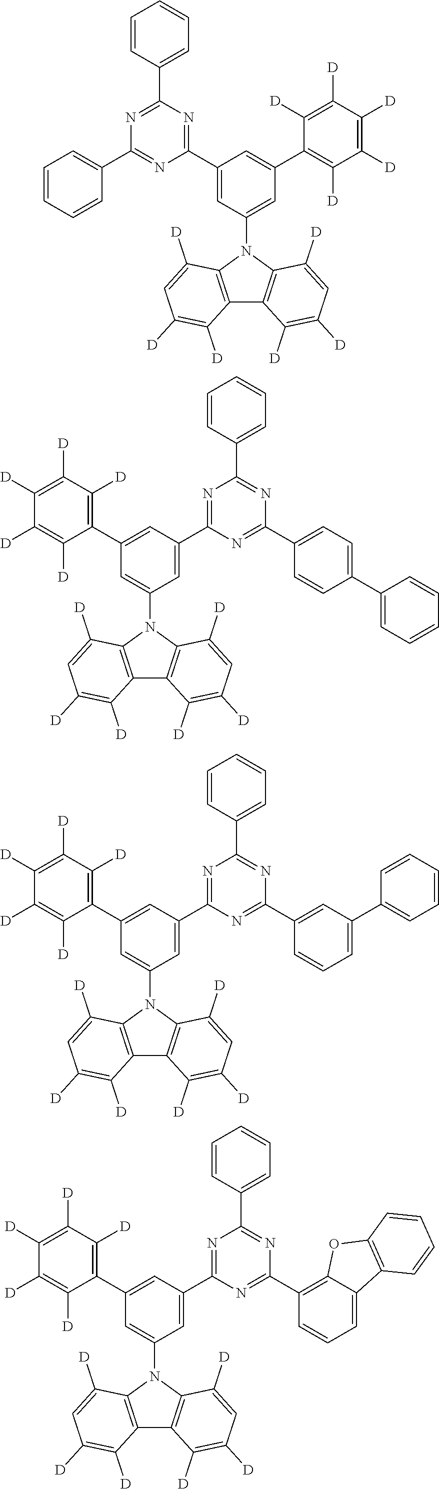

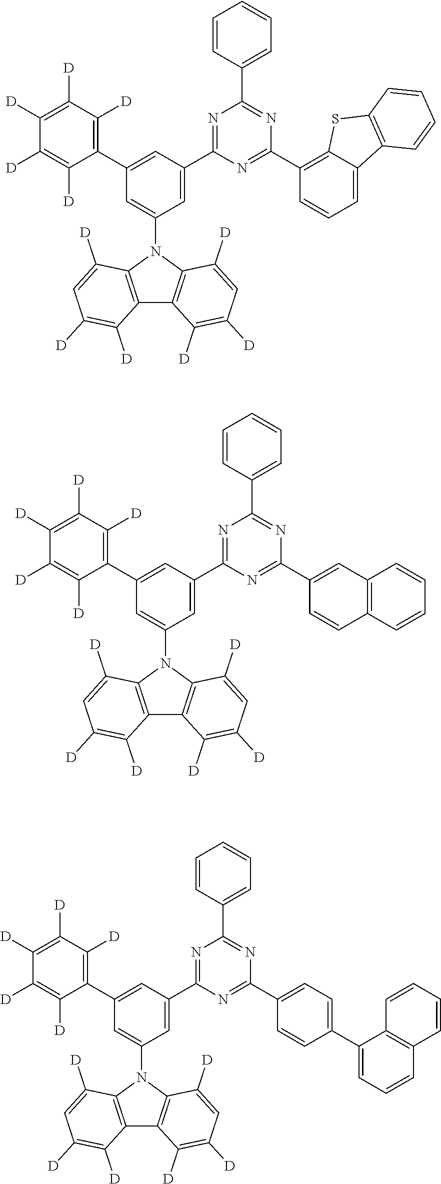 [SMILES: [2H]c1cc([2H])c2c(c1[2H])c1c([2H])c([2H])cc([2H])c1n2-c1cc(-c2nc(-c3ccccc3)nc(-c3cccc4c3sc3ccccc34)n2)cc(-c2c([2H])c([2H])c([2H])c([2H])c2[2H])c1 ; [2H]c1cc([2H])c2c(c1[2H])c1c([2H])c([2H])cc([2H])c1n2-c1cc(-c2nc(-c3ccccc3)nc(-c3ccc4ccccc4c3)n2)cc(-c2c([2H])c([2H])c([2H])c([2H])c2[2H])c1 ; [2H]c1cc([2H])c2c(c1[2H])c1c([2H])c([2H])cc([2H])c1n2-c1cc(-c2nc(-c3ccccc3)nc(-c3ccc(-c4cccc5ccccc45)cc3)n2)cc(-c2c([2H])c([2H])c([2H])c([2H])c2[2H])c1]

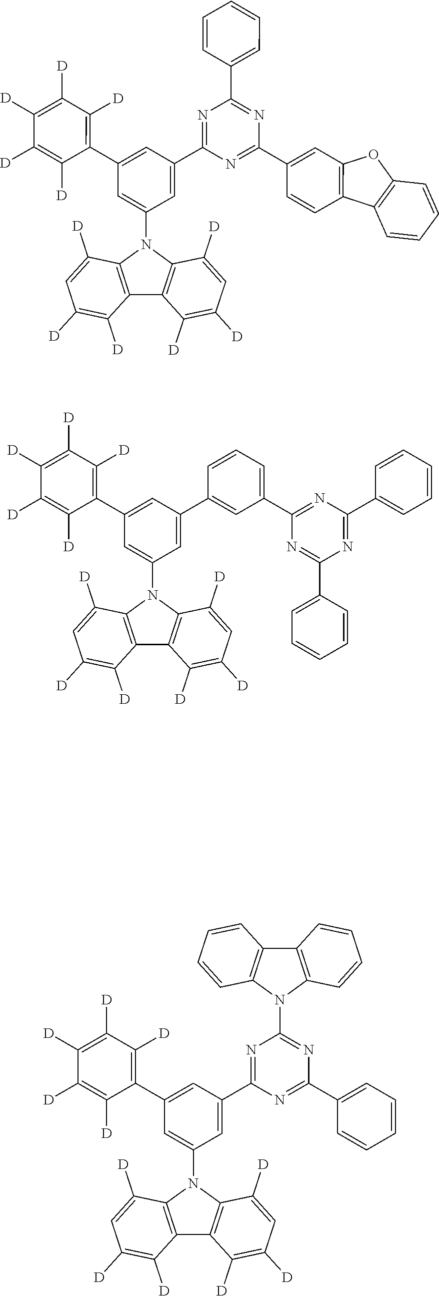

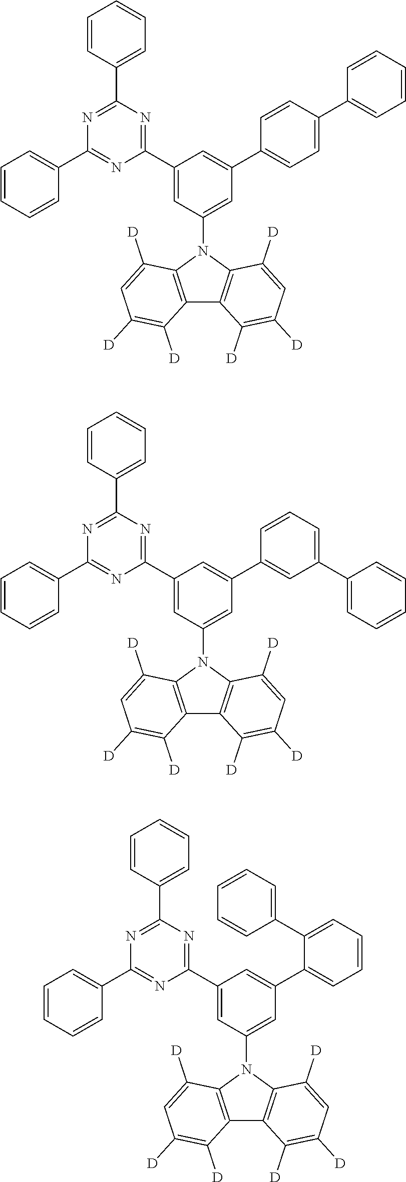 [SMILES: [2H]c1cc([2H])c2c(c1[2H])c1c([2H])c([2H])cc([2H])c1n2-c1cc(-c2ccc(-c3ccccc3)cc2)cc(-c2nc(-c3ccccc3)nc(-c3ccccc3)n2)c1 ; [2H]c1cc([2H])c2c(c1[2H])c1c([2H])c([2H])cc([2H])c1n2-c1cc(-c2cccc(-c3ccccc3)c2)cc(-c2nc(-c3ccccc3)nc(-c3ccccc3)n2)c1 ; [2H]c1cc([2H])c2c(c1[2H])c1c([2H])c([2H])cc([2H])c1n2-c1cc(-c2nc(-c3ccccc3)nc(-c3ccccc3)n2)cc(-c2ccccc2-c2ccccc2)c1]

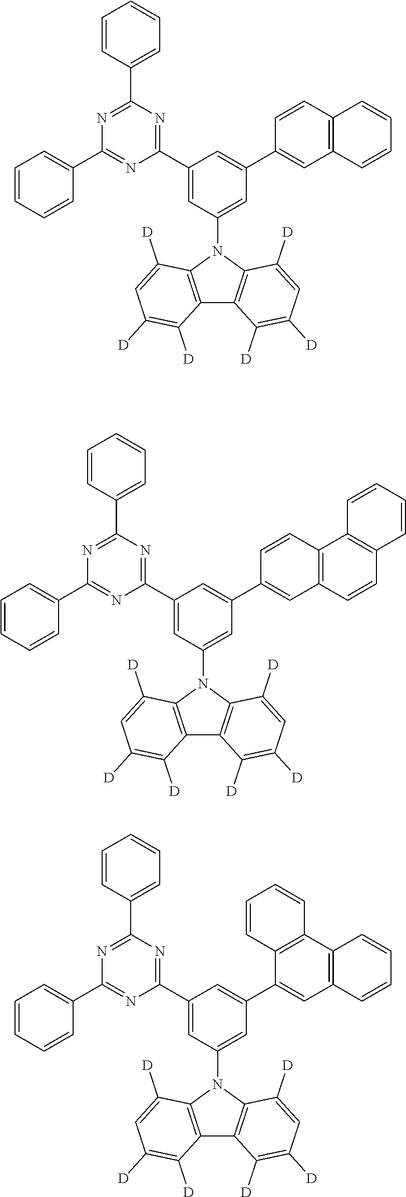 [SMILES: [2H]c1cc([2H])c2c(c1[2H])c1c([2H])c([2H])cc([2H])c1n2-c1cc(-c2ccc3ccccc3c2)cc(-c2nc(-c3ccccc3)nc(-c3ccccc3)n2)c1 ; [2H]c1cc([2H])c2c(c1[2H])c1c([2H])c([2H])cc([2H])c1n2-c1cc(-c2ccc3c(ccc4ccccc43)c2)cc(-c2nc(-c3ccccc3)nc(-c3ccccc3)n2)c1 ; [2H]c1cc([2H])c2c(c1[2H])c1c([2H])c([2H])cc([2H])c1n2-c1cc(-c2nc(-c3ccccc3)nc(-c3ccccc3)n2)cc(-c2cc3ccccc3c3ccccc23)c1]

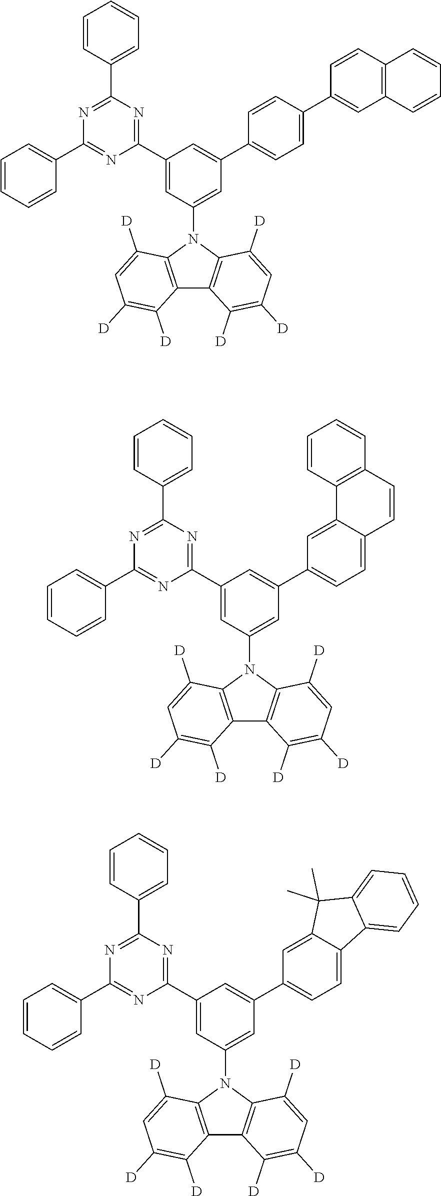 [SMILES: [2H]c1cc([2H])c2c(c1[2H])c1c([2H])c([2H])cc([2H])c1n2-c1cc(-c2ccc(-c3ccc4ccccc4c3)cc2)cc(-c2nc(-c3ccccc3)nc(-c3ccccc3)n2)c1 ; [2H]c1cc([2H])c2c(c1[2H])c1c([2H])c([2H])cc([2H])c1n2-c1cc(-c2ccc3ccc4ccccc4c3c2)cc(-c2nc(-c3ccccc3)nc(-c3ccccc3)n2)c1 ; [2H]c1cc([2H])c2c(c1[2H])c1c([2H])c([2H])cc([2H])c1n2-c1cc(-c2ccc3c(c2)C(C)(C)c2ccccc2-3)cc(-c2nc(-c3ccccc3)nc(-c3ccccc3)n2)c1]

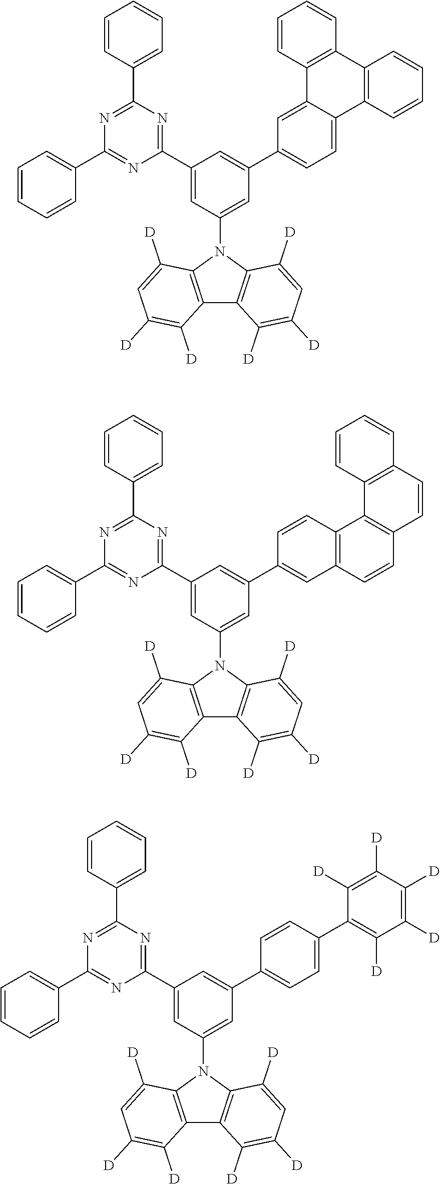 [SMILES: [2H]c1cc([2H])c2c(c1[2H])c1c([2H])c([2H])cc([2H])c1n2-c1cc(-c2ccc3c4ccccc4c4ccccc4c3c2)cc(-c2nc(-c3ccccc3)nc(-c3ccccc3)n2)c1 ; [2H]c1cc([2H])c2c(c1[2H])c1c([2H])c([2H])cc([2H])c1n2-c1cc(-c2ccc3c(ccc4ccc5ccccc5c43)c2)cc(-c2nc(-c3ccccc3)nc(-c3ccccc3)n2)c1 ; [2H]c1cc([2H])c2c(c1[2H])c1c([2H])c([2H])cc([2H])c1n2-c1cc(-c2ccc(-c3c([2H])c([2H])c([2H])c([2H])c3[2H])cc2)cc(-c2nc(-c3ccccc3)nc(-c3ccccc3)n2)c1]

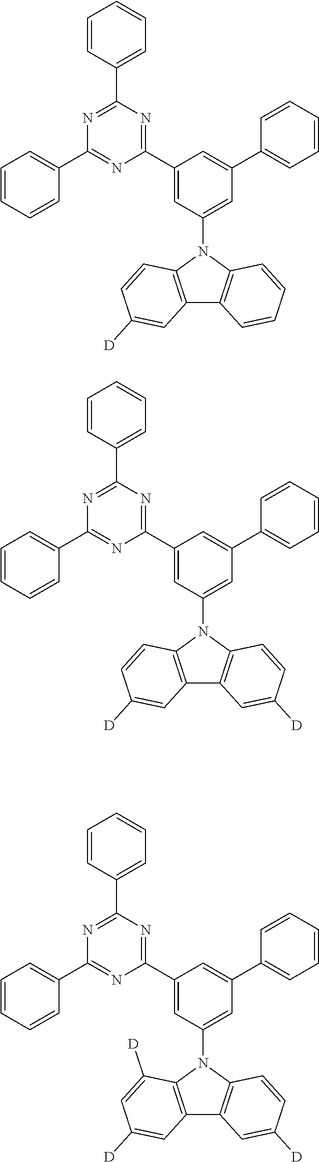

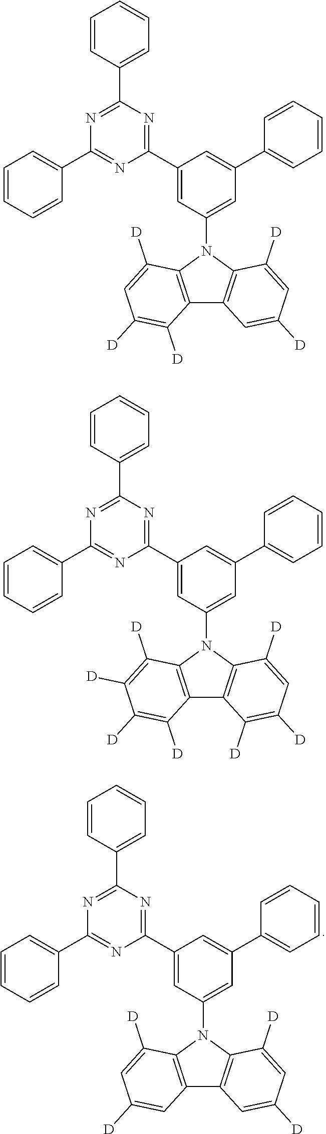

15. An organic light emitting device comprising:
a first electrode;
a second electrode that is opposite to the first electrode; and
one or more organic material layers that are between the first electrode and the second electrode, wherein one or more layers of the organic material layers comprises the compound of claim 1.

16. The organic light emitting device according to claim 15, wherein:
the organic material layer comprising the compound is a light emitting layer.

17. The organic light emitting device according to claim 16, wherein:
the light emitting layer further comprises a compound of Chemical Formula 2: 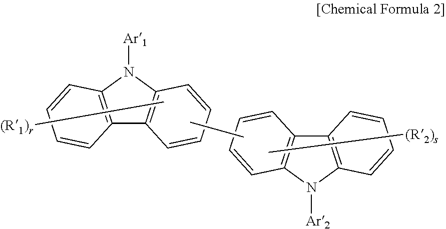
wherein, in Chemical Formula 2:
Ar′₁ and Ar′₂ are each independently a substituted or unsubstituted C₆₋₆₀ aryl or a substituted or unsubstituted C₂₋₆₀ heteroaryl containing one or more heteroatoms of N, O and S;
R′₁ and R′₂ are each independently hydrogen, deuterium, a C₁₋₆₀ alkyl, a C₆₋₆₀ aryl, or a C₂₋₆₀ heteroaryl containing one or more heteroatoms of N, O and S; and
r and s are each independently an integer of 0 to 7.

18. The organic light emitting device according to claim 17, wherein:
the compound of Chemical Formula 2 is any one compound selected from the group consisting of the following compounds: 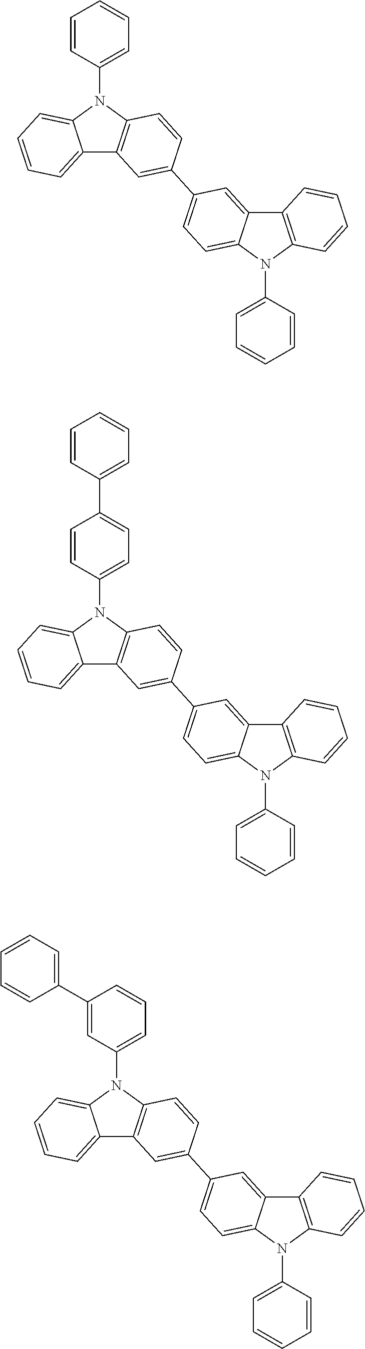 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1] 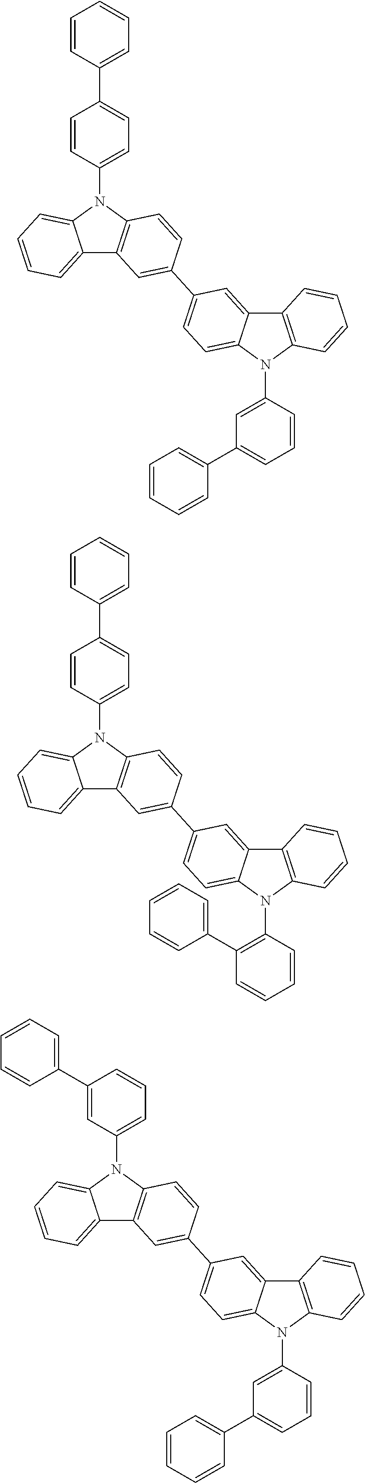 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1] 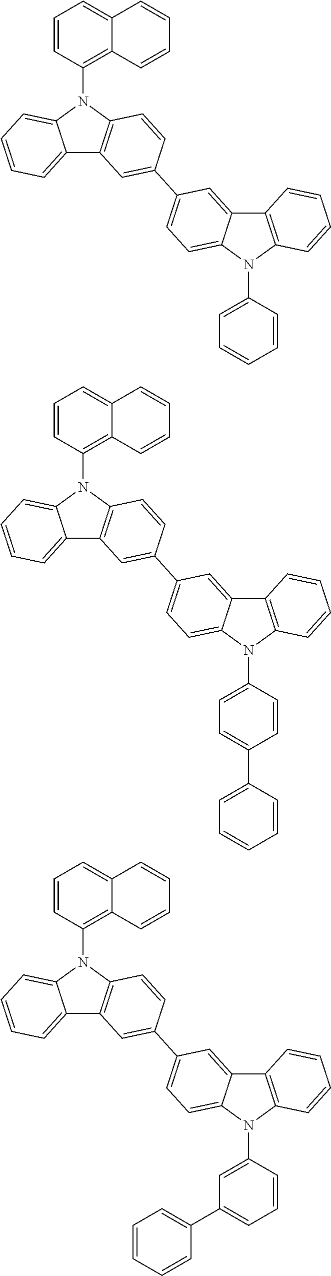 [SMILES: c1ccc(-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2cccc(-n3c4ccccc4c4cc(-c5ccc6c(c5)c5ccccc5n6-c5cccc6ccccc56)ccc43)c2)cc1] 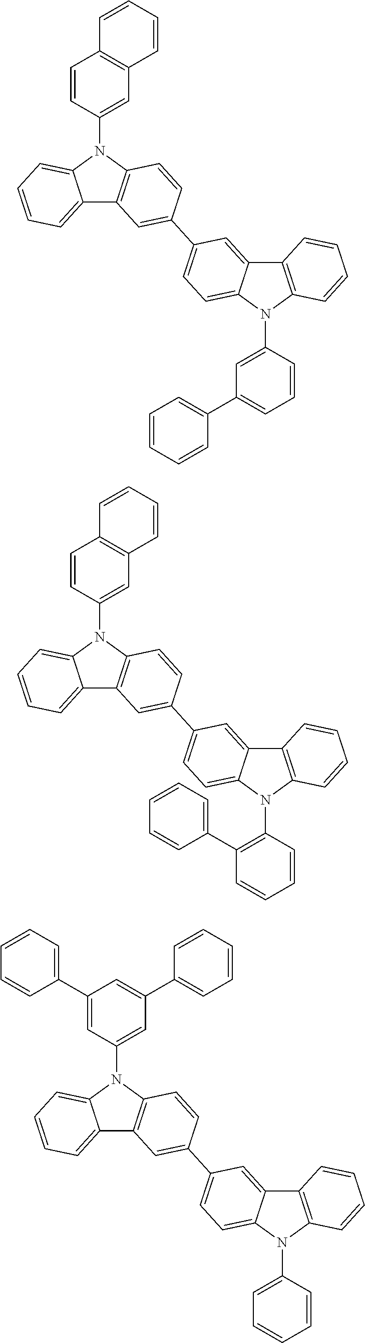 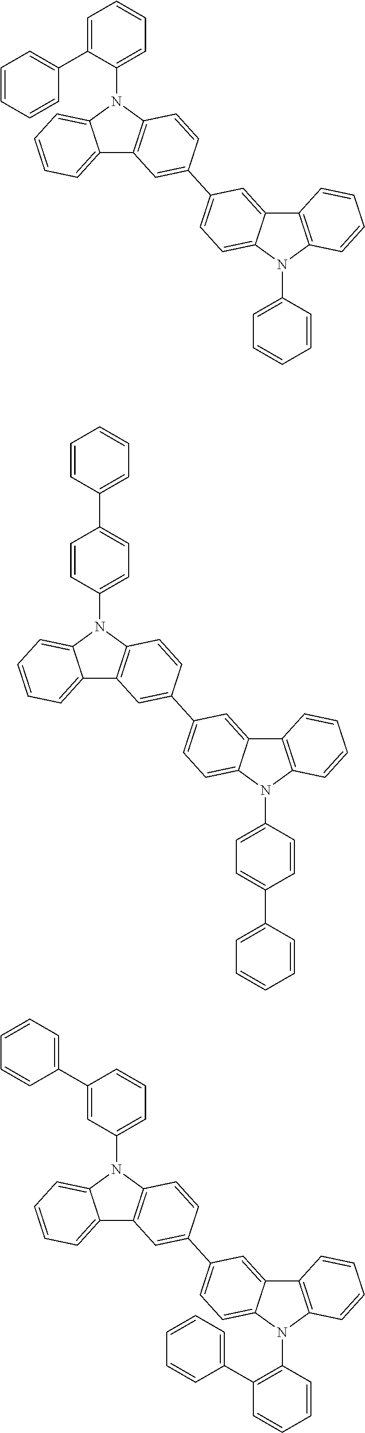 [SMILES: c1ccc(-c2ccccc2-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5ccccc5-c5ccccc5)ccc43)c2)cc1] 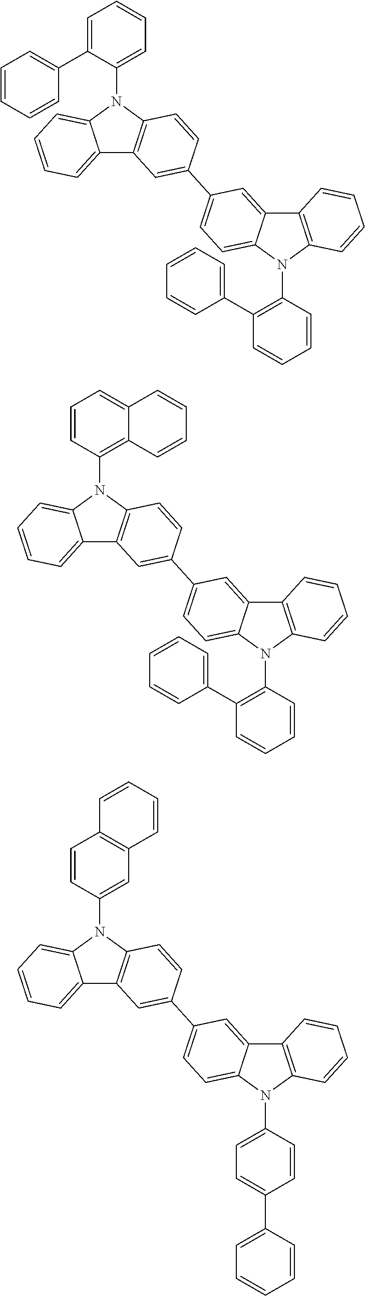 [SMILES: c1ccc(-c2ccccc2-n2c3ccccc3c3cc(-c4ccc5c(c4)c4ccccc4n5-c4ccccc4-c4ccccc4)ccc32)cc1 ; c1ccc(-c2ccccc2-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5ccc6ccccc6c5)ccc43)cc2)cc1] 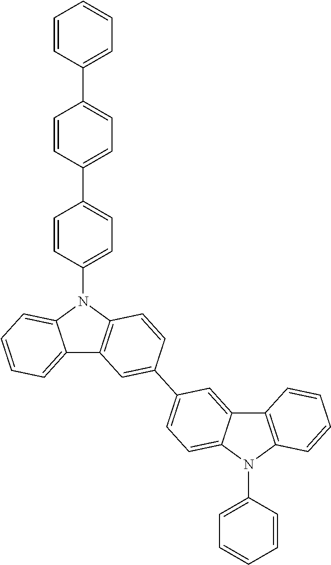 [SMILES: c1ccc(-c2ccc(-c3ccc(-n4c5ccccc5c5cc(-c6ccc7c(c6)c6ccccc6n7-c6ccccc6)ccc54)cc3)cc2)cc1] 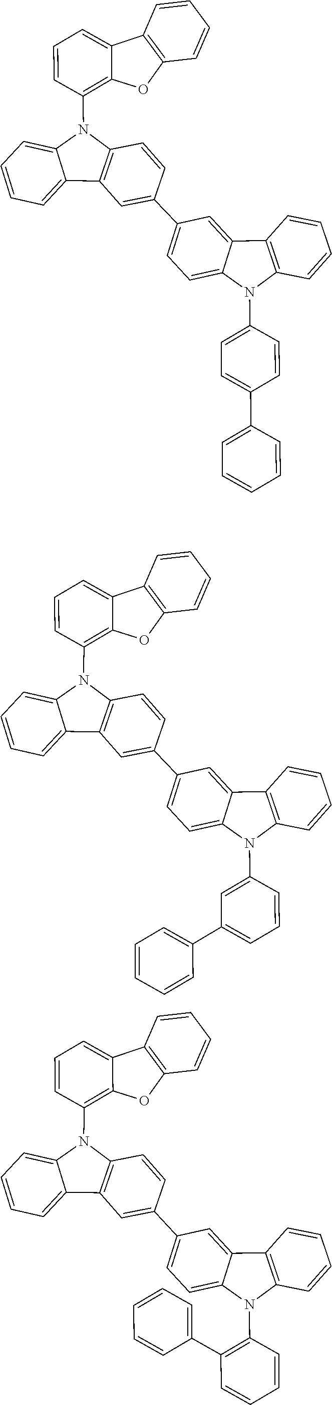 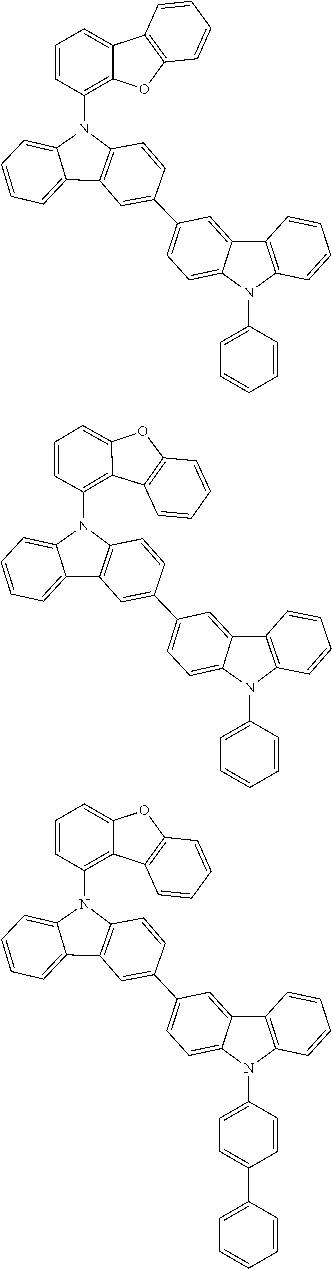 [SMILES: c1ccc(-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4cccc5oc6ccccc6c45)ccc32)cc1 ; c1ccc(-c2ccc(-n3c4ccccc4c4cc(-c5ccc6c(c5)c5ccccc5n6-c5cccc6oc7ccccc7c56)ccc43)cc2)cc1] 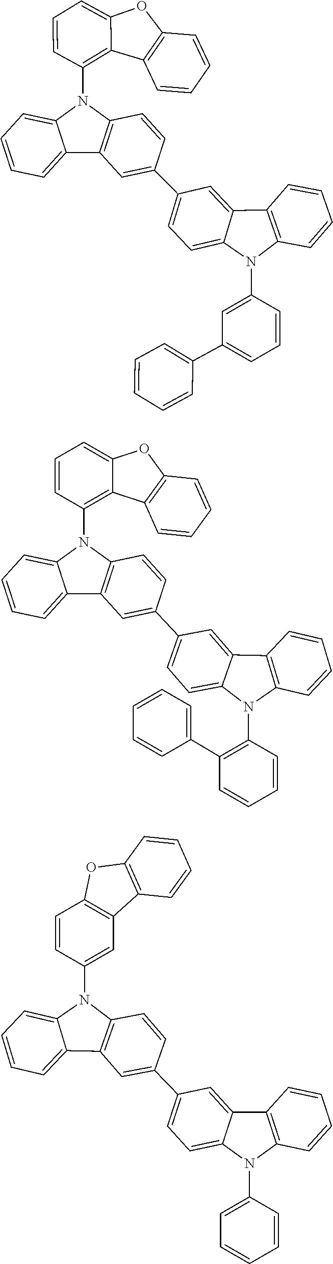 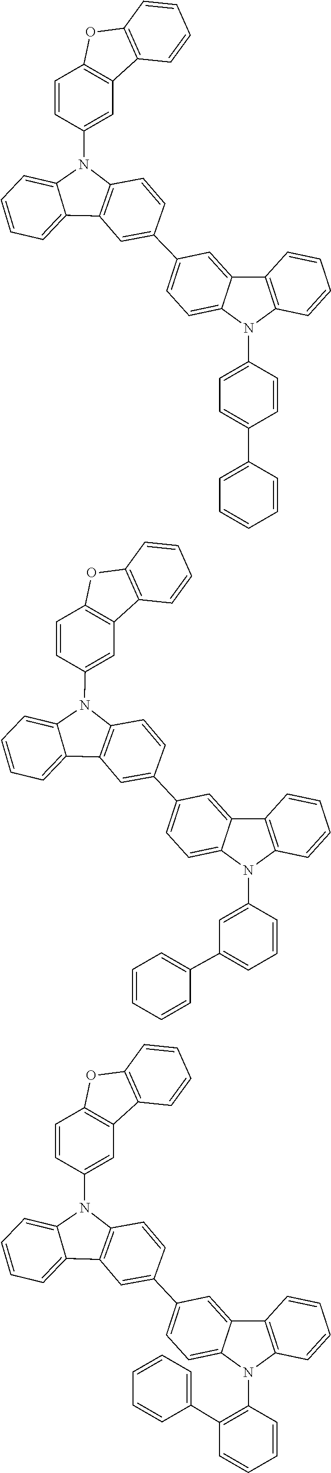 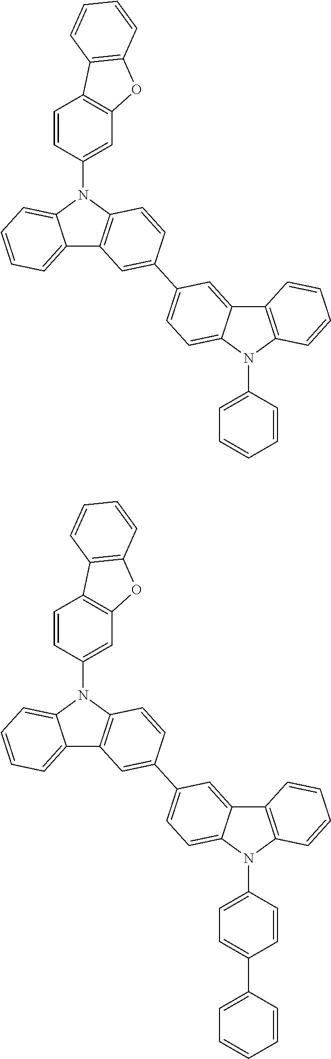 [SMILES: c1ccc(-n2c3ccccc3c3cc(-c4ccc5c(c4)c4ccccc4n5-c4ccc5c(c4)oc4ccccc45)ccc32)cc1 ; c1ccc(-c2ccc(-n3c4ccccc4c4cc(-c5ccc6c(c5)c5ccccc5n6-c5ccc6c(c5)oc5ccccc56)ccc43)cc2)cc1] 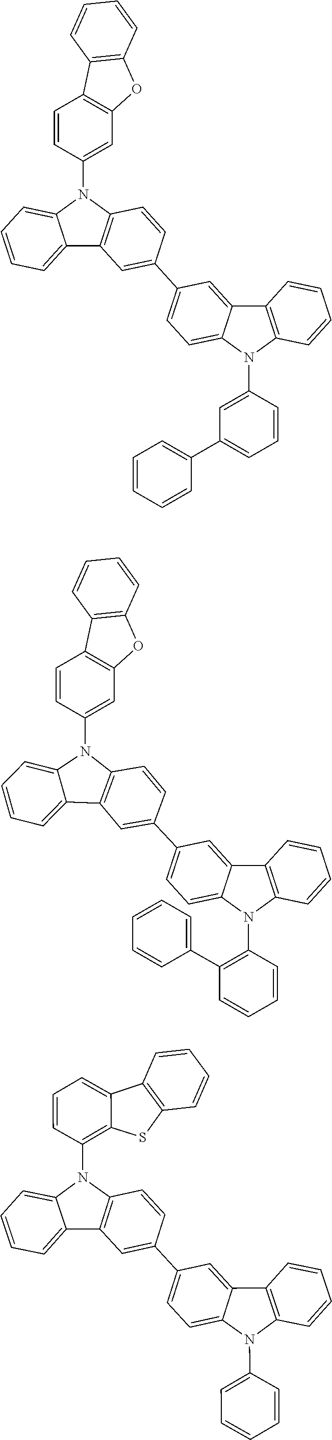 [SMILES: c1ccc(-c2cccc(-n3c4ccccc4c4cc(-c5ccc6c(c5)c5ccccc5n6-c5ccc6c(c5)oc5ccccc56)ccc43)c2)cc1 ; c1ccc(-c2ccccc2-n2c3ccccc3c3cc(-c4ccc5c(c4)c4ccccc4n5-c4ccc5c(c4)oc4ccccc45)ccc32)cc1 ; c1ccc(-n2c3ccccc3c3cc(-c4ccc5c(c4)c4ccccc4n5-c4cccc5c4sc4ccccc45)ccc32)cc1] 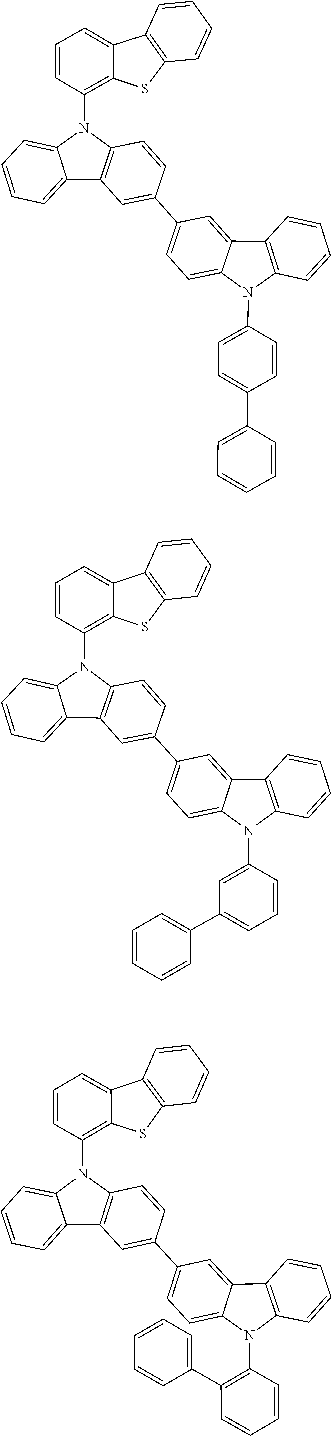 [SMILES: c1ccc(-c2ccc(-n3c4ccccc4c4cc(-c5ccc6c(c5)c5ccccc5n6-c5cccc6c5sc5ccccc56)ccc43)cc2)cc1 ; c1ccc(-c2cccc(-n3c4ccccc4c4cc(-c5ccc6c(c5)c5ccccc5n6-c5cccc6c5sc5ccccc56)ccc43)c2)cc1 ; c1ccc(-c2ccccc2-n2c3ccccc3c3cc(-c4ccc5c(c4)c4ccccc4n5-c4cccc5c4sc4ccccc45)ccc32)cc1] 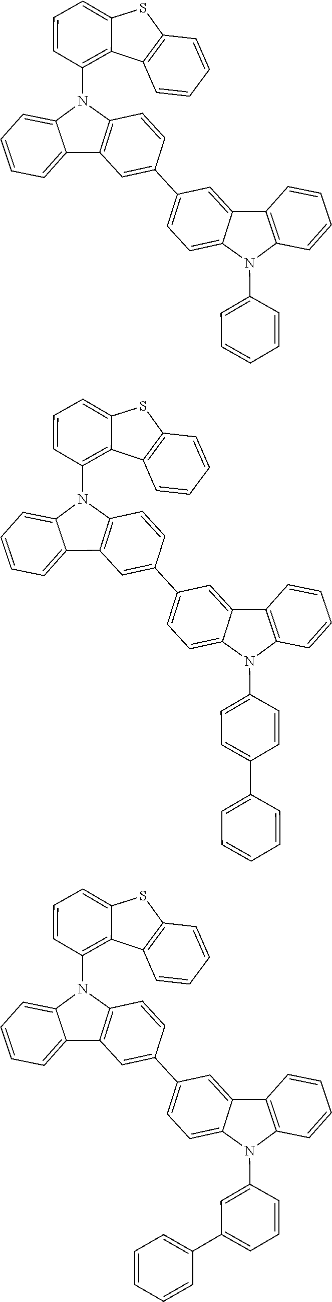 [SMILES: c1ccc(-n2c3ccccc3c3cc(-c4ccc5c(c4)c4ccccc4n5-c4cccc5sc6ccccc6c45)ccc32)cc1 ; c1ccc(-c2ccc(-n3c4ccccc4c4cc(-c5ccc6c(c5)c5ccccc5n6-c5cccc6sc7ccccc7c56)ccc43)cc2)cc1 ; c1ccc(-c2cccc(-n3c4ccccc4c4cc(-c5ccc6c(c5)c5ccccc5n6-c5cccc6sc7ccccc7c56)ccc43)c2)cc1] 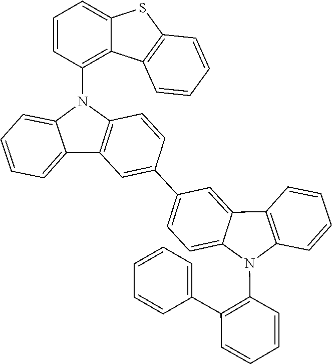 [SMILES: c1ccc(-c2ccccc2-n2c3ccccc3c3cc(-c4ccc5c(c4)c4ccccc4n5-c4cccc5sc6ccccc6c45)ccc32)cc1] 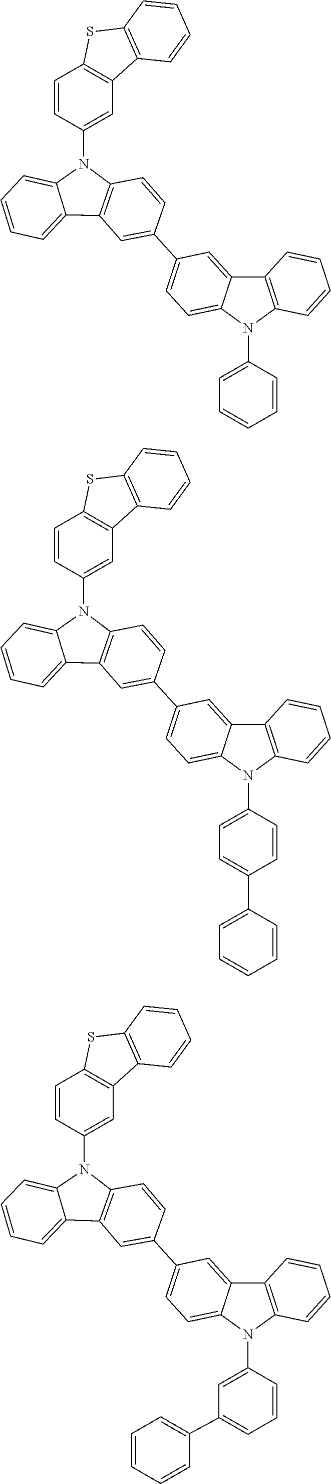 [SMILES: c1ccc(-n2c3ccccc3c3cc(-c4ccc5c(c4)c4ccccc4n5-c4ccc5sc6ccccc6c5c4)ccc32)cc1 ; c1ccc(-c2ccc(-n3c4ccccc4c4cc(-c5ccc6c(c5)c5ccccc5n6-c5ccc6sc7ccccc7c6c5)ccc43)cc2)cc1 ; c1ccc(-c2cccc(-n3c4ccccc4c4cc(-c5ccc6c(c5)c5ccccc5n6-c5ccc6sc7ccccc7c6c5)ccc43)c2)cc1] 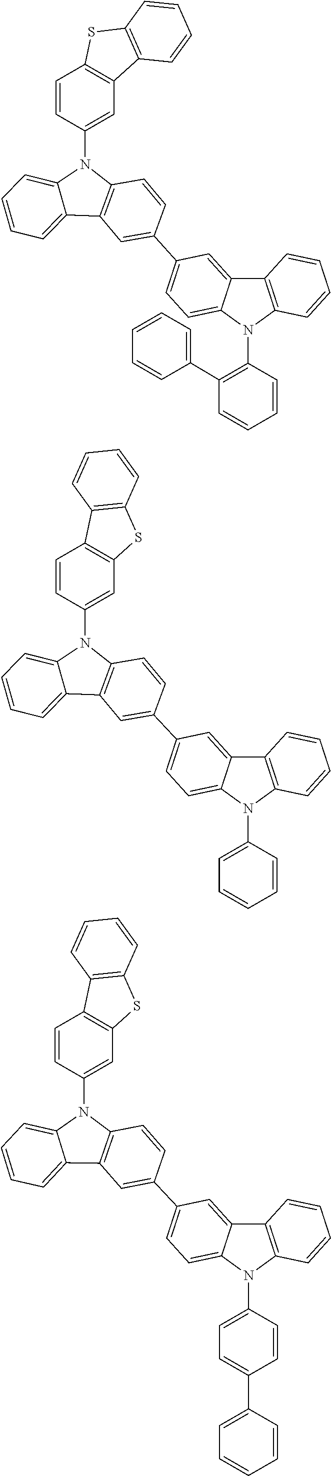 [SMILES: c1ccc(-c2ccccc2-n2c3ccccc3c3cc(-c4ccc5c(c4)c4ccccc4n5-c4ccc5sc6ccccc6c5c4)ccc32)cc1 ; c1ccc(-n2c3ccccc3c3cc(-c4ccc5c(c4)c4ccccc4n5-c4ccc5c(c4)sc4ccccc45)ccc32)cc1 ; c1ccc(-c2ccc(-n3c4ccccc4c4cc(-c5ccc6c(c5)c5ccccc5n6-c5ccc6c(c5)sc5ccccc56)ccc43)cc2)cc1] 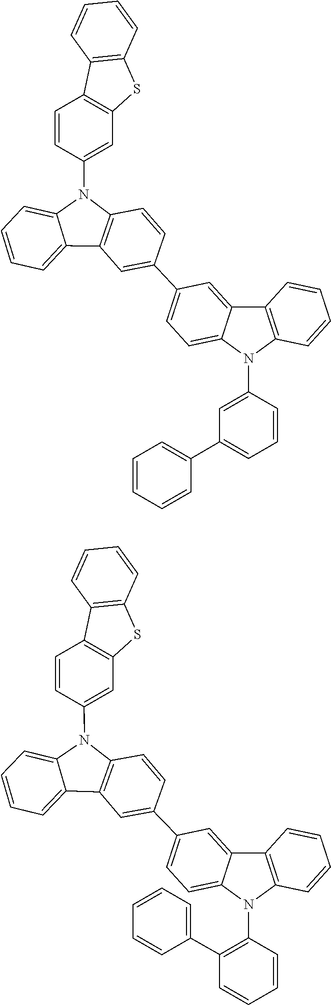 [SMILES: c1ccc(-c2cccc(-n3c4ccccc4c4cc(-c5ccc6c(c5)c5ccccc5n6-c5ccc6c(c5)sc5ccccc56)ccc43)c2)cc1 ; c1ccc(-c2ccccc2-n2c3ccccc3c3cc(-c4ccc5c(c4)c4ccccc4n5-c4ccc5c(c4)sc4ccccc45)ccc32)cc1] 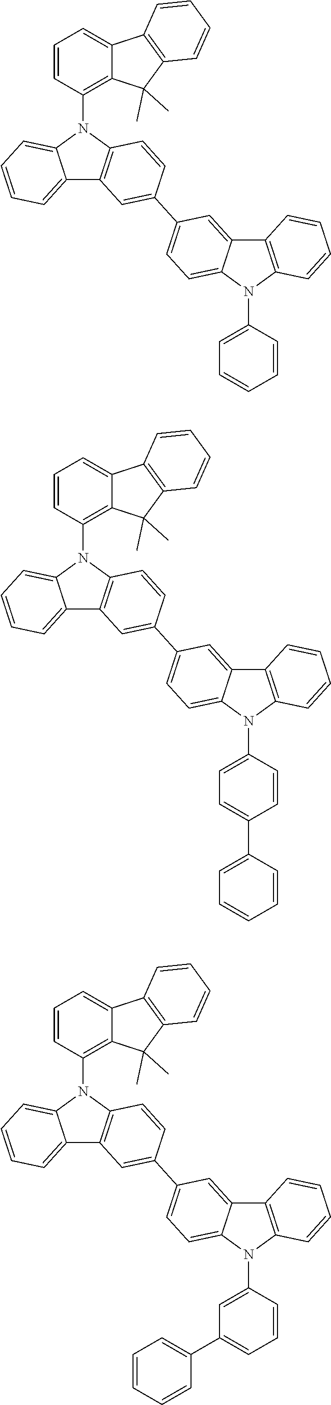 [SMILES: CC1(C)c2ccccc2-c2cccc(-n3c4ccccc4c4cc(-c5ccc6c(c5)c5ccccc5n6-c5ccccc5)ccc43)c21 ; CC1(C)c2ccccc2-c2cccc(-n3c4ccccc4c4cc(-c5ccc6c(c5)c5ccccc5n6-c5ccc(-c6ccccc6)cc5)ccc43)c21 ; CC1(C)c2ccccc2-c2cccc(-n3c4ccccc4c4cc(-c5ccc6c(c5)c5ccccc5n6-c5cccc(-c6ccccc6)c5)ccc43)c21] 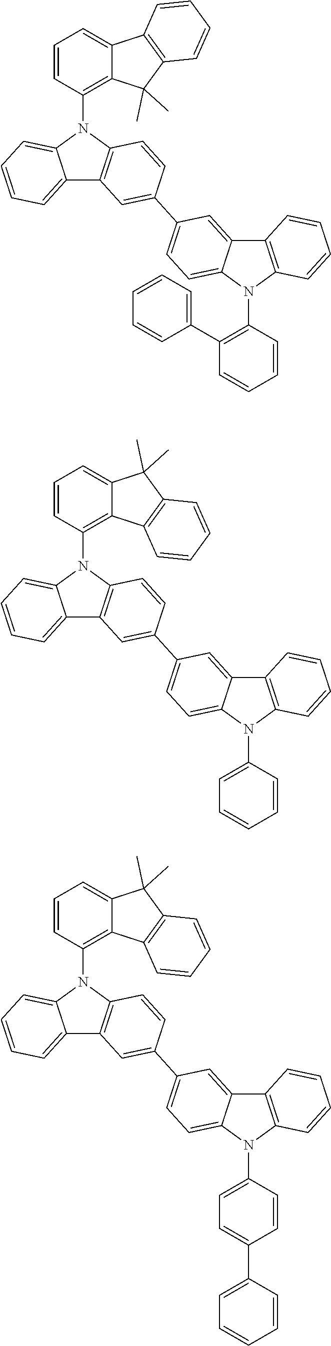 [SMILES: CC1(C)c2ccccc2-c2cccc(-n3c4ccccc4c4cc(-c5ccc6c(c5)c5ccccc5n6-c5ccccc5-c5ccccc5)ccc43)c21 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2c(-n3c4ccccc4c4cc(-c5ccc6c(c5)c5ccccc5n6-c5ccc(-c6ccccc6)cc5)ccc43)cccc21] 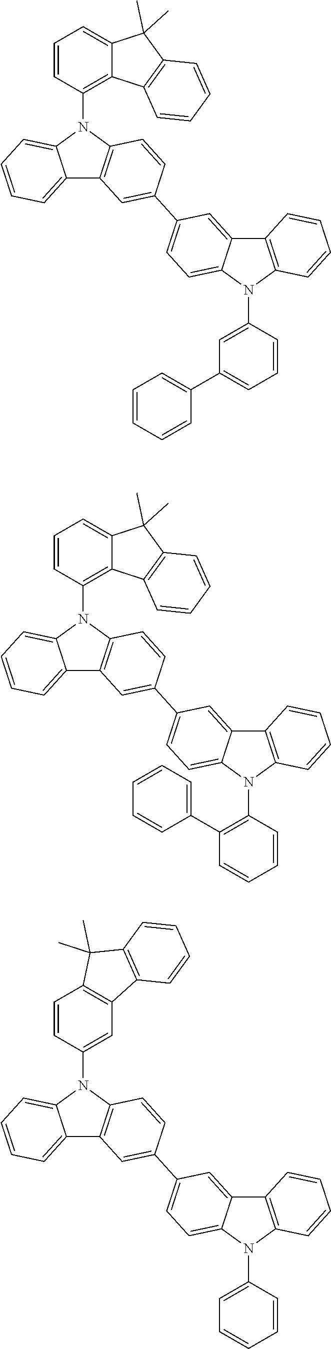 [SMILES: CC1(C)c2ccccc2-c2c(-n3c4ccccc4c4cc(-c5ccc6c(c5)c5ccccc5n6-c5cccc(-c6ccccc6)c5)ccc43)cccc21 ; CC1(C)c2ccccc2-c2c(-n3c4ccccc4c4cc(-c5ccc6c(c5)c5ccccc5n6-c5ccccc5-c5ccccc5)ccc43)cccc21 ; CC1(C)c2ccccc2-c2cc(-n3c4ccccc4c4cc(-c5ccc6c(c5)c5ccccc5n6-c5ccccc5)ccc43)ccc21] 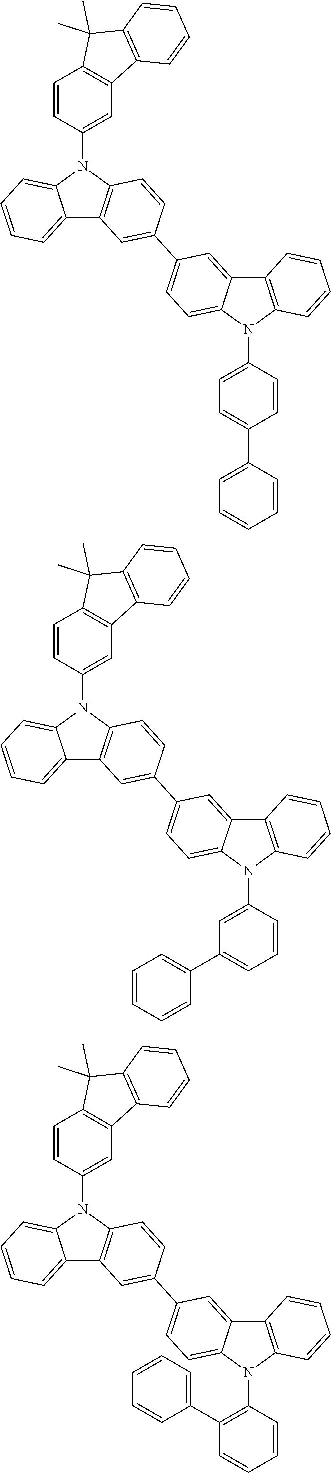 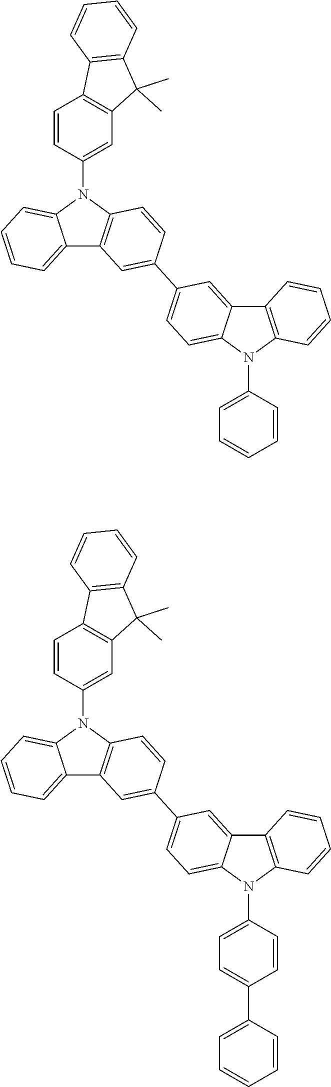 [SMILES: CC1(C)c2ccccc2-c2ccc(-n3c4ccccc4c4cc(-c5ccc6c(c5)c5ccccc5n6-c5ccccc5)ccc43)cc21 ; CC1(C)c2ccccc2-c2ccc(-n3c4ccccc4c4cc(-c5ccc6c(c5)c5ccccc5n6-c5ccc(-c6ccccc6)cc5)ccc43)cc21] 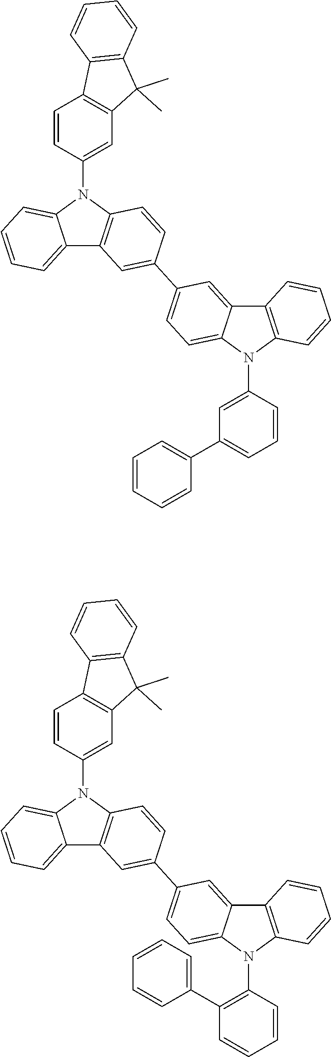 [SMILES: CC1(C)c2ccccc2-c2ccc(-n3c4ccccc4c4cc(-c5ccc6c(c5)c5ccccc5n6-c5cccc(-c6ccccc6)c5)ccc43)cc21 ; CC1(C)c2ccccc2-c2ccc(-n3c4ccccc4c4cc(-c5ccc6c(c5)c5ccccc5n6-c5ccccc5-c5ccccc5)ccc43)cc21] 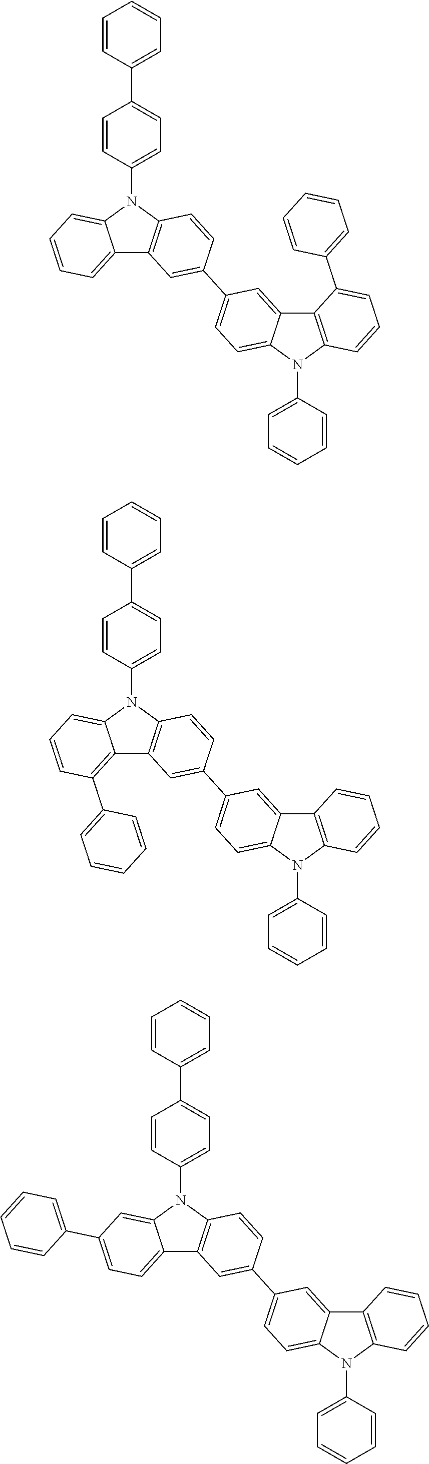 [SMILES: c1ccc(-c2ccc(-n3c4ccccc4c4cc(-c5ccc6c(c5)c5c(-c7ccccc7)cccc5n6-c5ccccc5)ccc43)cc2)cc1 ; c1ccc(-c2ccc(-n3c4ccc(-c5ccc6c(c5)c5ccccc5n6-c5ccccc5)cc4c4c(-c5ccccc5)cccc43)cc2)cc1 ; c1ccc(-c2ccc(-n3c4ccc(-c5ccc6c(c5)c5ccccc5n6-c5ccccc5)cc4c4ccc(-c5ccccc5)cc43)cc2)cc1] 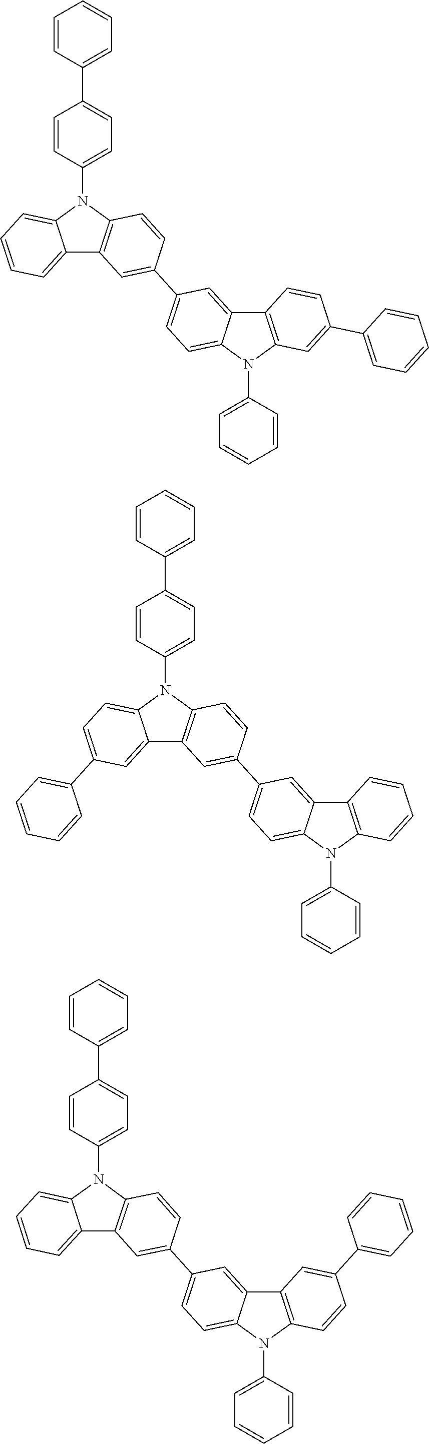 [SMILES: c1ccc(-c2ccc(-n3c4ccccc4c4cc(-c5ccc6c(c5)c5ccc(-c7ccccc7)cc5n6-c5ccccc5)ccc43)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-c2ccc(-n3c4ccccc4c4cc(-c5ccc6c(c5)c5cc(-c7ccccc7)ccc5n6-c5ccccc5)ccc43)cc2)cc1] 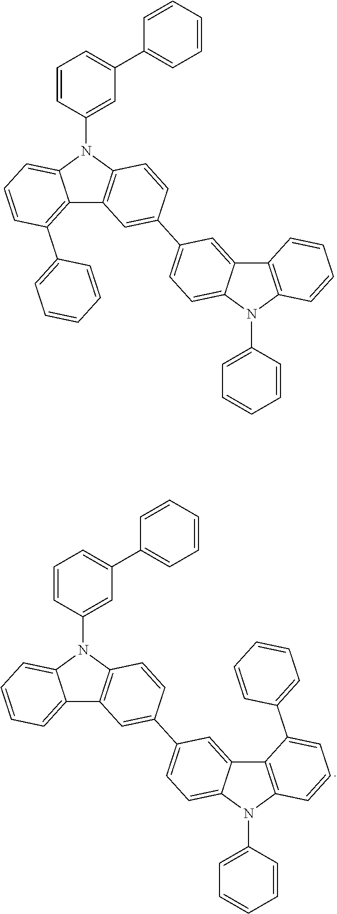 [SMILES: c1ccc(-c2cccc(-n3c4ccc(-c5ccc6c(c5)c5ccccc5n6-c5ccccc5)cc4c4c(-c5ccccc5)cccc43)c2)cc1 ; c1ccc(-c2cccc(-n3c4ccccc4c4cc(-c5ccc6c(c5)c5c(-c7ccccc7)cccc5n6-c5ccccc5)ccc43)c2)cc1]
